SEQUENCE LISTING (437 sequences)

SEQ ID NO: 1 - DNA, 30 bp, Homo sapiens
atttatcctgcagtgctttgctgcaagata

SEQ ID NO: 2 - DNA, 134 bp, Homo sapiens
aaataaaatatcagggatatgctcccccttgagactgaaggaactgaagattttaaaccttagtaagaaccacatttcatccctatcagagaactttcttgaggcttgtcctaaagtggagagtttcagtgcca

SEQ ID NO: 3 - DNA, 30 bp, Homo sapiens
gaatgaattttcttgctgctatgcctttct

SEQ ID NO: 4 - DNA, 85 bp, Homo sapiens
tgccaaggaagaccccctcctgacccctgttccggcttcagaaaacccgtttagggagaagaagtttttctgtgccatcctttaa

SEQ ID NO: 5 - DNA, 30 bp, Homo sapiens
tgcagatttgatggcctactgtgaagcaca

SEQ ID NO: 6 - DNA, 500 bp, Homo sapiens
gatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatccc

SEQ ID NO: 7 - DNA, 30 bp, Homo sapiens
atgaagaaaaacaaagtgcacagagacccg

SEQ ID NO: 8 - DNA, 30 bp, Homo sapiens
aggacagccctgggcagagatgaggcaggg

SEQ ID NO: 9 - DNA, 30 bp, Homo sapiens
taatgccaagacaaagccacgggaggagca

SEQ ID NO: 10 - DNA, 426 bp, Homo sapiens
cacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgccacggccggcaagcccccgctccccaggctctcggggtcgcgtgaggatgcttggcacgtaccccgtgtacatacttcccaggcacccagcatggaaataaagcacccagcgct

SEQ ID NO: 11 - DNA, 500 bp, Homo sapiens
tctctctttccaatcttacgccatggccatcagttcatttcagccttccagtgctacacccacttcttggctgacacacttctgctctaaggtgactggttttcttgccaattttcaaagagtggtactaacccccaacccgctttccgcaccccgtcctctccgccagcagtactggttgcactaactgtgagtgtcttgcatactgatggactcatttggtggcatggttggctaacagcatggcggggggtgttcagcttgagacccatgcctgtgttcatttcccatggagctggcagcctggtctaccccaagtgcatgccccgcctctcctctctcccttgggtctgcctgcgtgcatgcttctccagttgcgtctgcgaagctacctactttcttgggagggtcgaccttgatcatgaaacaataccatgagggggcctctgtcacctttgaaaagaacactttttgagcagcctcaaaaagctcatacatac

SEQ ID NO: 12 - DNA, 500 bp, Homo sapiens
tgggagggtcgaccttgatcatgaaacaataccatgagggggcctctgtcacctttgaaaagaacactttttgagcagcctcaaaaagctcatacataccagcgccttcttaaattggctctaatgtaaagattgttaatgtcatttatcaaaaccataggtgattatttggagggatttaaaaaacttaattactctcaggcctcatcccaagcttgacacatgctctgtaggttgaacacataatcacaaatattctagcaaatgctgccttggttgcagcctgcactgtagacccaagggttttgctgtggctcttcttatctcccttggctcataaagccccagatgatgccagagcttcaattagagccatcatcatcccaggcagggatatctttgagaaatgactcagttcagccccaggcccctgtgactctgcttaaagcacacatttctgctgactcttgtacctggggcagcaggataatcaccaacac

SEQ ID NO: 13 - DNA, 30 bp, Homo sapiens
gtcgcccagatcaaggctcatgtagcctca

SEQ ID NO: 14 - DNA, 30 bp, Homo sapiens
ggccgcatgcttggaaggtaaagtccatgg

SEQ ID NO: 15 - DNA, 30 bp, Homo sapiens
gtcgcccagagcaaggctcatgtagcctca

SEQ ID NO: 16 - DNA, 30 bp, Homo sapiens
tgaccggccaggaaacggtcgcccagatca

SEQ ID NO: 17 - DNA, 30 bp, Homo sapiens
caggccgcatgcttgaggtaaagtccatgg

SEQ ID NO: 18 - DNA, 30 bp, Homo sapiens
aggccgcatgcttggaggtaaagtccatgg

SEQ ID NO: 19 - DNA, 30 bp, Homo sapiens
ggccaggaaacggtcgcccagatcaaggta

SEQ ID NO: 20 - DNA, 30 bp, Homo sapiens
ggtcgcccagatcaagctcatgtagcctca

SEQ ID NO: 21 - DNA, 30 bp, Homo sapiens
gccgcatgcttggaggtaatgtccatggtt

SEQ ID NO: 22 - DNA, 30 bp, Homo sapiens
gtcgcccagatcaaggctctgtagcctcac

SEQ ID NO: 23 - DNA, 30 bp, Homo sapiens
gaagtagcaggccgcatgcttggaggtaaa

SEQ ID NO: 24 - DNA, 30 bp, Homo sapiens
aggccgatgcttggaggtaaagtccatggt

SEQ ID NO: 25 - DNA, 30 bp, Homo sapiens
cccgcatgcttggaggtaaagtccatggtt

SEQ ID NO: 26 - DNA, 30 bp, Homo sapiens
cggtcgcccagatcaaggctcatgtagcct

SEQ ID NO: 27 - DNA, 30 bp, Homo sapiens
ggccgcatgcttggggtaaagtccatggtt

SEQ ID NO: 28 - DNA, 30 bp, Homo sapiens
gccgcatgcttggaggtaacagtccatggt

SEQ ID NO: 29 - DNA, 30 bp, Homo sapiens
cgacacaccctagcggacaattttaaccct

SEQ ID NO: 30 - DNA, 30 bp, Homo sapiens
ttccattccatgcaggaaggctggaagcgc

SEQ ID NO: 31 - DNA, 30 bp, Homo sapiens
aatcctcagttcaaggttatcctacgcaga

SEQ ID NO: 32 - DNA, 30 bp, Homo sapiens
gactgacctttttccaaagacgagaatcca

SEQ ID NO: 33 - DNA, 30 bp, Homo sapiens
ctcgcagaaacccaaccgctccaccaccgt

SEQ ID NO: 34 - DNA, 30 bp, Homo sapiens
gtaggtaggttcgtagtagggttcgtaggt

SEQ ID NO: 35 - DNA, 500 bp, Homo sapiens
tagggctagtaggtagggctagtaggtagggctagtaggtagggctagtaggtagggctagtaggtagggctagtaggtagggctagtaggtagggttcgtaggtagggttcgtaggtagggttcgtaggtagggttagtagcgcgtctgtgctgcttccacctggtgcttcctgttcccaaatcacaagggcctgaaggtggtccctgctttctctttctctttctctgtgtctcagatggcgattttgctgacagctgccaagaaaatgcttcactcaacagtcctcatgtgcccagagatgtttatagaactgtttgaattgcagccatcccctgccccctcccaggctgaagatctgttctttttaagttgattcgggagtggcattcttttatacccaaagactgtagtgcatcttgaagagctcaaagcacatgaccgcacaaatgcttacagggtttcctcccgagtaatccaatctcactccccttgtaagg

SEQ ID NO: 36 - DNA, 217 bp, Homo sapiens
tagggttcgtaggtagggctagtaggtagggttagtaggtagggctagtaggtagggctagtaggtagggttagtaggtagggttcgtaggtagggctggtaggtagggttagtaggtagggctagtaggtagggctagtaggtagggctagtaggtagggttagtaggtagggctagtaggtagggctagtaggtagggttagtaggtagggttcg

SEQ ID NO: 37 - DNA, 30 bp, Homo sapiens
aggtagggttcgtaggtagggctagtaggt

SEQ ID NO: 38 - DNA, 36 bp, Homo sapiens
gtgaagaagatccatgagaatgagaagcgcctggag

SEQ ID NO: 39 - DNA, 30 bp, Homo sapiens
ctggaccagcaccccattgacgggtacctc

SEQ ID NO: 40 - DNA, 49 bp, Homo sapiens
catggagcattgcaccacccgctttttcgagacctgtgacctggacaat

SEQ ID NO: 41 - DNA, 119 bp, Homo sapiens
gagaatgagaagcgcctggaggcaggagaccaccccgtggagctgctggcccgggacttcgagaagaactataacatgtacatcttccctgtacactggcagttcggccagctggacca

SEQ ID NO: 42 - DNA, 30 bp, Homo sapiens
gcaccccattgacgggtacctctcccacac

SEQ ID NO: 43 - DNA, 30 bp, Homo sapiens
cgggtcagtgtacaggaagaggcaggcact

SEQ ID NO: 44 - DNA, 30 bp, Homo sapiens
tgctgtgaggcagaggaatgatggagaatc

SEQ ID NO: 45 - DNA, 500 bp, Homo sapiens
tacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaata

SEQ ID NO: 46 - DNA, 54 bp, Homo sapiens
ccacaggaaggaattctggaagatatgcctgtggatcctgacaatgaggcttat

SEQ ID NO: 47 - DNA, 30 bp, Homo sapiens
gaccagttgggcaagaatgaagaaggagcc

SEQ ID NO: 48 - DNA, 124 bp, Homo sapiens
taaaggaattcattagccatggatgtattcatgaaaggactttcaaaggccaaggagggagttgtggctgctgctgagaaaaccaaacagggtgtggcagaagcagcaggaaagacaaaagagg

SEQ ID NO: 49 - DNA, 30 bp, Homo sapiens
gtgttctctatgtaggctccaaaaccaagg

SEQ ID NO: 50 - DNA, 67 bp, Homo sapiens
aaaaccttttatgacaggggctgcagaacaaatcaagcacatccttgctaatttcaaaaactaccag

SEQ ID NO: 51 - DNA, 30 bp, Homo sapiens
agatcgcggacgggttgtgcctggaggtgg

SEQ ID NO: 52 - DNA, 30 bp, Homo sapiens
tccgacatctacaagatccgggagatcgcg

SEQ ID NO: 53 - DNA, 44 bp, Homo sapiens
ccgacatctacaagatccgggagatcgcggacgggttgtgcctg

SEQ ID NO: 54 - DNA, 98 bp, Homo sapiens
gcgccgctccggctgcaccgcgctcgctccgagtttcaggctcgtgctaagctagcgccgtcgtcgtctcccttcagtcgccatcatgattatctacc

SEQ ID NO: 55 - DNA, 30 bp, Homo sapiens
gaggggctgcggaggcagcgacccggaaac

SEQ ID NO: 56 - DNA, 30 bp, Homo sapiens
catgctctggcagaggatggctgaagtcca

SEQ ID NO: 57 - DNA, 30 bp, Homo sapiens
attaacaccactgactgtgctcaccacaca

SEQ ID NO: 58 - DNA, 30 bp, Homo sapiens
gtgtgccgctgattgtggaccctgatgaca

SEQ ID NO: 59 - DNA, 177 bp, Homo sapiens
gaggaggaagggagcggcagcagtgaaggatttgacccccctgccactgataggcagttctctggggcccggaatcagctgcgccgcccccagtatcgccctcagtaccggcagcggcggttcccgccttaccacgtgggacagacctttgaccgtcgctcacgggtcttaccccat

SEQ ID NO: 60 - DNA, 30 bp, Homo sapiens
cccaacagaatacaggctggtgagattgga

SEQ ID NO: 61 - DNA, 30 bp, Homo sapiens
tctctgctcttcaatggtatcatggcattg

SEQ ID NO: 62 - DNA, 30 bp, Homo sapiens
gacgcttctgagctaaaagcttccatgaag

SEQ ID NO: 63 - DNA, 30 bp, Homo sapiens
agcttggagggtgatgtgtggatgaggtca

SEQ ID NO: 64 - DNA, 205 bp, Homo sapiens
atttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaac

SEQ ID NO: 65 - DNA, 30 bp, Homo sapiens
agatagttaagtgggatcgagacatgtaag

SEQ ID NO: 66 - DNA, 30 bp, Homo sapiens
gactccaagcctgaggcagcagagaaacaa

SEQ ID NO: 67 - DNA, 30 bp, Homo sapiens
acacccagaccccaggaagagccaatgttt

SEQ ID NO: 68 - DNA, 30 bp, Homo sapiens
gccccaggaggagagcagtttaaagatttt

SEQ ID NO: 69 - DNA, 30 bp, Homo sapiens
gcagctgtctggaatgcagaggcagctgga

SEQ ID NO: 70 - DNA, 30 bp, Homo sapiens
ccatcggggcagaagaacttcatcacagct

SEQ ID NO: 71 - DNA, 30 bp, Homo sapiens
cggtcaactttcaagctccttaagccactg

SEQ ID NO: 72 - DNA, 30 bp, Homo sapiens
tcaaggccgcctggggatgttcctgtcctt

SEQ ID NO: 73 - DNA, 30 bp, Homo sapiens
gtggctctgaaacggggtgtgccgagttcc

SEQ ID NO: 74 - DNA, 30 bp, Homo sapiens
cagaggagaagaaagggtcaaggaggaggt

SEQ ID NO: 75 - DNA, 30 bp, Homo sapiens
ctaaggaacatcgacgaccacagccgcttt

SEQ ID NO: 76 - DNA, 30 bp, Homo sapiens
atcggctctttgcagtggtctaccatcacg

SEQ ID NO: 77 - DNA, 30 bp, Homo sapiens
cctgtcaacgcgcaggacttctgcacggac

SEQ ID NO: 78 - DNA, 30 bp, Homo sapiens
ggcaaggtggaacaggagacagaccaacta

SEQ ID NO: 79 - DNA, 30 bp, Homo sapiens
ggcaaggtggaacaggagacaggacaacta

SEQ ID NO: 80 - DNA, 30 bp, Homo sapiens
accaagacctacttcccggtcaacttcaag

SEQ ID NO: 81 - DNA, 30 bp, Homo sapiens
ggaggccctggagagctcctaagccactgc

SEQ ID NO: 82 - DNA, 30 bp, Homo sapiens
attgctgtgaaacaggggtatgatatcagc

SEQ ID NO: 83 - DNA, 30 bp, Homo sapiens
gcagttgccaccttccacgcctgagcgtgg

SEQ ID NO: 84 - DNA, 30 bp, Homo sapiens
gagcagacaagtttgactctgaacaggaag

SEQ ID NO: 85 - DNA, 500 bp, Homo sapiens
ctgtgactccaacctcacaacctgtgcgaaagtctacctttgatgcagccagtttcattggaggaattgtcctggtcttgggtgtgcaggctgtaattttctttctttataaattctgcaaatctaaagaacgaaattaccacactctgtaaacagacccattgaattaataaggactggtgattcatttgtgtaactcactgaagccaaaatactatcttttaagatgtcccacatggaagacgctattccaggatctttaaatttccatggatgcatataggatgtttgggagcatcatccgtgaagaaaaaatcaattaaatcattgtgttcaacaggaatatttaaaatattctgcatgaatcctgtggctgtcttattttaaatagctgctgctgtgggattatattttttttccttaacatgccaaatataactttctgaaagtgatggaaaatgttgtcttgtgcagacaacatcatggctcttggcagttta

SEQ ID NO: 86 - DNA, 30 bp, Homo sapiens
cagccaattctacagctaaacccacagttc

SEQ ID NO: 87 - DNA, 30 bp, Homo sapiens
caccctgagcagctcaccacccacaccacc

SEQ ID NO: 88 - DNA, 234 bp, Homo sapiens
cccagagtattaacgctccaagagtattattaacgctgctgtacctcgatctgaatctgccggggccccagcccactccaccctgccagcagcttccagccagtccccacagcctcatcagctctcttcaccgttttttgatactatcttcccccacccccagctacccataggggctgcagagttataagccccaaacaggtcatgctccaataaaaatgattctacctacaa

SEQ ID NO: 89 - DNA, 30 bp, Homo sapiens
tgcttcggaaggaacgagagctggaagagg

SEQ ID NO: 90 - DNA, 30 bp, Homo sapiens
aggaagaaatgcttcggaaggaacgagagc

SEQ ID NO: 91 - DNA, 30 bp, Homo sapiens
aggaggatgaggatggaggatgaagaaggt

SEQ ID NO: 92 - DNA, 30 bp, Homo sapiens
gatgttgcatgacaggggcactttgggcta

SEQ ID NO: 93 - DNA, 30 bp, Homo sapiens
aactctgtggtggagacaggaagctggggc

SEQ ID NO: 94 - DNA, 30 bp, Homo sapiens
cttgtgcctcaccagatccggggatttctt

SEQ ID NO: 95 - DNA, 30 bp, Homo sapiens
gaagtcagtgaggaggaagaagaatttgga

SEQ ID NO: 96 - DNA, 30 bp, Homo sapiens
aggatgaggatgaagaggaggaagaaggtg

SEQ ID NO: 97 - DNA, 30 bp, Homo sapiens
gaagtcagtgaggagaggaggaagaaggtg

SEQ ID NO: 98 - DNA, 30 bp, Homo sapiens
cattcttacatgctgaggaccggagaagtg

SEQ ID NO: 99 - DNA, 30 bp, Homo sapiens
ctgcagctggcagagaagaatggctacgtg

SEQ ID NO: 100 - DNA, 30 bp, Homo sapiens
ccacgagagtcggaggaaggagctgaagag

SEQ ID NO: 101 - DNA, 30 bp, Homo sapiens
tacatcagcttccagacccagacagtcatc

SEQ ID NO: 102 - DNA, 30 bp, Homo sapiens
ctccagtggaaccaggtttgctgtgaactt

SEQ ID NO: 103 - DNA, 30 bp, Homo sapiens
tagaagctggtggaggtgaggtccagagat

SEQ ID NO: 104 - DNA, 30 bp, Homo sapiens
caggcacattcacagccgcatctgccacaa

SEQ ID NO: 105 - DNA, 198 bp, Homo sapiens
aattttgaagctgagtcaattcaagataatgcgcatatggcagagggcacaggtttctatccctcagaacccatgctctgtagtgaatcggtggaagggcaagtgccacattcattagagaccttgtatcaatcagctgactgttctgatgccaatgatgccttgatagtgttgatacatcttctcatgttggagtca

SEQ ID NO: 106 - DNA, 30 bp, Homo sapiens
ccacctccctactacccaccggaagataag

SEQ ID NO: 107 - DNA, 30 bp, Homo sapiens
cacgcccggctgatgggaatttggtcttgc

SEQ ID NO: 108 - DNA, 55 bp, Homo sapiens
ggaaacagtgataagtaagctgaccacttgctgtaggagaagttccaacgtgtcc

SEQ ID NO: 109 - DNA, 500 bp, Homo sapiens
gacgcatctcaaaacagagggctgcattcgaagaaacccttgctgctttagtcccgatagggtatttgaccccgatatattttagcattttaattctctccccctatttattgactttgacaattactcaggtttgagaaaaaggaaaaaaaaacagccaccgtttcttcctgccagcaggggtgtgatgtaccagtttgtccatcttgagatggtgaggctgtcagtgtatggggcagcttccggcgggatgttgaactggtcattaatgtgtcccctgagttggagctcattctgtctcttttctcttttgctttctgtttcttaagggcacacacacgtgcgtgcgagcacacacacacatacgtgcacagggtccccgagtgcctaggttttggagagtttgcctgttctatgcctttagtcaggaatggctgcacctttttgcatgatatcttcaagcctgggcgtacagagcacatttgtcagtatttttgccg

SEQ ID NO: 110 - DNA, 500 bp, Homo sapiens
aaggaggtcaggccccactccttcctgattgtttacagtcattggaataaggcatggctcagatcggccacagggcggtaccttgtgcccagggttttgccccaagtcctcatttaaaagcataaggccggacgcatctcaaaacagagggctgcattcgaagaaacccttgctgctttagtcccgatagggtatttgaccccgatatattttagcattttaattctctccccctatttattgactttgacaattactcaggtttgagaaaaaggaaaaaaaaacagccaccgtttcttcctgccagcaggggtgtgatgtaccagtttgtccatcttgagatggtgaggctgtcagtgtatggggcagcttccggcgggatgttgaactggtcattaatgtgtcccctgagttggagctcattctgtctcttttctcttttgctttctgtttcttaagggcacacacacgtgcgtgcgagcacacacacacatacgtgc

SEQ ID NO: 111 - DNA, 30 bp, Homo sapiens
gtggctgatggaagagatccagctgcctcc

SEQ ID NO: 112 - DNA, 30 bp, Homo sapiens
agaagggctctgaagagaatctggacgagg

SEQ ID NO: 113 - DNA, 137 bp, Homo sapiens
gttaacctcaccctacagatgaagataatagagcaagaaaaagaaattgcagaactaaagaagcagctaaacctcctttctcaagacaaaggggtgagggatgacggaaaggatgggggcgtgggatgaaagtggac

SEQ ID NO: 114 - DNA, 30 bp, Homo sapiens
atggttcccaagtgtgtgtaagtgtgtgta

SEQ ID NO: 115 - DNA, 336 bp, Homo sapiens
tgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacct

SEQ ID NO: 116 - DNA, 30 bp, Homo sapiens
ggaaaacatctcaaggcctgaagctgccca

SEQ ID NO: 117 - DNA, 30 bp, Homo sapiens
gtactacatgcaatatttagcagccactgc

SEQ ID NO: 118 - DNA, 108 bp, Homo sapiens
ctgattccattcttctgttaccgactttttgacttcgtcctcagttgcctggttgctattagttctctcacctatttgccaagaatcaaagaatatctggatcaacta

SEQ ID NO: 119 - DNA, 62 bp, Homo sapiens
agcagtacctccctaaagcattttgaggtaggggaggtatccattcataaaatgaatgtggg

SEQ ID NO: 120 - DNA, 31 bp, Homo sapiens
cgagagcgcaggattgagatagaggagaaca

SEQ ID NO: 121 - DNA, 30 bp, Homo sapiens
agaaacagggcaagtacccagaagtccaca

SEQ ID NO: 122 - DNA, 138 bp, Homo sapiens
cagaagtccacattcccctcgagaatctacagcagtttcctgccttcaattcttatacctgctcctctgattctgctttgggtcccaccagcaccgagggccatggggccctctccatttctcctctcagcagaagtc

SEQ ID NO: 123 - DNA, 30 bp, Homo sapiens
caggttcccatgcagactttctgctgaccc

SEQ ID NO: 124 - DNA, 30 bp, Homo sapiens
tcagtgaattagcaggtcatcagactagtg

SEQ ID NO: 125 - DNA, 117 bp, Homo sapiens
gtcatcagactagtgctgagtcttggggtactggcagagctgtggctcgaattcccagagttcgaggtggtgggactcaccgctctggccagggtgcttttggaaacatgtgtcgtg

SEQ ID NO: 126 - DNA, 157 bp, Homo sapiens
gcgtgtgctcgcccactgatatcggtgtactccgaaaagggggagtcatctggcaaaaatgtcactttgcctgctgtattcaaggctcctattcgaccagatattgtgaactttgttcacaccaacttgcgcaaaaacaacagacagccctatgctg

SEQ ID NO: 127 - DNA, 30 bp, Homo sapiens
tggtcatgtctaaaggtcatcgtattgagg

SEQ ID NO: 128 - DNA, 30 bp, Homo sapiens
gatatcatttgtcagattgcttatgcccgt

SEQ ID NO: 129 - DNA, 30 bp, Homo sapiens
cacttccaccttcaaccttagctccagttt

SEQ ID NO: 130 - DNA, 30 bp, Homo sapiens
tcacacaggacagaggaggcagagcttctg

SEQ ID NO: 131 - DNA, 30 bp, Homo sapiens
ttcccaaatttctacagcctctttcctctt

SEQ ID NO: 132 - DNA, 30 bp, Homo sapiens
ggagtcaaacactggatgcagaaattttgg

SEQ ID NO: 133 - DNA, 30 bp, Homo sapiens
ttttgatgcacagagctaccaccacagtgc

SEQ ID NO: 134 - DNA, 95 bp, Homo sapiens
tctttttgctaaagaacatctgcagcacatgacagaaaagcagctgaacctctatgaccgcctgattaacgagcctagtaatgactgggatattt

SEQ ID NO: 135 - DNA, 105 bp, Homo sapiens
accagagaccttcccacctccaggagaggaagagggtgaggaagaagaggacaatgatgaggatgaagaggagatgctcagtgatgccagcttatggacctacag

SEQ ID NO: 136 - DNA, 30 bp, Homo sapiens
ccctgaccggcaagacatcactctggaggt

SEQ ID NO: 137 - DNA, 30 bp, Homo sapiens
caggtcaaaatgcagatcttcgtgaaaacc

SEQ ID NO: 138 - DNA, 30 bp, Homo sapiens
caggtcaaaatgcagatcttcgtgaagacc

SEQ ID NO: 139 - DNA, 358 bp, Homo sapiens
agatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtggctgttaattcttcagtcatggcattcgcagtgcccagtgatggcattactctgcactatagccatttgccccaacttaagtttagaaattacaagtttcagtaatagctgaacctgttcaaaatgttaataaagg

SEQ ID NO: 140 - DNA, 39 bp, Homo sapiens
tccctgtgggtggacgtggttggtgattggcaggatcct

SEQ ID NO: 141 - DNA, 30 bp, Homo sapiens
ggttggctttgttgggtgagcttgtttgtg

SEQ ID NO: 142 - DNA, 30 bp, Homo sapiens
cttagtggcatgttggatggtcttgttaat

SEQ ID NO: 143 - DNA, 158 bp, Homo sapiens
agagagaagatcactgaagaaacttctgctttaatgactttacaaagctggcaatattacaatccttgacctcagtgaaagcagtcatcttcagcgttttccagccctatagccaccccaagtgtggttatgcctcctcgattgctccgtactctaac

SEQ ID NO: 144 - DNA, 30 bp, Homo sapiens
atctagctggctttccctgtctattgcctt

SEQ ID NO: 145 - DNA, 162 bp, Homo sapiens
tcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctc

SEQ ID NO: 146 - DNA, 30 bp, Homo sapiens
gcagcaggatcacaggtggaggaaggagag

SEQ ID NO: 147 - DNA, 30 bp, Homo sapiens
gcagcaggatcacagaacagacccaggaaa

SEQ ID NO: 148 - DNA, 30 bp, Homo sapiens
tttctcacacaggcatactccaaatgcttc

SEQ ID NO: 149 - DNA, 30 bp, Homo sapiens
aaccactttgaccggggagaagcagaattt

SEQ ID NO: 150 - DNA, 87 bp, Homo sapiens
ggaactctctctctgatgctgatttgcactctgctggaattctgcctagctgtgctcactgctgtgctgcggtggaaacaggcttac

SEQ ID NO: 151 - DNA, 189 bp, Homo sapiens
tgccctggcattctggcagagaatcctcaccagttctcaccaaccttccccccaggcaagggcagctgccagcatggtgctctgccaggacaggtttccctgaaggaagctgctcacactgagatgagcctctcagggcaggacctcttcccaagccctgcacacccacccctgcagcccttttggctc

SEQ ID NO: 152 - DNA, 30 bp, Homo sapiens
gcttcagtccgccgagagcagtaccgtgtg

SEQ ID NO: 153 - DNA, 79 bp, Homo sapiens
ctgacccagataaaacaaaaagtggattctctcctggaaaacctggaaaaaatggaaaaggaacagagcaaacaagcag

SEQ ID NO: 154 - DNA, 30 bp, Homo sapiens
ggtgggggtggaagcagccgcaggagcaag

SEQ ID NO: 155 - DNA, 30 bp, Homo sapiens
tgttccctatggcagaaggcaaccacagaa

SEQ ID NO: 156 - DNA, 142 bp, Homo sapiens
gtcgctgagagcctcttcctgcccagaacagattacagcttccacaagttccattacctgacctttgtgccctcagcagaggacttctatgactgcagggtggagcactggggcttggaccagccgctcctcaagcactggg

SEQ ID NO: 157 - DNA, 54 bp, Homo sapiens
accctcatctgccacattgacaagttcttcccaccagtgctcaacgtcacgtgg

SEQ ID NO: 158 - DNA, 30 bp, Homo sapiens
aaggagcctgtggagctgggccagcccaac

SEQ ID NO: 159 - DNA, 30 bp, Homo sapiens
gctccctgttcttggatctgccatggaggg

SEQ ID NO: 160 - DNA, 74 bp, Homo sapiens
ccagggacggccacttcctggtccccgacgcaaccatggctgaagaacaaccgcaggtcgaattgttcgtgaag

SEQ ID NO: 161 - DNA, 30 bp, Homo sapiens
gggcagctcccattcctgctgtatggcact

SEQ ID NO: 162 - DNA, 30 bp, Homo sapiens
accggagggaagaaggtgtgctcagtgaag

SEQ ID NO: 163 - DNA, 45 bp, Homo sapiens
tggaaaggagaaggaataagacggcaggaggaagagagagagagg

SEQ ID NO: 164 - DNA, 30 bp, Homo sapiens
acctcatctcggccagtgctgacctggagg

SEQ ID NO: 165 - DNA, 143 bp, Homo sapiens
ttcctaaggacggtctgaagagccaagagctatttgatgaaattagaatgacctacatcaaagagctaggaaaagccattgtcaagagggaaggaaactccagccagaactggcagcggttttatcaactgacaaaactcttg

SEQ ID NO: 166 - DNA, 30 bp, Homo sapiens
agtgatatggttcaggaacacgatgaacct

SEQ ID NO: 167 - DNA, 53 bp, Homo sapiens
atgaatattttacaaatgaagtgctgacaaagacatacaggatgaggtcagaa

SEQ ID NO: 168 - DNA, 30 bp, Homo sapiens
gctggccagcctatgagttttgtcttagaa

SEQ ID NO: 169 - DNA, 127 bp, Homo sapiens
cctgcctagggtagttaaaagacgagtgaatgctctcaaaaacctgcaagttaaatgtgcacagatagaagccaaattctatgaggaagttcacgatcttgaaaggaagtatgctgttctctatcag

SEQ ID NO: 170 - DNA, 340 bp, Homo sapiens
gctatggtggggacagaggaggcggctatggaggagaccgaggaggtggctatggaggagatcgaggtggctatggaggagaccgaggtggaggctatggtggagaccgaggaggctatggaggagatcgaggaggttacggaggagatcgaggaggttatggaggagatcgaggaggctatggaggagacagaagccgggggggctatggaggagaccgtggtggtggcagtggctacggtggagaccgaagtggaggctatggaggagacaggagtggtggcggctatggaggagaccgaggtgggggctacggaggagaccgaggtggctatggagg

SEQ ID NO: 171 - DNA, 30 bp, Homo sapiens
gggacagaggcggcggctatggtggggaca

SEQ ID NO: 172 - DNA, 500 bp, Homo sapiens
gaccacagcaatgaccagccctcattagggccctggatgatttttggtctaataacgcatgctagtgttgatgttttttggtcagagggtatgaacaggaagaattaaatgcagcaggctttattttaaatgccgattcacattactctgttcaagctgcgttgagatgttaaactggcttactatagacttcgtaaaaatggctccagaaaagtaacaaactgaaatctttgagatcacacaggttggaaatatgtacataactgcacaaggtgtcaattctgctctacagtgcagttttagtcagttttagttgcataggtttccattgtatttatagtctgtttatgctaaatctggccaaagatgaacattgtccaccactaaaatgcctctgccactttgaattctgtgctaattttgtggccagaatgcggtgatcaaaacgctccatctttttacagtggcataggaagacggcaaaaatttcctaaagtgca

SEQ ID NO: 173 - DNA, 30 bp, Homo sapiens
accccttctgctcaggtgtggatggtattg

SEQ ID NO: 174 - DNA, 30 bp, Homo sapiens
actcgtatccccaagattcagaagcttctc

SEQ ID NO: 175 - DNA, 30 bp, Homo sapiens
actggccaggacctggaagcagacacctct

SEQ ID NO: 176 - DNA, 30 bp, Homo sapiens
ggaagacaaagaaggagagattcaagcagg

SEQ ID NO: 177 - DNA, 30 bp, Homo sapiens
tgatgagagtgagaggcagagacccgtgct

SEQ ID NO: 178 - DNA, 30 bp, Homo sapiens
tgatgagagtgagagaggatgctggaccag

SEQ ID NO: 179 - DNA, 30 bp, Homo sapiens
tcaatataaaacccccacctaccacacatt

SEQ ID NO: 180 - DNA, 30 bp, Homo sapiens
cttaaactccagcaccatcatagccaccat

SEQ ID NO: 181 - DNA, 30 bp, Homo sapiens
caccaatggaagcagtggacaagaaggctg

SEQ ID NO: 182 - DNA, 371 bp, Homo sapiens
catcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgag

SEQ ID NO: 183 - DNA, 232 bp, Homo sapiens
tgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaagg

SEQ ID NO: 184 - DNA, 84 bp, Homo sapiens
ctgagtccagattggcaggtggactacgagtcatacacatggcggaaactggatcctggcagcgaggagacccagacgctggtt

SEQ ID NO: 185 - DNA, 66 bp, Homo sapiens
cagcatgtgggcaaagccttcaatcagggcaagatcttcaagtgaacatctcttgccatcacctag

SEQ ID NO: 186 - DNA, 146 bp, Homo sapiens
gttctagagccttctttccgccaggccttcccaataccaaccgctggttcctcacctgcattaaccagccccagttccgggctgtcttgggcgaagtgaaactgtgtgagaagatggcccagtttgatgctaaaaagtttgcagag

SEQ ID NO: 187 - DNA, 155 bp, Homo sapiens
atttaagcgcaagtactccaatgaggacacactctctgtggcactgccatatttctgggagcactttgataaggacggctggtccctgtggtactcagagtatcgcttccctgaagaactcactcagaccttcatgagctgcaatctcatcactg

SEQ ID NO: 188 - DNA, 95 bp, Homo sapiens
tggacaagctgaggaagaatgccttcgccagtgtcatcctttttggaaccaacaatagcagctccatttctggagtctgggtcttccgaggccag

SEQ ID NO: 189 - DNA, 122 bp, Homo sapiens
caggtggactacgagtcatacacatggcggaaactggatcctggcagcgaggagacccagacgctggttcgagagtacttttcctgggagggggccttccagcatgtgggcaaagccttcaa

SEQ ID NO: 190 - DNA, 30 bp, Homo sapiens
gagcttgcctttccgctgagtccagattgg

SEQ ID NO: 191 - DNA, 113 bp, Homo sapiens
aaggaggagaaaaaggcggctgcccctgctcctgaggaggagatggatgaatgtgagcaggcgctggctgctgagcccaaggccaaggaccccttcgctcacctgcccaagag

SEQ ID NO: 192 - DNA, 100 bp, Homo sapiens
ttccgccaggcctttcccaataccaaccgctggttcctcacctgcattaaccagccccagttccgggctgtcttgggcgaagtgaaactgtgtgagaaga

SEQ ID NO: 193 - DNA, 39 bp, Homo sapiens
gttcacgggaagagaagcagaagccccaggctgagcgga

SEQ ID NO: 194 - DNA, 30 bp, Homo sapiens
aaagaatatctagccctctgttcaacacca

SEQ ID NO: 195 - DNA, 30 bp, Homo sapiens
cacgaaaagtagccgctctggttgaagctt

SEQ ID NO: 196 - DNA, 30 bp, Homo sapiens
ccaatttctttggcagcaacgctcagtata

SEQ ID NO: 197 - DNA, 30 bp, Homo sapiens
catggagaagatctgggcgcaccactggca

SEQ ID NO: 198 - DNA, 30 bp, Homo sapiens
agagagaacgcagaggccacggaagccctg

SEQ ID NO: 199 - DNA, 30 bp, Homo sapiens
atttcaacagctgaggaaggtgtcttgctg

SEQ ID NO: 200 - DNA, 161 bp, Homo sapiens
tttggcaggaaggtgtcttgctgcaggtaactaatgaagaagtggtcaaccacagagtcttcaagaaataagaaattctgtaccatctgaaagtagttcttgttggtgccttcatttaaaaagcactctttaaaataaaagggaaatgttttctgataaaa

SEQ ID NO: 201 - DNA, 500 bp, Homo sapiens
tttattttcaaatgcagtgtagagctagattaaaagcaactctttgccacctactctgcccttttggcaaagttaccttgaacaaagaatcttaagggtttattaagaactctttattttcttcataccctgttctctgcagtgctttctaacagcttctgggtgcagattttcttcggcatccttttgcactcagcttattacaggtaggtagtgcttaagaaaagtcatggaggactaaagcctaagtccttttcacttttcctccatctgaaggtaggtgagttcatcctcttcatagtaatgctgttttaccaagactttatagcagatggacccagaaagaattttctgctattgtgttcactacaacaggatagggacatcagacagccccagaaaccccttccagatctgatatgggactattaatttttatgctgttaattggtattcattcacaatgcagttgaagggggaaggctccactgcattctttg

SEQ ID NO: 202 - DNA, 30 bp, Homo sapiens
attgcaaaacgggtgtattatccaggtact

SEQ ID NO: 203 - DNA, 30 bp, Homo sapiens
cctttgaggcagcagatgaaagtgggaaca

SEQ ID NO: 204 - DNA, 30 bp, Homo sapiens
aaacaggaacaagaagcagcaaagctggcc

SEQ ID NO: 205 - DNA, 30 bp, Homo sapiens
ataccgtctgtgaagatgtggacgagtgca

SEQ ID NO: 206 - DNA, 57 bp, Homo sapiens
caggctggaagcccagacacggaatcccggataaccaaaaggacactgtctgtgaag

SEQ ID NO: 207 - DNA, 30 bp, Homo sapiens
caagggaccaaggtggagcagttgaaatct

SEQ ID NO: 208 - DNA, 32 bp, Homo sapiens
tgtctctggggatccctagtataacacatgca

SEQ ID NO: 209 - DNA, 30 bp, Homo sapiens
ccggctggtcaaagtgtgggtgctggcagc

SEQ ID NO: 210 - DNA, 30 bp, Homo sapiens
ggttcggggaccaggttaaccgttgtagag

SEQ ID NO: 211 - DNA, 372 bp, Homo sapiens
gaggacctgaacaaggtgttcccacccgaggtcgctgtgtttgagccatcagaagcagagatctcccacacccaaaaggccacactggtgtgcctggccacaggtatcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgag

SEQ ID NO: 212 - DNA, 30 bp, Homo sapiens
tctgtgccagcagccttggacagatttatg

SEQ ID NO: 213 - DNA, 30 bp, Homo sapiens
atatctctgcagcgtcgggggtcaatctgg

SEQ ID NO: 214 - DNA, 30 bp, Homo sapiens
ctgcagtgctagagatccggggtcccatca

SEQ ID NO: 215 - DNA, 30 bp, Homo sapiens
ccagatcgtcagcgccgaggcctggggtag

SEQ ID NO: 216 - DNA, 30 bp, Homo sapiens
cctctggcctcacagtgtcccaggagcaca

SEQ ID NO: 217 - DNA, 30 bp, Homo sapiens
tggaacttcaccaagttcctcatcgacaag

SEQ ID NO: 218 - DNA, 30 bp, Homo sapiens
ttccaactgtttaaaattgatcagggacca

SEQ ID NO: 219 - DNA, 30 bp, Homo sapiens
cagtatggtgaggaggtgtgagagggtcca

SEQ ID NO: 220 - DNA, 30 bp, Homo sapiens
tcagagtggaagcaggtgagaatggagggg

SEQ ID NO: 221 - DNA, 30 bp, Homo sapiens
ctttgggcgaggagagtgtgagagggtcca

SEQ ID NO: 222 - DNA, 30 bp, Homo sapiens
gcgaggagagtgcgtgtgtgagagggtcca

SEQ ID NO: 223 - DNA, 178 bp, Homo sapiens
ttccagaagccagagagaccaagtgttatgtaagaagtagtgtcggctgtgtagaaccactgactacacaggccgaagttactgagaacttggacagaaaaaatagccagcaagtgttcaaactactgaggaaaaaaaaaaattagatatgctgcacttaagaatactagggcaggtt

SEQ ID NO: 224 - DNA, 35 bp, Homo sapiens
gagaatgagaagcgctggaggcaggagacaccccg

SEQ ID NO: 225 - DNA, 102 bp, Homo sapiens
ttctttattggtgaaaacatgaatccagatggcatggttgctctattggactaccgtgaggatggtgtgaccccatatatgattttctttaaggatggttta

SEQ ID NO: 226 - DNA, 30 bp, Homo sapiens
gaaatggaaaaatgttaacaaatgtggcaa

SEQ ID NO: 227 - DNA, 194 bp, Homo sapiens
ttattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaac

SEQ ID NO: 228 - DNA, 37 bp, Homo sapiens
cgtcgtctcccttcagtcgccatcatgattatctacc

SEQ ID NO: 229 - DNA, 30 bp, Homo sapiens
gggacctcatcagccacgatgagatgttct

SEQ ID NO: 230 - DNA, 30 bp, Homo sapiens
tttatttcctaccagcaggaggagccagag

SEQ ID NO: 231 - DNA, 30 bp, Homo sapiens
cccaaggtaccgtagcaggggacctcctcg

SEQ ID NO: 232 - DNA, 74 bp, Homo sapiens
aataacccacggaaatatctgcgcagtgtaggagatggagaaactgtagagtttgatgtggttgaaggagagaa

SEQ ID NO: 233 - DNA, 30 bp, Homo sapiens
gtatttgtacatcagactgccatcaagaag

SEQ ID NO: 234 - DNA, 56 bp, Homo sapiens
gaagcagaagaggtgtgaaagaaggtgctgctgggaggggagtctgacaacccagc

SEQ ID NO: 235 - DNA, 312 bp, Homo sapiens
acgacatcgcagaaggtggctcggaaattctggtggaagaacgtgaagatgattgtccttatctgcgtgattgtttttatcatcatcctcttcattgtgctctttgccactggtgccttctcttaagtaacagggaacctctcccacctgcccttcttttcagggacaaccctccataaatgtgtgccaagagggtctcctttcctgtcttcctctacagagaatgctgctcggtcctcctacccctcttcccgaggcctgctgccacgttgtatgccccagaaggtaccttggtcccccggaaggagagaa

SEQ ID NO: 236 - DNA, 30 bp, Homo sapiens
gatctggaagccacatctgagcacttcaag

SEQ ID NO: 237 - DNA, 30 bp, Homo sapiens
agtacaagcagtctggtggatggaatggaa

SEQ ID NO: 238 - DNA, 264 bp, Homo sapiens
aggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggg

SEQ ID NO: 239 - DNA, 30 bp, Homo sapiens
tggaggctatccagcgtactccaaagattc

SEQ ID NO: 240 - DNA, 30 bp, Homo sapiens
gtggagaggatcaaggtctctcaggcagct

SEQ ID NO: 241 - DNA, 30 bp, Homo sapiens
tgactgtcacccacagcaatgagaagcacg

SEQ ID NO: 242 - DNA, 30 bp, Homo sapiens
cctcaagttctcgctagatgtctgcgaggc

SEQ ID NO: 243 - DNA, 30 bp, Homo sapiens
gccccaggaggagaggtgagctgaagatgg

SEQ ID NO: 244 - DNA, 30 bp, Homo sapiens
cacgggggaagagtggaccactcttcaact

SEQ ID NO: 245 - DNA, 30 bp, Homo sapiens
tcaagagtgagtgtttgcggagtcagacgc

SEQ ID NO: 246 - DNA, 30 bp, Homo sapiens
ctactcctgaggatggtgtgtatggcttcc

SEQ ID NO: 247 - DNA, 30 bp, Homo sapiens
gacgccagggtttgggtgctggagaatctg

SEQ ID NO: 248 - DNA, 30 bp, Homo sapiens
tgcttctccccgcaggatgttcctgtcctt

SEQ ID NO: 249 - DNA, 30 bp, Homo sapiens
aagttcacaattggcgaccacagccgcttt

SEQ ID NO: 250 - DNA, 96 bp, Homo sapiens
tttattcatggtggtcatactgccaagatatctgatttctcctggaatcccaatgaaccttgggtgatttgttctgtatcagaagacaatatcatg

SEQ ID NO: 251 - DNA, 30 bp, Homo sapiens
ccgtcgccatgacccgcggtaaccagcgtg

SEQ ID NO: 252 - DNA, 30 bp, Homo sapiens
cctttgtgcttgcagaagtttgcctgcaat

SEQ ID NO: 253 - DNA, 30 bp, Homo sapiens
aacagcttcaaacagtggttggcacttacc

SEQ ID NO: 254 - DNA, 30 bp, Homo sapiens
agcaaaaaatttttggaatggtcgttggag

SEQ ID NO: 255 - DNA, 30 bp, Homo sapiens
aactctgtggtggaggtgagctggggagga

SEQ ID NO: 256 - DNA, 30 bp, Homo sapiens
acagtggagcagaaggtgggtgcgggaagc

SEQ ID NO: 257 - DNA, 104 bp, Homo sapiens
aagacgaggacgatgaggatggtgaagaagaggagtttgatgaagaagatgatgaagatgaagatgtagaaggggatgaggacgacgatgaagtcagtgaggag

SEQ ID NO: 258 - DNA, 30 bp, Homo sapiens
aggaggaggacgaagaaggagaagatgagg

SEQ ID NO: 259 - DNA, 30 bp, Homo sapiens
gataagcccacccagagaagtgtttccaat

SEQ ID NO: 260 - DNA, 30 bp, Homo sapiens
agatgctccagaaggtgagtgtggctgcag

SEQ ID NO: 261 - DNA, 30 bp, Homo sapiens
aagatgctccagaaggtgagtgtggctgca

SEQ ID NO: 262 - DNA, 58 bp, Homo sapiens
  misc_feature (9)..(9): n is a, c, g, t or u
cgggctacnanatgcgcttggggggagccaggacggaggaagaggagagagaaagaga

SEQ ID NO: 263 - DNA, 30 bp, Homo sapiens
gtggacaatgggaagctgctggaagggcct

SEQ ID NO: 264 - DNA, 30 bp, Homo sapiens
aagcctacaagaaagtttgcctatctgggg

SEQ ID NO: 265 - DNA, 32 bp, Homo sapiens
catgctgttccttcctcgccaccctctgcttc

SEQ ID NO: 266 - DNA, 30 bp, Homo sapiens
tcgcggggcaaaatggagctcgaggccatg

SEQ ID NO: 267 - DNA, 30 bp, Homo sapiens
tgcgaccgcctacaggtgacctccaaagct

SEQ ID NO: 268 - DNA, 86 bp, Homo sapiens
actttctgctgacccctctgtcacccactgccagtcagggcagtccctcattctgcgttgggagtctggaagaagactctcccttc

SEQ ID NO: 269 - DNA, 30 bp, Homo sapiens
ggtgcttttggaaacatgtgtcgtggaggc

SEQ ID NO: 270 - DNA, 30 bp, Homo sapiens
tctctgggcaagcagaaagcaaaaggtgat

SEQ ID NO: 271 - DNA, 30 bp, Homo sapiens
gaatacaaggccaagaaagcaaaaggtgat

SEQ ID NO: 272 - DNA, 30 bp, Homo sapiens
aaggccatcgtatggaataatgatctcctt

SEQ ID NO: 273 - DNA, 30 bp, Homo sapiens
caacttcgacaaataccacccaggctactt

SEQ ID NO: 274 - DNA, 25 bp, Homo sapiens
gccacccaggctactttgggaaagt

SEQ ID NO: 275 - DNA, 30 bp, Homo sapiens
gccacggccgcataggcaagcaccggaagc

SEQ ID NO: 276 - DNA, 102 bp, Homo sapiens
ggagcattccatatagaaactgctgaaactgccacaggtgcttctccgaaaaccttacagttgtggcattgaatgttcagtatcgcttcctttctgcacacg

SEQ ID NO: 277 - DNA, 30 bp, Homo sapiens
ccaagttcctgaggaacatgcgctttgcca

SEQ ID NO: 278 - DNA, 86 bp, Homo sapiens
ttccgtctgattctaatagagagccggattcaccgtttggctcgatattataagaccaagcgagtcctccctcccaattggaaata

SEQ ID NO: 279 - DNA, 30 bp, Homo sapiens
cacagcagcatcaaggtggagtgcagcaac

SEQ ID NO: 280 - DNA, 30 bp, Homo sapiens
aattatgttcctgaggtctcagccttggat

SEQ ID NO: 281 - DNA, 135 bp, Homo sapiens
gccgcgttccaccaaaaccgtgaagaaagcggcccgggtcatcatagaaaagtactacacgcgcctgggcaacgacttccacacgaacaagcgcgtgtgcgaggagatcgccattatccccagcaaaaagctccg

SEQ ID NO: 282 - DNA, 30 bp, Homo sapiens
gcgagagtgtgtcgagtgagtgtgcgtctg

SEQ ID NO: 283 - DNA, 30 bp, Homo sapiens
gaaggcggaaaagaggtcaaaatgcagatc

SEQ ID NO: 284 - DNA, 30 bp, Homo sapiens
ggtatccgctaacaggtcaaaatgcagatc

SEQ ID NO: 285 - DNA, 30 bp, Homo sapiens
acttcagtgctggtggtgagggcatagcct

SEQ ID NO: 286 - DNA, 30 bp, Homo sapiens
gtgttaggagcggagataccttcacttgct

SEQ ID NO: 287 - DNA, 30 bp, Homo sapiens
cgcgtctcctttgagctgtttgcagacaag

SEQ ID NO: 288 - DNA, 30 bp, Homo sapiens
tcccaaagacagcagaaaattttcgtgctc

SEQ ID NO: 289 - DNA, 30 bp, Homo sapiens
cgcgtctcctttgaggtacggggcctggat

SEQ ID NO: 290 - DNA, 30 bp, Homo sapiens
attgttgaactggatgcggctgttgaagag

SEQ ID NO: 291 - DNA, 51 bp, Homo sapiens
gatcaacgttttcaaagggggtggcttgcaaagcaacgagctctatgccct

SEQ ID NO: 292 - DNA, 30 bp, Homo sapiens
gtcgcggccatgaaggtgggggagtggtac

SEQ ID NO: 293 - DNA, 30 bp, Homo sapiens
ttcttcttttcctaggggtctcgggaacag

SEQ ID NO: 294 - DNA, 30 bp, Homo sapiens
aagaaggatctgagtgtgagtgtgccagtg

SEQ ID NO: 295 - DNA, 29 bp, Homo sapiens
actggtccatcagtgacaaagacattgag

SEQ ID NO: 296 - DNA, 30 bp, Homo sapiens
aaaaaaagaagaaagaggcacaagtgaaag

SEQ ID NO: 297 - DNA, 30 bp, Homo sapiens
tgcgcggaacccgagatgagcagcaccagc

SEQ ID NO: 298 - DNA, 82 bp, Homo sapiens
cctgagtccaacacagctgggctggacatatttgccaaattttctgcctacatcaagaattcaaacccagcactcaatgaca

SEQ ID NO: 299 - DNA, 30 bp, Homo sapiens
cccaggtaccccaagctggcagctctgaac

SEQ ID NO: 300 - DNA, 30 bp, Homo sapiens
gctgtgccctcccaggtaccccaagctggc

SEQ ID NO: 301 - DNA, 30 bp, Homo sapiens
cccaaaacatgtgaggctggagtacaatgg

SEQ ID NO: 302 - DNA, 30 bp, Homo sapiens
gccacattagcccaggtatctatgccagca

SEQ ID NO: 303 - DNA, 30 bp, Homo sapiens
gcctaccttgggaagactgttaccaatgct

SEQ ID NO: 304 - DNA, 285 bp, Homo sapiens
gccaagaagcttgagagaagaaaaatttcagaaaaattgtctcaatttgactagaatatcaatgaaccaggaaaactgaagcaccttccctaaagaaaacttgggtatacaattactccacagacagagctgagggttttttacccaaatcagtcactggattttgctgcctgatacgtgaatcttcttggaatttttctcatgtggatctaaggggaatgctttattatggctgctgttgtccaacagaacgacctagtatttgaatttgctagtaacgtcatg

SEQ ID NO: 305 - DNA, 30 bp, Homo sapiens
ggcttcactgctcaggtgattatcctgaac

SEQ ID NO: 306 - DNA, 30 bp, Homo sapiens
agcttctcagactatccacctttgggtaag

SEQ ID NO: 307 - DNA, 85 bp, Homo sapiens
aagtggagcacgaaagcaggacccagaatgggaggagcttccagaggaccggaacacttgcctttgagcgggtctacactgccaa

SEQ ID NO: 308 - DNA, 43 bp, Homo sapiens
aagaaagatgaggcagaggtccaagtaaaccgctagcttgttg

SEQ ID NO: 309 - DNA, 30 bp, Homo sapiens
gaagcgaatgcgcaggctgaagcgcaaaag

SEQ ID NO: 310 - DNA, 122 bp, Homo sapiens
aatgcatatttaaactaaattgatcctgtagtgttcctggagaagctagagcctgattgtaggctactactcatcaattaacttctacagtggagactacttctgggactggaatataaaaa

SEQ ID NO: 311 - DNA, 80 bp, Homo sapiens
attaccgtttattccatatctggataatttgccgaacttcaatagatcagttgatggaccaatcaggctgccaattgtgg

SEQ ID NO: 312 - DNA, 33 bp, Homo sapiens
ttgtgtgagctattcaaactcttcaacccctga

SEQ ID NO: 313 - DNA, 30 bp, Homo sapiens
agccagttgtcatttttacaggattgtgtg

SEQ ID NO: 314 - DNA, 50 bp, Homo sapiens
agttcaggagccctggaaaggagaaggaataagacggcaggaggaagaga

SEQ ID NO: 315 - DNA, 30 bp, Homo sapiens
ctacctaattgattgcccggggccctgatt

SEQ ID NO: 316 - DNA, 63 bp, Homo sapiens
ggaagcgcgggtggtcggctggggtccggctcctggagaacatggcccggcctcccgggggct

SEQ ID NO: 317 - DNA, 30 bp, Homo sapiens
ctggtcccctcctcgttctaatacccgatt

SEQ ID NO: 318 - DNA, 70 bp, Homo sapiens
gtaattcaactgccaagtggtggaagagggaaatagaagaactatacaacaatccacagaatccagagag

SEQ ID NO: 319 - DNA, 30 bp, homo sapiens
tcacctgcccaagagtacctttgtgttgga

SEQ ID NO: 320 - DNA, 94 bp, homo sapiens
accctgtacacgtatcctgaaaactggagggccttcaaggctctcatcgctgctcagtacagcggggctcaggtccgcgtgcactccgcaccac

SEQ ID NO: 321 - DNA, 30 bp, homo sapiens
ttgcccccccgccagcaacgctcagtatat

SEQ ID NO: 322 - DNA, 30 bp, homo sapiens
gtcgccgcctgcccgagtgtgtggcaggcc

SEQ ID NO: 323 - DNA, 500 bp, homo sapiens
cccagaaagaattttctgctattgtgttcactacaacaggatagggacatcagacagccccagaaaccccttccagatctgatatgggactattaatttttatgctgttaattggtattcattcacaatgcagttgaagggggaaggctccactgcattctttggctaaggcctgaatgcttgctcatctgtaagatctatactcgaggttttgttttccttttaaaattctttagggagagagggatggtttctgaggggttctgaaagtatgattcaatgtgcaacatacaggtaggtcttcagcataagctgaaatatatgcatgtaaaaactttgacatctttttttttaattttccactttcttcttaactttacttctctttttgtcccccccccatcttacagaagttgaggccaagggagaatggtaggcacagaagaaacatggcaaactgctctgtgctttcaaaccaaagtgttccccccaaccccaaa

SEQ ID NO: 324 - DNA, 30 bp, homo sapiens
caacttcatcagctgccaggcccaggagga

SEQ ID NO: 325 - DNA, 42 bp, homo sapiens
gccaaggtcatccatgacaactttggtatcgtggaaggactc

SEQ ID NO: 326 - DNA, 30 bp, homo sapiens
gatctgcataaacagggttcctgagctcac

SEQ ID NO: 327 - DNA, 239 bp, homo sapiens
tcgttgtcaaaacagagatgtccgtcagtatgttcaaggttgtggagtgtaactccagaattttccttcttcagctcattttgtctctctcacattaagggagtaggaattaagtgaaaggtcacactaccattatttccccttcaaacaaataatatttttacagaagcaggagcaaaatatggcctttcttctaagagatataatgttcactaatgtggttattttacattaagcct

SEQ ID NO: 328 - DNA, 30 bp, homo sapiens
gaaacttgaagaacagagaccagaaagagt

SEQ ID NO: 329 - DNA, 30 bp, homo sapiens
caaacctgctttgctgaaagttgcagaaaa

SEQ ID NO: 330 - DNA, 236 bp, homo sapiens
aatatgaaatttttaaaggagtagaataccaaatgatagaaacagactgcctgaattgagaattttgatttcttaaagtgtgtttctttctaaattgctgttccttaatttgattaatttaattcatgtattatgattaaatctgaggcagatgagcttacaagtattgaaataattactaattaatcacaaatgtgaagttatgcatgatgtaaaaaatacaaacattctaatta

SEQ ID NO: 331 - DNA, 500 bp, homo sapiens
ggagcaaaatatggcctttcttctaagagatataatgttcactaatgtggttattttacattaagcctacaacatttttcagtttgcaaatagaactaatactggtgaaaatttacctaaaaccttggttatcaaatacatctccagtacattccgttcttttttttttttgagacagtctcgctctgtcgcccaggctggagtgcagtggcgcaatctcggctcactgcaacctccacctcccgggttcacgccattctcctgcctcagcctcccgagtagctgggattacgggcgcccgccaccacgcccggctaattttttgtatttttagtagagacagggtttcaccgtgttagccaggatggtctcgatctcctgaccttgtgatccacccacctcggcctcccaaagtgctgggattacaggcgtgagccactgcgcccggccacattcagttcttatcaaagaaataacccagacttaatcttgaatgatac

SEQ ID NO: 332 - DNA, 30 bp, homo sapiens
ttgtggtttctatgcccgcctccgcctccc

SEQ ID NO: 333 - DNA, 67 bp, homo sapiens
gcaacagtgcgacgggcggccattacactacagacgtcttccagatcggtctgaatggctggctgcg

SEQ ID NO: 334 - DNA, 30 bp, homo sapiens
gtgctgccggcggcggctgcgggcctctcg

SEQ ID NO: 335 - DNA, 30 bp, homo sapiens
atcgcctccggaatggcctatttgcactct

SEQ ID NO: 336 - DNA, 30 bp, homo sapiens
gcgtctgtttctcagccagcgggagccgcg

SEQ ID NO: 337 - DNA, 49 bp, homo sapiens
ctgtcagttgattatggcaagaaatccaagctggagttctccatttacc

SEQ ID NO: 338 - DNA, 30 bp, homo sapiens
gacatcgacagccagggtgcagccccgacg

SEQ ID NO: 339 - DNA, 146 bp, homo sapiens
acatcactgagtatcagtgtcaccagtacattaccaagatgacggccatcatttttagtgattaccgtttaatctgtggcttcatggatgactgcaaaaatgacatcaacattctgaaatgtggcagtattcggcttggagaaaag

SEQ ID NO: 340 - DNA, 62 bp, homo sapiens
ggcgccagctacgccgctgccgctgtcactatggcccattacaaagccgccgactcgaagcg

SEQ ID NO: 341 - DNA, 30 bp, homo sapiens
ctgagagaatggctgataatgcctgtgttc

SEQ ID NO: 342 - DNA, 106 bp, homo sapiens
cgttccaatcccaaaatctggaatgttcatagtgtcctcaatgtccttcattccctggtagacaaatccaacatcaaccgacagttggaggtatacacaagcggag

SEQ ID NO: 343 - DNA, 500 bp, homo sapiens
tttgtaattagttattataagaagatctagatcctagatattagaataaaatttattttctactgtatccatttcaaatgttaaaatattgtttaatatttttgaaatccctgagtatcaggccttgttataaataagctgcataatcaataaatagaacaagggactttttgttgataatccaaatactcaaagtttacgtaatgaaaattatagcgtgtgtgcaaactcttgagggttgattatgctgcaatttagcatgttggaacgtctagggagaaggttgactttttgcacttctgtatatagtcaaaagagagaaacctgtataatagtaagatcttattttgaataaaaacgtctataattacaaggagttttgttaaggctaatacaatgacagactgagcaaaattgcttgcaaaagtggcacagagttagcactccataccccttcaaacatgttgctttgctttcttgtggacagcttgtagtttgcc

SEQ ID NO: 344 - DNA, 30 bp, homo sapiens
atcatccgacagcagctccacgcccacctg

SEQ ID NO: 345 - DNA, 84 bp, homo sapiens
ttcaggccattaagaaggagctgacccagataaaacaaaaagtggattctctcctggaaaacctggaaaaaatggaaaaggaac

SEQ ID NO: 346 - DNA, 136 bp, homo sapiens
tgtgagaatacagaaccagtggaaacttcttctcgaaccaccacaaccataggagcgacaaccacccagttcagggtcctaactaccaccagaagagcagtgacttctcagtttcccaccagcctccctacagaag

SEQ ID NO: 347 - DNA, 500 bp, homo sapiens
gtagcctcactggagggcattgccccggaagatcaagtcgtgctcctggcaggcgcgcccctggaggatgaggccactctgggccagtgcggggtggaggccctgactaccctggaagtagcaggccgcatgcttggaggtgagtgagagaggaatgttctttgaagtaccggtaagcgtctagtgagtgtggggtgcatagtcctgacagctgagtgtcacacctatggtaatagagtacttctcactgtcttcagttcagagtgattcttcctgtttacatccctcatgttgaacacagacgtccatgggagactgagccagagtgtagttgtatttcagtcacatcacgagatcctagtctggttatcagcttccacactaaaattaggtcagaccagggcccccaaagtgctctataaaattagaagctggaagatcctgaaatgaaacttaagatttcaaggtcaaatatctgcaactttgttctcattacctat

SEQ ID NO: 348 - DNA, 30 bp, homo sapiens
ctggctgactgcaacctgttgccaaagtta

SEQ ID NO: 349 - DNA, 48 bp, homo sapiens
attctggcacacagcctggtggtgttccgagtgctcccactggccccc

SEQ ID NO: 350 - DNA, 30 bp, homo sapiens
ttgcccctcctgaagttcctgagagtggag

SEQ ID NO: 351 - DNA, 30 bp, homo sapiens
cggcccaacaagaggtgccccaagcccgtg

SEQ ID NO: 352 - DNA, 30 bp, homo sapiens
aacacaaactaccacctactcatgcaccta

SEQ ID NO: 353 - DNA, 30 bp, homo sapiens
actaaagtcctcctgccaggtagttcccac

SEQ ID NO: 354 - DNA, 500 bp, homo sapiens
tcccatctcagcttcaagggaggtgtcagcagtattatctccactttcaatctccctccaagctctactctggaggagtctgtcccactctgtcaagtggaatccttccctttccaactctacctccctcactcagctcctttcccctgatcagagaaagggatcaagggggttgggaggggggaaagagaccagccttggtccctaagcctccagaaacgtcttcttaatccccaccttttcttactcccaaaaaagaatgaacacccctgactctggagtggtgtatactgccacatcagtgtttgagtcagtccccagaggagaggggaaccctcctccatcttttttgcaacatctcatttcttccttttgctgttgcttcccccctcacacacttggttttgttctatcctacatttgagatttctattttatgttgaacttgctgctttttttcatattgaaaagatgacatcgccccaagagccaaaaataaa

SEQ ID NO: 355 - DNA, 67 bp, homo sapiens
gaggaactgctcagttaggacccagacggaaccatggaagccccagcgcagcttctcttcctcctgc

SEQ ID NO: 356 - DNA, 34 bp, homo sapiens
atggaaaccccagcgcagcttctcttcctcctgc

SEQ ID NO: 357 - DNA, 30 bp, homo sapiens
caaggcaccagactcacagttgtagaggac

SEQ ID NO: 358 - DNA, 25 bp, homo sapiens
tgccatcaagtggaacttcaccaag

SEQ ID NO: 359 - DNA, 30 bp, homo sapiens
aagcagaagctgcgggtgaagaagatccat

SEQ ID NO: 360 - DNA, 176 bp, homo sapiens
atggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaa

SEQ ID NO: 361 - DNA, 117 bp, homo sapiens
gcagagcttcaacagtactgtatgcagaatgcctgcaaggatgccctgctggtgggtgttccagctggaagtaaccccttccgggagcctagatcctgtgctttactctgaagactc

SEQ ID NO: 362 - DNA, 30 bp, homo sapiens
gcagatctacacttggatggatgcaacctt

SEQ ID NO: 363 - DNA, 49 bp, homo sapiens
tataacgatggagaggtagatgacgaggaagatgaagaagagcttggtg

SEQ ID NO: 364 - DNA, 76 bp, homo sapiens
cgcccccgcccacgcctgggtctgtgttaggtgggcggcctggcggcggtgagggcctcctgcctggactttctgc

SEQ ID NO: 365 - DNA, 30 bp, homo sapiens
gaggaggagctgctggtgcgcgtgcaggcc

SEQ ID NO: 366 - DNA, 102 bp, homo sapiens
ccgacccaggacaccctgacctctggagtcccccatcccaggcccctgtctcccaccctgctcattgtccacccaaggagttcctgtctcaacgccgtccct

SEQ ID NO: 367 - DNA, 30 bp, homo sapiens
ccttcctttgcccagatgctgagggttgga

SEQ ID NO: 368 - DNA, 30 bp, homo sapiens
tttttcacattacagtggcctgaagcacga

SEQ ID NO: 369 - DNA, 160 bp, homo sapiens
tgggaaagttggtatgaagcattaccacttaaagaggaaccagagcttctgcccaactgtcaaccttgacaaattgtggactttggtcagtgaacagacacgggtgaatgctgctaaaaacaagactggggctgctcccatcattgatgtggtgcgatcg

SEQ ID NO: 370 - DNA, 36 bp, homo sapiens
atggacacggatgatttccgcgcctgcctgatctcc

SEQ ID NO: 371 - DNA, 108 bp, homo sapiens
gatgtaattggctgtactcaggagatggatttcattctttggcctcggaatgatattgaaaaaatcgtctgtctcctgttttctaggtggaaagaatctgatgagcct

SEQ ID NO: 372 - DNA, 30 bp, homo sapiens
aggtggagatcagacgaactgtggctgcac

SEQ ID NO: 373 - DNA, 28 bp, homo sapiens
gacgggccagcccgacctcacactgcct

SEQ ID NO: 374 - DNA, 63 bp, homo sapiens
gaacgcagtagacgaaggcggcggcgtaggcggcggggatagtggcttctcgcagactccgcg

SEQ ID NO: 375 - DNA, 100 bp, homo sapiens
gccactgtgaaggagatgatgagagccccctgatcaccccctgccactgcacaggaagcctccacttcgtgcaccaggcctgcctgcagcagtggatcaa

SEQ ID NO: 376 - DNA, 110 bp, homo sapiens
gggagcagatatctgtgggttctttcaagatgctgaatatgagatgtgtgttcgctggatgcagctgggggccttttaccccttctcaagaaaccacaacaccattggga

SEQ ID NO: 377 - DNA, 69 bp, homo sapiens
tgccatcacctagctgcctgcacctgcccttcagggagatgggggtcattaaaggaaactgaacattga

SEQ ID NO: 378 - DNA, 198 bp, homo sapiens
ctctggttgaagctttgcttattgtaacaggcttttatttccaggtaatatgtcttggaagacttaattctgattagagatatagatattactggaaactaattgttttttttctattgtactctgctttatcaaagaagtaaaacatttaaatcgtactacagaaattaagatgttgtcttgcgatccttaataaat

SEQ ID NO: 379 - DNA, 30 bp, homo sapiens
ttttcctttggcaggaaggtgtcttgctgc

SEQ ID NO: 380 - DNA, 500 bp, homo sapiens
ctggagggcattgccccggaagatcaagtcgtgctcctggcaggcgcgcccctggaggatgaggccactctgggccagtgcggggtggaggccctgactaccctggaagtagcaggccgcatgcttggaggtgagtgagagaggaatgttctttgaagtaccggtaagcgtctagtgagtgtggggtgcatagtcctgacagctgagtgtcacacctatggtaatagagtacttctcactgtcttcagttcagagtgattcttcctgtttacatccctcatgttgaacacagacgtccatgggagactgagccagagtgtagttgtatttcagtcacatcacgagatcctagtctggttatcagcttccacactaaaattaggtcagaccagggcccccaaagtgctctataaaattagaagctggaagatcctgaaatgaaacttaagatttcaaggtcaaatatctgcaactttgttctcattacctattgggcgcag

SEQ ID NO: 381 - DNA, 390 bp, homo sapiens
ttccctggcccgtgctggaaaagtgagaggtcagactcctaaggtgagtgagagtattagtggtcatggtgttaggaccttttttcctttcacagctaaaccaagtccctgggctcttactcggtttgccttctccctccctggagatgagcctgagggaagggatgctaggtgtggaagacaggaaccagggcctgattaaccttcccttctccaggtggccaaacaggagaagaagaagaagaagacaggtcgggctaagcggcggatgcagtacaaccggcgctttgtcaacgttgtgcccacctttggcaagaagaagggccccaatgccaactcttaagtcttttgtaattctggctttctctaataaaaaagccacttagttca

SEQ ID NO: 382 - DNA, 30 bp, homo sapiens
ttgccatgaatgttggcaaggcccgaggct

SEQ ID NO: 383 - DNA, 30 bp, homo sapiens
agattacatgaaatcaatcaaaggggaact

SEQ ID NO: 384 - DNA, 76 bp, homo sapiens
tgaagaacagagaccagaaagagtaaaaccttttatgacaggggctgcagaacaaatcaagcacatccttgctaat

SEQ ID NO: 385 - DNA, 30 bp, homo sapiens
aggcattagagcatgggtggcatggagaaa

SEQ ID NO: 386 - DNA, 30 bp, homo sapiens
ggccctccccggaatgctggtgagattgga

SEQ ID NO: 387 - DNA, 30 bp, homo sapiens
agcagcatcatggaggtttgaagatgccgc

SEQ ID NO: 388 - DNA, 195 bp, homo sapiens
cctcctgagtagctaggactgcaggtgctccaccacgcccggctaatttttgtatttttagtagagatggggtttccccatgttggccaggctggtctcgaactcctggcctcaggtgtgatccgcccgcctccgcctccccaagcgctgagattacaggtgtgagccaccgtgcccaggccctcagtaggtttt

SEQ ID NO: 389 - DNA, 30 bp, homo sapiens
gctgggattacaggtgtgagccaccgcgcc

SEQ ID NO: 390 - DNA, 500 bp, homo sapiens
ccctttgaagttcctttttcattgttaaattaaaatttttttttttacttggatggcttaacatttttgcaagaaaaataggaagatatgaagatgatgttttggtttgtttatgaaatgcatatggcttgtcagagctcattcgacagttaaagccattgtttaaagaaatggtgctttgctctgtgtttgtgctcctgatttccctggaggttctggatgaaggctgaacacaggcttgttaatgtcagtctgtgctgaggacctcagggacttgaggttgcatttttgagcatggggtgcaggagcctttctggatttggatgtggctatggaaagaacacagaagccaaggtcatgtgcatgaaatgaggagtttgagttagtcacctcggggattttttccattttgcagtaaaatgttaaattaatgtagcctgcctctatttgttgggcaggtaatttcaaagggttatttgcctcatctcctatctttagtg

SEQ ID NO: 391 - DNA, 180 bp, homo sapiens
tatattaaattctgaatgcaatttttttttgttcccttgagaccaaaatttaagttaactgttgctggcagtctaagtgtaaatgttaacagcaggagaagttaagaattgagcagttctgttgcatgatttcccaaatgaaatactgccttggctagagtttgaaaaactaattgagcc

SEQ ID NO: 392 - DNA, 30 bp, homo sapiens
tgtgcctggctagaaaacaagcgtttattt

SEQ ID NO: 393 - DNA, 155 bp, homo sapiens
tgggctttccgggaacctggaccagactctggcccagtaggatgtccccgtgtcctccccagcagagcaggaacagggtgatacagctgtccacttcagagctaggagagatggaactgacttggcaggagatcatgtccatcaccgagctgcag

SEQ ID NO: 394 - DNA, 30 bp, homo sapiens
gtgactccaccacaggtttctagagccatc

SEQ ID NO: 395 - DNA, 30 bp, homo sapiens
aatgacgacagtatgttagggcctagtcaa

SEQ ID NO: 396 - DNA, 126 bp, homo sapiens
atacattatggtgccgagtggtaacatgggagtgtttgatccaacagaaatacacaatcgagggcagctgaagtcacacatgaaagaagccatgatcaagcttggtttccacttgctctgcttctt

SEQ ID NO: 397 - DNA, 79 bp, homo sapiens
ggcagctgaagtcacacatgaaagaagccatgatcaagcttggtttccacttgctctgcttcttcatgtatctttatag

SEQ ID NO: 398 - DNA, 76 bp, homo sapiens
gtagtaaacctattgatggcaattttgctgactgtggaagtgactcatccaaactccatgccagctgtcaacattc

SEQ ID NO: 399 - DNA, 500 bp, homo sapiens
cccagctccaacccccatccttgcctcagtttcaaccccagcttctgtcaccattcttgcctcagcctcaattcccattcttgcttcagccctagcatcaacttcagctccaacgccagccccagcagcctcttccccagctgccccagtcatcacagcaccaactatcccagcctcagccccaactgcctcagtcccacttgcccctgcctcagcttcagccccagccccagcccctaccccagtctcagccccaaatcctgccccacctgccccagcccagactcaggcacagacccacaaaccagtccagaatccactacagactacatctcagtcttcaaaacaaccaccaccatcaattaggctgccttcagctcaaacacctaatggcacagattatgtagcctcaggaaaatccatccagaccccacagtcacatggcactctgacagctgaattatgggataacaaggtggccccaccagctgtgctgaatg

SEQ ID NO: 400 - DNA, 30 bp, homo sapiens
ttctgagattctttccttgtgatctgaatg

SEQ ID NO: 401 - DNA, 271 bp, homo sapiens
gtatttgtgcagatcctggccagtacaaagtcgttgctcttgtcttatcttctcttacagagtctccctccctttatagaatgtcaaccaaagagtgccctcctcccctctcagcctcctctttagctagcctccccatctcatcacaacgcatgtctgtgacctttggtaatcatttacagtgccacacggaaccctgtattttgcacacagcaaaacaaacaatgtttagctttatttatggtatttgatgctgtaaatggaaataaat

SEQ ID NO: 402 - DNA, 30 bp, homo sapiens
tgctgcacgctcactgtatttgtgcagatc

SEQ ID NO: 403 - DNA, 104 bp, homo sapiens
agtttgatgctccaagccctctcccagagactacagagaacgtggtgtgtgccctgggcctgactgtgggtctggtgggcatcattattgggaccatcttcatc

SEQ ID NO: 404 - DNA, 500 bp, homo sapiens
tcagtggtttattgttcacaaaaaaatcttcaaaacaagtattgactttcacaaaatttaaatcataaacaggcaaaccaaacagcacactgtagctatagttgttatgtgattgttttttaattgctgtaggatcctgttctttcagcaggtgaaaaataaaacgcagttcaaatttcatggttttaattttcaactcagaagcactcaaaaatgcaaaatgtgataatgggcacttgtttaaaagaattagtgtatccagccttcactccagctggttaaaaatgttgcacttatcagcaaccctaccactttcatctgctgaaaggacaaatgtgcttggttttactattatgtaatcacaacttactttctgcttgtagttgcttaaaattatgtattttgtcttgggctgcaatttgttttatgcttattttattattactgcagtagttgactttgctgtatggaaaaataaagtgaaattgccctaataaaac

SEQ ID NO: 405 - DNA, 30 bp, homo sapiens
tctgacttccctgggagtgtacttttcctg

SEQ ID NO: 406 - DNA, 500 bp, homo sapiens
ttgcacttgctggtctttgtagcagcattcagcacaggtgccaaaatatgcttcattttgggggcagatctattttgacagtatttgactacatatagcaagagtttgaaatatgttaaacactagacatcctggttatcaaaaccaatgagcattactttcatggcagcaagtgtcatgcagttattttctgaatttgtcaaagaggcagtagtttctaacccctgttctatagtagttacaacaatttcacaacctatgtttacagattcttcataaatacatgcatactgacactataatcatgggaggtgtaaccatgattagtaggcgaggtacctaccacttttttttttttcttcccctggctacttgagtagaatgcattataccagatctggtcactttcattgaaatggtttctaattttcttcccaagtgctgttgggtttttttcttcttaaggaaaacgttgtcacttttatgttataaacttgaat

SEQ ID NO: 407 - DNA, 209 bp, homo sapiens
gcctctgcccttgcactaccttgtctgtcaccccatcccgtgtcccctcgtcccccagcctgactcctgcctgatagctcctgtgtccccatgctggtcctcctggcccaggctgcaggagccaggctggggggcctccgcacccccttgctgcgtgtgggtaattgtgttttgggggaaagtggggaatttaataaatttctggtgct

SEQ ID NO: 408 - DNA, 30 bp, homo sapiens
ggagtccacagccagacgctttcccgattc

SEQ ID NO: 409 - DNA, 30 bp, homo sapiens
gtagaagccctcatgctgagctttgtgtcc

SEQ ID NO: 410 - DNA, 30 bp, homo sapiens
cgtagggagatttgggtagaagccctcatg

SEQ ID NO: 411 - DNA, 30 bp, homo sapiens
agccctcatgctgagctttgtgtccctggt

SEQ ID NO: 412 - DNA, 30 bp, homo sapiens
agcagctggcttcactgctcaggtgattat

SEQ ID NO: 413 - DNA, 151 bp, homo sapiens
gtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattct

SEQ ID NO: 414 - DNA, 192 bp, homo sapiens
gctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaag

SEQ ID NO: 415 - DNA, 500 bp, homo sapiens
tcatgtggcattctctctgctcagtgatctcacttaaatctatatacaaagccttggtcccgtgaaaacactcgtgtgcccaccagcggccttgaagaggcaggtctgggccagatgctgggcaggaaaccccagcggcagatgggcctgtgtgcacccaacgtgatgctatgcatgtctgaccgacgatccctcgaccagaatcagattcaggagctcagtttctttttcacttgggtctctggattcctgtcatagggaaggtatatcaggaggggaagaggcctttctagaattttctttgagcaggtttacaatttagcttacatttttcgactgtgaacgtgaataggctgctttttgctttcttctttccagaccccacagtagagcacttttcacttatttgggggaggcttcaggggactgttctcaccttaactcagccagaaagatgccctagttgtgatcaaaggtaactcgaggtggagggtagccct

SEQ ID NO: 416 - DNA, 132 bp, homo sapiens
gctccgggcagcatcagtgtgacagctccaccgtctgcttcaacaccgtgggttcatacagctgccgctgccgcccaggctggaagcccagacacggaatcccgaataaccaaaaggacactgtctgtgaag

SEQ ID NO: 417 - DNA, 187 bp, homo sapiens
ccccggaaagccagcgccacatgcagttcggccacagcagcagcctccagtggcctggaggagtggactagccggtccccgcggcagcggagttcagcctcgtcacctgagcaccctgagctgcatgctgaattgggcatgaagcccccttccccaggcactgttttagctcttgccaaacctcctt

SEQ ID NO: 418 - DNA, 30 bp, homo sapiens
agattacatgaaatcaatcaaagggaaact

SEQ ID NO: 419 - DNA, 160 bp, homo sapiens
cccacgacctgccccagcagttggagaggctgaagataaagaaaatcagcaagccaccagtggtccaaaccagccgtctgttcgccgtggatatcggcgtccctacaattaccggcgtcgcccgcgtcctcctaacgctccttcacaagatggcaaagag

SEQ ID NO: 420 - DNA, 108 bp, homo sapiens
ggtgcagaagctgccaatgtgactggcccggatggagttcctgtggaagggagtcgttacgctgcagatcggcgccgttacagacgtggctactatggaaggcgccgt

SEQ ID NO: 421 - DNA, 50 bp, homo sapiens
tctgtgagatgaatttaaaagtgcctcagaatgcagttgcccttattgtg

SEQ ID NO: 422 - DNA, 208 bp, homo sapiens
cgtgaatgcatctcagtccacgtggggcaggcaggtgtccagatgggcaatgcctgctgggagctctattgcttggaacatgggattcagcctgatgggcagatgcccagtgacaagaccattggtggaggggacgactccttcaccaccttcttctgtgaaactggtgctggaaaacacgtaccccgggcagtttttgtggatctgg

SEQ ID NO: 423 - DNA, 89 bp, homo sapiens
cctgccccgagcccctgttcagtgagctaggccgctctcgtctggcaaaggtggtgaagacgttgaaggagattcaccttgccttcctc

SEQ ID NO: 424 - DNA, 30 bp, homo sapiens
cttttacccgacaagcactgaacctaccag

SEQ ID NO: 425 - DNA, 30 bp, homo sapiens
cctgaggaggccaagtacaagttgtgcaaa

SEQ ID NO: 426 - DNA, 30 bp, homo sapiens
caacaagatagcaggttatgtcacgcatct

SEQ ID NO: 427 - DNA, 30 bp, homo sapiens
agccttccttcctgggcatggagtcctgtg

SEQ ID NO: 428 - DNA, 30 bp, homo sapiens
ttccagccttccttccctgggcatggagtc

SEQ ID NO: 429 - DNA, 51 bp, homo sapiens
cggggtcaatgtcagatttagtcatgctgttattttagcccagtggtccag

SEQ ID NO: 430 - DNA, 116 bp, homo sapiens
gtgcggagctctggactgggattaagaaggggcccccacgcaaactcaaattccctgagcctcaagaggtggtggaagagttgaagaagtacctgtcgtagggagatttgggtaga

SEQ ID NO: 431 - DNA, 30 bp, homo sapiens
gcccggacggcagcagtgcggagctctgga

SEQ ID NO: 432 - DNA, 220 bp, homo sapiens
catccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgg

SEQ ID NO: 433 - DNA, 89 bp, homo sapiens
aaaattatatagatatttgcttttctgctggttttttttttttaattgcaactgcttttctgccgtgcctctcttccctacccgtgatg

SEQ ID NO: 434 - DNA, 55 bp, homo sapiens
gacgatgccccgaattcccaccctgaagaacctagaggatcttgttactgaatac

SEQ ID NO: 435 - DNA, 30 bp, homo sapiens
cacgggaacttttcggcctggagtggtgtg

SEQ ID NO: 436 - DNA, 57 bp, homo sapiens
aggcattgtttttaagaaaaacatgtcatgtaggttgtctaaaaataaaatgcattt

SEQ ID NO: 437 - DNA, 30 bp, homo sapiens
aaactcatttgagagaatgccttttagttt

(19) United States
(12) Patent Application Publication
(10) Pub. No.: US 2009/0269744 A1
(43) Pub. Date: Oct. 29, 2009

(54) CANCER DETECTION METHOD

(75) Inventors: Alexander Krause, Grenoble (FR); Philippe Leissner, Eybens (FR); Malick Paye, Lyon (FR); Bruno Mougin, Lyon (FR); Fabien Schweighoffer, Nogent Sur Marne (FR); Laurent Bracco, La Garenne Colombes (FR)

Correspondence Address: DON J. PELTO;Sheppard, Mullin, Richter & Hampton LLP, 1300 I STREET, NW, 11TH FLOOR EAST, WASHINGTON, DC, 20005, US

(73) Assignees: EXONHIT THERAPEUTICS SA, Paris (FR); BIOMERIEUX SA, Marcy L'Etoile (FR)

(21) Appl. No.: 12/091,835

(22) PCT Filed: Oct. 26, 2006
(86) PCT No.: PCT/FR2006/051108

§ 371 Date: May 7, 2009

(30) Foreign Application Priority Data
Oct. 28, 2005 (FR) 0511080
Mar. 31, 2006 (FR) 0602824

(51) Int. Cl. C12Q 1/68; C07H 21/00

(52) U.S. Cl. 435/6; 536/24.3

(57) ABSTRACT

The present application concerns methods and compositions which can be used to detect cancer in mammals, in particular in humans. It notably describes serum markers of cancers and their uses in diagnosis methods. It also concerns tools and/or kits which can be used to implement these methods (reagents, probes, primers, antibodies, chips, cells, etc.), their preparation and their uses. The invention can be used to detect the presence or the progression of a cancer, particularly breast cancer, including at an early stage.

The present application relates to methods and compositions which can be used to detect cancer in mammals, humans in particular. It notably describes serum markers for cancers and their uses in diagnostic methods. It also concerns tools and/or kits which can be used to implement these methods (reagents, probes, primers, antibodies, chips, cells, etc.) their preparation and their uses. The invention can be used to detect the presence or the progression of a cancer in mammals, particularly breast cancer, including the early phase thereof.

In women, breast cancer is the leading cause of cancer deaths in industrialized countries. Age is the greatest risk factor. Risk increases by 0.5% per year of age in Western countries. Other risk factors are known such as the number of pregnancies and the age of the first pregnancy, breast feeding, onset of puberty and menopause, estrogen treatments after the menopause, stress and nutrition.

The diagnosis of breast cancer is generally made by mammography. It is estimated however that the minimum tumor size which can be detected by mammography is 1 cm, which represents a past progression of 8 years on average at the time of diagnosis. Small tumors are much less malignant than can be extrapolated from their size: the aggressiveness of large tumors is not only due to their size but also to their <<inherent aggressiveness >>, which increases with the age of a tumor (Bucchi et al., Br J Cancer 2005, p. 156-161; Norden T, Eur J Cancer 1997, p. 624-628).

The benefit of mammography has been demonstrated in hundreds of thousands of patients these last 30 years. However, these tests have biases such as age dependence on sensitivity, hormone therapy, the number of mammographies performed, experience of the medical practitioner and others (see Fletcher and Elmore, NEJM 2003, p. 1672f or Baines C J, Breast J 2005, S7-10).

Analysis of the expression of a panel of target genes is also relevant in the fight against breast cancer, and particular mention may be made of the analysis of a panel of 176 genes which are expressed differentially between patients expressing the ER receptor and patients not expressing the ER receptor (Bertucci et al, Human Molecular Genetics, 2000; 9: 2981-2991). The analysis may also be cited of a panel of 37 genes which can be used for early diagnosis of breast cancer (Sharma et al, Breast cancer Research 2005, 7: R634-R644). However, the patients included in this study were all suspected of having breast disease (<<suspect initial mammogram <<), and this panel of genes could be ill-adapted for routine tests to diagnose breast cancer prior to any mammography.

There is therefore a major need for diagnostic tools and tests able to detect cancer reliably, simply and at an early stage.

The present application provides a group of biological markers which can be used alone or in combination(s), preferably in combination(s), to detect, characterize or follow up, in reliable manner, the presence or progression of a cancer in mammals, preferably breast cancer. The invention is particularly advantageous in that it can be implemented using whole blood, without requiring tissue biopsy or separation steps.

More particularly, the present application results from the identification of serum genetic markers characteristic of human patients having breast cancer. These markers correspond for example to variations in splicing or in gene expression levels and, either alone or advantageously in combination, can allow the detection in patients of the presence or stage of progression of a cancer. They advantageously enable the detection of the presence of a breast cancer right from its early stages (namely stages I and II, i.e. in particular at a stage when mammography is ineffective), in a manner that is reliable and simple using a sample of whole blood. They can also be used to detect early stage breast cancers which cannot be detected by mammography since they lie below its detection threshold.

One subject-matter of the present application concerns a method (in vitro or ex vivo) to detect the presence or risk of developing a cancer in a mammal, comprising the determination in a biological sample of the mammal, of the presence (or absence or (relative) quantity) of one or preferably several target molecules chosen from among:
a) nucleic acids containing a sequence chosen from among SEQ ID NOs: 1-437 or a distinctive fragment thereof having at least 15, preferably at least 16, 17, 18, 19, 20, 25 or 30 consecutive bases,
b) nucleic acids having a sequence complementary to a sequence according to a),
c) functional analogs of nucleic acids according to a) or b), or
d) polypeptides encoded by nucleic acids according to a) to c),
   the presence (or absence or (relative) quantity) of said target molecules in the sample being an indication of the presence or the risk of developing a cancer in said mammal.

In one particular variant of embodiment, the method comprises the combined determination of the presence, or absence, or (relative) quantity of at least 5, 10, 15, 20, 30, 40, 50, 60, 70 or more target molecules such as defined above. “Combined” determination designates the fact that a hybridization profile (or signature) involving several markers is determined. Combined determination is typically performed simultaneously i.e. by global measurement of an expression profile. Nonetheless, combined determination may also be performed by parallel or sequential measurements of several markers, leading to identification of a profile. With the invention, it is effectively possible to establish and determine a hybridization profile (or signature) on a group of markers in order to assess the presence or risk of developing a cancer in a mammal. The hybridization profile is typically performed using a combination of several markers chosen from among the above-indicated targets, for example containing all these targets.

In one particular embodiment, the method of the invention comprises the determination of the presence (or absence or (relative) quantity) in a mammalian biological sample of at least 5 separate target molecules chosen from among those defined above, preferably at least 10.

In preferred embodiments, the method of the invention comprises the combined determination of the presence (or absence or (relative) quantity) in a mammalian biological sample of particular sub-groups of target molecules chosen from among those indicated above. Said sub-groups, described in the examples, are particularly adapted for the detection, notably at an early stage, of the presence of a breast cancer in patients on the basis of a sample of whole blood.

Therefore, in one particular embodiment, the method of the invention comprises the combined determination, in a mammalian biological sample, of the presence (or absence or (relative) quantity) of the nucleic acids of an entire panel of targets (or signatures) comprising markers such as defined under items a) to d) above, preferably all the molecules of one of panels 1 to 11 defined in the present application.

Therefore in one particular embodiment, the method of the invention comprises the combined determination in a mammalian biological sample of the presence (or absence or (relative) quantity) of all the nucleic acids of Panel 1, comprising the sequences shown Table 4, column 1, or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by these nucleic acids. The examples given in the present application effectively show that this panel of markers can be used for the predictive detection of the presence, the risk of developing, or the stage of progression of a cancer. In one particular embodiment, the method also comprises the detection of one or more of the other target molecules such as defined previously.

In another particular embodiment, the method of the invention comprises the combined determination in a mammalian biological sample of the presence (or absence or (relative) quantity) of nucleic acids containing the sequences indicated in SEQ ID NOs: 18, 19, 23, 26, 51, 52, 53, 54, 55, 69, 80, 125, 145, 148, 225, 228, 240 and 312 (PANEL 2) or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by these nucleic acids. The examples given in the present application effectively show that this panel of markers enables predictive detection of the presence, risk of developing, or stage of progression of a cancer. In one particular embodiment, the method also comprises the detection of one or more other target molecules such as those defined previously.

In another particular embodiment, the method of the invention comprises the combined determination, in a mammalian biological sample, of the presence (or absence or (relative) quantity) of nucleic acids containing the sequences indicated in SEQ ID NOs: 18, 19, 23, 26, 27, 51, 52, 53, 54, 55, 69, 80, 125, 145, 148, 161, 188, 225, 228, 240, 280 and 312 (PANEL 3) or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by the nucleic acids. The examples given in the present application effectively show that this panel of markers enables the predictive detection of the presence, risk of developing, or stage of progression of a cancer. In one particular embodiment, the method also comprises the detection of one or more other target molecules, such as those defined previously.

In another particular embodiment, the method of the invention comprises the combined determination, in a mammalian biological sample, of the presence (or absence or (relative) quantity) of nucleic acids containing the sequences indicated in SEQ ID NOs: 13, 16-19, 23, 26-28, 47, 51-55, 58, 69, 80, 81, 89, 116, 121, 125, 145, 148, 158, 160, 161, 164, 189, 190, 225, 229, 240, 248, 280, 281, 284, 299, 300, 310 and 312 (PANEL 4) or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by these nucleic acids. The examples given in the present application effectively show that this panel of markers enables the predictive detection of the presence, risk of developing, or stage of progression of a cancer. In one particular embodiment the method also comprises the detection of one or more of the other target molecule such as defined previously.

In another particular embodiment the method of the invention comprises the combined determination, in a mammalian biological sample, of the presence (or absence or (relative) quantity) of nucleic acids containing the sequences indicated in SEQ ID NOs: 7, 13, 14, 16-19, 23-28, 47, 51-55, 58, 69, 80, 81, 89, 116, 121, 125, 137, 139, 145, 148, 158, 160, 161, 164, 189, 190, 225, 228, 229, 240, 245, 248, 252, 280, 281, 284, 290, 298-300, 310 and 312 (PANEL 5) or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by these nucleic acids. The examples given in the present application effectively show that this panel of markers enables the predictive detection of the presence, risk of developing, or stage of progression of a cancer. In one particular embodiment the method also comprises the detection of one or more other target molecules such as defined previously.

In another particular embodiment, the method of the invention comprises the combined determination, in a mammalian biological sample, of the presence (or absence or (relative) quantity) of the nucleic acids containing the sequences indicated in SEQ ID NOs: 5, 7, 13, 14, 16-20, 23-28, 47, 51-55, 58, 64, 69, 80, 81, 88-90, 116, 121, 125, 137, 139, 145, 148, 158, 160, 161, 164, 188-191, 208, 222, 225, 228, 229, 236, 240, 242, 245, 248, 252, 280, 281, 284, 290, 298-300 and 309-312 (PANEL 6) or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by these nucleic acids. The examples given in the present application effectively show that this panel of markers enables the predictive detection of the presence, risk of developing, or stage of progression of a cancer. In another particular embodiment, the method also comprises the detection of one or more other target molecules such as defined previously.

In another particular embodiment, the method of the invention comprises the combined determination, in a mammalian biological sample, of the presence (or absence or (relative) quantity) of all the nucleic acids of Panel 7, comprising the sequences indicated in Table 4, or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by these nucleic acids. The examples given in the present application effectively show that this panel of markers enables the predictive detection of the presence, risk of developing, or stage of progression of a cancer. In one particular mode, the method also comprises the detection of one or more other target molecules such as defined previously.

In another particular embodiment, the method of the invention comprises the combined determination, in a mammalian biological sample, of the presence (or absence or (relative) quantity) of all the nucleic acids in Panel 8, comprising the sequences indicated in Table 4, or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by these nucleic acids. The examples given in the present application effectively show that this panel of markers enables the predictive detection of the presence, risk of developing, or stage of progression of a cancer. In one particular embodiment, the method aloes comprises the detection of one or more of the other target molecules such as defined previously.

In another particular embodiment, the method of the invention comprises the combined determination, in a mammalian biological sample, of the presence (or absence or (relative) quantity) of all the nucleic acids in Panel 9, comprising the sequences indicated in Table 4, or a distinctive fragment thereof having at least 15, preferably at least 16, 17, 18, 19, 20, 25 or 30 consecutive bases, or having a sequence complementary thereto and/or functional analogs thereof and/or polypeptides encoded by these nucleic acids. The examples given in the present application effectively show that this panel of markers enables the predictive detection of the presence, risk of developing, or stage of progression of a cancer. In one particular embodiment, the method also comprises the detection of one or more of the other target molecules such as defined previously.

In another particular embodiment, the method of the invention comprises the combined determination, in a mammalian biological sample, of the presence (or absence or (relative) quantity) of all the nucleic acids in Panel 10, comprising the sequences indicated in Table 4, or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by these nucleic acids. The examples given in the present application effectively show that this panel of markers enables the predictive detection of the presence, risk of developing, or stage of progression of a cancer. In one particular embodiment, the method also comprises the detection of one or more of the other target molecules such as defined previously.

In another particular embodiment, the method of the invention comprises the combined determination, in a mammalian biological sample, of the presence (or absence or (relative) quantity) of the nucleic acids comprising the sequences indicated in SEQ ID No: 23, 52, 53, 148 and 225 (PANEL 11), or a distinctive fragment thereof having at least 15, preferably at least 16, 17, 18, 19, 20, 25 or 30 consecutive bases, or having a sequence complementary thereto and/or functional analogs thereof derived from other species, and/or polypeptides encoded by these nucleic acids. The examples given in the present application effectively show that this panel of markers enables the predictive detection of the presence, risk of developing, or stage of progression of a cancer. In one particular embodiment, the method also comprises the detection of one or more of the other target molecules such as defined previously.

In one specific embodiment, the method of the invention comprises the determination, in a mammalian biological sample, of the presence (or absence or (relative) quantity) of the nucleic acids respectively comprising the sequences indicated in SEQ ID NOs: 1-437 or a distinctive fragment thereof having at least 15, preferably at least 16, 17, 18, 19, 20, 25 or 30 consecutive bases, or nucleic acids having a complementary sequence thereof.

A further particular subject of the invention lies in a method of detecting the presence or risk of developing a cancer in a mammal, which comprises contacting, under conditions allowing hybridization between complementary sequences, the nucleic acids derived from a blood sample of the mammal with a set of probes specific to the following target molecules:
a) the nucleic acids comprising the sequences indicated in one of PANELS 1 to 11 such as defined in the foregoing, or a distinctive fragment thereof having at least 15, preferably at least 16, 17, 18, 19, 20, 25 or 30 consecutive bases, and/or
b) the nucleic acids having a sequence complementary to sequences according to a), and/or
c) functional analogs of the nucleic acids according to a) or b) derived from another species,
   to obtain a hybridization profile, the hybridization profile being characteristic of the presence, or risk of developing a breast cancer in this mammal.

Also, analysis of the different genes identified in the invention, whose expression is altered in patients, shows that they belong to families of genes involved in cell signaling pathways or in common regulating mechanisms. Therefore, in particular this analysis evidences numerous genes involved in signaling cascades used for the transducing of messages initiated by TLR stimulation, in the secretion of cytokins or in the activation of T lymphocytes. The invention therefore evidences the fact that alterations in these signaling cascades occur in patients suffering from cancer, and that any gene or RNA taking part in these cascades or any deregulation of these genes or RNA may form a marker of the presence of or the predisposition to cancer. Said alterations may occur during oxidative stress imposed by tumor cells on monocytes, macrophages and dendritic cells. This stress is part of the consequences of the different cell interactions occurring between the immune system and the cancer cells at the tumor. The evidencing, in blood circulation, of molecular events revealing alterations in the signaling cascades involved in immune responses therefore represents a new tool and a novel approach allowing the evidencing of a tumor in the body on the basis of a blood sample.

Therefore one particular subject-matter of the invention concerns a method (in vitro ou ex vivo) to detect the presence or risk of developing a cancer in a mammal, comprising the determination of the presence (or absence) in a mammalian biological sample, preferably a blood (derived) sample, of an alteration in a gene or RNA taking part in a signaling pathway involved in immune response (innate or acquired), the presence of said alteration being indicative of the presence or risk of developing a cancer in this mammal. The signaling pathway involved in immune response is advantageously chosen from among TLR stimulation, cytokin secretion or T-lymphocyte activation.

Initial stimulation of the dendritic cells represents innate response and is made via toll-like receptors (TLRs). Multiple TLRs react to different ligands which may be carried by pathogens or tumor cells and induce the production of different pro-inflammatory cytokins by the dendritic cells. This phenomenon is accompanied by the presentation of antigens to naïve T-cells, thereby initiating a specific, acquired immune response. Among the molecular actors of these signaling cascades, those which may be preferably cited are receptors, adaptors, enzymes, factors involved in the regulation of gene expression, chemokins, cytokins and interleukins.

One particular subject of the invention is a method for the in vitro or ex vivo detection of the presence or risk of developing a cancer in a mammal, comprising the determination of the presence (or absence) in a mammalian biological sample, preferably a blood (derived) sample, of an alteration in a gene or RNA involved in regulating the signaling pathway which controls the phenomenon of innate immunity. More particularly, this phenomenon mobilizes the cascade initiated by the TLRs and regulates the activity of the macrophage and dendritic cells.

Another particular subject of the invention is a method for the in vitro or ex vivo detection of the presence or risk of developing a cancer in a mammal, comprising the determination of the presence (or absence) in a mammalian biological sample, preferably a blood (derived) sample, of an alteration in a gene or RNA involved in regulating the signaling pathway which controls the phenomenon of acquired or adaptive immunity. More particularly, this phenomenon involves T-lymphocyte receptors (TCRs).

A further particular subject of the invention is a method for the in vitro or ex vivo detection of the presence or risk of developing a cancer in a mammal, comprising the determination of the presence (or absence) in a mammalian biological sample, preferably a blood (derived) sample, of an alteration in a gene or RNA involved in the transition, coordination between innate and acquired immunities. More particularly, these genes are involved in the biosynthesis of lipid molecules from precursors such as arachidonic acid.

One particular subject of the invention therefore lies in a method (in vitro or ex vivo) to detect the presence or risk of developing a cancer in a mammal, comprising the determination of the presence (or absence) in a mammalian biological sample, preferably a blood (derived) sample, of an alteration in a gene or RNA involved in the stimulation of TLRs, in the secretion of cytokins, or in the activation of T-lymphocytes, said gene or RNA advantageously being chosen from among receptors, adaptors, enzymes, factors involved in the regulation of gene expression, chemokins, cytokins and interleukins, the presence of said alteration being indicative of the presence or risk of developing a cancer in this mammal.

Alteration of a gene or RNA in the meaning of the invention is any altered expression, namely deregulation of splicing in particular, leading to the onset of particular spliced forms or to a change in the (relative) quantity or ratio between the different splice forms.

As is described in the remainder of the text, the present application describes the identification of splicing deregulations in the actors of signaling cascades involved in innate and acquired immunities, found in the blood of cancer patients (Table 5). Oligonucleotides, derived from RNA sequences whose expressions are affected by splicing alterations, can be deposited or synthesized on any solid carrier and hybridized with nucleic probes derived from control blood samples and blood samples from cancer patients allowing the selection of the most discriminating oligonucleotides. More broadly, the oligonucleotides can be chosen to represent any mRNA encoding any protein involved in innate and acquired immunities. More particularly, these oligonucleotides may derive from the genes indicated in Table 6. Alterations may also be detected at the structure or expression levels of polypeptides encoded by these genes or RNA, for example using specific antibodies as is described in detail in the remainder of the text.

One particular subject of the invention therefore lies in a method (in vitro ou ex vivo) to detect the presence or risk of developing a cancer in a mammal, comprising the determination of the presence (or absence) in a mammalian biological sample, preferably a blood (derived) sample, of an alteration in at least one, preferably at least 2, 3, 4, 5, 6, 7, 8, 9 or 10 genes or corresponding RNAs indicated in Table 5, in particular altered splicing of said gene or RNA, the presence of said alteration being an indication of the presence or risk of developing a cancer in this mammal.

Another particular subject of the invention therefore lies in a method (in vitro or ex vivo) to detect the presence or risk of developing a cancer in a mammal, comprising the determination of the presence (or absence) in a mammalian biological sample, preferably a blood (derived) sample, of an alteration in at least one, preferably at least 2, 3, 4, 5, 6, 7, 8, 9 or 10 genes or corresponding RNAs indicated in Table 6, in particular altered splicing of said gene or RNA, the presence of said alteration being an indication of the presence of risk of developing a cancer in this mammal.

### Target Molecule

The present invention is based on the evidencing and characterizing of serum biological events characteristic of the presence of a breast cancer in a human patient. These events form (bio)markers whose detection in a patient, preferably in combination, allows the determination, even at an early stage, of the risk of developing said cancer or the presence of said cancer. In the meaning of the invention, the terms markers and transcripts are used interchangeably, except when the context gives them a specific meaning.

The identified biological events typically correspond to changes in the regulation of gene expression. This may concern partial or full inhibition of the expression of genes or RNA, or some forms of genes of RNA, an increase in the expression of genes or some forms of genes or RNA, the onset or disappearance of gene splicing forms, etc.

The invention is therefore based on the detection, in a sample, of one or more target molecules representing biological events thus identified. As indicated above, these target molecules may be chosen from among:
a) the nucleic acids comprising a sequence chosen from among SEQ ID NOs: 1-437 or a distinctive fragment thereof having at least 15, preferably at least 16, 17, 18, 19, 20, 25 or 30 consecutive bases,
b) nucleic acids having a sequence complementary to a sequence according to a),
c) functional analogs of nucleic acids according to a) or b), or
d) polypeptides encoded by the nucleic acids according to a) to c).

The target molecule can be the complete sequence of the gene or RNA or protein corresponding to sequences SEQ ID NOs: 1-437, or a distinctive fragment thereof i.e. a fragment whose sequence is specific to said gene or RNA, or to said protein, and/or comprises a variability domain (splicing, deletion, polymorphism, etc.) representing the biological event to be detected. The complete list of markers and the corresponding genes are indicated in Table 1.

The term <<functional analog >> designates an analog derived from another mammalian species. Sequences SEQ ID NOs: 1-437 were identified from humans, and these sequences form efficient markers adapted for the detection of cancer in human patients. Nonetheless, for application of the methods of the invention to other species of mammals, it is generally preferable to use functional analogs of these sequences, characterized in the species under consideration. These analogs can be identified using any technique known to persons skilled in the art, notably having regard to the sequences provided in the application and the names of the corresponding genes.

In one particular embodiment, the method comprises the determination of the presence of at least one nucleic acid according to a) to c).

In one very particular embodiment, the method is used to detect a cancer in a human individual and comprises the determination of the presence of at least one nucleic acid according to a) or b). Further preferably, the method comprises the combined detection of the presence (or absence) or (relative or absolute) quantity of a panel of target markers, such as defined in the present application (Panels 1 to 11 or Tables 5 and 6).

One particular embodiment of the invention lies in a method to detect the presence or risk of developing a breast cancer in a mammal, comprising the combined determination of the presence or (relative or absolute) quantity, in a mammalian blood sample, of a group of molecules comprising at least the following target molecules:
a) the nucleic acids comprising the sequences of PANEL 1 or 11 given in table 4, or a distinctive fragment thereof having at least 15, preferably at least 16, 17, 18, 19, 20, 25 or 30 consecutive bases, and/or
b) the nucleic acids having a sequence complementary to sequences according to a), and/or
c) functional analogs of nucleic acids according to a) or b) derived from another species, and/or
d) polypeptides encoded by the nucleic acids according to a) to c),
   the presence of said target molecules in the sample being an indication of the presence or risk of developing a breast cancer in this mammal.

One particular embodiment of the invention lies in a method to detect the presence or risk of developing a breast cancer in a mammal, comprising the combined determination of the presence or quantity, in a blood sample of the mammal, of the following target molecules:
a) the nucleic acids comprising the sequences indicated in SEQ ID NOs: 18, 19, 23, 26, 51, 52, 53, 54, 55, 69, 80, 125, 145, 148, 225, 228, 240 and 312 (PANEL 2) or a distinctive fragment thereof having at least 15, preferably at least 16, 17, 18, 19, 20, 25 or 30 consecutive bases, and/or
b) the nucleic acids having a sequence complementary to sequences according to a), and/or
c) functional analogs of the nucleic acids according to a) or b) derived from another species, and/or
d) polypeptides encoded by the nucleic acids according to a) to c),
   the presence of said molecule sin the sample being an indication of the presence or risk of developing a breast cancer in this mammal.

One particular embodiment of the invention lies in a method to detect the presence or risk of developing a breast cancer in a mammal, comprising the combined determination of the presence or quantity, in a blood sample of the mammal, of the following target molecules:
a) the nucleic acids comprising the sequences of PANEL 10 indicated in Table 4, or a distinctive fragment thereof having at least 15, preferably at least 16, 17, 18, 19, 20, 25 or 30 consecutive bases, and/or
b) nucleic acids having a sequence complementary to sequences according to a), and/or
c) functional analogs of the nucleic acids according to a) or b) derived from another species, and/or
d) polypeptides encoded by the nucleic acids according to a) to c),
   the presence of said target molecules in the sample being an indication of the presence or risk of developing a breast cancer in this mammal.

A further specific subject of the invention lies in a method to detect the presence or risk of developing a breast cancer in a mammal, comprising the detection, in a blood sample of the mammal, of the following target molecules:
a) the nucleic acids comprising the sequences indicated in SEQ ID NOs: 18, 19, 23, 26, 51, 52, 53, 54, 55, 69, 80, 125, 145, 148, 225, 228, 240 and 312 (PANEL 2) or a distinctive fragment thereof having at least 15, preferably at least 16, 17, 18, 19, 20, 25 or 30 consecutive bases, and/or
b) the nucleic acids having a sequence complementary to sequences according to a), and/or
c) the functional analogs of nucleic acids according to a) or b) derived from another species, and/or
d) the polypeptides encoded by the nucleic acids according to a) to c),
   the presence, the absence or the (relative) quantity of these target molecules in the sample being an indication of the presence or risk of developing a breast cancer in this mammal.

The invention also enables the defining of additional panels, comprising at least some markers such as defined previously, which may optionally be combined with other markers. Said panels may be obtained by testing the presence or absence of these markers in patient samples, to define other predictive combinations, possibly specific to particular pathologies.

### Detection of a Nucleic Acid

Different techniques allowing the detection of a species of nucleic acid in a sample can be used in the present invention, such as Northern Blot for example, or selective hybridization, the use of carriers coated with probe oligonucleotides, nucleic acid amplification such as RT-PCR, quantitative PCR or ligation-PCR, etc. These methods may comprise the use of a nucleic probe (e.g. an oligonucleotide) capable of detecting, either selectively or specifically, the target nucleic acid in the sample. Amplification can be conducted according to different methods known per se to persons skilled in the art, such as PCR, LCR, transcription mediated amplification (TMA), strand displacement amplification (SDA), NASBA, the use of allele-specific oligonucloetides (ASO), allele specific amplification, Southern Blot, single strand conformation analysis SSCA, in situ hybridization (e.g., FISH), gel migration, analysis of heteroduplexes, etc.

According to one preferred embodiment, the method comprises the detection of the presence or absence or (relative) quantity of a nucleic acid according to a) to c) by selective hybridization or selective amplification.

Selective hybridization is typically performed using nucleic probes, preferably immobilized on a carrier such as a solid or semi-solid support having at least one surface, whether planar or not, allowing the immobilization of nucleic probes. Said supports are for example a slide, bead, membrane, filter, column, plate etc. They may be made in any compatible material in particular glass, silica, plastic, fiber, metal, polymer, etc. The nucleic probes may be any nucleic acid (DNA, RNA, PNA, etc.), preferably single strand, comprising a sequence specific to a target molecule such as defined under a) to c) above. The probes typically comprise 5 to 400 bases, preferably 8 to 200, more preferably less than 100, and further preferably less than 75, 60, 50, 40 or even 30 bases. The probes may be synthetic oligonucleotides produced on the basis of the sequences of target molecules of the invention using conventional synthesis techniques. Said oligonucleotides typically comprise 10 to 50 bases, preferably 20 to 40, for example around 25 bases. In one particularly advantageous embodiment, several different oligonucleotides (or probes) are used to detect the same target molecule. These may be oligonucleotides specific to different regions of the same target molecule, or aligned differently on one same region. It is also possible to use pairs of probes, of which one member is fully matched with the target molecule, and another is mismatched thereby enabling background noise to be estimated. In the following examples, 6 to 11 pairs of oligonucleotides with 25 bases were used for each target molecule.

The probes may be previously synthesized then deposited on the carrier, or synthesized directly in situ on the carrier, using methods known per se to those skilled in the art. The probes may also be made using genetic techniques e.g. by amplification, recombination, ligation, etc.

The probes so defined form another subject of the present application, as well as their uses (essentially in vitro) for cancer detection. In particularly preferred manner, a set of nucleic probes is used comprising all or a fragment of at least 15 consecutive bases, preferably at least 17, 19, 20, 22 or 25 consecutive bases of each of sequences SEQ ID NO: 1-437, or a complementary strand thereof, advantageously immobilized on a carrier.

Hybridization can be performed under conventional conditions, known to persons skilled in the art and which may be adjusted by such persons (Sambrook, Fritsch, Maniatis (1989) Molecular Cloning, Cold Spring Harbor Laboratory Press). In particular, hybridization can be conducted under conditions of strict, medium or low stringency depending on the desired level of sensitivity, the quantity of material available etc. For example, appropriate conditions for hybridization include a temperature of between 55 and 63° C. for 2 to 18 hours on low density carriers. Other hybridization conditions may be necessary for high density carriers, such as a hybridization temperature of between 45 and 55° C. After hybridization, different washings may be performed to remove non-hybridized molecules, typically in SSC buffers containing SDS such as a buffer containing 0.1 to 10×SSC and 0.5-0.01% SDS. Other washing buffers containing SSPE, MES, NaCl or EDTA may also be used.

In one typical embodiment, the nucleic acids (or chips or carriers) are pre-hybridized in a hybridization buffer (Rapid Hybrid Buffer, Amersham) typically containing 100 μg/ml salmon sperm DNA at 65° C. for 30 min. The nucleic acids of the sample are then contacted with the probes (typically applied to the carrier or chip) at 65° C. for 2 to 18 hours. Preferably, the nucleic acids of the sample are previously labeled using any known labeling (radioactive, enzymatic, fluorescent, luminescent, etc.). The carriers are then washed in a 5×SSC, 0.1% SDS buffer at 65° C. for 30 min, then in a 0.2×SSC, 0.1% SDS buffer. The hybridization profile is analyzed using conventional techniques e.g. by measuring the labeling on the carrier using an appropriate instrument (e.g. InstantImager, Packard Instruments). Hybridization conditions can evidently be adjusted by those skilled in the art, for example by modifying the hybridization temperature and/or saline concentration of the buffer, and through the addition of auxiliary substances such as formamide or single-strand DNA.

One particular subject of the invention lies in a method to detect the presence or risk of developing a breast cancer in a mammal, comprising the contacting, under conditions enabling hybridization between complementary sequences, of nucleic acids derived from a blood sample of the mammal with a set of probes specific to at least the following target molecules:
a) the nucleic acids containing the sequences of one of panels 1 to 11 defined previously, or a distinctive fragment thereof having at least 15, preferably at least 16, 17, 18, 19, 20, 25 or 30 consecutive bases, and/or
b) the nucleic acids having a sequence complementary to sequences according to a), and/or
c) the functional analogs of nucleic acids according to a) or b) derived from another species,
   to obtain a hybridization profile, the hybridization profile being characteristic of the presence or risk of developing a breast cancer in this mammal.

One particular subject of the invention therefore lies in a method to detect the presence or risk of developing a breast cancer in a mammal, comprising the contacting, under conditions enabling hybridization between complementary sequences, of nucleic acids derived from a blood sample of the mammal with a set of probes specific to the following target molecules:
a) the nucleic acids containing the sequences indicated in one of PANELS 1, 2 10 or 11 such as defined above, or a distinctive fragment thereof having at least 15, preferably at least 16, 17, 18, 19, 20, 25 or 30 consecutive bases, and/or
b) the nucleic acids having a sequence complementary to sequences according to a), and/or
c) the functional analogs of the nucleic acids according to a) or b) derived from another species,
   to obtain a hybridization profile, the hybridization profile being characteristic of the presence or risk of developing a breast cancer in this mammal.

In particular embodiments, the methods of the invention also use other target molecules and/or other probes, in particular the sub-groups of target molecules mentioned in the present application.

Therefore, one other particular subject of the invention lies in a method to detect the presence or risk of developing a cancer in a mammal, comprising the contacting under conditions enabling hybridization between complementary sequences, of nucleic acids derived from a blood sample of the mammal with a set of probes specific to at least two separate molecules chosen from among the following targets:
a) the nucleic acid containing the sequences indicated in SEQ ID NOs: 1-437 or a distinctive fragment thereof having at least 15, preferably at least 16, 17, 18, 19, 20, 25 or 30 consecutive bases, and/or b) the nucleic acids having a sequence complementary to sequences according to a), and/or
c) the functional analogs of the nucleic acids according to a) or b) derived from another species,
   to obtain a hybridization profile, the hybridization profile being characteristic of the presence or risk of developing a cancer in this mammal.

The hybridization profile can be compared with one or more reference profiles, in particular a reference profile characteristic of healthy individuals and/or individuals suffering from cancer, the comparison allowing determination of the probability or risk that the tested patient has a cancer. Typically, the comparison is made using computer programs known per se to those skilled in the art.

Selective amplification is preferably performed using a primer or pair of primers allowing amplification of all or part of one of the target nucleic acids in the sample, if any. The primer may be specific to a target sequence according to SEQ ID NO: 1-437, or to a region flanking the target sequence in a nucleic acid of the sample. The primer typically comprises a single-strand nucleic acid, whose length is advantageously between 5 and 50 bases, preferably between 5 and 30. Said primer forms another subject of the present application, and its use (essentially in vitro) for the detection of a cancer in an individual.

In this respect, a further subject of the invention lies in the use of a nucleotide primer or a set of nucleotide primers allowing amplification of all or part of one or preferably several genes or RNAs containing a target sequence according to SEQ ID NO: 1-437, for the detection of a cancer in a mammal, preferably breast cancer, particularly in a human being.

Another particular subject of the invention lies in a method to detect the presence or risk of developing a cancer in a mammal, comprising the contacting, under conditions enabling amplification, of the nucleic acids derived from a blood sample of the mammal with a set of primers specific to at least two separate molecules chosen from among the following targets:
a) the nucleic acids containing the sequences indicated in SEQ ID NOs: 1-437 or a distinctive fragment thereof having at least 15, preferably at least 16, 17, 18, 19, 20, 25 or 30 consecutive bases, and/or
b) the nucleic acids having a sequence complementary to sequences according to a), and/or
c) the functional analogs of nucleic acids according to a) or b) derived from another species,
   to obtain an amplification profile, the amplification profile being characteristic of the presence or risk of developing a cancer in this mammal.

### Detection of a Polypeptide

Under another embodiment, the method comprises the determination of the presence of a polypeptide according to d). The evidencing of a polypeptide in a sample can be performed using any technique known per se, in particular using a specific ligand e.g. an antibody or an antibody fragment of derivative. Preferably, the ligand is an antibody specific to the polypeptide, or a fragment of said antibody (e.g. a Fab, Fab′, CDR, etc.), or a derivative of said antibody (e.g a single-chain antibody, ScFv). The ligand is typically immobilized on a carrier, such as a slide, ead, column, plate, etc. The presence of the target polypeptide in the sample can be detected by evidencing a complex between the target and the ligand, for example using a labeled ligand, using a second labeled developing ligand, etc. Immunology techniques which can be used and are well known are ELISA, RIA techniques, etc.

Antibodies specific to the target polypeptides may be produced using conventional techniques, in particular by immunizing a non-human animal with an immunogen containing the polypeptide (or an immunogenic fragment thereof) and collecting the (polyclonal) antibodies or producer cells (to produce monoclonals). Techniques for the production of poly- or monoclonal antibodies, of ScFV fragments, of human or humanized antibodies are described for example in Harlow et al., Antibodies: A Laboratory Manual, CSH Press, 1988; Ward et al., Nature 341 (1989) 544; Bird et al., Science 242 (1988) 423; WO94/02602; U.S. Pat. No. 5,223,409; U.S. Pat. No. 5,877,293; WO93/01288. The immunogen may be produced by synthesis, or by the expression, in a suitable host, of a target nucleic acid such as defined above. Said monoclonal or polyclonal antibody, and its derivatives, having the same antigenic specificity, also form a subject of the present application, as well as their use for cancer detection.

### Implementation of the Method

The method of the invention can be applied to any biological sample of the tested mammal, in particular any sample containing nucleic acids or polypeptides. A sample of blood, plasma, platelet, saliva, urine, stools, etc. may advantageously be cited and more generally any tissue, organ or advantageously any biological fluid containing nucleic acids or polypeptides.

In one preferred; particularly advantageous embodiment of implementation, the sample is a blood or plasma sample. The invention follows from the identification of blood markers of cancer, and therefore allows detection of these pathologies without any tissue biopsy, and solely using blood samples.

The sample can be obtained using any technique known per se, for example by taking a sample, using non-invasive techniques, from collections or banks of samples, etc. The sample may also be pre-treated to facilitate accessibility of the target molecules, for example by lysis (mechanical, chemical, enzymatic, etc.), purification, centrifugation, separation, etc. Advantageously the PaxGene system is used (Feezor et al., Physiol Genomics 2004: pp. 247-254). The sample may also be labeled to facilitate determination of the presence of target molecules (fluorescent, radioactive, luminescent, chemical, enzymatic labeling, etc.).

In one preferred embodiment, the biological sample is a sample of whole blood i.e. which has not undergone any separation step, and may optionally be diluted.

The invention can be applied to any mammal, preferably humans. The method of the invention is particularly useful for the detection of breast cancer, in particular detection of the presence, risk of developing, or stage of progression of a breast cancer in a human being. Therefore the data given in the examples show that the invention allows the detection of the presence of a breast cancer with a sensitivity of over 92% and a specificity of more than 86%. It is particularly adapted for screening breast cancer at early stages i.e. stages I or II (such as defined under the “TNM Classification of Malignant Tumors”) developed and maintained by the UICC (“International Union Against Cancer”). The TNM classification is also used by AJCC (“American Joint Committee on Cancer”) and by IFGO (“International Federation of Gynecology and Obstetrics”).

One particular subject-matter of the present application concerns a method to detect the presence, progression, or risk of developing a breast cancer in a human individual, comprising the combined determination, in a biological sample from a human individual, of the presence (or absence or (relative) quantity) of target molecules chosen from among:
a) the nucleic acids containing a sequence chosen from among SEQ ID NO: 1-437 or a fragment thereof having at least 15, preferably at least 16, 17, 18, 19, 20, 25 or 30 consecutive bases,
b) the nucleic acids having a sequence complementary to a sequence according to a), and
c) the polypeptides encoded by the nucleic acids according to a) or b).

Preferably, the method comprises the combined determination of the presence, absence of quantity of 5, 10, 20, 30, 40, 50 or 60 target molecules such as defined above.

A further particular subject-matter of the present application concerns a method to detect the presence, progression, or risk of developing a breast cancer in a human individual, comprising the contacting of a biological sample of the individual containing nucleic acids with a product comprising a carrier on which nucleic acids are immobilized containing a sequence complementary to and/or specific to one or preferably several target molecules chosen from among (i) the nucleic acids containing a sequence chosen from among SEQ ID NO: 1-437 or a fragment thereof having at least 15, preferably at least 16, 17, 18, 19, 20, 25 or 30 consecutive bases and (ii) nucleic acids having a sequence complementary to a sequence according to (i), and determination of the hybridization profile, the profile indicating the presence, stage, of risk of developing a breast cancer in said human individual. Preferably, the product contains separate nucleic acids containing a sequence complementary to and/or specific to at least 5, 10, 20, 30, 40, 50, 60 or more different target molecules such as mentioned above.

A further subject of the present application concerns a product comprising a carrier on which nucleic acids are immobilized containing a sequence complementary to and/or specific to one or preferably several target molecules chosen from among (i) the nucleic acids containing a sequence chosen from among SEQ ID NO: 1-437 or a fragment thereof having at least 15, preferably at least 16, 17, 18, 19, 20, 25 or 30 consecutive bases and (ii) the nucleic acids having a sequence complementary to a sequence according to (i). Preferably, the product comprises separate nucleic acids containing a sequence complementary to and/or specific to at least 5, 10, 20, 30, 40, 50, 60 or more different target molecules such as mentioned above. Preferably, it comprises separate nucleic acids containing a sequence complementary to and/or specific to one of the panels of markers such as defined in the present application.

A further subject of the present application concerns a product containing a carrier on which at least one preferably several nucleic acids are immobilized containing a sequence chosen from among SEQ ID NO: 1-437, or a functional analog thereof. Preferably the product comprises at least 5, 10, 20, 30, 40, 50, 60 or more different nucleic acids chosen from among the nucleic acids mentioned above. In one particular embodiment, the product comprises each of the nucleic acids of sequences SEQ ID NO: 1-376.

A further subject of the present application concerns a product comprising a carrier on which at least one ligand is immobilized of a polypeptide encoded by a target nucleic acid such as defined above i.e. a nucleic acid containing a sequence chosen from among SEQ ID NO: 1-437, a distinctive fragment thereof having at least 15, preferably at least 16, 17, 18, 19, 20, 25 or 30 consecutive bases, a nucleic acid having a sequence complementary thereto or a functional analog thereof. Preferably, the product contains at least 5, 10, 20, 30, 40, 50, 60 or more ligands of different polypeptides chosen from among the polypeptides mentioned above.

The carrier may be any solid or semi-solid carrier having at least one surface whether planar or not, allowing the immobilization of nucleic acids or polypeptides. Said carriers are for example a slide, bead, membrane, filter; column, plate etc. They may be made in any compatible material such as glass, silica, plastic, fiber, metal, polymer, polystyrene, Teflon etc. The reagents can be immobilized on the surface of the carrier using known techniques or, for nucleic acids, synthesized directly in situ on the carrier. Immobilization techniques include passive adsorption (Inouye et al., J. Clin. Microbiol. 28 (1990) 1469), covalent binding. Some techniques are described for example in WO90/03382, WO99/46403. The immobilized reagents on the carrier may be placed in a pre-established order to facilitate the detection and identification of the formed complexes, and according to a variable, adaptable density.

In one embodiment, the product of the invention comprises a plurality of synthetic oligonucleotides between 5 and 100 bases in length, specific to one or more target nucleic acids defined under a) to c).

The products of the invention typically comprise control molecules, used to standardize and/or normalize results.

A further subject of the present application concerns a kit comprising a compartment or container containing at least one, preferably several nucleic acids, comprising a sequence complementary to and/or specific to a target nucleic acid such as defined above and/or one, preferably several ligands of a target polypeptide such as defined previously. Preferably, the product contains at least 5, 10, 20, 30, 40, 50, 60 or more different nucleic acids and/or ligands chosen from the above-mentioned nucleic acids and ligands. In one particular embodiment, the product comprises each of the nucleic acids of sequence SEQ ID NO: 1-437 or a ligand for each of the target polypeptides such as defined above. The kit may also contain reagents for a hybridization or immunological reaction and, optionally, controls and/or instructions.

A further subject of the invention concerns the use of a product or kit such as defined above for the detection of a cancer in a mammal, preferably a human individual, in particular to detect breast cancer.

A further subject of the invention concerns a nucleic acid having a sequence chosen from among SEQ ID NO: 1-437, or a distinctive fragment thereof comprising at least 15 consecutive bases, preferably at least 16, 17, 18, 19, 20, 25 or 30, or a nucleic acid having a sequence complementary thereto or a functional analog thereof. The invention also concerns a cloning or expression vector containing these nucleic acids, and any recombinant cell containing said vector or nucleic acid.

A further subject of the invention concerns the use of a nucleic acid comprising a sequence chosen from among SEQ ID NO: 1-437, or a distinctive fragment thereof containing at least 15 consecutive bases, preferably at least 16, 17, 18, 19, 20, 25 or 30, a nucleic acid having a sequence complementary thereto, or a functional analog thereof, for the detection (essentially in vitro detection) of a cancer in a mammal.

In one particular example of embodiment of the invention, a blood sample is taken from a mammal to be tested. The blood sample may optionally be treated to make the nucleic acids more accessible, and they are labeled. The nucleic acids are then applied to a product such as above-defined and the hybridization profile is determined, permitting the diagnosis of whether or not cancer is present in the tested mammal. The method of the invention is simple, performed ex vivo and allows early detection of a cancer from a blood sample.

Evidently any equivalent technique may be used within the scope of the present application to determine the presence of a target molecule.

Other aspects and advantages of the invention will become apparent on reading the following examples which are to be construed as illustrative and non-limiting.

### KEYS TO FIGURES

FIG. 1: PCR amplification of cDNAs DATAS derived from n°1 profiling (PBMN) using 10 pairs of semi-degenerative primers. DATAS profiling was performed in both directions (stage I and II cancers versus controls, and controls versus stage I and II cancers). A positive control using cDNA derived from HepG2 cells is included.

FIG. 2: PCR amplification of cDNAs DATAS derived from n°2 profiling (PBMO) using 10 pairs of semi-degenerative primers. DATAS profiling was performed in both directions (stage III & IV cancers versus controls, and controls versus stage III & IV cancers). A positive control included cDNA derived from HepG2 cells is included.

FIG. 3: PCR amplification of cDNAs DATAS derived from n°3 profiling (PBMP) using 10 pairs of semi-degenerative primers. DATAS profiling was performed in both directions (stage 1 and IV cancers versus controls, and controls versus stage I and IV cancers). A positive control using cDNA derived from HepG2 cells is included.

FIG. 4: Specific configuration of probes to measure the expression of variants generated by alternative splicing. Probe A, common to both isoforms measures the expression of both variants. Probe B, specific to the additional sequence of the long form, and probes C and D specific to the junction sequences around this additional sequence measure the expression of the long isoform. Probe E, specific to the junction derived from absence of the additional sequence, measures the expression of the short isoform.

FIG. 5: Definition of <<target >> sequences. 15 nucleotides either side of each junction and up to 500 nucleotides for the upstream additional and common sequences are captured.

FIG. 6: <<Target>> sequences and associated data. Description of columns: idn°: identification n° of splicing event; Description: type of splicing event; long form: accession n° of long form; short form: accession n° of short form; target A to E: Sequences of targets A to E; long: size of sequences in preceding column.

FIG. 7: Hierarchical clustering of 37 controls and 55 patients using 100 oligonucleotides.

### EXAMPLES

### 1. Characteristics of Biological Samples

The examples given below were initially made from 92 blood samples (5 ml of whole blood, taken in two PaxGene tubes). These samples grouped together 37 blood samples from healthy control patients (H, obtained from Etablissement Francais du Sang) and 55 samples from patients suffering from stage I/II breast cancers (CI/II).

### 2. Extraction of Total RNA from the Blood Sample

The blood samples were directly collected in PAXGene™ Blood RNA tubes (PreAnalytix, Hombrechtikon, CH). After the blood sampling stage and to obtain total cell lysis, the tubes were left at room temperature for 4 h then stored at −20° C. until extraction of the biological material. More precisely, in this protocol, total RNA was extracted using PAXGene Blood RNA® kits (PreAnalytix) following the manufacturer's instructions. In short, the tubes were centrifuged (15 min, 3000 g) to obtain a residue of nucleic acids. This residue was washed and dissolved in a buffer containing protinease K required for digestion of the proteins (10 min at 55° C.). Further centrifuging (5 min, 19 000 g) was performed to remove cell debris and ethanol was added to optimize the fixing conditions of the nucleic acids. Total RNA fixed specifically fixed onto PAXgene RNA spin columns and, before elution thereof, digestion of contaminant DNA was conducted using the RNAse free DNAse set (Qiagen, Hilden, Germany). The quality of total RNA was analyzed on an AGILENT 2100 biolyzer (Agilent Technologies, Waldbronn, Germany).

### 3. DATAS Profiling Experiments

Three series of <<DATAS >> profiling were conducted between the following groups:

DATAS n°1: Early stage breast cancers (Stage I and II) versus Control group (PBMN study)

DATAS n°2: Late stage breast cancers (Stage III and IV) versus Control group (PBMO study)

DATAS n°3: Breast cancers (Stages I, II, III and IV) versus Control group (PMNP study)

DATAS is a profiling technology of gene expression between two samples, which is able to characterize qualitative differences at messenger RNA level, such as those generated by alternative splicing. This patented technology is described in U.S. Pat. No. 6,251,590.

Total RNA corresponding to the two situations, one normal (mN) the other pathological (mP), is isolated from blood samples using the above-described PAXgene system (PreAnalytix). This RNAs (50 μg per group) is converted into complementary DNA (cN) and (cP) using reverse transcriptase (RT) (Invitrogen) and a biotinylated oligonucleotide oligodT25 (Invitrogen). The samples which contributed to the 50 μg per group are indicated in tables A to F.

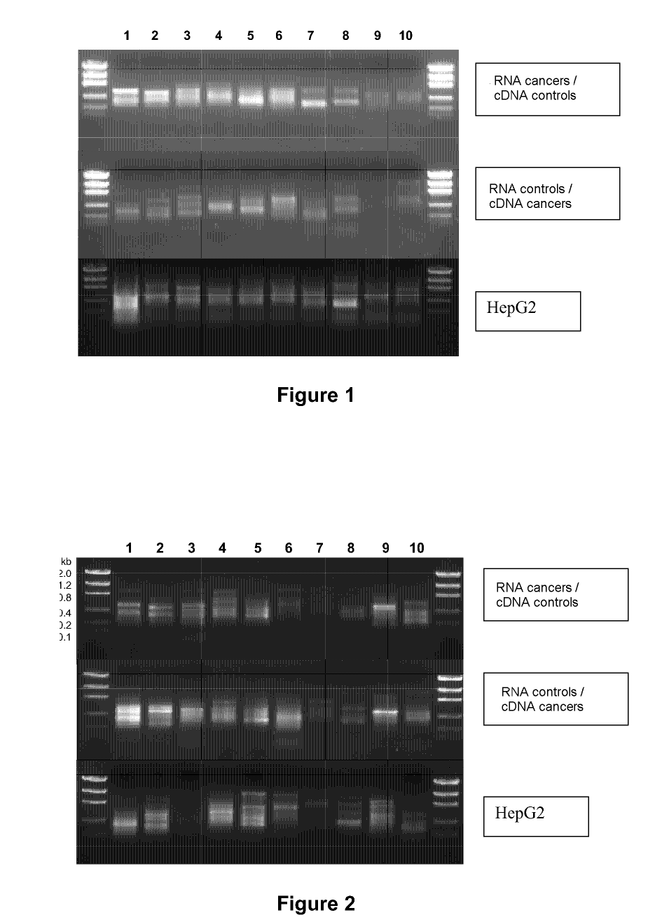

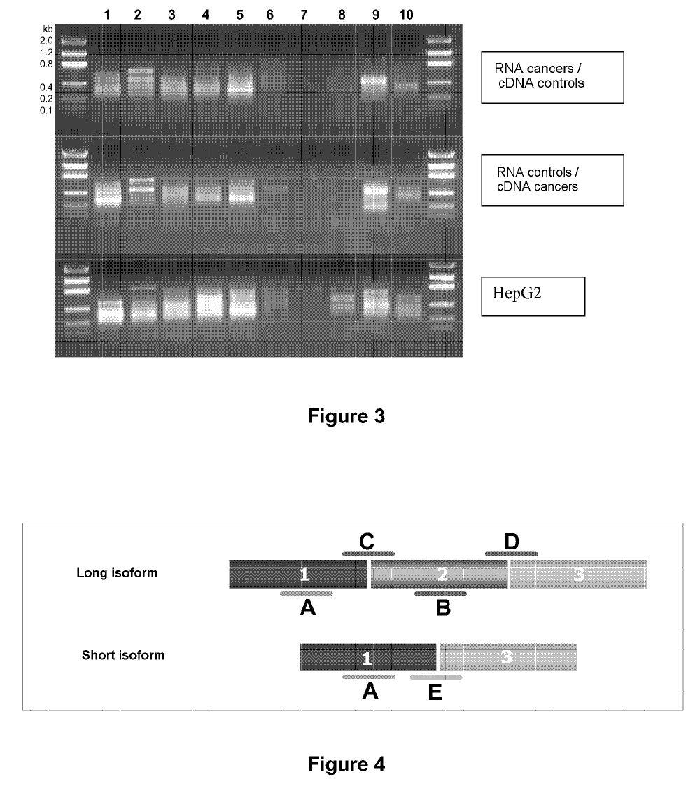

Hybrids mN/cP and cN/mP are then produced in liquid phase. After ethanol precipitation of mN and cP and of cN and mP, the precipitates are dissolved in 30 μl of hybridization solution containing 80% formamide and 0.1% SDS for overnight incubation at 40° C. The heteroduplexes are then captured using magnetic Streptavidin beads (Dynal). A magnet is used to hold the beads in the tube during rinsing operations. The beads/heteroduplexes are then dissolved in 50 μl of RNAseH buffer and incubated with RNaseH (Invitrogen) 30 minutes at 37° C. The supernatant is collected after further magnet application. The residual DNA is removed by action of DNAseI (Ambion). After inactivation of the enzyme, the RNA fragments are precipitated with ethanol and dissolved in water treated with DPEC supplemented with RNAse out (Ambion). The RNA fragments are then reverse transcribed (Reverse transcription TaqMan kit, Applied Biosystem) using random hexamer oligonucleotides. The complementary DNAs obtained are then PCR amplified using semi-degenerative primers. 10 pairs of primers are generally used. The amplicons obtained can be visualized by electrophoresis on agarose gel (FIGS. 1, 2 and 3). Heterogeneous amplified populations can be observed whose type and intensity vary according the primers and samples used.

These amplified populations are cloned in a TOPO TA (Invitrogen) cloning vector for transformation in a strain of competent E. Coli bacteria (Invitrogen). The colonies are transferred to 96-well plates and cultivated overnight in an ampicillin-supplemented 2XTY medium. A glycerolated stock (50%) is then taken. Generally a 96-well plate is processed per pair of primers in one of the two directions. This gives 96×10×2=1920 colonies to be characterized.

1728 clones were sequenced for DATAS n°1 profiling, 1920 were cloned and sequenced for DATAS n° 2 profiling, and 1920 were cloned and sequenced for DATAS n° 3 profiling.

### 4. Bio-Computerized Analysis

Table G summarizes the number of clones characterized in the three profiling banks. A clone designated as a <<singleton >> means that the sequence of this clone was only identified once in the bank and that no other clone overlaps this sequence. A <<cluster >> designates a group of clones whose sequences overlap.

The three DATAS profilings subsequently generated 1741 non-redundant clones.

TABLE G
Characterization of the clones obtained in the three DATAS banks

| | | Cancers | | |
|---|---|---|---|---|
| | Cancers I & | III & | All | Consolidation |
| | II vs. | IV vs. | cancers vs. | over the three |
| DATAS banks | controls | controls | controls | banks |
| Number of clones | 1728 | 1920 | 1920 | 5568 |
| sequenced | | | | |
| Number of | 267 | 347 | 303 | 886 |
| <<clusters>> | | | | |
| Number of | 239 | 362 | 440 | 855 |
| <<singletons>> | | | | |
| Number of non- | 506 | 709 | 743 | 1741⁽*⁾ |
| redundant clones | | | | |
| in bank | | | | |

Bio-computerized analysis was performed on the 1741 clones to identify the genes with which they are associated and the known splicing events in public banks of corresponding nucleic acids.

The 1741 DATAS clones were able to be associated with 1170 different genes, some non-overlapping DATAS fragments being associated with different regions of the same gene.

### 5. Choice and Design of Target Events for Microarray Configuration

The detection and quantification of the expression of splicing variants per microarray requires the use of a particular probe configuration. Any control messenger RNA/splicing variant pair can be modeled as a long isoform/short isoform (FIG. 4). Therefore a splicing variant associated with an exon skip will be the short isoform relative to the control variant. A splicing variant containing a novel exon or intron retention will be the long isoform relative to the control variant. The other alternative splicing events (uses of 5′ or 3′ cryptic splice sites) may also be modeled in similar manner.

The set of probes needed to measure the expression of splicing variants is also indicated in FIG. 4. This set consists of two conventional <<exonic >> probes A and B and of three junction probes of exon-exon type or exon-intron type C, D and E. Probe A measures the expression of the two isoforms, probes B, C and D the expression of the long isoform and probe E the expression of the short form.

For the design of all these probes, the splicing events corresponding to the DATAS fragments must be identified, followed by identification of the <<target >> regions from which the probes will be designed. The target sequences corresponding to the junction probes C, D and E are defined by a length of 30 nucleotides, 15 nucleotides either side of the junction. It is therefore possible to <<cover >> any junction by probes of 25 nucleotides of the type: 10/15, 11/14, 12/13, 13/12, 14/11 et 15/10 (the sign / representing the junction area).

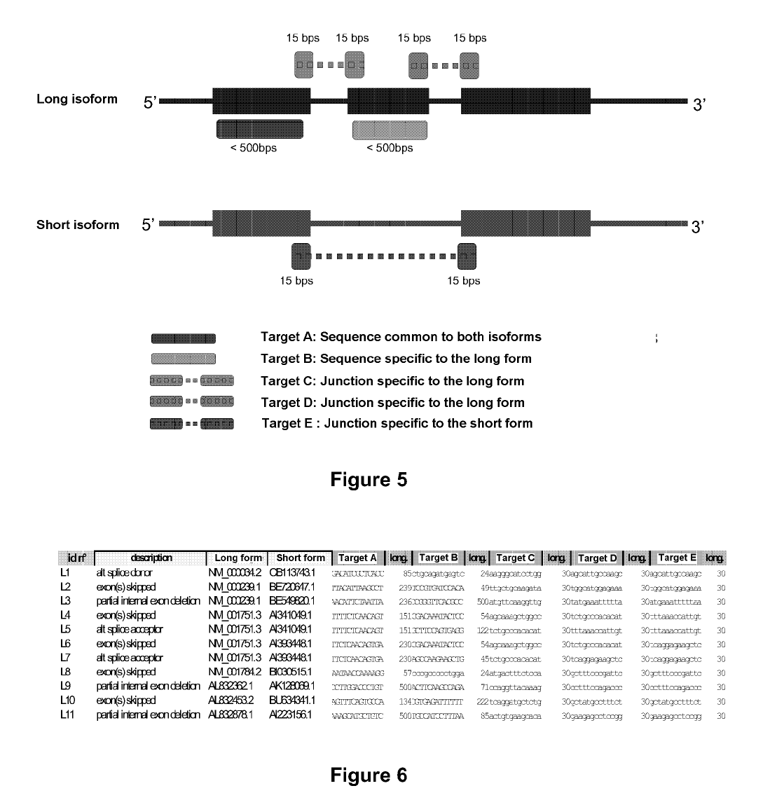

Constraints are less severe on the <<exonic >> probes A and B. The additional sequence, specific to the long form (sequence 2 in FIG. 4) and the common sequence upstream of the splice site (sequence 1 in FIG. 4) define the target sequences for these probes with a ceiling however of 500 nucleotides for those sequences which may exceed this size. The definition of target sequences is summarized in FIG. 5.

Therefore the 1170 genes corresponding to the 1741 DATAS clones were used to identify, for each thereof, the cDNAs et ESTs held in public databanks of sequences, potentially having qualitative differences in sequences, a source of splicing events. The events chosen are located at less than 100 nucleotides from the 5′ or 3′ ends of the DATAS fragments.

2108 events could therefore be chosen and listed in an Excel file of which one part describing the extracted data is given in FIG. 6. These data for a given event comprise: an identification number of the event, the type of event, the accession numbers of the long and short forms, the target sequences A to E and the size of these target sequences. Strict quality control was applied for the defining of these sequences. The ambiguities regarding some nucleotides were corrected through their substitution by corresponding nucleotides on control RNA, RefSeq, or from genomic DNA. All the target sequences were realigned to confirm their attachment to the appropriate short or long forms.

### 6. Design of the Probes and Chip

So as to be able to measure the expression of the 2108 events previously described, a customized DNA chip was designed. On this chip each event was characterized by its five target sequences A-E. For each sequence A and B, 11 pairs of probes of 25 nucleotides were designed, whereas the target sequences of type C, D, or E were detected with 6 pairs of probes with 25 nucleotides.

By pair of probes is meant a first probe which hybridizes perfectly (called PM probes for perfect match) with one of the cDNAs derived from a target transcript, and a second probe, identical to the first probe except for mismatch (i.e. MM probe for mismatched) in the center of the probe. Each MM probe was used to estimate background noise corresponding to hybridization between two nucleotide fragments of non-complementary sequence (Affymetrix technical note “Statistical Algorithms Reference Guide”; Lipshutz, et al (1999) Nat. Genet. 1 Suppl., 20-24). If the design of at least 6 probes for sequences A and B or of at least 4 probes for sequences C, D, et E was impossible, these sequences were not included on the customized chip. Said situation may result from sequences of low complexity containing repeat structures or <<hairpin >> structures whether consecutive or non-consecutive. A sequence size for A-E of less than 30 nucleotides also led to the exclusion of these probes. Solely sequences of good quality, oriented in direction 5′->3′, were used for the design of the probes in accordance with Affymetrix recommendations.

### 7. Synthesis of cRNA, Obtaining and Labeling cDNA and Fragmentation

To analyze the expression of target transcripts according to the invention, the complementary DNA (cDNA) of the mRNA contained in total RNA, such as purified above, was obtained from 400 ng of total RNAs through the use of a Klenow 3′-5′-exonuclease enzyme, 100 units of SuperScript II reverse transcription enzyme (Invitrogen), 10 units of the RNAse inhibitor H Superase-IN (Ambion, Huntigdon, UK) and 200 pmol of <<random >> primer containing the T7 promoter (RP-T7-primer, Eurogenetec, Seraing, Belgium).

All the cDNA thus obtained then underwent in-vitro transcription, conducted using a MEGAscript T7 kit (Ambion) for 16 h at 37° C. The resulting cRNA was subsequently purified on a column with an RNeasy Mini kit (Invitrogen), and the quality of the cRNA obtained was analyzed using the AGILENT 2100 bioanalyzer. The purified cRNA was then quantified by spectrophotometry, and the solution of cRNA was adjusted to a concentration of 1.24 μg/μl cRNA. Twenty-six micrograms of cRNA were then dispatched into two Eppendorf tubes, and 3 μg <<random >> primers were added to each tube. Reverse transcription was performed with 800 units of SuperScriptII (Invitrogen) and 10 units of an inhibitor of RNAse H, in the presence of Klenow enzyme, for 1 h at 37° C. The double-strand cDNA resulting from this approach was then purified using the QIAquick PCR Purification Kit (Qiagen) and quantified by spectrophotometry. Sixteen micrograms of cRNA distributed over three Eppendorf tubes were then fragmented with 0.6 units of DNAse I per tube for 10 minutes at 37° C. The efficacy of fragmentation was verified using the 2100 bioanalyzer (Agilent). The fragmented cDNA was then labeled with biotin using 330 units of terminal transferase (Roche Molecular Biochemicals, Meylan, France) and 1 μl of DNA Labeling Reagent (DLR-1a, 5 mM) [Affymetrix] per microgram of cDNA for 60 min at 37° C.

The entirety of the fragmented and labeled cDNA was finally hybridized on the custom DNA chip (called <<A520138F>>, cf example 6) following a standard hybridization protocol adapted for 11 μm chips.

### 8. Evidencing of Expression Profiles for the Diagnosis of Breast Cancer from Blood Samples

8.1. Evidencing an Expression Profile of Transcripts Enabling Discrimination Between Control Patients (S) and Patients Suffering from a Stage I/II Cancer

The expression of around 2000 variants of RNA, representing around 800 genes, was analyzed and compared between S patients and C I/II patients. For this purpose, 16 μg of fragmented cDNA derived from each sample were added to a hybridization buffer (Affymetrix) and 200 μl of this solution was contacted for 16 h at 50° C. on expression chips. To record the best hybridization and washing performance levels, biotinylated RNAs qualified as <<controls >> (bioB, bioC, bioD and cre) and oligonucleotides (oligo B2) were also included in the hybridization buffer. After the hybridization step, the biotinylated cDNAs hybridized on the chip were developed through the use of a streptavidin-phycoerythrin solution and the signal was amplified using anti-streptavidin antibodies. Hybridization was conducted in a <<GeneChip Hybridisation oven >> (Affymetrix), and the Affymetric protocol followed was the Euk GE-WS2 protocol. The washing and development steps were conducted on a <<Fluidics Station 450>> (Affymetrix). Each chip was then analyzed under an Affymetrix G3000 GeneArray Scanner at a resolution of 1.5 microns to identify the hybridized areas on the chip. This scanner enables detection of the signal emitted by fluorescent molecules after excitation by argon laser using the epifluorescence microscope technique. Therefore, for each position, a signal is obtained that is proportional to the quantity of fixed cDNA. The signal was then analyzed using GeneChip Operating Software (GCOS 1.2, Affymetrix).

To provide against the variations obtained though the use of different chips, a normalization approach was followed using the <<Bioconductor >> tool which harmonizes the mean distribution of raw data obtained for each chip. The results obtained on one chip can then be compared with the results obtained on another chip. With the GCOS 1.2 software it was also possible to include a statistical algorithm to determine whether a transcript was or was not expressed.

From the 6,242 groups of probes of the chip, representing around 2,000 transcripts, the inventors selected the relevant transcripts which were correlated with the development of a breast cancer. Those transcripts whose level of expression on the majority of chips was too low and those transcripts which did not show any substantial variation between the different chips, were excluded. (Li et al, 2001, Bioinformatics, 17: 1131-1142). The search for a panel of transcripts discriminating between groups of EFS and CI/II patients was conducted using a Data Mining technique called <<random forest algorithm >> (http://ligarto.org/rdiaz/Papers/jornadas.bioinfo.randomForest.pdf). In addition to data analysis using the random forest algorithm, which is an analysis of multivariate type, a so-called <<univariate >> analysis was also used to identify those transcripts differentially expressed between EFS and CI/II patients. This analysis called SAM (<<Significance Analysis of Microarrays >>) is chiefly based on a modified version of Student's t test providing against the bias introduced by genes with low variability. With this approach, it is possible to control the percentage of false positive genes in a univariate analysis.

With all the above-mentioned analyses, it was possible to evidence a first panel of transcripts comprising 318 relevant transcripts according to the invention (cf. Table 1, SEQ ID Nos: 1-318). The increase or decrease in the expression of each of the transcripts observed in healthy patients (S) compared with C I/II patients is given in Table 1.

The inventors have examined the simultaneous expression of 318 transcripts to obtain an expression profile. Using the random forest method, 90% of patients were correctly classified. More precisely, 32 of the 37 controls and 51 of the 55 patients were correctly classified, which corresponds to a sensitivity of 92.7% and a specificity of 86.4%. In addition to the analysis on the 92 initial samples, an additional analysis was conducted to validate the relevance of the above-identified signature: an independent cohort of five healthy controls and 16 stage I/II breast cancer patients underwent a blind study. The analysis of an independent cohort is one of the best ways to test the predictive value of a signature of genes or transcripts (cf. The SMRS working group, Nat Biotech 2005, 7: p. 833-838). Based on sequences SEQ ID Nos: 1-318, the random forest algorithm correctly classified five controls out of five and 13 patients out of sixteen (86% classification).

Through additional hybridization and analysis experiments, in 188 patients, 119 additional targets could be identified corresponding to sequences SEQ ID NO: 319-437 (cf. Table 1).

### 8.2. Identification of a Predictive Panel of 100 Markers (PANEL 7)

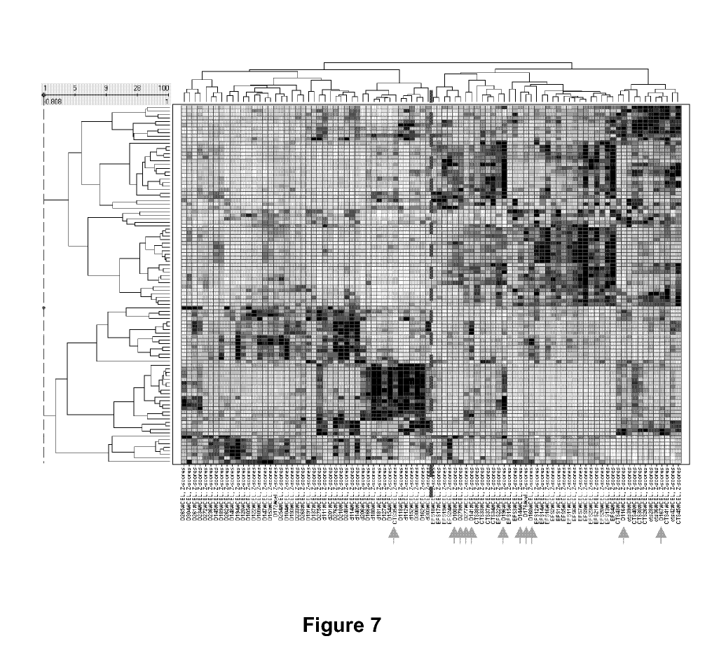

The inventors also studied the simultaneous expression of 100 transcripts of nucleotide sequences chosen from among the sequences given in Table 2 to obtain an expression profile. Using the random forest method, 89% of patients were correctly classified. More precisely, 31 out of the 37 controls and 51 out of the 55 patients were correctly classified which corresponds to a sensitivity of 92.7% and a specificity of 83.7%. These results were confirmed by another analysis technique, the hierarchical cluster technique. In this non-supervised analysis, one Control positions itself among the patients (on the left of the red dotted line, cf. FIG. 7), and 10 cancer patients figure among the healthy controls (on the right of the red dotted line). FIG. 7 shows hierarchical cluster analysis of blood samples obtained from 55 patients suffering from early stage breast cancer (C I/II, also called D) and 37 Controls (healthy donors) using the expression of 100 genes identified by algorithmic analysis. The hierarchical clustering function of the Spotfire software organizes C I/II patients and controls in columns, and the genes in lines so as to show patients or genes having comparable expression profiles in adjacent positions. Pearson's coefficient of correlation was used as index of similarity for the genes and the patients. The results correspond at Affymetrix fluorescence level normalized with the <<bioconductor >> tool. To take into consideration constituent expression differences between genes, the expression levels of each gene were normalized by calculating a reduced aligned variable. Low expression levels are shown in white, intermediate levels in gray and strongest levels in black. The height of the dendrogram branches indicates the index of similarity between the expression profiles.

In addition to the analysis on the 92 initial samples, an additional analysis was conducted to validate the relevance of the above-identified signature: a blind study was conducted in an independent cohort of five controls and 16 stage I/II breast cancer patients. Based on the top 100, the random forest algorithm correctly classified five controls out of five and 14 patients out of sixteen (90% classification).

Amongst this combination of 100 marker genes, the inventors evidenced that smaller panels also enabled a discrimination to be made between control patients and breast cancer patients as described in the following examples.

### 8.3. Identification of a Predictive Panel of 66 Markers (PANEL 6)

The inventors have evidenced a combination of 66 markers, based on sequences SEQ ID Nos: 5, 7, 13, 14, 16-20, 23-28, 47, 51-55, 58, 64, 69, 80, 81, 88-90, 116, 121, 125, 137, 139, 145, 148, 158, 160, 161, 164, 188-191, 208, 222, 225, 228, 229, 236, 240, 242, 245, 248, 252, 280, 281, 284, 290, 298-300 and 309-312 (see Table 3). With this combination it is possible to classify correctly more than 80% of samples.

### 8.4. Identification of a Predictive Panel of 53 Markers (PANEL 5)

The inventors have also evidenced a combination of 53 markers, based on sequences SEQ ID Nos: 7, 13, 14, 16-19, 23-28, 47, 51-55, 58, 69, 80, 81, 89, 116, 121, 125, 137, 139, 145, 148, 158, 160, 161, 164, 189, 190, 225, 228, 229, 240, 245, 248, 252, 280, 281, 284, 290, 298-300, 310 and 312. With this combination it is also possible to classify correctly more than 80% of samples.

### 8.5. Identification of a Predictive Panel of 42 Markers (PANEL 4)

The inventors have also evidenced a combination of 42 markers, based on sequences SEQ ID Nos: 13, 16-19, 23, 26-28, 47, 51-55, 58, 69, 80, 81, 89, 116, 121, 125, 145, 148, 158, 160, 161, 164, 189, 190, 225, 229, 240, 248, 280, 281, 284, 299, 300, 310 and 312. With this combination it is also possible to classify correctly more than 80% of samples.

### 8.6. Identification of a Predictive Panel of 22 Markers (PANEL 3)

The inventors have evidenced a combination of 22 markers, based on sequences SEQ ID Nos: 18, 19, 23, 26, 27, 51, 52, 53, 54, 55, 69, 80, 125, 145, 148, 161, 188, 225, 228, 240, 280 and 312. With this combination it is also possible to classify correctly 76% of samples.

### 8.7. Identification of a Predictive Panel of 18 Markers (PANEL 2)

The inventors have also evidenced a combination of 18 markers based on target sequences SEQ ID NOs: 18, 19, 23, 26, 51, 52, 53, 54, 55, 69, 80, 125, 145, 148, 225, 228, 240 and 312 given in Table 3. With this combination it is possible to classify correctly 76% of samples.

This confirms that analysis of the expression of these 18 markers is a good tool to discriminate between patients carrying a high risk of relapse and those with a low risk of relapse. The use of a restricted panel of genes is particularly suitable to obtain a detection and prognosis tool. Analysis of the expression of a dozen markers does not require the custom fabrication of a DNA chip, and can be implemented directly using PCR or NASBA techniques, or a low density chip, which is a considerable economic advantage with simplified implementation.

### 8.8. Identification of Predictive Panels of Markers (PANELS 1 and 7-9)

The inventors have evidenced combinations of 100, 104 and 110 markers, based on the target sequences SEQ ID Nos: 1-437, allowing detection of the presence of a breast cancer in individuals. These panels are described in Table 4. Panel 1 comprises all the sequences common to Panels 7-9.

### 8.9. Identification of a Predictive Panel of 90 Markers (PANELS 10 and 11)

Through additional hybridization and analysis experiments, conducted in 188 patients, the inventors have evidenced a combination of 90 markers, based on sequences SEQ ID Nos: 11; 12; 18; 23; 51 to 53; 59; 60; 105; 148; 150; 191; 195; 206; 225 a 227; 229; 280; 281; 308 to 310; 312; 327; 343; 360; 367; 377 to 437 (see Table 4).

With this combination, it is possible to classify correctly 86.1% of samples of early stage breast cancers. The genes in this panel are specific to early stage breast cancers. In additional tests, 19 blood samples taken from women suffering from colon cancer and 20 blood samples from women suffering from metastatic breast cancer were analyzed on custom chips such as described above. For each of these two diseases, only 3 patients showed a similar expression of the 90 markers included in Panel 10. Therefore, 84.2% (16 out of 19) of colon cancers and 85% (17 out of 20) metastatic breast cancers were not confused with early stage breast cancer.

Panel 11 comprises all the sequences common to all Panels 1 to 10 i.e. the nucleic acids comprising the sequences indicated in SEQ ID No: 23, 52, 53, 148 et 225 (see Table 4).

### 8.10. Identification of Signaling Cascades of Immune Response

Analysis of the different genes identified in the invention, having altered expression in patients, shows that they belong to families of genes involved in cell signaling pathways or in common regulating mechanisms. In particular this analysis shows numerous genes involved in the signaling cascades used in immune response (innate or acquired), and notably in the transduction of messages initiated by stimulation of TLRs, in the secretion of cytokins or in lymphocyte-T activation.

The applicants have therefore defined panels of genes related to or taking part in signaling pathways of immune response, which form targets of interest for cancer detection. These panels are given in Tables 5 and 6.

TABLE A
Samples selected for the early stage breast cancer
group for DATAS n^{o}1 (PBMN)

| | Clinical | Quantity |
|---|---|---|
| Sample | stage | used (ug) |
| D104 | I | 10.0 |
| D105 | I | 5.0 |
| D106 | I | 2.0 |
| D111 | I | 2.0 |
| D114 | I | 3.0 |
| D118 | I | 5.0 |
| D123 | I | 6.0 |
| D117 | II | 2.0 |
| D121 | II | 5.0 |
| D130 | II | 10.0 |

TABLE B
Samples selected for the control group for DATAS n^{o}1 (PBMN)

| | Clinical | Quantity |
|---|---|---|
| Sample | stage | used (ug) |
| D1 | DFS > 5 | 1.66 |
| D3 | DFS > 5 | 1.66 |
| D9 | DFS > 5 | 1.66 |
| D10 | DFS > 5 | 1.66 |
| D17 | DFS > 5 | 1.66 |
| D28 | DFS > 5 | 1.66 |
| D36 | DFS > 5 | 1.66 |
| D63 | DFS > 5 | 1.66 |
| D75 | DFS > 5 | 1.66 |
| D76 | DFS > 5 | 1.66 |
| D11 | DFS < 5 | 1.66 |
| D14 | DFS < 5 | 1.66 |
| D15 | DFS < 5 | 1.66 |
| D16 | DFS < 5 | 1.66 |
| D27 | DFS < 5 | 1.66 |
| D31 | DFS < 5 | 1.66 |
| D41 | DFS < 5 | 1.66 |
| D66 | DFS < 5 | 1.66 |
| D102 | DFS < 5 | 1.66 |
| D103 | DFS < 5 | 1.66 |
| D67 | Benign | 1.66 |
| D69 | Benign | 1.66 |
| D70 | Benign | 1.66 |
| D71 | Benign | 1.66 |
| D72 | Benign | 1.66 |
| D73 | Benign | 1.66 |
| D74 | Benign | 1.66 |
| D79 | Benign | 1.66 |
| D115 | Benign | 1.66 |
| D125 | Benign | 1.66 |

TABLE C
Samples selected for the late stage breast
cancer group for DATAS n^{o}2 (PBMO)

| | Clinical | Quantity |
|---|---|---|
| Sample | stage | used (ug) |
| D55 | S III | 3.33 |
| D59 | S III | 3.33 |
| D90 | S III | 3.33 |
| D134 | S III | 3.33 |
| D168 | S III | 3.33 |
| D178 | S III | 3.33 |
| D126 | S III | 3.33 |
| D132 | S III | 3.33 |
| D91 | S IV | 3.33 |
| D92 | S IV | 3.33 |
| D101 | S IV | 3.33 |
| D138 | S IV | 3.33 |
| D161 | S IV | 3.33 |
| D170 | S IV | 3.33 |
| D99 | S IV | 3.33 |

TABLE D
Samples selected for the control group for DATAS n^{o}2 (PBMO)

| | Clinical | Quantity |
|---|---|---|
| Sample | stage | used (ug) |
| D1 | DFS > 5 | 1.66 |
| D3 | DFS > 5 | 1.66 |
| D9 | DFS > 5 | 1.66 |
| D10 | DFS > 5 | 1.66 |
| D17 | DFS > 5 | 1.66 |
| D28 | DFS > 5 | 1.66 |
| D36 | DFS > 5 | 1.66 |
| D63 | DFS > 5 | 1.66 |
| D75 | DFS > 5 | 1.66 |
| D76 | DFS > 5 | 1.66 |
| D11 | DFS < 5 | 1.66 |
| D14 | DFS < 5 | 1.66 |
| D15 | DFS < 5 | 1.66 |
| D16 | DFS < 5 | 1.66 |
| D27 | DFS < 5 | 1.66 |
| D31 | DFS < 5 | 1.66 |
| D41 | DFS < 5 | 1.66 |
| D66 | DFS < 5 | 1.66 |
| D102 | DFS < 5 | 1.66 |
| D103 | DFS < 5 | 1.66 |
| D67 | Benign | 1.66 |
| D69 | Benign | 1.66 |
| D70 | Benign | 1.66 |
| D71 | Benign | 1.66 |
| D72 | Benign | 1.66 |
| D73 | Benign | 1.66 |
| D74 | Benign | 1.66 |
| D79 | Benign | 1.66 |
| D115 | Benign | 1.66 |
| D125 | Benign | 1.66 |

TABLE E
Samples selected for the breast cancer group
all stages for DATAS n^{o}3 (PBMP).

| | Clinical | Quantity |
|---|---|---|
| Sample | stage | used (ug) |
| D91 | S IV | 1.3 |
| D92 | S IV | 1.3 |
| D101 | S IV | 1.3 |
| D138 | S IV | 1.3 |
| D161 | S IV | 1.3 |
| D170 | S IV | 1.3 |
| D99 | S IV | 1.3 |
| D195 | S IV | 1.3 |
| D197 | S IV | 1.3 |
| D205 | S IV | 1.3 |
| D55 | S III | 1.3 |
| D59 | S III | 1.3 |
| D90 | S III | 1.3 |
| D134 | S III | 1.3 |
| D168 | S III | 1.3 |
| D178 | S III | 1.3 |
| D126 | S III | 1.3 |
| D132 | S III | 1.3 |
| D135 | S III | 1.3 |
| D185 | S III | 1.3 |
| D108 | S II | 1.3 |
| D109 | S II | 1.3 |
| D148 | S II | 1.3 |
| D156 | S II | 1.3 |
| D160 | S II | 1.3 |
| D162 | S II | 1.3 |
| D163 | S II | 1.3 |
| D166 | S II | 1.3 |
| D167 | S II | 1.3 |
| D172 | S II | 1.3 |
| D112 | S I | 1.3 |
| D120 | S I | 1.3 |
| D122 | S I | 1.3 |
| D127 | S I | 1.3 |
| D131 | S I | 1.3 |
| D145 | S I | 1.3 |
| D147 | S I | 1.3 |
| D153 | S I | 1.3 |
| D173 | S I | 1.3 |
| D176 | S I | 1.3 |

TABLE F
Samples selected for the control group for DATAS n^{o}3 (PBMP)

| | Clinical | Quantity |
|---|---|---|
| Sample | stage | used (ug) |
| D1 | DFS > 5 | 1.66 |
| D3 | DFS > 5 | 1.66 |
| D9 | DFS > 5 | 1.66 |
| D10 | DFS > 5 | 1.66 |
| D17 | DFS > 5 | 1.66 |
| D28 | DFS > 5 | 1.66 |
| D36 | DFS > 5 | 1.66 |
| D63 | DFS > 5 | 1.66 |
| D75 | DFS > 5 | 1.66 |
| D76 | DFS > 5 | 1.66 |
| D11 | DFS < 5 | 1.66 |
| D14 | DFS < 5 | 1.66 |
| D15 | DFS < 5 | 1.66 |
| D16 | DFS < 5 | 1.66 |
| D27 | DFS < 5 | 1.66 |
| D31 | DFS < 5 | 1.66 |
| D41 | DFS < 5 | 1.66 |
| D66 | DFS < 5 | 1.66 |
| D102 | DFS < 5 | 1.66 |
| D103 | DFS < 5 | 1.66 |
| D67 | Benign | 1.66 |
| D69 | Benign | 1.66 |
| D70 | Benign | 1.66 |
| D71 | Benign | 1.66 |
| D72 | Benign | 1.66 |
| D73 | Benign | 1.66 |
| D74 | Benign | 1.66 |
| D79 | Benign | 1.66 |
| D115 | Benign | 1.66 |
| D125 | Benign | 1.66 |

TABLE 1

List of 437 transcripts expressed differentially during breast cancer
development.

| SEQ | | | | C I/II | |
|---|---|---|---|---|---|
| ID | Description | Genbank N^{o} | Genbank N^{o} | vs. | |
| N^{o} | of sequence | (reference) | (variant) | Healthy | Target Sequence |
| 1 | lysozyme | NM_000239.1 | BE720647.1 | 0.8 | ATTTATCCTGCAGTGctttgctgcaagata |
| 2 | cDNA | AL832453.2 | BU634341.1 | 0.8 | AAATAAAATATCAGGGATATGCTCC |
| | DKFZp451G151 | | | | CCCTTGAGACTGAAGGAACTGAAGA |
| | (Leucine- | | | | TTTTAAACCTTAGTAAGAACCACAT |
| | rich repeat | | | | TTCATCCCTATCAGAGAACTTTCTT |
| | kinase 2) | | | | GAGGCTTGTCCTAAAGTGGAGAGTT |
| | | | | | TCAGTGCCA |
| 3 | cDNA | AL832453.2 | BU634341.1 | 0.8 | GAATGAATTTTCTTGctgctatgcctttct |
| | DKFZp451G151 | | | | |
| | (Leucine- | | | | |
| | rich repeat | | | | |
| | kinase 2) | | | | |
| 4 | cDNA | AL832878.1 | AI223156.1 | 0.8 | TGCCAAGGAAGACCCCCTCCTGAC |
| | DKFZp667I093 | | | | CCCTGTTCCGGCTTCAGAAAACCC |
| | (Guanine | | | | GTTTAGGGAGAAGAAGTTTTTCTG |
| | nucleotide | | | | TGCCATCCTTTAA |
| | binding | | | | |
| | protein, | | | | |
| | gamma 2) | | | | |
| 5 | cDNA | AL832878.1 | AI223156.1 | 0.7 | TGCAGATTTGATGGCctactgtgaagcaca |
| | DKFZp667I093 | | | | |
| | (Guanine | | | | |
| | nucleotide | | | | |
| | binding | | | | |
| | protein, | | | | |
| | gamma 2) | | | | |
| 6 | chemokine | NM_001838.2 | BI910219.1 | 0.8 | GATGAGGTCACGGACGATTACATCG |
| | (C-C motif) | | | | GAGACAACACCACAGTGGACTACAC |
| | receptor 7 | | | | TTTGTTCGAGTCTTTGTGCTCCAAG |
| | | | | | AAGGACGTGCGGAACTTTAAAGCCT |
| | | | | | GGTTCCTCCCTATCATGTACTCCAT |
| | | | | | CATTTGTTTCGTGGGCCTACTGGGC |
| | | | | | AATGGGCTGGTCGTGTTGACCTATA |
| | | | | | TCTATTTCAAGAGGCTCAAGACCAT |
| | | | | | GACCGATACCTACCTGCTCAACCTG |
| | | | | | GCGGTGGCAGACATCCTCTTCCTCC |
| | | | | | TGACCCTTCCCTTCTGGGCCTACAG |
| | | | | | CGCGGCCAAGTCCTGGGTCTTCGGT |
| | | | | | GTCCACTTTTGCAAGCTCATCTTTG |
| | | | | | CCATCTACAAGATGAGCTTCTTCAG |
| | | | | | TGGCATGCTCCTACTTCTTTGCATC |
| | | | | | AGCATTGACCGCTACGTGGCCATCG |
| | | | | | TCCAGGCTGTCTCAGCTCACCGCCA |
| | | | | | CCGTGCCCGCGTCCTTCTCATCAGC |
| | | | | | AAGCTGTCCTGTGTGGGCATCTGGA |
| | | | | | TACTAGCCACAGTGCTCTCCATCCC |
| 7 | Homo sapiens | BC009917.1 | BC028225.1 | 1.4 | ATGAAGAAAAACAAAgtgcacagagacccg |
| | hypothetical | | | | |
| | protein | | | | |
| | DKEZp761A052 | | | | |
| 8 | cDNA clone | BC038965.1 | N70893.1 | 1.2 | AGGACAGCCCTGGGCagagatgaggcaggg |
| | IMAGE: | | | | |
| | 3920936 | | | | |
| | (High | | | | |
| | density | | | | |
| | lipoprotein | | | | |
| | binding | | | | |
| | protein | | | | |
| | (vigilin)) | | | | |
| 9 | cDNA clone | BC040042.1 | B1001496.1 | 1.3 | TAATGCCAAGACAAAgccacgggaggagca |
| | IMAGE: | | | | |
| | 5207605 | | | | |
| | (Immuno- | | | | |
| | globulin | | | | |
| | heavy | | | | |
| | constant | | | | |
| | gamma 2 | | | | |
| | (G2m | | | | |
| | marker)) | | | | |
| 10 | cDNA clone | BC040042.1 | BF841656.1 | 1.3 | CACAGGTGTACACCCTGCCCCCATC |
| | IMAGE: | | | | CCGGGAGGAGATGACCAAGAACCAG |
| | 5207605 | | | | GTCAGCCTGACCTGCCTGGTCAAAG |
| | (Immuno- | | | | GCTTCTACCCCAGCGACATCGCCGT |
| | globulin | | | | GGAGTGGGAGAGCAATGGGCAGCCG |
| | heavy | | | | GAGAACAACTACAAGACCACACCTC |
| | constant | | | | CCATGCTGGACTCCGACGGCTCCTT |
| | gamma 2 | | | | CTTCCTCTACAGCAAGCTCACCGTG |
| | (G2m | | | | GACAAGAGCAGGTGGCAGCAGGGGA |
| | marker)) | | | | ACGTCTTCTCATGCTCCGTGATGCA |
| | | | | | TGAGGCTCTGCACAACCACTACACG |
| | | | | | CAGAAGAGCCTCTCCCTGTCTCCGG |
| | | | | | GTAAATGAGTGCCACGGCCGGCAAG |
| | | | | | CCCCCGCTCCCCAGGCTCTCGGGGT |
| | | | | | CGCGTGAGGATGCTTGGCACGTACC |
| | | | | | CCGTGTACATACTTCCCAGGCACCC |
| | | | | | AGCATGGAAATAAAGCACCCAGCGC |
| | | | | | T |
| 11 | Microtubule | BC048206.1 | AW015234.1 | 1.3 | TCTCTCTTTCCAATCTTACGCCATG |
| | associated | | | | GCCATCAGTTCATTTCAGCCTTCCA |
| | monoxygenase, | | | | GTGCTACACCCACTTCTTGGCTGAC |
| | calponin and | | | | ACACTTCTGCTCTAAGGTGACTGGT |
| | LIM domain | | | | TTTCTTGCCAATTTTCAAAGAGTGG |
| | containing 2 | | | | TACTAACCCCCAACCCGCTTTCCGC |
| | | | | | ACCCCGTCCTCTCCGCCAGCAGTAC |
| | | | | | TGGTTGCACTAACTGTGAGTGTCTT |
| | | | | | GCATACTGATGGACTCATTTGGTGG |
| | | | | | CATGGTTGGCTAACAGCATGGCGGG |
| | | | | | GGGTGTTCAGCTTGAGACCCATGCC |
| | | | | | TGTGTTCATTTCCCATGGAGCTGGC |
| | | | | | AGCCTGGTCTACCCCAAGTGCATGC |
| | | | | | CCCGCCTCTCCTCTCTCCCTTGGGT |
| | | | | | CTGCCTGCGTGCATGCTTCTCCAGT |
| | | | | | TGCGTCTGCGAAGCTACCTACTTTC |
| | | | | | TTGGGAGGGTCGACCTTGATCATGA |
| | | | | | AACAATACCATGAGGGGGCCTCTGT |
| | | | | | CACCTTTGAAAAGAACACTTTTTGA |
| | | | | | GCAGCCTCAAAAAGCTCATACATAC |
| 12 | Microtubule | BC048206.1 | AF052170.1 | 1.4 | TGGGAGGGTCGACCTTGATCATGAA |
| | associated | | | | ACAATACCATGAGGGGGCCTCTGTC |
| | monoxygenase, | | | | ACCTTTGAAAAGAACACTTTTTGAG |
| | calponin and | | | | CAGCCTCAAAAAGCTCATACATACC |
| | LIM domain | | | | AGCGCCTTCTTAAATTGGCTCTAAT |
| | containing 2 | | | | GTAAAGATTGTTAATGTCATTTATC |
| | | | | | AAAACCATAGGTGATTATTTGGAGG |
| | | | | | GATTTAAAAAACTTAATTACTCTCA |
| | | | | | GGCCTCATCCCAAGCTTGACACATG |
| | | | | | CTCTGTAGGTTGAACACATAATCAC |
| | | | | | AAATATTCTAGCAAATGCTGCCTTG |
| | | | | | GTTGCAGCCTGCACTGTAGACCCAA |
| | | | | | GGGTTTTGCTGTGGCTCTTCTTATC |
| | | | | | TCCCTTGGCTCATAAAGCCCCAGAT |
| | | | | | GATGCCAGAGCTTCAATTAGAGCCA |
| | | | | | TCATCATCCCAGGCAGGGATATCTT |
| | | | | | TGAGAAATGACTCAGTTCAGCCCCA |
| | | | | | GGCCCCTGTGACTCTGCTTAAAGCA |
| | | | | | CACATTTCTGCTGACTCTTGTACCT |
| | | | | | GGGGCAGCAGGATAATCACCAACAC |
| 13 | cellular | NM_001997.2 | NM_001997.2 | 0.5 | GTCGCCCAGATCAAGgctcatgtagcctca |
| | homolog | | | | |
| | of the fox | | | | |
| | sequence in | | | | |
| | the Finkel- | | | | |
| | Biskis- | | | | |
| | Reilly | | | | |
| | murine | | | | |
| | sarcoma | | | | |
| | virus | | | | |
| 14 | cellular | NM_001997.2 | W17004.1 | 0.6 | GGCCGCATGCTTGGAaggtaaagtccatgg |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 15 | cellular | NM_001997.2 | AU098396.1 | 0.6 | GTCGCCCAGAGCAAGgctcatgtagcctca |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 16 | cellular | NM_001997.2 | AA063591.1 | 0.7 | TGACCGGCCAGGAAAcggtcgcccagatca |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 17 | cellular | NM_001997.2 | AA094898.1 | 0.6 | CAGGCCGCATGCTTGaggtaaagtccatgg |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 18 | cellular | NM_001997.2 | AA187006.1 | 0.7 | AGGCCGCATGCTTGGaggtaaagtccatgg |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 19 | cellular | NM_001997.2 | AA225636.1 | 0.6 | GGCCAGGAAACGGTCgcccagatcaaggta |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 20 | cellular | NM_001997.2 | BM820687.1 | 0.5 | GGTCGCCCAGATCAAgctcatgtagcctca |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 21 | cellular | NM_001997.2 | AA491544.1 | 0.7 | GCCGCATGCTTGGAGgtaatgtccatggtt |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 22 | cellular | NM_001997.2 | AV743892.1 | 0.5 | GTCGCCCAGATCAAGgctctgtagcctcac |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 23 | cellular | NM_001997.2 | BU603086.1 | 0.6 | GAAGTAGCAGGCCGCatgcttggaggtaaa |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 24 | cellular | NM_001997.2 | BF218408.1 | 0.6 | AGGCCGATGCTTGGAggtaaagtccatggt |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 25 | cellular | NM_001997.2 | AI499403.1 | 0.7 | CCCGCATGCTTGGAGgtaaagtccatggtt |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 26 | cellular | NM_001997.2 | AW795076.1 | 0.6 | CGGTCGCCCAGATCAaggctcatgtagcct |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 27 | cellular | NM_001997.2 | D52122.1 | 0.6 | GGCCGCATGCTTGGggtaaagtccatggtt |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 28 | cellular | NM_001997.2 | BE535673.1 | 0.6 | GCCGCATGCTTGGAGgtaacagtccatggt |
| | homolog | | | | |
| | of the fox | | | | |
| | sequence in | | | | |
| | the Finkel- | | | | |
| | Biskis-Reilly | | | | |
| | murine sarcoma | | | | |
| | virus | | | | |
| 29 | mannose-6- | NM_002355.2 | CA430891.1 | 0.8 | CGACACACCCTAGCGgacaattttaaccct |
| | phosphate | | | | |
| | receptor | | | | |
| | (cation | | | | |
| | dependent) | | | | |
| 30 | mitogen- | NM_002419.2 | AK090614.1 | 1.2 | TTCCATTCCATGCAGgaaggctggaagcgc |
| | activated | | | | |
| | protein | | | | |
| | kinase | | | | |
| | kinase | | | | |
| | kinase 11 | | | | |
| 31 | proteoglycan | NM_002727.1 | BQ051861.1 | 0.8 | AATCCTCAGTTCAAGgttatcctacgcaga |
| | 1, | | | | |
| | secretory | | | | |
| | granule | | | | |
| 32 | proteoglycan | NM_002727.1 | BQ051861.1 | 0.8 | GACTGACCTTTTTCCaaagacgagaatcca |
| | 1, | | | | |
| | secretory | | | | |
| | granule | | | | |
| 33 | protein | NM_002743.1 | BU631834.1 | 1.5 | CTCGCAGAAACCCAAccgctccaccaccgt |
| | kinase C | | | | |
| | substrate | | | | |
| | 80K-H | | | | |
| 34 | SEC14-like | NM_003003.1 | CD366399.1 | 1.6 | GTAGGTAGGTTCGTAgtagggttcgtaggt |
| | 1 (S. | | | | |
| | cerevisiae) | | | | |
| 35 | SEC14-like | NM_003003.1 | AK130317.1 | 1.3 | TAGGGCTAGTAGGTAGGGCTAGTA |
| | 1 (S. | | | | GGTAGGGCTAGTAGGTAGGGCTAG |
| | cerevisiae) | | | | TAGGTAGGGCTAGTAGGTAGGGCT |
| | | | | | AGTAGGTAGGGCTAGTAGGTAGGG |
| | | | | | TTCGTAGGTAGGGTTCGTAGGTAG |
| | | | | | GGTTCGTAGGTAGGGTTAGTAGCG |
| | | | | | CGTCTGTGCTGCTTCCACCTGGTG |
| | | | | | CTTCCTGTTCCCAAATCACAAGGG |
| | | | | | CCTGAAGGTGGTCCCTGCTTTCTC |
| | | | | | TTTCTCTTTCTCTGTGTCTCAGAT |
| | | | | | GGCGATTTTGCTGACAGCTGCCAA |
| | | | | | GAAAATGCTTCACTCAACAGTCCT |
| | | | | | CATGTGCCCAGAGATGTTTATAGA |
| | | | | | ACTGTTTGAATTGCAGCCATCCCC |
| | | | | | TGCCCCCTCCCAGGCTGAAGATCT |
| | | | | | GTTCTTTTTAAGTTGATTCGGGAG |
| | | | | | TGGCATTCTTTTATACCCAAAGAC |
| | | | | | TGTAGTGCATCTTGAAGAGCTCAA |
| | | | | | AGCACATGACCGCACAAATGCTTA |
| | | | | | CAGGGTTTCCTCCCGAGTAATCCA |
| | | | | | ATCTCACTCCCCTTGTAAGG |
| 36 | SEC14-like | NM_003003.1 | AK130317.1 | 1.4 | TAGGGTTCGTAGGTAGGGCTAGTAG |
| | 1 (S. | | | | GTAGGGTTAGTAGGTAGGGCTAGTA |
| | cerevisiae) | | | | |
| | | | | | GGTAGGGCTAGTAGGTAGGGTTAGT |
| | | | | | AGGTAGGGTTCGTAGGTAGGGCTGG |
| | | | | | TAGGTAGGGTTAGTAGGTAGGGCTA |
| | | | | | GTAGGTAGGGCTAGTAGGTAGGGCT |
| | | | | | AGTAGGTAGGGTTAGTAGGTAGGGC |
| | | | | | TAGTAGGTAGGGCTAGTAGGTAGGG |
| | | | | | TTAGTAGGTAGGGTTCG |
| 37 | SEC14-like | NM_003003.1 | AK130317.1 | 1.5 | AGGTAGGGTTCGTAGgtagggctagtaggt |
| | 1 (S. | | | | |
| | cerevisiae) | | | | |
| 38 | cysteine-rich | NM_003118.1 | BG325726.1 | 1.4 | GTGAAGAAGATCCATGAGAATGAGA |
| | acidic | | | | AGCGCCTGGAG |
| | secreted | | | | |
| | protein | | | | |
| | (osteonectin) | | | | |
| 39 | cysteine-rich | NM_003118.1 | BG325726.1 | 1.3 | CTGGACCAGCACCCCattgacgggtacctc |
| | acidic | | | | |
| | secreted | | | | |
| | protein | | | | |
| | (osteonectin) | | | | |
| 40 | cysteine-rich | NM_003118.1 | AA325849.1 | 1.3 | CATGGAGCATTGCACCACCCGCTTT |
| | acidic | | | | TTCGAGACCTGTGACCTGGACAAT |
| | secreted | | | | |
| | protein | | | | |
| | (osteonectin) | | | | |
| 41 | cysteine-rich | NM_003118.1 | AA325849.1 | 1.3 | GAGAATGAGAAGCGCCTGGAGGCAG |
| | acidic | | | | GAGACCACCCCGTGGAGCTGCTGGC |
| | secreted | | | | CCGGGACTTCGAGAAGAACTATAAC |
| | protein | | | | ATGTACATCTTCCCTGTACACTGGC |
| | (osteonectin) | | | | AGTTCGGCCAGCTGGACCA |
| 42 | cysteine-rich | NM_003118.1 | AA325849.1 | 1.3 | GCACCCCATTGACGGgtacctctcccacac |
| | acidic | | | | |
| | secreted | | | | |
| | protein | | | | |
| | (osteonectin) | | | | |
| 43 | nuclear factor | NM_003204.1 | BM973053.1 | 1.5 | CGGGTCAGTGTACAGgaagaggcaggcact |
| | (erythroid- | | | | |
| | derived | | | | |
| | 2)-like 1 | | | | |
| 44 | nuclear factor | NM_003204.1 | BM97053.1 | 1.5 | TGCTGTGAGGCAGAGgaatgatggagaatc |
| | (erythroid- | | | | |
| | derived | | | | |
| | 2)-like 1 | | | | |
| 45 | synuclein, | NM_000345.2 | NM_007308.1 | 1.2 | TACGAACCTGAAGCCTAAGAAATAT |
| | alpha | | | | CTTTGCTCCCAGTTTCTTGAGATCT |
| | (non A4 | | | | GCTGACAGATGTTCCATCCTGTACA |
| | component | | | | AGTGCTCAGTTCCAATGTGCCCAGT |
| | of amyloid | | | | CATGACATTTCTCAAAGTTTTTACA |
| | precursor) | | | | GTGTATCTCGAAGTCTTCCATCAGC |
| | | | | | AGTGATTGAAGTATCTGTACCTGCC |
| | | | | | CCCACTCAGCATTTCGGTGCTTCCC |
| | | | | | TTTCACTGAAGTGAATACATGGTAG |
| | | | | | CAGGGTCTTTGTGTGCTGTGGATTT |
| | | | | | TGTGGCTTCAATCTACGATGTTAAA |
| | | | | | ACAAATTAAAAACACCTAAGTGACT |
| | | | | | ACCACTTATTTCTAAATCCTCACTA |
| | | | | | TTTTTTTGTTGCTGTTGTTCAGAAG |
| | | | | | TTGTTAGTGATTTGCTATCATATAT |
| | | | | | TATAAGATTTTTAGGTGTCTTTTAA |
| | | | | | TGATACTGTCTAAGAATAATGACGT |
| | | | | | ATTGTGAAATTTGTTAATATATATA |
| | | | | | ATACTTAAAAATATGTGAGCATGAA |
| | | | | | ACTATGCACCTATAAATACTAAATA |
| 46 | synuclein, | NM_000345.2 | NM_007308.1 | 1.4 | CCACAGGAAGGAATTCTGGAAGATA |
| | alpha | | | | TGCCTGTGGATCCTGACAATGAGGC |
| | (non A4 | | | | TTAT |
| | component | | | | |
| | of amyloid | | | | |
| | precursor) | | | | |
| 47 | synuclein, | NM_000345.2 | NM_007308.1 | 1.5 | GACCAGTTGGGCAAGaatgaagaaggagcc |
| | alpha | | | | |
| | (non A4 | | | | |
| | component | | | | |
| | of amyloid | | | | |
| | precursor) | | | | |
| 48 | synuclein, | NM_000345.2 | L36674.1 | 1.4 | TAAAGGAATTCATTAGCCATGGATG |
| | alpha | | | | TATTCATGAAAGGACTTTCAAAGGC |
| | (non A4 | | | | CAAGGAGGGAGTTGTGGCTGCTGCT |
| | component | | | | GAGAAAACCAAACAGGGTGTGGCAG |
| | of amyloid | | | | AAGCAGCAGGAAAGACAAAAGAGG |
| | precursor) | | | | |
| 49 | synuclein, | NM_000345.2 | L36674.1 | 1.3 | GTGTTCTCTATGTAGgctccaaaaaccaagg |
| | alpha | | | | |
| | (non A4 | | | | |
| | component | | | | |
| | of amyloid | | | | |
| | precursor) | | | | |
| 50 | transla- | NM_003295.1 | AA223997.1 | 0.5 | AAAACCTTTTATGACAGGGGCTGCA |
| | tionally | | | | GAACAAATCAAGCACATCCTTGCTA |
| | controlled | | | | ATTTCAAAAACTACCAG |
| | tumor | | | | |
| | protein 1 | | | | |
| 51 | transla- | NM_003295.1 | CA848049.1 | 0.7 | AGAtcgcggacgggttgtgcctggaggTGG |
| | tionally | | | | |
| | controlled | | | | |
| | tumor | | | | |
| | protein 1 | | | | |
| 52 | transla- | NM_003295.1 | CA848049.1 | 0.5 | TCCGACATCTACAAGatccgggagatcgcg |
| | tionally | | | | |
| | controlled | | | | |
| | tumor | | | | |
| | protein 1 | | | | |
| 53 | transla- | NM_003295.1 | BC022436.1 | 0.6 | CCGACATCTACAAGATCCGGGAGAT |
| | tionally | | | | CGCGGACGGGTTGTGCCTG |
| | controlled | | | | |
| | tumor | | | | |
| | protein 1 | | | | |
| 54 | transla- | NM_003295.1 | BC040008.1 | 0.5 | GCGCCGCTCCGGCTGCACCGCGCTC |
| | tionally | | | | GCTCCGAGTTTCAGGCTCGTGCTAA |
| | controlled | | | | GCTAGCGCCGTCGTCGTCTCCCTTC |
| | tumor | | | | AGTCGCCATCATGATTATCTACC |
| | protein 1 | | | | |
| 55 | TYRO protein | NM_003332.1 | BF092099.1 | 0.8 | GAGGGGCTGCGGAGGcagcgacccggaaac |
| | tyrosine | | | | |
| | kinase | | | | |
| | binding | | | | |
| | protein | | | | |
| 56 | triosephos- | NM_000365.3 | BI226906.1 | 1.3 | CATGCTCTGGCAGAGgatggctgaagtcca |
| | phate | | | | |
| | isomerase 1 | | | | |
| 57 | transgelin 2 | NM_003564.1 | AA428309.1 | 1.3 | ATTAACACCACTGACtgtgctcaccacaca |
| 58 | uroporphy- | NM_000374.2 | BQ008745.1 | 1.5 | GTGTGCCGCTGATTGtggaccctgatgaca |
| | rinogen | | | | |
| | decarboxylase | | | | |
| 59 | cold shock | NM_003651.3 | BC009744.1 | 1.3 | GAGGAGGAAGGGAGCGGCAGCAGT |
| | domain | | | | GAAGGATTTGACCCCCCTGCCACT |
| | protein A | | | | GATAGGCAGTTCTCTGGGGCCCGG |
| | | | | | AATCAGCTGCGCCGCCCCCAGTAT |
| | | | | | CGCCCTCAGTACCGGCAGCGGCGG |
| | | | | | TTCCCGCCTTACCACGTGGGACAG |
| | | | | | ACCTTTGACCGTCGCTCACGGGTC |
| | | | | | TTACCCCAT |
| 60 | cold shock | NM_003651.3 | BC009744.1 | 1.5 | CCCAACAGAATACAGgctggtgagattgga |
| | domain | | | | |
| | protein A | | | | |
| 61 | solute | NM_003982.2 | AW798524.1 | 0.8 | TCTCTGCTCTTCAATggtatcatggcattg |
| | carrier | | | | |
| | family 7 | | | | |
| | (cationic | | | | |
| | amino acid | | | | |
| | transporter, | | | | |
| | y+ system), | | | | |
| | member 7 | | | | |
| 62 | annexin A2 | NM_004039.1 | AV709641.1 | 0.9 | GACGCTTCTGAGCTaaaagcttccatgaag |
| 63 | annexin A2 | NM_004039.1 | BF244428.1 | 1.6 | AGCTTGGAGGGTGATgtgtggatgaggtca |
| 64 | beta-2- | NM_004048.2 | AV734235.1 | 0.7 | ATTTGGATTGGATGAATTCCAAATTC |
| | micro- | | | | TGCTTGCTTGCTTTTTAATATTGATA |
| | globulin | | | | TGCTTATACACTTACACTTTATGCAC |
| | | | | | AAAATGTAGGGTTATAATAATGTTAA |
| | | | | | CATGGACATGATCTTCTTTATAATTC |
| | | | | | TACTTTGAGTGCTGTCTCCATGTTTG |
| | | | | | ATGTATCTGAGCAGGTTGCTCCACA |
| | | | | | GGTAGCTCTAGGAGGGCTGGCAAC |
| 65 | beta2- | NM_004048.2 | BF912731.1 | 0.8 | AGATAGTTAAGTGGGatcgagacatgtaag |
| | micro- | | | | |
| | globulin | | | | |
| 66 | calreticulin | NM_004343.2 | CA306742.1 | 1.4 | GACTCCAAGCCTGAGgcagcagagaaacaa |
| 67 | CD74 antigen | NM_004355.1 | BQ029721.1 | 1.5 | ACACCCAGACCCCAGgaagagccaatgttt |
| 68 | HLA-B | NM_004640.3 | BM980603.1 | 1.4 | GCCCCAGGAGGAGAGcagtttaaagatttt |
| | transcript 1 | | | | |
| 69 | SEC24 | NM_004922.1 | AW449995.1 | 1.8 | GCAGCTGTCTGGAATgcagaggcagctgga |
| | related | | | | |
| | gene family, | | | | |
| | member C | | | | |
| | (S. | | | | |
| | cerevisiae) | | | | |
| 70 | actinin, | NM_004924.3 | CB051741.1 | 0.7 | CCATCGGGGCAGAAGaacttcatcacagct |
| | alpha 4 | | | | |
| 71 | hemoglobin, | NM_000517.3 | H78334.1 | 1.4 | CGGTCAACTTTCAAGctccttaagccactg |
| | alpha ½ | | | | |
| 72 | hemoglobin, | NM_000517.3 | H55830.1 | 1.4 | TCAAGGCCGCCTGGGgatgttcctgtcctt |
| | alpha ½ | | | | |
| 73 | cyclin- | NM_004935.1 | B1669825.1 | 0.8 | GTGGCTCTGAAACGGggtgtgccgagttcc |
| | dependent | | | | |
| | kinase 5 | | | | |
| 74 | histone | NM_004964.2 | BF204295.1 | 1.7 | CAGAGGAGAAGAAAGggtcaaggaggaggt |
| | deacetylase | | | | |
| | 1 | | | | |
| 75 | WD repeat | NM_005112.3 | BC030541.1 | 1.4 | CTAAGGAACATCGACgaccacagccgcttt |
| | domain 1 | | | | |
| 76 | ubiquitin | NM_005153.1 | W19112.1 | 0.7 | ATCGGCTCTTTGCAGtggtctaccatcacg |
| | specific | | | | |
| | protease 10 | | | | |
| 77 | v-fos FBJ | NM_005252.2 | BG541010.1 | 0.8 | CCTGTCAACGCGCAGgacttctgcacggac |
| | murine | | | | |
| | osteosarcoma | | | | |
| | viral | | | | |
| | oncogene | | | | |
| | homolog | | | | |
| 78 | v-fos FBJ | NM_005252.2 | BG541010.1 | 1.5 | GGCAAGGTGGAACAGgagacagaccaacta |
| | murine | | | | |
| | osteosarcoma | | | | |
| | viral | | | | |
| | oncogene | | | | |
| | homolog | | | | |
| 79 | v-fos FBJ | NM_005252.2 | B1325046.1 | 1.5 | GGCAAGGTGGAACAGgagacaggacaacta |
| | murine | | | | |
| | osteosarcoma | | | | |
| | viral | | | | |
| | oncogene | | | | |
| | homolog | | | | |
| 80 | hemoglobin, | NM_000558.3 | R91899.1 | 1.7 | ACCAAGACCTACTTCccggtcaacttcaag |
| | alpha ½ | | | | |
| 81 | hemoglobin, | NM_000558.3 | H58664.1 | 1.7 | GGAGGCCCTGGAGAGctcctaagccactgc |
| | alpha ½ | | | | |
| 82 | actin | NM_005718.2 | AI470163.1 | 0.7 | ATTGCTGTGAAACAGgggtatgatatcagc |
| | related | | | | |
| | protein ⅔ | | | | |
| | complex, | | | | |
| | subunit 4, | | | | |
| | 20 kDa | | | | |
| 83 | B-cell | NM_005745.5 | AI962313.1 | 1.3 | GCAGTTGCCACCTTCcacgcctgagcgtgg |
| | receptor- | | | | |
| | associated | | | | |
| | protein 31 | | | | |
| 84 | RNA binding | NM_005778.1 | CA488450.1 | 0.8 | GAGCAGACAAGTTTGactctgaacaggaag |
| | motif | | | | |
| | protein 5 | | | | |
| 85 | CD164 | NM_006016.3 | AF299342.1 | 0.8 | CTGTGACTCCAACCTCACAACCTGT |
| | antigen, | | | | GCGAAAGTCTACCTTTGATGCAGCC |
| | sialomucin | | | | AGTTTCATTGGAGGAATTGTCCTGG |
| | | | | | TCTTGGGTGTGCAGGCTGTAATTTT |
| | | | | | CTTTCTTTATAAATTCTGCAAATCT |
| | | | | | AAAGAACGAAATTACCACACTCTGT |
| | | | | | AAACAGACCCATTGAATTAATAAGG |
| | | | | | ACTGGTGATTCATTTGTGTAACTCA |
| | | | | | CTGAAGCCAAAATACTATCTTTTAA |
| | | | | | GATGTCCCACATGGAAGACGCTATT |
| | | | | | CCAGGATCTTTAAATTTCCATGGAT |
| | | | | | GCATATAGGATGTTTGGGAGCATCA |
| | | | | | TCCGTGAAGAAAAAATCAATTAAAT |
| | | | | | CATTGTGTTCAACAGGAATATTTAA |
| | | | | | AATATTCTGCATGAATCCTGTGGCT |
| | | | | | GTCTTATTTTAAATAGCTGCTGCTG |
| | | | | | TGGGATTATATTTTTTTTCCTTAAC |
| | | | | | ATGCCAAATATAACTTTCTGAAAGT |
| | | | | | GATGGAAAATGTTGTCTTGTGCAGA |
| | | | | | CAACATCATGGCTCTTGGCAGTTTA |
| 86 | CD164 | NM_006016.3 | AF299343.1 | 0.8 | CAGCCAATTCTACAGctaaacccacagttc |
| | antigen, | | | | |
| | sialomucin | | | | |
| 87 | ubiquitous | NM_006082.1 | BG981396.1 | 1.3 | CACCCTGAGCAGCTCaccacccacaccacc |
| | alpha | | | | |
| | tubulin | | | | |
| 88 | talin 1 | NM_006289.2 | AI393487.1 | 1.4 | CCCAGAGTATTAACGCTCCAAGAGT |
| | | | | | ATTATTAACGCTGCTGTACCTCGAT |
| | | | | | CTGAATCTGCCGGGGCCCCAGCCCA |
| | | | | | CTCCACCCTGCCAGCAGCTTCCAGC |
| | | | | | CAGTCCCCACAGCCTCATCAGCTCT |
| | | | | | CTTCACCGTTTTTTGATACTATCTT |
| | | | | | CCCCCACCCCCAGCTACCCATAGGG |
| | | | | | GCTGCAGAGTTATAAGCCCCAAACA |
| | | | | | GGTCATGCTCCAATAAAAATGATTC |
| | | | | | TACCTACAA |
| 89 | talin 1 | NM_006289.2 | AI393487.1 | 1.4 | TGCTTCGGAAGGAACgagagctggaagagg |
| 90 | talin 1 | NM_006289.2 | AI417760.1 | 1.4 | AGGAAGAAATGCTTCggaaggaacgagagc |
| 91 | acidic | NM_006327.1 | AW577170.1 | 1.4 | AGGAGGATGAGGATGgaggatgaagaaggt |
| | (leucine- | | | | |
| | rich) | | | | |
| | nuclear | | | | |
| | phospho- | | | | |
| | protein | | | | |
| | 32 family, | | | | |
| | member A | | | | |
| 92 | translocase | NM_006327.1 | AU142330.1 | 0.7 | GATGTTGCATGACAGgggcactttgggcta |
| | of inner | | | | |
| | mitochondrial | | | | |
| | membrane 23 | | | | |
| | homolog | | | | |
| | (yeast) | | | | |
| 93 | microspherule | NM_006337.3 | CD369365.1 | 1.5 | AACTCTGTGGTGGAGacaggaagctggggc |
| | protein 1 | | | | |
| 94 | APG7 | NM_006395.1 | BC000091.1 | 0.7 | CTTGTGCCTCACCAGatccggggatttctt |
| | autophagy | | | | |
| | 7-like (S. | | | | |
| | cerevisiae) | | | | |
| 95 | acidic | NM_006401.1 | Y07570.1 | 1.5 | GAAGTCAGTGAGGAGgaagaagaatttgga |
| | (leucine | | | | |
| | rich) | | | | |
| | nuclear | | | | |
| | phospho- | | | | |
| | protein 32 | | | | |
| | family, | | | | |
| | member B | | | | |
| 96 | acidic | NM_006401.1 | Y07570.1 | 1.4 | AGGATGAGGATGAAGaggaggaagaaggtg |
| | (leucine | | | | |
| | rich) | | | | |
| | nuclear | | | | |
| | phospho- | | | | |
| | protein 32 | | | | |
| | family, | | | | |
| | member B | | | | |
| 97 | acidic | NM_006401.1 | BF195216.1 | 1.6 | GAAGTCAGTGAGGAGaggaggaagaaggtg |
| | (leucine | | | | |
| | rich) | | | | |
| | nuclear | | | | |
| | phospho- | | | | |
| | protein 32 | | | | |
| | family, | | | | |
| | member B | | | | |
| 98 | butyrophilin, | NM_007049.2 | BQ929466.1 | 1.3 | CATTCTTACATGCTGaggaccggagaagtg |
| | subfamily 2, | | | | |
| | member A1 | | | | |
| 99 | EAP30 subunit | NM_007241.2 | BF525899.1 | 1.2 | CTGCAGCTGGCAGAGagaatggctacgtg |
| | of ELL | | | | |
| | complex | | | | |
| 100 | coatomer | NM_007263.2 | BM798704.1 | 1.5 | CCACGAGAGTCGGAGgaaggagctgaagag |
| | protein | | | | |
| | complex, | | | | |
| | subunit | | | | |
| | epsilon | | | | |
| 101 | soluble | NM_009587.1 | BG390210.1 | 1.2 | TACATCAGCTTCCAGacccagacagtcatc |
| | galactoside- | | | | |
| | binding | | | | |
| | lectin 9 | | | | |
| | (galectin 9) | | | | |
| 102 | soluble | NM_009587.1 | BG698264.1 | 0.8 | CTCCAGTGGAACCAGgtttgctgtgaactt |
| | galactoside- | | | | |
| | binding | | | | |
| | lectin 9 | | | | |
| | (galectin 9) | | | | |
| 103 | CDKN1A | NM_012127.1 | AK027287.1 | 1.3 | TAGAAGCTGGTGGAGgtgaggtccagagat |
| | interacting | | | | |
| | zinc | | | | |
| | finger | | | | |
| | protein 1 | | | | |
| 104 | CDKN1A | NM_012127.1 | AK027287.1 | 1.2 | CAGGCACATTCACAGccgcatctgccacaa |
| | interacting | | | | |
| | zinc | | | | |
| | finger | | | | |
| | protein 1 | | | | |
| 105 | F-box | NM_012179.2 | BE905968.1 | 1.3 | AATTTTGAAGCTGAGTCAATTCAAGA |
| | protein 7 | | | | TAATGCGCATATGGCAGAGGGCACAG |
| | | | | | GTTTCTATCCCTCAGAACCCATGCTC |
| | | | | | TGTAGTGAATCGGTGGAAGGGCAAGT |
| | | | | | GCCACATTCATTAGAGACCTTGTATC |
| | | | | | AATCAGCTGACTGTTCTGATGCCAAT |
| | | | | | GATGCCTTGATAGTGTTGATACATCT |
| | | | | | TCTCATGTTGGAGTCA |
| 106 | WW domain | NM_012478.2 | BG820375.1 | 1.2 | CCACCTCCCTACTACccaccggaagataag |
| | binding | | | | |
| | protein 2 | | | | |
| 107 | px19-like | NM_013237.2 | BM787853.1 | 1.3 | CACGCCCGGCTGATGggaatttggtcttgc |
| | protein | | | | |
| 108 | insulin-like | NM_000876.1 | BM787853.1 | 1.4 | GGAAACAGTGATAAGTAAGCTGACC |
| | growth | | | | ACTTGCTGTAGGAGAAGTTCCAACG |
| | factor 2 | | | | TGTCC |
| | receptor | | | | |
| 109 | insulin-like | NM_000876.1 | BM787853.1 | 1.3 | GACGCATCTCAAAACAGAGGGCTG |
| | growth | | | | CATTCGAAGAAACCCTTGCTGCTT |
| | factor 2 | | | | TAGTCCCGATAGGGTATTTGACCC |
| | receptor | | | | |
| | | | | | CGATATATTTTAGCATTTTAATTC |
| | | | | | TCTCCCCCTATTTATTGACTTTGA |
| | | | | | CAATTACTCAGGTTTGAGAAAAAG |
| | | | | | GAAAAAAAAACAGCCACCGTTTCT |
| | | | | | TCCTGCCAGCAGGGGTGTGATGTA |
| | | | | | CCAGTTTGTCCATCTTGAGATGGT |
| | | | | | GAGGCTGTCAGTGTATGGGGCAGC |
| | | | | | TTCCGGCGGGATGTTGAACTGGTC |
| | | | | | ATTAATGTGTCCCCTGAGTTGGAG |
| | | | | | CTCATTCTGTCTCTTTTCTCTTTT |
| | | | | | GCTTTCTGTTTCTTAAGGGCACAC |
| | | | | | ACACGTGCGTGCGAGCACACACAC |
| | | | | | ACATACGTGCACAGGGTCCCCGAG |
| | | | | | TGCCTAGGTTTTGGAGAGTTTGCC |
| | | | | | TGTTCTATGCCTTTAGTCAGGAAT |
| | | | | | GGCTGCACCTTTTTGCATGATATC |
| | | | | | TTCAAGCCTGGGCGTACAGAGCAC |
| | | | | | ATTTGTCAGTATTTTTGCCG |
| 110 | insulin-like | NM_000876.1 | BF222741.1 | 1.3 | AAGGAGGTCAGGCCCCACTCCTTC |
| | growth | | | | CTGATTGTTTACAGTCATTGGAAT |
| | factor 2 | | | | AAGGCATGGCTCAGATCGGCCACA |
| | receptor | | | | GGGCGGTACCTTGTGCCCAGGGTT |
| | | | | | TTGCCCCAAGTCCTCATTTAAAAG |
| | | | | | CATAAGGCCGGACGCATCTCAAAA |
| | | | | | CAGAGGGCTGCATTCGAAGAAACC |
| | | | | | CTTGCTGCTTTAGTCCCGATAGGG |
| | | | | | TATTTGACCCCGATATATTTTAGC |
| | | | | | ATTTTAATTCTCTCCCCCTATTTA |
| | | | | | TTGACTTTGACAATTACTCAGGTT |
| | | | | | TGAGAAAAAGGAAAAAAAAACAGC |
| | | | | | CACCGTTTCTTCCTGCCAGCAGGG |
| | | | | | GTGTGATGTACCAGTTTGTCCATC |
| | | | | | TTGAGATGGTGAGGCTGTCAGTGT |
| | | | | | ATGGGGCAGCTTCCGGCGGGATGT |
| | | | | | TGAACTGGTCATTAATGTGTCCCC |
| | | | | | TGAGTTGGAGCTCATTCTGTCTCT |
| | | | | | TTTCTCTTTTGCTTTCTGTTTCTT |
| | | | | | AAGGGCACACACACGTGCGTGCGA |
| | | | | | GCACACACACACATACGTGC |
| 111 | insulin-like | NM_000876.1 | BF222741.1 | 1.3 | GTGGCTGATGGAAGAgatccagctgcctcc |
| | growth | | | | |
| | factor 2 | | | | |
| | receptor | | | | |
| 112 | adducin 1 | NM_014190.2 | CA396829.1 | 1.3 | AGAAGGGCTCTGAAGagaatctggacgagg |
| | (alpha)/ | | | | |
| | adducin 1 | | | | |
| | (alpha) | | | | |
| 113 | eukaryotic | NM_014413.2 | BM142283.1 | 1.3 | GTTAACCTCACCCTACAGATGAAGA |
| | translation | | | | TAATAGAGCAAGAAAAAGAAATTGC |
| | initiation | | | | AGAACTAAAGAAGCAGCTAAACCTC |
| | factor 2- | | | | CTTTCTCAAGACAAAGGGGTGAGGG |
| | alpha | | | | ATGACGGAAAGGATGGGGGCGTGGG |
| | kinase 1 | | | | ATGAAAGTGGAC |
| 114 | KIAA0040 | NM_014656.1 | CB050264.1 | 1.7 | ATGGTTCCCAAGTGTgtgtaagtgtgtgta |
| 115 | Homocysteine- | NM_014685.1 | BG828243.1 | 0.8 | TGCATCAGGGGCTTTTGTTCCACCA |
| | inducible, | | | | CCAAGTGCACAAGAGATACCTGTGG |
| | endoplasmic | | | | TCTCTGCACCTGCTCCAGCCCCTAT |
| | reticulum | | | | TCACAACCAGTTTCCAGCTGAAAAC |
| | stress- | | | | CAGCCTGCCAATCAGAATGCTGCTC |
| | inducible, | | | | CTCAAGTGGTTGTTAATCCTGGAGC |
| | ubiquitin- | | | | CAATCAAAATTTGCGGATGAATGCA |
| | like | | | | CAAGGTGGCCCTATTGTGGAAGAAG |
| | domain | | | | ATGATGAAATAAATCGAGATTGGTT |
| | member 1 | | | | GGATTGGACCTATTCAGCAGCTACA |
| | | | | | TTTTCTGTTTTTCTCAGTATCCTCT |
| | | | | | ACTTCTACTCCTCCCTGAGCAGATT |
| | | | | | CCTCATGGTCATGGGGGCCACCGTT |
| 116 | Homocysteine- | NM_014685.1 | BG28243.1 | 0.7 | GGAAAACATCTCAAGgcctgaagctgccca |
| | inducible, | | | | |
| | endoplasmic | | | | |
| | reticulum | | | | |
| | stress- | | | | |
| | inducible, | | | | |
| | ubiquitin- | | | | |
| | like | | | | |
| | domain | | | | |
| | member 1 | | | | |
| 117 | Homocysteine- | NM_014685.1 | BG282243.1 | 0.8 | GTACTACATGCAATAtttagcagccactgc |
| | inducible, | | | | |
| | endoplasmic | | | | |
| | reticulum | | | | |
| | stress- | | | | |
| | inducible, | | | | |
| | ubiquitin- | | | | |
| | like | | | | |
| | domain | | | | |
| | member 1 | | | | |
| 118 | lysosomal- | NM_014713.2 | AL039105.1 | 0.8 | CTGATTCCATTCTTCTGTTACCGACT |
| | associated | | | | TTTTGACTTCGTCCTCAGTTGCCTGG |
| | protein | | | | TTGCTATTAGTTCTCTCACCTATTTG |
| | transmembrane | | | | CCAAGAATCAAAGAATATCTGGATCA |
| | 4 alpha | | | | ACTA |
| 119 | DAZ | NM_014764.1 | AU118651.1 | 0.8 | AGCAGTACCTCCCTAAAGCATTTTG |
| | associated | | | | AGGTAGGGGAGGTATCCATTCATAA |
| | protein 2 | | | | AATGAATGTGGG |
| 120 | ring finger | NM_014868.3 | BU626650.1 | 1.5 | CGAGAGCGCAGGATTGAGATAGAG |
| | protein 10 | | | | GAGAACA |
| 121 | ring finger | NM_014868.3 | BU626650.1 | 1.5 | AGAAACAGGGCAAGTacccagaagtccaca |
| | protein 10 | | | | |
| 122 | ring finger | NM_014868.3 | BF815780.1 | 1.3 | CAGAAGTCCACATTCCCCTCGAGAAT |
| | protein 10 | | | | CTACAGCAGTTTCCTGCCTTCAATTC |
| | | | | | TTATACCTGCTCCTCTGATTCTGCTT |
| | | | | | TGGGTCCCACCAGCACCGAGGGCCAT |
| | | | | | GGGGCCCTCTCCATTTCTCCTCTCAG |
| | | | | | CAGAAGTC |
| 123 | ring finger | NM_014868.3 | BF815780.1 | 1.4 | CAGGTTCCCATGCAGactttctgctgaccc |
| | protein 10 | | | | |
| 124 | ribosomal | NM_000968.2 | BM846228.1 | 0.7 | TCAGTGAATTAGCAGgtcatcagactagtg |
| | protein L4 | | | | |
| 125 | ribosomal | NM_000968.2 | CB141160.1 | 0.7 | GTCATCAGACTAGTGCTGAGTCTTG |
| | protein L4 | | | | GGGTACTGGCAGAGCTGTGGCTCGA |
| | | | | | ATTCCCAGAGTTCGAGGTGGTGGGA |
| | | | | | CTCACCGCTCTGGCCAGGGTGCTTT |
| | | | | | TGGAAACatgtgtcgtg |
| 126 | ribosomal | NM_000968.2 | CD686462.1 | 0.6 | GCGTGTGCTCGCCCACTGATATCGG |
| | protein L4 | | | | TGTACTCCGAAAAGGGGGAGTCATC |
| | | | | | TGGCAAAAATGTCACTTTGCCTGCT |
| | | | | | GTATTCAAGGCTCCTATTCGACCAG |
| | | | | | ATATTGTGAACTTTGTTCACACCAA |
| | | | | | CTTGCGCAAAAACAACAGACAGCCC |
| | | | | | TATGCTG |
| 127 | ribosomal | NM_000968.2 | CD686462.1 | 0.7 | TGGTCATGTCTAAAGgtcatcgtattgagg |
| | protein L4 | | | | |
| 128 | ribosomal | NM_000969.2 | BE879402.1 | 0.8 | GATATCATTTGTCAGattgcttatgcccgt |
| | protein L5 | | | | |
| 129 | BAT2 domain | NM_015172.1 | AB029019.2 | 1.4 | CACTTCCACCTTCAAccttagctccagttt |
| | containing 1 | | | | |
| 130 | adenosine | NM_015841.1 | AA096321.1 | 1.3 | TCACACAGGACAGAGgaggcagagcttctg |
| | deaminase, | | | | |
| | RNA-specific | | | | |
| 131 | endoplasmic | NM_015913.2 | CA418006.1 | 0.8 | TTCCCAAATTTCTACagcctctttcctctt |
| | reticulum | | | | |
| | thioredoxin | | | | |
| | superfamily | | | | |
| | member, 18 | | | | |
| | kDa | | | | |
| 132 | hypothetical | NM_016127.3 | AA576624.1 | 0.8 | GGAGTCAAACACTGGatgcagaaattttgg |
| | protein | | | | |
| | MGC8721 | | | | |
| 133 | tetratrico- | NM_017775.2 | BE164618.1 | 1.5 | TTTTGATGCACAGAGctaccaccacagtgc |
| | peptide | | | | |
| | repeat | | | | |
| | domain 19 | | | | |
| 134 | hypothetical | NM_017841.1 | BI553945.1 | 0.8 | TCTTTTTGCTAAAGAACATCTGCAGC |
| | protein | | | | ACATGACAGAAAAGCAGCTGAACCTC |
| | FLJ20487 | | | | TATGACCGCCTGATTAACGAGCCTAG |
| | | | | | TAATGACTGGGATATTT |
| 135 | hypothetical | NM_017865.2 | AK001070.1 | 1, 6 | ACCAGAGACCTTCCCACCTCCAGGAG |
| | protein | | | | AGGAAGAGGGTGAGGAAGAAGAGGAC |
| | FLJ20531 | | | | AATGATGAGGATGAAGAGGAGATGCT |
| | | | | | CAGTGATGCCAGCTTATGGACCTACA |
| | | | | | G |
| 136 | ubiquitin B | NM_018955.2 | BE708511.1 | 1, 4 | CCCTGACCGGCAAGAcatcactctggaggt |
| 137 | ubiquitin B | NM_018955.2 | AA206538.1 | 1, 4 | CAGGTCAAAATGCAGatcttcgtgaaaacc |
| 138 | ubiquitin B | NM_018955.2 | AA206538.1 | 1, 3 | CAGGTCAAAATGCAGatcttcgtgaagacc |
| 139 | ubiquitin B | NM_018955.2 | AA340917.1 | 1, 4 | AGATCTTCGTGAAGACCCTGACCGG |
| | | | | | CAAGACCATCACTCTGGAGGTGGAG |
| | | | | | CCCAGTGACACCATCGAAAATGTGA |
| | | | | | AGGCCAAGATCCAAGATAAAGAAGG |
| | | | | | CATCCCCCCCGACCAGCAGAGGCTC |
| | | | | | ATCTTTGCAGGCAAGCAGCTGGAAG |
| | | | | | ATGGCCGCACTCTTTCTGACTACAA |
| | | | | | CATCCAGAAAGAGTCGACCCTGCAC |
| | | | | | CTGGTCCTGCGCCTGAGGGGTGGCT |
| | | | | | GTTAATTCTTCAGTCATGGCATTCG |
| | | | | | CAGTGCCCAGTGATGGCATTACTCT |
| | | | | | GCACTATAGCCATTTGCCCCAACTT |
| | | | | | AAGTTTAGAAATTACAAGTTTCAGT |
| | | | | | AATAGCTGAACCTGTTCAAAATGTT |
| | | | | | AATAAAGG |
| 140 | ubiquitin B | NM_018955.2 | BU661443.1 | 1, 4 | TCCCTGTGGGTGGACGTGGTTGGT |
| | | | | | GATTGGCAGGATCCT |
| 141 | ubiquitin B | NM_018955.2 | BU661443.1 | 1, 5 | GGTTGGCTTTGTTGGgtgagcttgtttgtg |
| 142 | DnaJ (Hsp40) | NM_018981.1 | AF490904.1 | 1, 3 | CTTAGTGGCATGTTGgatggtcttgttaat |
| | homolog, | | | | |
| | subfamily C, | | | | |
| | member 10 | | | | |
| 143 | major histo- | NM_019111.2 | AA505585.1 | 0, 8 | AGAGAGAAGATCACTGAAGAAACTT |
| | compatibility | | | | CTGCTTTAATGACTTTACAAAGCTG |
| | complex, | | | | GCAATATTACAATCCTTGACCTCAG |
| | class II, | | | | TGAAAGCAGTCATCTTCAGCGTTTT |
| | DR alpha | | | | CCAGCCCTATAGCCACCCCAAGTGT |
| | | | | | GGTTATGCCTCCTCGATTGCTCCGT |
| | | | | | ACTCTAAC |
| 144 | major histo- | NM_019111.2 | AA505585.1 | 0, 8 | ATCTAGCTGGCTTTCcctgtctattgcctt |
| | compatibility | | | | |
| | complex, | | | | |
| | class II, | | | | |
| | DR alpha | | | | |
| 145 | major histo- | NM_019111.2 | CD686254.1 | 0, 7 | TCAGAGACAGTCTTCCTGCCCAGGG |
| | compatibility | | | | AAGACCACCTTTTCCGCAAGTTCCA |
| | complex, | | | | CTATCTCCCCTTCCTGCCCTCAACT |
| | class II, | | | | GAGGACGTTTACGACTGCAGGGTGG |
| | DR alpha | | | | AGCACTGGGGCTTGGATGAGCCTCT |
| | | | | | TCTCAAGCACTGGGagtttgatgct |
| | | | | | ccaagccctctc |
| 146 | KIAA1191 | NM_020444.2 | BE313670.1 | 1, 7 | GCAGCAGGATCACAGgtggaggaaggagag |
| | protein | | | | |
| 147 | KIAA1191 | NM_020444.2 | BI254429.1 | 1, 5 | GCAGCAGGATCACAGaacagacccaggaaa |
| | protein | | | | |
| 148 | mesoderm | NM_020948.1 | AY124186.1 | 0, 7 | TTTCTCACACAGGCAtactccaaatgcttc |
| | induction | | | | |
| | early | | | | |
| | response 1 | | | | |
| 149 | Actinin, | NM_001102.2 | BG036045.1 | 0, 7 | AACCACTTTGACCGGggagaagcagaattt |
| | alpha 1 | | | | |
| 150 | membrane- | NM_022349.2 | NM_152851.1 | 0, 8 | GGAACTCTCTCTCTGATGCTGATTT |
| | spanning 4- | | | | GCACTCTGCTGGAATTCTGCCTAGC |
| | domains, | | | | TGTGCTCACTGCTGTGCTGCGGTG |
| | subfamily A, | | | | GAAACAGGCTTAC |
| | member 6A | | | | |
| 151 | myelin | NM_024569.2 | AI693779.1 | 0, 9 | TGCCCTGGCATTCTGGCAGAGAATC |
| | protein | | | | CTCACCAGTTCTCACCAACCTTCCC |
| | zero-like 1 | | | | CCCAGGCAAGGGCAGCTGCCAGCAT |
| | | | | | GGTGCTCTGCCAGGACAGGTTTCCC |
| | | | | | TGAAGGAAGCTGCTCACACTGAGAT |
| | | | | | GAGCCTCTCAGGGCAGGACCTCTTC |
| | | | | | CCAAGCCCTGCACACCCACCCCTGC |
| | | | | | AGCCCTTTTGGCTC |
| 152 | likely | NM_030915.1 | W45195.1 | 1, 1 | GCTTCAGTCCGCCGAgagcagtaccgtgtg |
| | ortholog of | | | | |
| | mouse limb- | | | | |
| | bud and | | | | |
| | heart gene | | | | |
| 153 | heterogeneous | NM_031314.1 | CF128443.1 | 1, 2 | CTGACCCAGATAAAACAAAAAGTGG |
| | nuclear | | | | ATTCTCTCCTGGAAAACCTGGAAAA |
| | ribonucleo- | | | | AATGGAAAAGGAACAGAGCAAACAA |
| | protein | | | | GCAG |
| | C (C1/C2) | | | | |
| 154 | polymerase | NM_032940.1 | NM_002694.2 | 1, 3 | GGTGGGGGTGGAAGCagccgcaggagcaag |
| | (RNA) II | | | | |
| | (DNA | | | | |
| | directed) | | | | |
| | polypeptide | | | | |
| | C, 33 kDa | | | | |
| 155 | caspase 4, | NM_033306.1 | NM_001225.2 | 1, 2 | TGTTCCCTATGGCAGaaggcaaccacagaa |
| | apoptosis- | | | | |
| | related | | | | |
| | cysteine | | | | |
| | protease | | | | |
| 156 | major histo- | NM_033554.2 | BU621846.1 | 0, 8 | GTCGCTGAGAGCCTCTTCCTGCCCA |
| | compatibility | | | | GAACAGATTACAGCTTCCACAAGTT |
| | complex, | | | | CCATTACCTGACCTTTGTGCCCTCA |
| | class II, | | | | GCAGAGGACTTCTATGACTGCAGGG |
| | DP alpha 1 | | | | TGGAGCACTGGGGCTTGGACCAGCC |
| | | | | | GCTCCTCAAGCACTGGG |
| 157 | major histo- | NM_033554.2 | BU621846.1 | 0, 7 | ACCCTCATCTGCCACATTGACAAGT |
| | compatibility | | | | TCTTCCCACCAGTGCTCAACGTCAC |
| | complex, | | | | GTGG |
| | class II, | | | | |
| | DP alpha 1 | | | | |
| 158 | major histo- | NM_033554.2 | BU621846.1 | 0, 7 | AAGGAGCCTGTGGAGctgggccagcccaac |
| | compatibility | | | | |
| | complex, | | | | |
| | class II, | | | | |
| | DP alpha 1 | | | | |
| 159 | FK506 binding | NM_054014.1 | NM_000801.2 | 0, 8 | GCTCCCTGTTCTTGGatctgccatggaggg |
| | protein 1A, | | | | |
| | 12 kDa | | | | |
| 160 | chloride | NM_001288.3 | BG491600.1 | 0, 8 | CCAGGGACGGCCACTTCCTGGTCC |
| | intracellular | | | | CCGACGCAACCATGGCTGAAGAAC |
| | channel 1 | | | | AACCGCAGGTCGAATTGTTCGTGA |
| | | | | | AG |
| 161 | chloride | NM_001288.3 | AV683308.1 | 0.7 | GGGCAGCTCCCATTCctgctgtatggcact |
| | intracellular | | | | |
| | channel 1 | | | | |
| 162 | vacuolar | NM_080631.1 | BX648347.1 | 1.4 | ACCGGAGGGAAGAAGgtgtgctcagtgaag |
| | protein | | | | |
| | sorting 41 | | | | |
| | (yeast) | | | | |
| 163 | zinc finger | NM_133476.2 | BC053361.1 | 1.5 | TGGAAAGGAGAAGGAATAAGACGG |
| | protein 384 | | | | CAGGAGGAAGAGAGAGAGAGG |
| 164 | chromosome 19 | NM_138774.2 | AI375989.1 | 1.4 | ACCTCATCTCGGCCAgtgctgacctggagg |
| | open reading | | | | |
| | frame 22 | | | | |
| 165 | nuclear | NM_000176.1 | X03348.1 | 0.8 | TTCCTAAGGACGGTCTGAAGAGCCA |
| | receptor | | | | AGAGCTATTTGATGAAATTAGAATG |
| | subfamily 3, | | | | ACCTACATCAAAGAGCTAGGAAAAG |
| | group C, | | | | CCATTGTCAAGAGGGAAGGAAACTC |
| | member 1 | | | | CAGCCAGAACTGGCAGCGGTTTTAT |
| | (glucocorti- | | | | CAACTGACAAAACTCTTG |
| | coid | | | | |
| | receptor) | | | | |
| 166 | Nucleosome | NM_139207.1 | BU620919.1 | 0.8 | AGTGATATGGTTCAGgaacacgatgaacct |
| | assembly | | | | |
| | protein | | | | |
| | 1-like 1 | | | | |
| 167 | Nucleosome | NM_139207.1 | AU117948.1 | 0.8 | ATGAATATTTTACAAATGAAGTGCTG |
| | assembly | | | | ACAAAGACATACAGGATGAGGTCAGA |
| | protein | | | | A |
| | 1-like 1 | | | | |
| 168 | Nucleosome | NM_139207.1 | AU117948.1 | 0.8 | GCTGGCCAGCCTATGagttttgtcttagaa |
| | assembly | | | | |
| | protein | | | | |
| | 1-like 1 | | | | |
| 169 | Nucleosome | NM_139207.1 | AK122670.1 | 0.8 | CCTGCCTAGGGTAGTTAAAAGACGA |
| | assembly | | | | GTGAATGCTCTCAAAAACCTGCAAG |
| | protein | | | | TTAAATGTGCACAGATAGAAGCCAA |
| | 1-like 1 | | | | ATTCTATGAGGAAGTTCACGATCTT |
| | | | | | GAAAGGAAGTATGCTGTTCTCTATC |
| | | | | | AG |
| 170 | TAF15 RNA | NM_139215.1 | BF812650.1 | 1.3 | GCTATGGTGGGGACAGAGGAGGCG |
| | polymerase | | | | GCTATGGAGGAGACCGAGGAGGTG |
| | II, TATA box | | | | GCTATGGAGGAGATCGAGGTGGCT |
| | binding | | | | ATGGAGGAGACCGAGGTGGAGGCT |
| | protein | | | | ATGGTGGAGACCGAGGAGGCTATG |
| | (TBP)- | | | | GAGGAGATCGAGGAGGTTACGGAG |
| | associated | | | | GAGATCGAGGAGGTTATGGAGGAG |
| | factor, | | | | ATCGAGGAGGCTATGGAGGAGACA |
| | 68 kDa | | | | GAAGCCGGGGGGGCTATGGAGGAG |
| | | | | | ACCGTGGTGGTGGCAGTGGCTACG |
| | | | | | GTGGAGACCGAAGTGGAGGCTATG |
| | | | | | GAGGAGACAGGAGTGGTGGCGGCT |
| | | | | | ATGGAGGAGACCGAGGTGGGGGCT |
| | | | | | ACGGAGGAGACCGAGGTGGCTATG |
| | | | | | GAGG |
| 171 | TAF15 RNA | NM_139215.1 | BF812650.1 | 1.3 | GGGACAGAGGCGGCGgctatggtggggaca |
| | polymerase | | | | |
| | II, TATA box | | | | |
| | binding | | | | |
| | protein | | | | |
| | (TBP)- | | | | |
| | associated | | | | |
| | factor, | | | | |
| | 68 kDa | | | | |
| 172 | DEAD (Asp- | NM_001356.2 | BE000596.1 | 0.8 | GACCACAGCAATGACCAGCCCTCAT |
| | Glu-Ala- | | | | TAGGGCCCTGGATGATTTTTGGTCT |
| | Asp) box | | | | AATAACGCATGCTAGTGTTGATGTT |
| | polypeptide | | | | TTTTGGTCAGAGGGTATGAACAGGA |
| | 3, X-linked | | | | AGAATTAAATGCAGCAGGCTTTATT |
| | | | | | TTAAATGCCGATTCACATTACTCTG |
| | | | | | TTCAAGCTGCGTTGAGATGTTAAAC |
| | | | | | TGGCTTACTATAGACTTCGTAAAAA |
| | | | | | TGGCTCCAGAAAAGTAACAAACTGA |
| | | | | | AATCTTTGAGATCACACAGGTTGGA |
| | | | | | AATATGTACATAACTGCACAAGGTG |
| | | | | | TCAATTCTGCTCTACAGTGCAGTTT |
| | | | | | TAGTCAGTTTTAGTTGCATAGGTTT |
| | | | | | CCATTGTATTTATAGTCTGTTTATG |
| | | | | | CTAAATCTGGCCAAAGATGAACATT |
| | | | | | GTCCACCACTAAAATGCCTCTGCCA |
| | | | | | CTTTGAATTCTGTGCTAATTTTGTG |
| | | | | | GCCAGAATGCGGTGATCAAAACGCT |
| | | | | | CCATCTTTTTACAGTGGCATAGGAA |
| | | | | | GACGGCAAAAATTTCCTAAAGTGCA |
| 173 | copine II | NM_152727.4 | AK094867.1 | 1.4 | ACCCCTTCTGCTCAGgtgtggatggtattg |
| 174 | heat shock | NM_153201.1 | BU731317.1 | 0.8 | ACTCGTATCCCCAAGattcagaagcttctc |
| | 70 kDa | | | | |
| | protein 8 | | | | |
| 175 | hypothetical | NM_153233.1 | BC038360.1 | 1.4 | ACTGGCCAGGACCTGgaagcagacacctct |
| | protein | | | | |
| | FLJ36445 | | | | |
| 176 | dynein, | NM_001378.1 | U39575.1 | 1.4 | GGAAGACAAAGAAGGagagattcaagcagg |
| | cytoplasmic, | | | | |
| | intermediate | | | | |
| | polypeptide | | | | |
| | 2/dynein, | | | | |
| | cytoplasmic, | | | | |
| | intermediate | | | | |
| | polypeptide 2 | | | | |
| 177 | tropomyosin3 | NM_153649.1 | BM006741.1 | 1.3 | TGATGAGAGTGAGAGgcagagacccgtgct |
| 178 | tropomyosin 3 | NM_153649.1 | BM006741.1 | 1.3 | TGATGAGAGTGAGAGaggatgctggaccag |
| 179 | EST BE881352.1 | | BE881352.1 | 1.3 | TCAATATAAAACCCCcacctaccacacatt |
| 180 | EST AW368637.1 | — | AW368637.1 | 1.4 | CTTAAACTCCAGCACcatcatagccaccat |
| 181 | eukaryotic | NM_001402.4 | AU146228.1 | 1.5 | CACCAATGGAAGCAGtggacaagaaggctg |
| | translation | | | | |
| | elongation | | | | |
| | factor 1 | | | | |
| | alpha 1 | | | | |
| 182 | eukaryotic | NM_001402.4 | BU580573.1 | 0.8 | CATCAAAGCAGTGGACAAGAAGGCT |
| | translation | | | | GCTGGAGCTGGCAAGGTCACCAAGT |
| | elongation | | | | CTGCCCAGAAAGCTCAGAAGGCTAA |
| | factor 1 | | | | ATGAATATTATCCCTAATACCTGCC |
| | alpha 1 | | | | ACCCCACTCTTAATCAGTGGTGGAA |
| | | | | | GAACGGTCTCAGAACTGTTTGTTTC |
| | | | | | AATTGGCCATTTAAGTTTAGTAGTA |
| | | | | | AAAGACTGGTTAATGATAACAATGC |
| | | | | | ATCGTAAAACCTTCAGAAGGAAAGG |
| | | | | | AGAATGTTTTGTGGACCACTTTGGT |
| | | | | | TTTCTTTTTTGCGTGTGGCAGTTTT |
| | | | | | AAGTTATTAGTTTTTAAAATCAGTA |
| | | | | | CTTTTTAATGGAAACAACTTGACCA |
| | | | | | AAAATTTGTCACAGAATTTTGAGAC |
| | | | | | CCATTAAAAAAGTTAAATGAG |
| 183 | eukaryotic | NM_001402.4 | AA595862.1 | 0.8 | TGCGGTGGGTGTCATCAAAGCAGTG |
| | translation | | | | GACAAGAAGGCTGCTGGAGCTGGCA |
| | elongation | | | | AGGTCACCAAGTCTGCCCAGAAAGC |
| | factor 1 | | | | GCTCAGAAGGCTAAATGAATATTAT |
| | alpha 1 | | | | CCCTAATACCTGCCACCCCACTCTT |
| | | | | | AATCAGTGGTGGAAGAACGGTCTCA |
| | | | | | GAACTGTTTGTTTCAATTGGCCATT |
| | | | | | TAAGTTTAGTAGTAAAAGACTGGTT |
| | | | | | AATGATAACAATGCATCGTAAAACC |
| | | | | | TTCAGAAGG |
| 184 | eukaryotic | NM_001404.3 | AA206367.1 | 0.7 | CTGAGTCCAGATTGGCAGGTGGACT |
| | translation | | | | ACGAGTCATACACATGGCGGAAACT |
| | elongation | | | | GGATCCTGGCAGCGAGGAGACCCAG |
| | factor 1 | | | | ACGCTGGTT |
| | gamma | | | | |
| 185 | eukaryotic | NM_001404.3 | AA206367.1 | 0.6 | CAGCATGTGGGCAAAGCCTTCAATC |
| | translation | | | | AGGGCAAGATCTTCAAGTGAACATC |
| | elongation | | | | TCTTGCCATCACCTAG |
| | factor 1 | | | | |
| | gamma | | | | |
| 186 | eukaryotic | NM_001404.3 | BQ375267.1 | 0.8 | GTTCTAGAGCCTTCTTTCCGCCAGG |
| | translation | | | | CCTTCCCAATACCAACCGCTGGTTC |
| | elongation | | | | CTCACCTGCATTAACCAGCCCCAGT |
| | factor 1 | | | | TCCGGGCTGTCTTGGGCGAAGTGAA |
| | gamma | | | | ACTGTGTGAGAAGATGGCCCAGTTT |
| | | | | | GATGctaaaaagtttgcagag |
| 187 | eukaryotic | NM_001404.3 | BU783548.1 | 0.6 | ATTTAAGCGCAAGTACTCCAATGAG |
| | translation | | | | GACACACTCTCTGTGGCACTGCCAT |
| | elongation | | | | ATTTCTGGGAGCACTTTGATAAGGA |
| | factor 1 | | | | CGGCTGGTCCCTGTGGTACTCAGAG |
| | gamma | | | | TATCGCTTCCCTGAAGAACTCACTC |
| | | | | | AGACCTTCATGAGCTGCAATCTCAT |
| | | | | | CACTG |
| 188 | eukaryotic | NM_001404.3 | BE502067.1 | 0.6 | TGGACAAGCTGAGGAAGAATGCCTT |
| | translation | | | | CGCCAGTGTCATCCTTTTTGGAACC |
| | elongation | | | | AACAATAGCAGCTCCATTTCTGGAG |
| | factor 1 | | | | TCTGGGTCTTCCGAGGCCAG |
| | gamma | | | | |
| 189 | eukaryotic | NM_001404.3 | BE502067.1 | 0.6 | CAGGTGGACTACGAGTCATACACAT |
| | translation | | | | GGCGGAAACTGGATCCTGGCAGCGA |
| | elongation | | | | GGAGACCCAGACGCTGGTTCGAGAG |
| | factor 1 | | | | TACTTTTCCTGGGAGGGGGCCTTCC |
| | gamma | | | | AGCATGTGGGCAAAGCCTTCAA |
| 190 | eukaryotic | NM_001404.3 | BE502067.1 | 0.6 | GAGCTTGCCTTTCCGctgagtccagattgg |
| | translation | | | | |
| | elongation | | | | |
| | factor 1 | | | | |
| | gamma | | | | |
| 191 | eukaryotic | NM_001404.3 | BG533219.1 | 0.8 | AAGGAGGAGAAAAAGGCGGCTGCC |
| | translation | | | | CCTGCTCCTGAGGAGGAGATGGAT |
| | elongation | | | | GAATGTGAGCAGGCGCTGGCTGCT |
| | factor 1 | | | | GAGCCCAAGGCCAAGGACCCCTTC |
| | gamma | | | | GCTCACCTGCCCAAGAG |
| 192 | eukaryotic | NM_001404.3 | BG615194.1 | 0.7 | TTCCGCCAGGCCTTTCCCAATACC |
| | translation | | | | AACCGCTGGTTCCTCACCTGCATT |
| | elongation | | | | AACCAGCCCCAGTTCCGGGCTGTC |
| | factor 1 | | | | TTGGGCGAAGTGAAACTGTGTGAG |
| | gamma | | | | AAGA |
| 193 | eukaryotic | NM_001404.3 | BG702200.1 | 0.8 | GTTCACGGGAAGAGAAGCAGAAGC |
| | translation | | | | CCCAGGCTGAGCGGA |
| | elongation | | | | |
| | factor 1 | | | | |
| | gamma | | | | |
| 194 | O-linked N- | NM_181673.1 | AW002377.1 | 0.8 | AAAGAATATCTAGCCctctgttcaacacca |
| | acetylgluco- | | | | |
| | samine | | | | |
| | (GlcNAc) | | | | |
| | transferase | | | | |
| 195 | O-linked N- | NM_181673.1 | AW002377.1 | 0.8 | CACGAAAAGTAGCCGctctggttgaagctt |
| | acetylgluco- | | | | |
| | samine | | | | |
| | (GlcNAc) | | | | |
| | transferase | | | | |
| 196 | WD and | AB028960.1 | AK023778.1 | 1.3 | CCAATTTCTTTGGCAgcaacgctcagtata |
| | tetratrico- | | | | |
| | peptide | | | | |
| | repeats 1 | | | | |
| 197 | actin, | NM_001614.2 | CD687776.1 | 1.2 | CATGGAGAAGATCTGggcgcaccactggca |
| | gamma 1 | | | | |
| 198 | arachidonate | NM_001629.2 | BX366320.1 | 1.3 | AGAGAGAACGCAGAGgccacggaagccctg |
| | 5-lipoxy- | | | | |
| | genase | | | | |
| | activating | | | | |
| | protein | | | | |
| 199 | Homo sapiens | AK026373.1 | AV725084.1 | 1.2 | ATTTCAACAGCTGAGgaaggtgtcttgctg |
| | cDNA: | | | | |
| | FLJ22720 | | | | |
| | fis, clone | | | | |
| | HSI14320 | | | | |
| 200 | Homo sapiens | AK026373.1 | BQ276346.1 | 0.8 | TTTGGCAGGAAGGTGTCTTGCTGCA |
| | cDNA: | | | | GGTAACTAATGAAGAAGTGGTCAAC |
| | FLJ22720 | | | | CACAGAGTCTTCAAGAAATAAGAAA |
| | fis, clone | | | | TTCTGTACCATCTGAAAGTAGTTCT |
| | HSI14320 | | | | TGTTGGTGCCTTCATTTAAAAAGCA |
| | | | | | CTCTTTAAAATAAAAGGGAAATGTT |
| | | | | | TTCTGATAAAA |
| 201 | DKFZp586K2322 | AL080113.1 | AV751235.1 | 0.8 | TTTATTTTCAAATGCAGTGTAGAGC |
| | (NM_006386 or | | | | TAGATTAAAAGCAACTCTTTGCCAC |
| | NM_030881) | | | | CTACTCTGCCCTTTTGGCAAAGTTA |
| | | | | | CCTTGAACAAAGAATCTTAAGGGTT |
| | | | | | TATTAAGAACTCTTTATTTTCTTCA |
| | | | | | TACCCTGTTCTCTGCAGTGCTTTCT |
| | | | | | AACAGCTTCTGGGTGCAGATTTTCT |
| | | | | | TCGGCATCCTTTTGCACTCAGCTTA |
| | | | | | TTACAGGTAGGTAGTGCTTAAGAAA |
| | | | | | AGTCATGGAGGACTAAAGCCTAAGT |
| | | | | | CCTTTTCACTTTTCCTCCATCTGAA |
| | | | | | GGTAGGTGAGTTCATCCTCTTCATA |
| | | | | | GTAATGCTGTTTTACCAAGACTTTA |
| | | | | | TAGCAGATGGACCCAGAAAGAATTT |
| | | | | | TCTGCTATTGTGTTCACTACAACAG |
| | | | | | GATAGGGACATCAGACAGCCCCAGA |
| | | | | | AACCCCTTCCAGATCTGATATGGGA |
| | | | | | CTATTAATTTTTATGCTGTTAATTG |
| | | | | | GTATTCATTCACAATGCAGTTGAAG |
| | | | | | GGGGAAGGCTCCACTGCATTCTTTG |
| 202 | calmodulin 2 | NM_001743.3 | BF701704.1 | 0.8 | ATTGCAAAACGGGTGtattatccaggtact |
| | (phosphorylase | | | | |
| | kinase, delta) | | | | |
| 203 | calpain, small | NM_001749.1 | BE907701.1 | 1.4 | CCTTTGAGGCAGCAGatgaaagtgggaaca |
| | subunit 1/ | | | | |
| | calpain, | | | | |
| | small | | | | |
| | subunit 1 | | | | |
| 204 | Cysteinyl- | NM_001751.3 | AK125503.1 | 1.5 | AAACAGGAACAAGAAgcagcaaagctggcc |
| | tRNA | | | | |
| | synthetase | | | | |
| 205 | CD97 antigen | NM_001784.2 | NM_078481.1 | 1.3 | ATACCGTCTGTGAAGatgtggacgagtgca |
| 206 | CD97 antigen | NM_001784.2 | BI028545.1 | 1.2 | CAGGCTGGAAGCCCAGACACGGAAT |
| | | | | | CCCGGATAACCAAAAGGACACTGTC |
| | | | | | TGTGAAG |
| 207 | Homo sapiens | BC034141.1 | AV688287.1 | 1.4 | CAAGGGACCAAGGTGgagcagttgaaatct |
| | immuno- | | | | |
| | globulin | | | | |
| | kappa | | | | |
| | constant, | | | | |
| | mRNA | | | | |
| 208 | ferritin, | NM_002032.1 | BG248923.1 | 0.6 | TGTCTCTGGGGATCCCTAGTATAAC |
| | heavy poly- | | | | ACATGCA |
| | peptide 1 | | | | |
| 209 | Homo sapiens | BC053635.1 | AI806846.1 | 1.5 | CCGGCTGGTCAAAGTgtgggtgctggcagc |
| | chromosome 4 | | | | |
| | open reading | | | | |
| | frame 9 | | | | |
| 210 | Homo sapiens | K02885.1 | U85050.1 | 0.8 | GGTTCGGGGACCAGGttaaccgttgtagag |
| | T-cell | | | | |
| | receptor | | | | |
| | active beta- | | | | |
| | chain V-D-J- | | | | |
| | beta-1.2-C- | | | | |
| | beta-1 (TCRB) | | | | |
| | mRNA | | | | |
| 211 | Homo sapiens | K02885.1 | AF043180.1 | 0.8 | GAGGACCTGAACAAGGTGTTCCCAC |
| | T-cell | | | | CCGAGGTCGCTGTGTTTGAGCCATC |
| | receptor | | | | AGAAGCAGAGATCTCCCACACCCAA |
| | active beta- | | | | AAGGCCACACTGGTGTGCCTGGCCA |
| | chain V-D-J- | | | | CAGGTATCTTCCCTGACCACGTGGA |
| | beta-1.2-C- | | | | GCTGAGCTGGTGGGTGAATGGGAAG |
| | beta-1 (TCRB) | | | | GAGGTGCACAGTGGGGTCAGCACGG |
| | mRNA | | | | ACCCGCAGCCCCTCAAGGAGCAGCC |
| | | | | | CGCCCTCAATGACTCCAGATACTGC |
| | | | | | CTGAGCAGCCGCCTGAGGGTCTCGG |
| | | | | | CCACCTTCTGGCAGAACCCCCGCAA |
| | | | | | CCACTTCCGCTGTCAAGTCCAGTTC |
| | | | | | TACGGGCTCTCGGAGAATGACGAGT |
| | | | | | GGACCCAGGATAGGGCCAAACCCGT |
| | | | | | CACCCAGATCGTCAGCGCCGAG |
| 212 | Homo sapiens | K02885.1 | AF317590.1 | 1.2 | TCTGTGCCAGCAGCCttggacagatttatg |
| | T-cell | | | | |
| | receptor | | | | |
| | active beta- | | | | |
| | chain V-D-J- | | | | |
| | beta-1.2-C- | | | | |
| | beta-1 (TCRB) | | | | |
| | mRNA | | | | |
| 213 | Homo sapiens | K02885.1 | AF327908.1 | 0.7 | ATATCTCTGCAGCGTcgggggtcaatctgg |
| | T-cell | | | | |
| | receptor | | | | |
| | active beta- | | | | |
| | chain V-D-J- | | | | |
| | beta-1.2-C- | | | | |
| | beta-1 (TCRB) | | | | |
| | mRNA | | | | |
| 214 | Homo sapiens | K02885.1 | AF327022.1 | 0, 7 | CTGCAGTGCTAGAGAtccggggtcccatca |
| | T-cell | | | | |
| | receptor | | | | |
| | active beta- | | | | |
| | chain V-D-J- | | | | |
| | beta-1.2-C- | | | | |
| | beta-1 (TCRB) | | | | |
| | mRNA | | | | |
| 215 | Human T-cell | M12886.1 | BC036926.1 | 0.8 | CCAGATCGTCAGCGCGcgaggcctggggtag |
| | receptor | | | | |
| | active beta- | | | | |
| | chain mRNA | | | | |
| 216 | Human | U42391.1 | BF948234.1 | 1.3 | CCTCTGGCCTCACAGtgtcccaggagcaca |
| | myosin-IXb | | | | |
| | mRNA | | | | |
| 217 | glutathione | NM_002085.1 | BI254515.1 | 0.8 | TGGAACTTCACCAAGttcctcatcgacaag |
| | peroxidase 4 | | | | |
| | (phospholipid | | | | |
| | hydroperoxi- | | | | |
| | dase) | | | | |
| 218 | Poly (A) | NM_002568.1 | AA033548.1 | 0.9 | TTCCAACTGTTTAAAattgatcagggacca |
| | binding | | | | |
| | protein, | | | | |
| | cytoplasmic 1 | | | | |
| 219 | prohibitin | NM_002634.2 | BI261762.1 | 1.4 | CAGTATGGTGAGGAGgtgtgagagggtcca |
| 220 | prohibitin | NM_002634.2 | BM014639.1 | 1.6 | TCAGAGTGGAAGCAGgtgagaatggagggg |
| 221 | prohibitin | NM_002634.2 | BM014639.1 | 1.4 | CTTTGGGCGAGGAGAgtgtgagagggtcca |
| 222 | prohibitin | NM_002634.2 | BE536369.1 | 1.4 | GCGAGGAGAGTGCGTgtgtgagagggtcca |
| 223 | spermidine/ | NM_002970.1 | CD108391.1 | 0.8 | TTCCAGAAGCCAGAGAGACCAAGTG |
| | spermine N1- | | | | TTATGTAAGAAGTAGTGTCGGCTGT |
| | acetyl- | | | | GTAGAACCACTGACTACACAGGCCG |
| | transferase | | | | AAGTTACTGAGAACTTGGACAGAAA |
| | | | | | AAATAGCCAGCAAGTGTTCAAACTA |
| | | | | | CTGAGGAAAAAAAAAAATTAGATAT |
| | | | | | GCTGCACTTAAGAATACTAGGGCAG |
| | | | | | GTT |
| 224 | cysteine-rich | NM_003118.1 | BG424815.1 | 1.4 | GAGAATGAGAAGCGCTGGAGGCAG |
| | acidic | | | | GAGACACCCCG |
| | secreted | | | | |
| | protein | | | | |
| | (osteonectin) | | | | |
| 225 | transla- | NM_003295.1 | BG284235.1 | 0.6 | TTCTTTATTGGTGAAAACATGAATC |
| | tionally- | | | | CAGATGGCATGGTTGCTCTATTGGA |
| | controlled | | | | CTACCGTGAGGATGGTGTGACCCCA |
| | tumor | | | | TATATGATTTTCTTTAAGGATGGTT |
| | protein 1 | | | | TA |
| 226 | transla- | NM_003295.1 | BG284235.1 | 0.7 | GAAATGGAAAAATGTtaacaaatgtggcaa |
| | tionally- | | | | |
| | controlled | | | | |
| | tumor | | | | |
| | protein 1 | | | | |
| 227 | transla- | NM_003295.1 | CD641954.1 | 0.7 | TTATTTTGGATCTATCACCTGTCATC |
| | tionally- | | | | ATAACTG GCTTCTGCTTGTCATCCA |
| | controlled | | | | CACAACACCAGGACTTAAGACAAATG |
| | tumor | | | | GGACTGATGTCATCTTGAGCTCTTCA |
| | protein 1 | | | | CATTTATTTTGACTGTGATTTATTTG |
| | | | | | GAGTGGAGGCATTGTTTTTAAGAAAA |
| | | | | | ACATGTCATGTAGGTTGTCTAAAAAT |
| | | | | | TAAAATGCATTTAAAC |
| 228 | transla- | NM_003295.1 | AV749932.1 | 0.6 | CGTCGTCTCCCTTCAGTCGCCATCA |
| | tionally- | | | | TGATTATCTACC |
| | controlled | | | | |
| | tumor | | | | |
| | protein 1 | | | | |
| 229 | transla- | NM_003295.1 | AV749932.1 | 0.6 | GGGACCTCATCAGCCacgatgagatgttct |
| | tionally- | | | | |
| | controlled | | | | |
| | tumor | | | | |
| | protein 1 | | | | |
| 230 | vasodilator- | NM_003370.1 | BQ340296.1 | 1.5 | TTTATTTCCTACCAGcaggaggagccagag |
| | stimulated | | | | |
| | phospho- | | | | |
| | protein | | | | |
| 231 | cold shock | NM_003651.3 | BG739968.1 | 1.2 | CCCAAGGTACCGTAGcaggggacctcctcg |
| | domain | | | | |
| | protein A | | | | |
| 232 | cold shock | NM_003651.3 | BE935120.1 | 1.5 | AATAACCCACGGAAATATCTGCGCA |
| | domain | | | | GTGTAGGAGATGGAGAAACTGTAGA |
| | protein A | | | | GTTTGATGTGGTTGAAGGAGAGAA |
| 233 | cold shock | NM_003651.3 | BE935120.1 | 1.5 | GTATTTGTACATCAGactgccatcaagaag |
| | domain | | | | |
| | protein A | | | | |
| 234 | chromosome 22 | NM_003678.2 | AK122673.1 | 1.5 | GAAGCAGAAGAGGTGTGAAAGAAG |
| | open reading | | | | GTGCTGCTGGGAGGGGAGTCTGAC |
| | frame 19 | | | | AACCCAGC |
| 235 | Homo sapiens | NM_003761.2 | BG623073.1 | 0.8 | ACGACATCGCAGAAGGTGGCTCGGA |
| | vesicle- | | | | AATTCTGGTGGAAGAACGTGAAGAT |
| | associated | | | | GATTGTCCTTATCTGCGTGATTGTT |
| | membrane | | | | TTTATCATCATCCTCTTCATTGTGC |
| | protein 8 | | | | TCTTTGCCACTGGTGCCTTCTCTTA |
| | (endobrevin) | | | | AGTAACAGGGAACCTCTCCCACCTG |
| | | | | | CCCTTCTTTTCAGGGACAACCCTCC |
| | | | | | ATAAATGTGTGCCAAGAGGGTCTCC |
| | | | | | TTTCCTGTCTTCCTCTACAGAGAAT |
| | | | | | GCTGCTCGGTCCTCCTACCCCTCTT |
| | | | | | CCCGAGGCCTGCTGCCACGTTGTAT |
| | | | | | GCCCCAGAAGGTACCTTGGTCCCCC |
| | | | | | GGAAGGAGAGAA |
| 236 | Homo sapiens | NM_003761.2 | BG623073.1 | 0.7 | GATCTGGAAGCCACAtctgagcacttcaag |
| | vesicle- | | | | |
| | associated | | | | |
| | membrane | | | | |
| | protein 8 | | | | |
| | (endobrevin) | | | | |
| 237 | CASP8 and | NM_003879.3 | BI871546.1 | 1.3 | AGTACAAGCAGTCTGgtggatggaatggaa |
| | FADD-like | | | | |
| | apoptosis | | | | |
| | regulator | | | | |
| 238 | beta-2- | NM_004048.2 | BM831738.1 | 0.8 | AGGTTTACTCACGTCATCCAGCAGA |
| | micro- | | | | GAATGGAAAGTCAAATTTCCTGAAT |
| | globulin | | | | TGCTATGTGTCTGGGTTTCATCCAT |
| | | | | | CCGACATTGAAGTTGACTTACTGAA |
| | | | | | GAATGGAGAGAGAATTGAAAAAGTG |
| | | | | | GAGCATTCAGACTTGTCTTTCAGCA |
| | | | | | AGGACTGGTCTTTCTATCTCTTGTA |
| | | | | | CTACACTGAATTCACCCCCACTGAA |
| | | | | | AAAGATGAGTATGCCTGCCGTGTGA |
| | | | | | ACCATGTGACTTTGTCACAGCCCAA |
| | | | | | GATAGTTAAGTGGG |
| 239 | beta-2- | NM_004048.2 | BM831738.1 | 0.7 | TGGAGGCTATCCAGCgtactccaaagattc |
| | micro- | | | | |
| | globulin | | | | |
| 240 | Guanine | NM_004125.2 | BC015206.1 | 0.6 | GTGGAGAGGATCAAGgtctctcaggcagct |
| | nucleotide | | | | |
| | binding | | | | |
| | protein, | | | | |
| | gamma 10 | | | | |
| 241 | BCL2- | NM_004323.2 | BI826041.1 | 1.2 | TGACTGTCACCCACAgcaatgagaagcacg |
| | associated | | | | |
| | athanogene | | | | |
| 242 | c-src | NM_004383.1 | BG953215.1 | 1.1 | CCTCAAGTTCTCGCTagatgtctgcgaggc |
| | tyrosine | | | | |
| | kinase | | | | |
| 243 | HLA-B | NM_004640.3 | CB148695.1 | 1.4 | GCCCCAGGAGGAGAGgtgagctgaagatgg |
| | associated | | | | |
| | transcript 1 | | | | |
| 244 | family with | NM_004699.1 | AA024425.1 | 0.8 | CACGGGGGAAGAGTGgaccactcttcaact |
| | sequence | | | | |
| | similarity | | | | |
| | 50, member A | | | | |
| 245 | family with | NM_004699.1 | AA584911.1 | 1.6 | TCAAGAGTGAGTGTTtgcggagtcagacgc |
| | sequence | | | | |
| | similarity | | | | |
| | 50, member A | | | | |
| 246 | GNAS complex | NM_000516.3 | BU784018.1 | 1.5 | CTACTCCTGAGGATGgtgtgtatggcttcc |
| | locus | | | | |
| 247 | GNAS complex | NM_000516.3 | BG911454.1 | 1.4 | GACGCCAGGGTTTGGgtgctggagaatctg |
| | locus | | | | |
| 248 | hemoglobin, | NM_00517.3 | AA343446.1 | 1.7 | TGCTTCTCCCCGCAGgatgttcctgtcctt |
| | alpha ½ | | | | |
| 249 | WD repeat | NM_005112.3 | BX648190.1 | 1.2 | AAGTTCACAATTGGCgaccacagccgcttt |
| | domain 1 | | | | |
| 250 | retino- | NM_005610.1 | BF475362.1 | 0.8 | TTTATTCATGGTGGTCATACTGCCA |
| | blastoma | | | | AGATATCTGATTTCTCCTGGAATCC |
| | binding | | | | CAATGAACCTTGGGTGATTTGTTCT |
| | protein 4 | | | | G |
| 251 | small EDRK- | NM_005770.2 | BG435668.1 | 0.9 | CCGTCGCCATGACCCgcggtaaccagcgtg |
| | rich | | | | |
| | factor 2 | | | | |
| 252 | putative | NM_005801.2 | BG655736.1 | 0.7 | CCTTTGTGCTTGCAGaagtttgcctgcaat |
| | translation | | | | |
| | initiation | | | | |
| | factor | | | | |
| 253 | membrane | NM_005898.2 | AA437165.1 | 0.8 | AACAGCTTCAAACAGtggttggcacttacc |
| | component, | | | | |
| | chromosome | | | | |
| | 11, surface | | | | |
| | marker 1 | | | | |
| 254 | capping | NM_006136.1 | BG702980.1 | 0.7 | AGCAAAAAATTTTTGgaatggtcgttggag |
| | protein | | | | |
| | (actin | | | | |
| | filament) | | | | |
| | muscle | | | | |
| | Z-line, | | | | |
| | alpha 2 | | | | |
| 255 | microspherule | NM_006337.3 | CB110581.1 | 1.4 | AACTCTGTGGTGGAGgtgagctggggagga |
| | protein 1 | | | | |
| 256 | Transforming, | NM_006342.1 | BM313828.1 | 1.3 | ACAGTGGAGCAGAAGgtgggtgcgggaagc |
| | acidic | | | | |
| | coiled-coil | | | | |
| | containing | | | | |
| | protein 3 | | | | |
| 257 | acidic | NM_006401.1 | AI446778.1 | 1.2 | AAGACGAGGACGATGAGGATGGTG |
| | (leucine- | | | | AAGAAGAGGAGTTTGATGAAGAAG |
| | rich) nuclear | | | | ATGATGAAGATGAAGATGTAGAAG |
| | phosphoprotein | | | | GGGATGAGGACGACGATGAAGTCA |
| | 32 family, | | | | GT |
| | member B | | | | |
| 258 | acidic | NM_006401.1 | AI446778.1 | 1.3 | AGGAGGAGGACGAAGaaggagaagatgagg |
| | (leucine- | | | | |
| | rich) nuclear | | | | |
| | phosphoprotein | | | | |
| | 32 family, | | | | |
| | member B | | | | |
| 259 | granulysin/ | NM_006433.2 | BI838502.1 | 0.8 | GATAAGCCCACCCAGagaagtgtttccaat |
| | granulysin | | | | |
| 260 | lysosomal | NM_006762.1 | BQ006415.1 | 1.4 | agatgctccagaaggtgagtgtggctgcag |
| | associated | | | | |
| | multispanning | | | | |
| | membrane | | | | |
| | protein 5 | | | | |
| 261 | lysosomal | NM_006762.1 | BQ006415.1 | 1.5 | AAGATGCTCCAGAAGgtgagtgtggctgca |
| | associated | | | | |
| | multispanning | | | | |
| | membrane | | | | |
| | protein 5 | | | | |
| 262 | transforming | NM_000660.1 | A1610679.1 | 1.4 | CGGGCTACNANATGCGCTTGGGGG |
| | growth | | | | GAGCCAGGACGGAGGAAGAGGAGA |
| | factor, | | | | GAGAAAGAGA |
| | beta 1 | | | | |
| | (Camurati- | | | | |
| | Engelmann | | | | |
| | disease) | | | | |
| 263 | myosin IF | NM_012335.2 | BF823263.1 | 1.3 | GTGGACAATGGGAAGctgctggaagggcct |
| 264 | ribosomal | NM_012423.2 | BM826692.1 | 0.8 | AAGCCTACAAGAAAGtttgcctatctgggg |
| | protein | | | | |
| | L13a | | | | |
| 265 | glutathione | NM_000852.2 | N59567.1 | 1.4 | CATGCTGTTCCTTCCTCGCCACCCT |
| | S-transferase | | | | CTGCTTC |
| | pi | | | | |
| 266 | chromosome 11 | NM_014206.1 | BE041814.1 | 0.8 | TCGCGGGGCAAAATGgagctcgaggccatg |
| | open reading | | | | |
| | frame 10 | | | | |
| 267 | integrin, | NM_000887.3 | AA251543.1 | 1.4 | TGCGACCGCCTACAGgtgacctccaaagct |
| | alpha X | | | | |
| | (antigen | | | | |
| | CD11C | | | | |
| | (p150), alpha | | | | |
| | polypeptide) | | | | |
| 268 | ring finger | NM_014868.3 | BM471027.1 | 1.3 | ACTTTCTGCTGACCCCTCTGTCACC |
| | protein 10 | | | | CACTGCCAGTCAGGGCAGTCCCTCA |
| | | | | | TTCTGCGTTGGGAGTCTGGAAGAAG |
| | | | | | ACTCTCCCTTC |
| 269 | ribosomal | NM_000968.2 | CB164625.1 | 0.7 | GGTGCTTTTGGAAACatgtgtcgtggaggc |
| | protein L4 | | | | |
| 270 | KIAA0690 | NM_015179.2 | BG506709.1 | 1.6 | TCTCTGGGCAAGCAGaaagcaaaaggtgat |
| 271 | KIAA0690 | NM_015179.2 | BG506709.1 | 1.6 | GAATACAAGGCCAAGaaagcaaaaggtgat |
| 272 | bridging | NM_016187.1 | AK093638.1 | 0.9 | AAGGCCATCGTATGGaataatgatctcctt |
| | integrator 2 | | | | |
| 273 | ribosomal | NM_000990.2 | BG824148.1 | 0.8 | CAACTTCGACAAATAccacccaggctactt |
| | protein L27a | | | | |
| 274 | ribosomal | NM_000990.2 | B1906450.1 | 0.8 | gccacccaggctactttgggaaagt |
| | protein L27a | | | | |
| 275 | ribosomal | NM_000990.2 | CB119057.1 | 0.7 | GCCACGGCCGCATAGgcaagcaccggaagc |
| | protein L27a | | | | |
| 276 | ankyrin | NM_017664.1 | BC039715.1 | 0.7 | GGAGCATTCCATATAGAAACTGCTG |
| | repeat | | | | AAACTGCCACAGGTGCTTCTCCGAA |
| | domain 10 | | | | AACCTTACAGTTGTGGCATTGAATG |
| | | | | | TTCAGTATCGCTTCCTTTCTGCACA |
| | | | | | CG |
| 277 | ribosomal | NM_000992.2 | BQ335217.1 | 0.7 | CCAAGTTCCTGAGGAacatgcgctttgcca |
| | protein L29 | | | | |
| 278 | ribosomal | NM_001017.2 | CA843486.1 | 0.8 | TTCCGTCTGATTCTAATAGAGAGCC |
| | protein S13 | | | | GGATTCACCGTTTGGCTCGATATTA |
| | | | | | TAAGACCAAGCGAGTCCTCCCTCCC |
| | | | | | AATTGGAAATA |
| 279 | CNDP dipepti- | NM_018235.1 | AW502844.1 | 1.5 | CACAGCAGCATCAAGgtggagtgcagcaac |
| | dase 2 | | | | |
| | (metallo- | | | | |
| | peptidase | | | | |
| | M20 family) | | | | |
| 280 | ribosomal | NM_001021.2 | BE731466.1 | 0.5 | AATTATGTTCCTGAGgtctcagccttggat |
| | protein S17 | | | | |
| 281 | ribosomal | NM_001021.2 | AV752729.1 | 0.6 | GCCGCGTTCCACCAAAACCGTGAA |
| | protein S17 | | | | GAAAGCGGCCCGGGTCATCATAGA |
| | | | | | AAAGTACTACACGCGCCTGGGCAA |
| | | | | | CGACTTCCACACGAACAAGCGCGT |
| | | | | | GTGCGAGGAGATCGCCATTATCCC |
| | | | | | CAGCAAAAAGCTCCG |
| 282 | sulfatase 2 | NM_018837.1 | AB033073.2 | 1.5 | GCGAGAGTGTGTCGAgtgagtgtgcgtctg |
| 283 | ubiquitin B | NM_018955.2 | BG286180.1 | 1.6 | GAAGGCGGAAAAGAGgtcaaaatgcagatc |
| 284 | ubiquitin B | NM_018955.2 | BG286180.1 | 1.4 | GGTATCCGCTAACAGgtcaaaatgcagatc |
| 285 | glycogen | NM_019884.1 | BQ927367.1 | 1.3 | ACTTCAGTGCTGGTGgtgagggcatagcc |
| | synthase | | | | |
| | kinase 3 | | | | |
| | alpha | | | | |
| 286 | KIAA1185 | NM_020710.1 | BQ352354.1 | 1.1 | GTGTTAGGAGCGGAGataccttcacttgct |
| | protein | | | | |
| 287 | peptidyl- | NM_021130.1 | AA340318.1 | 0.8 | CGCGTCTCCTTTGAGctgtttgcagacaag |
| | prolyl | | | | |
| | isomerase A | | | | |
| | (cyclophilin | | | | |
| | A) | | | | |
| 288 | peptidyl- | NM_021130.1 | AA357116.1 | 0.8 | TCCCAAAGACAGCAGaaaattttcgtgctc |
| | prolyl | | | | |
| | isomerase A | | | | |
| | (cyclophilin | | | | |
| | A) | | | | |
| 289 | peptidyl- | NM_021130.1 | BE293161.1 | 0.7 | CGCGTCTCCTTTGAGgtacggggcctggat |
| | prolyl | | | | |
| | isomerase A | | | | |
| | (cyclophilin | | | | |
| | A) | | | | |
| 290 | hypothetical | NM_024841.1 | BQ919225.1 | 1.4 | ATTGTTGAACTGGATgcggctgttgaagag |
| | protein | | | | |
| | FLJ14213 | | | | |
| 291 | hypothetical | NM_024841.1 | AK055042.1 | 0.8 | GATCAACGTTTTCAAAGGGGGTGGC |
| | protein | | | | TTGCAAAGCAACGAGCTCTATGCCC |
| | FLJ14213 | | | | T |
| 292 | ring finger | NM_025126.2 | NM_194271.1 | 0.9 | GTCGCGGCCATGAAGgtgggggagtggtac |
| | protein 34 | | | | |
| 293 | t-complex 1 | NM_030752.1 | AA160249.1 | 0.7 | TTCTTCTTTTCCTAGgggtctcgggaacag |
| 294 | RAB34, member | NM_031934.3 | W32152.1 | 1.6 | AAGAAGGATCTGAGTgtgagtgtgccagtg |
| | RAS oncogene | | | | |
| | family | | | | |
| 295 | zinc finger, | NM_032226.1 | BG530408.1 | 0.8 | ACTGGTCCATCAGTGACAAAGACAT |
| | CCHC domain | | | | TGAG |
| | containing 7 | | | | |
| 296 | guanylate | NM_052942.2 | AA164465.1 | 1.5 | AAAAAAAGAAGAAAGaggcacaagtgaaag |
| | binding | | | | |
| | protein 5 | | | | |
| 297 | ubiquitin | NM_058167.2 | BG766070.1 | 1.4 | TGCGCGGAACCCGAGatgagcagcaccagc |
| | conjugating | | | | |
| | enzyme E2, J2 | | | | |
| 298 | chloride | NM_001288.3 | AA126087.1 | 0.8 | CCTGAGTCCAACACAGCTGGGCTGG |
| | intracellular | | | | ACATATTTGCCAAATTTTCTGCCTA |
| | channel 1 | | | | CATCAAGAATTCAAACCCAGCACTC |
| | | | | | AATGACA |
| 299 | chloride | NM_001288.3 | AA126087.1 | 0.7 | CCCAGGTACCCCAAGctggcagctctgaac |
| | intracellular | | | | |
| | channel 1 | | | | |
| 300 | chloride | NM_001288.3 | AA126087.1 | 0.8 | GCTGTGCCCTCCCAGgtaccccaagctggc |
| | intracellular | | | | |
| | channel 1 | | | | |
| 301 | peptidylprolyl | NM_130906.1 | BU195819.1 | 1.4 | CCCAAAACATGTGAGgctggagtacaatgg |
| | isomerase | | | | |
| | (cyclophilin)- | | | | |
| | like 3 | | | | |
| 302 | cAMP | NM_134442.2 | B1831922.1 | 0.8 | GCCACATTAGCCCAGgtatctatgccagca |
| | responsive | | | | |
| | element | | | | |
| | binding | | | | |
| | protein 1 | | | | |
| 303 | heat shock | NM_153201.1 | CD655642.1 | 0.8 | GCCTACCTTGGGAAGactgttaccaatgct |
| | 70 kDa | | | | |
| | protein 8 | | | | |
| 304 | E74-like | NM_172373.2 | AA251399.1 | 0.8 | GCCAAGAAGCTTGAGAGAAGAAAAA |
| | factor 1 | | | | TTTCAGAAAAATTGTCTCAATTTGA |
| | (ets domain | | | | CTAGAATATCAATGAACCAGGAAAA |
| | transcription | | | | CTGAAGCACCTTCCCTAAAGAAAAC |
| | factor) | | | | TTGGGTATACAATTACTCCACAGAC |
| | | | | | AGAGCTGAGGGTTTTTTACCCAAAT |
| | | | | | CAGTCACTGGATTTTGCTGCCTGAT |
| | | | | | ACGTGAATCTTCTTGGAATTTTTCT |
| | | | | | CATGTGGATCTAAGGGGAATGCTTT |
| | | | | | ATTATGGCTGCTGTTGTCCAACAGA |
| | | | | | ACGACCTAGTATTTGAATTTGCTAG |
| | | | | | TAACGTCATG |
| 305 | eukaryotic | NM_001402.4 | BX454657.1 | 0.8 | GGCTTCACTGCTCAGgtgattatcctgaac |
| | translation | | | | |
| | elongation | | | | |
| | factor 1 | | | | |
| | alpha 1 | | | | |
| 306 | eukaryotic | NM_001402.4 | BQ365319.1 | 0.8 | AGCTTCTCAGACTATccacctttgggtaag |
| | translation | | | | |
| | elongation | | | | |
| | factor 1 | | | | |
| | alpha 1 | | | | |
| 307 | arachidonate | NM_001629.2 | BF892107.1 | 0.9 | AAGTGGAGCACGAAAGCAGGACCC |
| | 5-lipoxy- | | | | AGAATGGGAGGAGCTTCCAGAGGA |
| | genase- | | | | CCGGAACACTTGCCTTTGAGCGGG |
| | activating | | | | TCTACACTGCCAA |
| | protein | | | | |
| 308 | ribosomal | NM_021104.1 | EXH-001 | 0.7 | AAGAAAGATGAGGCAGAGGTCCAA |
| | protein L41 | | | | GTAAACCGCTAGCTTGTTG |
| 309 | ribosomal | NM_021104.1 | EXH-002 | 0.6 | GAAGCGAATGCGCAGgctgaagcgcaaaag |
| | protein L41 | | | | |
| 310 | transla- | NM_003295.1 | EXH-003 | 0.5 | AATGCATATTTAAACTAAATTGATC |
| | tionally- | | | | CTGTAGTGTTCCTGGAGAAGCTAGA |
| | controlled | | | | GCCTGATTGTAGGCTACTACTCATC |
| | tumor | | | | AATTAACTTCTACAGTGGAGACTAC |
| | protein 1 | | | | TTCTGGGACTGGAATATAAAAA |
| 311 | G1 to S | NM_002094.1 | EXH-004 | 1.5 | ATTACCGTTTATTCCATATCTGGATA |
| | phase | | | | ATTTGCCGAACTTCAATAGATCAGTT |
| | transition | | | | GATGGACCAATCAGGCTGCCAATTGT |
| | 1/G1 to S | | | | GG |
| | phase | | | | |
| | transition 1 | | | | |
| 312 | cyclin T2 | NM_001241.2 | EXH-005 | 0.5 | ttgtgtgagctattcaaactcttcaacccctga |
| 313 | cyclin T2 | NM_001241.2 | EXH-006 | 0.7 | AGCCAGTTGTCATTTttacaggattgtgtg |
| 314 | zinc finger | NM_133476.2 | EXH-007 | 1.5 | AGTTCAGGAGCCCTGGAAAGGAGA |
| | protein 384 | | | | AGGAATAAGACGGCAGGAGGAAGA |
| | | | | | GA |
| 315 | TBC1 domain | NM_020773.1 | EXH-008 | 0.7 | CTACCTAATTGATTGcccggggccctgatt |
| | family, | | | | |
| | member 14 | | | | |
| 316 | F-box | NM_012179.2 | EXH-009 | 1.3 | GGAAGCGCGGGTGGTCGGCTGGGG |
| | protein 7 | | | | TCCGGCTCCTGGAGAACATGGCCC |
| | | | | | GGCCTCCCGGGGGCT |
| 317 | F-box | NM_012179.2 | EXH-010 | 1.4 | CTGGTCCCCTCCTCGttctaatacccgatt |
| | protein 7 | | | | |
| 318 | maltase- | NM_004668.1 | EXH-011 | 1.4 | GTAATTCAACTGCCAAGTGGTGGAA |
| | glucoamylase | | | | GAGGGAAATAGAAGAACTATACAAC |
| | (alpha- | | | | AATCCACAGAATCCAGAGAG |
| | glucosidase) | | | | |
| 319 | Eukaryotic | NM_001404.3 | BU783548.1 | 0.6 | TCACCTGCCCAAGAGtacctttgtgttgga |
| | translation | | | | |
| | elongation | | | | |
| | factor 1 | | | | |
| | gamma | | | | |
| 320 | Eukaryotic | NM_001404.3 | BG615194.1 | 0.8 | ACCCTGTACACGTATCCTGAAAAC |
| | translation | | | | TGGAGGGCCTTCAAGGCTCTCATC |
| | elongation | | | | GCTGCTCAGTACAGCGGGGCTCAG |
| | factor 1 | | | | GTCCGCGTGCACTCCGCACCAC |
| | gamma | | | | |
| 321 | KIAA1037 | AB028960.1 | NM_015023.2 | 1.1 | TTGCCCCCCCGCCAGcaacgctcagtatat |
| 323 | integrin, | NM_000211.1 | AU185877.1 | 1.3 | GTCGCCGCCTGCCCGagtgtgtggcaggcc |
| | beta 2 | | | | |
| 323 | DKFZp586K2322 | AL080113.1 | AI741782.1 | 0.7 | CCCAGAAAGAATTTTCTGCTATTG |
| | | | | | TGTTCACTACAACAGGATAGGGAC |
| | | | | | ATCAGACAGCCCCAGAAACCCCTT |
| | | | | | CCAGATCTGATATGGGACTATTAA |
| | | | | | TTTTTATGCTGTTAATTGGTATTC |
| | | | | | ATTCACAATGCAGTTGAAGGGGGA |
| | | | | | AGGCTCCACTGCATTCTTTGGCTA |
| | | | | | AGGCCTGAATGCTTGCTCATCTGT |
| | | | | | AAGATCTATACTCGAGGTTTTGTT |
| | | | | | TTCCTTTTAAAATTCTTTAGGGAG |
| | | | | | AGAGGGATGGTTTCTGAGGGGTTC |
| | | | | | TGAAAGTATGATTCAATGTGCAAC |
| | | | | | ATACAGGTAGGTCTTCAGCATAAG |
| | | | | | CTGAAATATATGCATGTAAAAACT |
| | | | | | TTGACATCTTTTTTTTTAATTTTC |
| | | | | | CACTTTCTTCTTAACTTTACTTCT |
| | | | | | CTTTTTGTCCCCCCCCCATCTTAC |
| | | | | | AGAAGTTGAGGCCAAGGGAGAATG |
| | | | | | GTAGGCACAGAAGAAACATGGCAA |
| | | | | | ACTGCTCTGTGCTTTCAAACCAAA |
| | | | | | GTGTTCCCCCCAACCCCAAA |
| 324 | calpain, | NM_001749.1 | BF939173.1 | 1.2 | CAACTTCATCAGCTGccaggcccaggagga |
| | small | | | | |
| | subunit 1 | | | | |
| 325 | glyceralde- | NM_002046.2 | BE255095.1 | 0.8 | GCCAAGGTCATCCATGACAACTTTG |
| | hyde-3- | | | | GTATCGTGGAAGGACTC |
| | phosphate | | | | |
| | dehydrogenase | | | | |
| 326 | major histo- | NM_002121.4 | BG759187.1 | 0.7 | GATCTGCATAAACAGggttcctgagctcac |
| | compatibility | | | | |
| | complex, | | | | |
| | class II, | | | | |
| | DP beta 1 | | | | |
| 327 | lysozyme | NM_000239.1 | BE720647.1 | 0.8 | TCGTTGTCAAAACAGAGATGTCCGT |
| | (renal | | | | CAGTATGTTCAAGGTTGTGGAGTGT |
| | amyloidosis) | | | | AACTCCAGAATTTTCCTTCTTCAGC |
| | | | | | TCATTTTGTCTCTCTCACATTAAGG |
| | | | | | GAGTAGGAATTAAGTGAAAGGTCAC |
| | | | | | ACTACCATTATTTCCCCTTCAAACA |
| | | | | | AATAATATTTTTACAGAAGCAGGAG |
| | | | | | CAAAATATGGCCTTTCTTCTAAGAG |
| | | | | | ATATAATGTTCACTAATGTGGTTAT |
| | | | | | TTTACATTAAGCCT |
| 328 | tumor protein, | NM_003295.1 | AA223997.1 | 0.8 | GAAACTTGAAGAACAgagaccagaaagagt |
| | transla- | | | | |
| | tionally | | | | |
| | controlled 1 | | | | |
| 329 | Homo sapiens | BC014203.1 | AK093557.1 | 0.8 | CAAACCTGCTTTGCTgaaagttgcagaaaa |
| | family with | | | | |
| | sequence | | | | |
| | similarity | | | | |
| | 101, member | | | | |
| | B | | | | |
| 330 | lysozyme | NM_000239.1 | BE549820.1 | 0.7 | AATATGAAATTTTTAAAGGAGTAGA |
| | (renal | | | | ATACCAAATGATAGAAACAGACTGC |
| | amyloidosis) | | | | CTGAATTGAGAATTTTGATTTCTTA |
| | | | | | AAGTGTGTTTCTTTCTAAATTGCTG |
| | | | | | TTCCTTAATTTGATTAATTTAATTC |
| | | | | | ATGTATTATGATTAAATCTGAGGCA |
| | | | | | GATGAGCTTACAAGTATTGAAATAA |
| | | | | | TTACTAATTAATCACAAATGTGAAG |
| | | | | | TTATGCATGATGTAAAAAATACAAA |
| | | | | | CATTCTAATTA |
| 331 | lysozyme | NM_000239.1 | BE549820.1 | 0.7 | GGAGCAAAATATGGCCTTTCTTCTA |
| | (renal | | | | AGAGATATAATGTTCACTAATGTGG |
| | amyloidosis) | | | | TTATTTTACATTAAGCCTACAACAT |
| | | | | | TTTTCAGTTTGCAAATAGAACTAAT |
| | | | | | ACTGGTGAAAATTTACCTAAAACCT |
| | | | | | TGGTTATCAAATACATCTCCAGTAC |
| | | | | | ATTCCGTTCTTTTTTTTTTTTGAGA |
| | | | | | CAGTCTCGCTCTGTCGCCCAGGCTG |
| | | | | | GAGTGCAGTGGCGCAATCTCGGCTC |
| | | | | | ACTGCAACCTCCACCTCCCGGGTTC |
| | | | | | ACGCCATTCTCCTGCCTCAGCCTCC |
| | | | | | CGAGTAGCTGGGATTACGGGCGCCC |
| | | | | | GCCACCACGCCCGGCTAATTTTTTG |
| | | | | | TATTTTTAGTAGAGACAGGGTTTCA |
| | | | | | CCGTGTTAGCCAGGATGGTCTCGAT |
| | | | | | CTCCTGACCTTGTGATCCACCCACC |
| | | | | | TCGGCCTCCCAAAGTGCTGGGATTA |
| | | | | | CAGGCGTGAGCCACTGCGCCCGGCC |
| | | | | | ACATTCAGTTCTTATCAAAGAAATA |
| | | | | | ACCCAGACTTAATCTTGAATGATAC |
| 332 | Homo sapiens | NM_004368.1 | BU630244.1 | 1.4 | TTGTGGTTTCTATGCccgcctccgcctccc |
| | calponin 2 | | | | |
| | (CNN2), | | | | |
| | transcript | | | | |
| | variant 1 | | | | |
| 333 | ubiquitin | NM_005153.1 | W19112.1 | 0.8 | GCAACAGTGCGACGGGCGGCCATT |
| | specific | | | | ACACTACAGACGTCTTCCAGATCG |
| | peptidase 10 | | | | GTCTGAATGGCTGGCTGCG |
| 334 | SUMO-1 | NM_005500.1 | BI463391.1 | 1.1 | GTGCTGCCGGCGGCGgctgcgggcctctcg |
| | activating | | | | |
| | enzyme | | | | |
| | subunit 1 | | | | |
| 335 | Homo sapiens | NM_005569.2 | BX368816.1 | 1.2 | ATCGCCTCCGGAATGgcctatttgcactct |
| | LIM domain | | | | |
| | kinase 2 | | | | |
| | (LIMK2), | | | | |
| | transcript | | | | |
| | variant 2a | | | | |
| 336 | actin related | NM_005720.2 | BI160852.1 | 0.9 | GCGTCTGTTTCTCAGccagcgggagccgcg |
| | protein ⅔ | | | | |
| | complex, | | | | |
| | subunit | | | | |
| | 1B, 41 kDa | | | | |
| 337 | tubulin, | NM_006082.1 | BE278798.1 | 0.6 | CTGTCAGTTGATTATGGCAAGAAAT |
| | alpha, | | | | CCAAGCTGGAGTTCTCCATTTACC |
| | ubiquitous | | | | |
| 338 | mannosidase, | NM_006122.1 | AI480034.1 | 1.5 | GACATCGACAGCCAGggtgcagccccgacg |
| | alpha, | | | | |
| | class 2A, | | | | |
| | member 2 | | | | |
| 339 | goigi | NM_012201.1 | BC031836.1 | 1.2 | ACATCACTGAGTATCAGTGTCACCA |
| | apparatus | | | | GTACATTACCAAGATGACGGCCATC |
| | protein 1 | | | | ATTTTTAGTGATTACCGTTTAATCT |
| | | | | | GTGGCTTCATGGATGACTGCAAAAA |
| | | | | | TGACATCAACATTCTGAAATGTGGC |
| | | | | | AGTATTCGGCTTGGAGAAAAG |
| 340 | c-myc | NM_012333.2 | AI291757.1 | 1.4 | GGCGCCAGCTACGCCGCTGCCGCTG |
| | binding | | | | TCACTATGGCCCATTACAAAGCCGC |
| | protein | | | | CGACTCGAAGCG |
| 341 | lysosomal- | NM_014713.2 | R61059.1 | 0.7 | CTGAGAGAATGGCTGataatgcctgtgttc |
| | associated | | | | |
| | protein | | | | |
| | transmembrane | | | | |
| | 4 alpha | | | | |
| 342 | eukaryotic | NM_016091.1 | CD389782.1 | 0.7 | CGTTCCAATCCCAAAATCTGGAATG |
| | translation | | | | TTCATAGTGTCCTCAATGTCCTTCA |
| | initiation | | | | TTCCCTGGTAGACAAATCCAACATC |
| | factor 3, | | | | AACCGACAGTTGGAGGTATACACAA |
| | subunit 6 | | | | GCGGAG |
| | interacting | | | | |
| | protein | | | | |
| 343 | Homo sapiens | NM_021038.1 | BM083621.1 | 0.6 | TTTGTAATTAGTTATTATAAGAAGA |
| | muscleblind- | | | | TCTAGATCCTAGATATTAGAATAAA |
| | like | | | | ATTTATTTTCTACTGTATCCATTTC |
| | (Drosophila) | | | | AAATGTTAAAATATTGTTTAATATT |
| | (MBNL1), | | | | TTTGAAATCCCTGAGTATCAGGCCT |
| | transcript | | | | TGTTATAAATAAGCTGCATAATCAA |
| | variant 1 | | | | TAAATAGAACAAGGGACTTTTTGTT |
| | | | | | GATAATCCAAATACTCAAAGTTTAC |
| | | | | | GTAATGAAAATTATAGCGTGTGTGC |
| | | | | | AAACTCTTGAGGGTTGATTATGCTG |
| | | | | | CAATTTAGCATGTTGGAACGTCTAG |
| | | | | | GGAGAAGGTTGACTTTTTGCACTTC |
| | | | | | TGTATATAGTCAAAAGAGAGAAACC |
| | | | | | TGTATAATAGTAAGATCTTATTTTG |
| | | | | | AATAAAAACGTCTATAATTACAAGG |
| | | | | | AGTTTTGTTAAGGCTAATACAATGA |
| | | | | | CAGACTGAGCAAAATTGCTTGCAAA |
| | | | | | AGTGGCACAGAGTTAGCACTCCATA |
| | | | | | CCCCTTCAAACATGTTGCTTTGCTT |
| | | | | | TCTTGTGGACAGCTTGTAGTTTGCC |
| 344 | amino-terminal | NM_001130.4 | BE857026.1 | 1.3 | ATCATCCGACAGCAGctccacgcccacctg |
| | enhancer of | | | | |
| | split (AES), | | | | |
| | transcript | | | | |
| | variant 2 | | | | |
| 345 | heterogeneous | NM_031314.1 | BU570812.1 | 0.8 | TTCAGGCCATTAAGAAGGAGCTGAC |
| | nuclear | | | | CCAGATAAAACAAAAAGTGGATTCT |
| | ribonucleo- | | | | CTCCTGGAAAACCTGGAAAAAATGG |
| | protein C | | | | AAAAGGAAC |
| | (C1/C2) | | | | |
| | (HNRPC), | | | | |
| | transcript | | | | |
| | variant 1, | | | | |
| | mRNA | | | | |
| 346 | Homo sapiens | NM_032812.6 | AK027529.1 | 0.9 | TGTGAGAATACAGAACCAGTGGAAA |
| | plexin domain | | | | CTTCTTCTCGAACCACCACAACCAT |
| | containing 2 | | | | AGGAGCGACAACCACCCAGTTCAGG |
| | (PLXDC2) | | | | GTCCTAACTACCACCAGAAGAGCAG |
| | | | | | TGACTTCTCAGTTTCCCACCAGCCT |
| | | | | | CCCTACAGAAG |
| 347 | Homo sapiens | BC051834.1 | AA225636.1 | 0.7 | GTAGCCTCACTGGAGGGCATTGCC |
| | cDNA clone | | | | CCGGAAGATCAAGTCGTGCTCCTG |
| | IMAGE: | | | | GCAGGCGCGCCCCTGGAGGATGAG |
| | 5212028, | | | | GCCACTCTGGGCCAGTGCGGGGTG |
| | partial cds | | | | GAGGCCCTGACTACCCTGGAAGTA |
| | | | | | GCAGGCCGCATGCTTGGAGGTGAG |
| | | | | | TGAGAGAGGAATGTTCTTTGAAGT |
| | | | | | ACCGGTAAGCGTCTAGTGAGTGTG |
| | | | | | GGGTGCATAGTCCTGACAGCTGAG |
| | | | | | TGTCACACCTATGGTAATAGAGTA |
| | | | | | CTTCTCACTGTCTTCAGTTCAGAG |
| | | | | | TGATTCTTCCTGTTTACATCCCTC |
| | | | | | ATGTTGAACACAGACGTCCATGGG |
| | | | | | AGACTGAGCCAGAGTGTAGTTGTA |
| | | | | | TTTCAGTCACATCACGAGATCCTA |
| | | | | | GTCTGGTTATCAGCTTCCACACTA |
| | | | | | AAATTAGGTCAGACCAGGGCCCCC |
| | | | | | AAAGTGCTCTATAAAATTAGAAGC |
| | | | | | TGGAAGATCCTGAAATGAAACTTA |
| | | | | | AGATTTCAAGGTCAAATATCTGCA |
| | | | | | ACTTTGTTCTCATTACCTAT |
| 348 | chloride | NM_001288.3 | AI334086.1 | 0.9 | CTGGCTGACTGCAACctgttgccaaagtta |
| | intracellular | | | | |
| | channel 1 | | | | |
| 349 | HLA-B | NM_080703.1 | BF025832.1 | 1.3 | ATTCTGGCACACAGCCTGGTGGTGT |
| | associated | | | | TCCGAGTGCTCCCACTGGCCCCC |
| | transcript 3 | | | | |
| | (BAT3), | | | | |
| | transcript | | | | |
| | variant 3 | | | | |
| 350 | nucleosome | NM_139207.1 | BG110891.1 | 0.8 | TTGCCCCTCCTGAAGttcctgagagtggag |
| | assembly | | | | |
| | protein | | | | |
| | 1-like 1 | | | | |
| | (NAP1L1), | | | | |
| | transcript | | | | |
| | variant 1 | | | | |
| 351 | enoyl Coenzyme | NM_001398.1 | BG333307.1 | 1.4 | CGGCCCAACAAGAGGtgccccaagcccgtg |
| | A hydratase 1, | | | | |
| | peroxisomal | | | | |
| 352 | EST clone | NM_173703.1 | CB962365.1 | 1.1 | AACACAAACTACCACctactcatgcaccta |
| | CB962365.1 | | | | |
| 353 | Dicer1, | NM_177438.1 | BQ009528.1 | 0.8 | ACTAAAGTCCTCCTGccaggtagttcccac |
| | Dcr-1 | | | | |
| | homolog | | | | |
| | (Drosophila) | | | | |
| 354 | Beta tubulin | NM_178014.2 | N85882.1 | 0.7 | TCCCATCTCAGCTTCAAGGGAGGTG |
| | (TUBB) | | | | TCAGCAGTATTATCTCCACTTTCAA |
| | | | | | TCTCCCTCCAAGCTCTACTCTGGAG |
| | | | | | GAGTCTGTCCCACTCTGTCAAGTGG |
| | | | | | AATCCTTCCCTTTCCAACTCTACCT |
| | | | | | CCCTCACTCAGCTCCTTTCCCCTGA |
| | | | | | TCAGAGAAAGGGATCAAGGGGGTTG |
| | | | | | GGAGGGGGGAAAGAGACCAGCCTTG |
| | | | | | GTCCCTAAGCCTCCAGAAACGTCTT |
| | | | | | CTTAATCCCCACCTTTTCTTACTCC |
| | | | | | CAAAAAAGAATGAACACCCCTGACT |
| | | | | | CTGGAGTGGTGTATACTGCCACATC |
| | | | | | AGTGTTTGAGTCAGTCCCCAGAGGA |
| | | | | | GAGGGGAACCCTCCTCCATCTTTTT |
| | | | | | TGCAACATCTCATTTCTTCCTTTTG |
| | | | | | CTGTTGCTTCCCCCCTCACACACTT |
| | | | | | GGTTTTGTTCTATCCTACATTTGAG |
| | | | | | ATTTCTATTTTATGTTGAACTTGCT |
| | | | | | GCTTTTTTTCATATTGAAAAGATGA |
| | | | | | CATCGCCCCAAGAGCCAAAAATAAA |
| 355 | Immuno- | BC034142.1 | BC030813.1 | 1.2 | GAGGAACTGCTCAGTTAGGACCCA |
| | globulin | | | | GACGGAACCATGGAAGCCCCAGCG |
| | kappa | | | | CAGCTTCTCTTCCTCCTGC |
| | variable | | | | |
| | chain 1-5, | | | | |
| | mRNA | | | | |
| 356 | Immuno- | BC034142.1 | AF027158.2 | 1.2 | ATGGAAACCCCAGCGCAGCTTCTCT |
| | globulin | | | | TCCTCCTGC |
| | variable | | | | |
| | chain 1-5, | | | | |
| | mRNA | | | | |
| 357 | Homo sapiens | K028855.1 | AF327294.1 | 0.8 | CAAGGCACCAGACTCacagttgtagaggac |
| | T-cell | | | | |
| | receptor | | | | |
| | active beta- | | | | |
| | chain-V-D-J- | | | | |
| | beta-1.2-C- | | | | |
| | beta-1 (TCRB | | | | |
| 358 | glutathione | NM_002085.1 | AI188779.1 | 0.8 | tgccatcaagtggaacttcaccaag |
| | peroxidase 4 | | | | |
| | (phospholipid | | | | |
| | hydroper- | | | | |
| | oxidase) | | | | |
| 359 | Secreted | NM_003118.1 | BG424815.1 | 1.1 | AAGCAGAAGCTGCGGgtgaagaagatccat |
| | protein, | | | | |
| | acidic, | | | | |
| | cysteine- | | | | |
| | rich | | | | |
| | (osteonectin) | | | | |
| 360 | tumor | NM_003295.1 | BI909906.1 | 0.7 | ATGGTCAGTAGGACAGAAGGTAACA |
| | protein, | | | | TTGATGACTCGCTCATTGGTGGAAA |
| | transla- | | | | TGCCTCCGCTGAAGGCCCCGAGGGC |
| | tionally- | | | | GAAGGTACCGAAAGCACAGTAATCA |
| | controlled 1 | | | | CTGGTGTCGATATTGTCATGAACCA |
| | | | | | TCACCTGCAGGAAACAAGTTTCACA |
| | | | | | AAAGAAGCCTACAAGAAGTACATCA |
| | | | | | A |
| 361 | hypothetical | NM_004125.2 | U31383.1 | 0.8 | GCAGAGCTTCAACAGTACTGTATGC |
| | protein | | | | AGAATGCCTGCAAGGATGCCCTGCT |
| | LOC552891 | | | | GGTGGGTGTTCCAGCTGGAAGTAAC |
| | (LOC552891) | | | | CCCTTCCGGGAGCCTAGATCCTGTG |
| | | | | | CTTTACTCTGAAGACTC |
| 362 | sin3- | NM_005870.3 | AA346556.1 | 0.8 | GCAGATCTACACTTGgatggatgcaacctt |
| | associated | | | | |
| | polypeptide, | | | | |
| | 18 kDa | | | | |
| 363 | Acidic | NM_006305.2 | BF038728.1 | 1.2 | TATAACGATGGAGAGGTAGATGACG |
| | (leucine- | | | | AGGAAGATGAAGAAGAGCTTGGTG |
| | rich) nuclear | | | | |
| | phosphoprotein | | | | |
| | 32 family, | | | | |
| | member A | | | | |
| 364 | syntaxin | NM_006949.1 | BQ002314.1 | 1.3 | CGCCCCCGCCCACGCCTGGGTCTG |
| | binding | | | | TGTTAGGTGGGCGGCCTGGCGGCG |
| | protein 2 | | | | GTGAGGGCCTCCTGCCTGGACTTT |
| | | | | | CTGC |
| 365 | anaphase | NM_013366.2 | BQ045327.1 | 1.4 | GAGGAGGAGCTGCTGgtgcgcgtgcaggcc |
| | promoting | | | | |
| | complex | | | | |
| | subunit 2 | | | | |
| 366 | integrin, | NM_000887.3 | AA251543.1 | 1.3 | CCGACCCAGGACACCCTGACCTCT |
| | alpha X | | | | GGAGTCCCCCATCCCAGGCCCCTG |
| | (antigen CD11C | | | | TCTCCCACCCTGCTCATTGTCCAC |
| | (p150), alpha | | | | CCAAGGAGTTCCTGTCTCAACGCC |
| | polypeptide) | | | | GTCCCT |
| 367 | ring finger | NM_014868.3 | BM471027.1 | 1.4 | CCTTCCTTTGCCCAGatgctgagggttgga |
| | protein 10 | | | | |
| 368 | SERPINE1 mRNA | NM_015640.1 | BC003049.1 | 1.1 | TTTTTCACATTACAGtggcctgaagcacga |
| | binding | | | | |
| | protein 1 | | | | |
| | (SERBP1), | | | | |
| | transcript | | | | |
| | variant 4 | | | | |
| 369 | ribosomal | NM_000990.2 | BG824148.1 | 0.8 | TGGGAAAGTTGGTATGAAGCATTAC |
| | protein L27a | | | | CACTTAAAGAGGAACCAGAGCTTCT |
| | | | | | GCCCAACTGTCAACCTTGACAAATT |
| | | | | | GTGGACTTTGGTCAGTGAACAGACA |
| | | | | | CGGGTGAATGCTGCTAAAAACAAGA |
| | | | | | CTGGGGCTGCTCCCATCATTGATGT |
| | | | | | GGTGCGATCG |
| 370 | Actinin, | NM_001102.2 | AK098203.1 | 1.3 | ATGGACACGGATGATTTCCGCGCCT |
| | alpha 1 | | | | GCCTGATCTCC |
| 371 | chromosome 6 | NM_030939.2 | BE813002.1 | 0.8 | GATGTAATTGGCTGTACTCAGGAGA |
| | open reading | | | | TGGATTTCATTCTTTGGCCTCGGAA |
| | frame 62 | | | | TGATATTGAAAAAATCGTCTGTCTC |
| | | | | | CTGTTTTCTAGGTGGAAAGAATCTG |
| | | | | | ATGAGCCT |
| 372 | Homo sapiens | BC034141.1 | BF870126.1 | 1.1 | AGGTGGAGATCAGACgaactgtggctgcac |
| | immuno- | | | | |
| | globulin | | | | |
| | kappa | | | | |
| | constant | | | | |
| | chain (cDNA | | | | |
| | clone MGC | | | | |
| | 32713) | | | | |
| 373 | cleft lip | NM_001294.1 | AK027698.1 | 1.3 | GACGGGCCAGCCCGACCTCACACT |
| | and palate | | | | GCCT |
| | associated | | | | |
| | transmembrane | | | | |
| | protein 1 | | | | |
| 374 | enoyl Coenzyme | NM_001398.1 | CB268506.1 | 1.0 | GAACGCAGTAGACGAAGGCGGCGG |
| | A hydratase 1, | | | | CGTAGGCGGCGGGGATAGTGGCTT |
| | peroxisomal | | | | CTCGCAGACTCCGCG |
| 375 | membrane- | NM_145021 | EXH- | 1.5 | GCCACTGTGAAGGAGATGATGAGA |
| | associated | | PBMP0572-01 | | GCCCCCTGATCACCCCCTGCCACT |
| | ring finger | | | | GCACAGGAAGCCTCCACTTCGTGC |
| | (C3HC4) | | | | ACCAGGCCTGCCTGCAGCAGTGGA |
| | | | | | TCAA |
| 376 | maltase- | NM_004668 | EXH- | 1.3 | GGGAGCAGATATCTGTGGGTTCTT |
| | glucoamylase | | PBMO1078-01 | | TCAAGATGCTGAATATGAGATGTG |
| | (alpha- | | | | TGTTCGCTGGATGCAGCTGGGGGC |
| | glucosidase) | | | | CTTTTACCCCTTCTCAAGAAACCA |
| | | | | | CAACACCATTGGGA |
| 377 | eukaryotic | NM_001404.3 | BE502067.1 | 0.8 | TGCCATCACCTAGCTGCCTGCACC |
| | translation | | | | TGCCCTTCAGGGAGATGGGGGTCA |
| | elongation | | | | TTAAAGGAAACTGAACATTGA |
| | factor 1 | | | | |
| | gamma | | | | |
| 378 | O-linked N- | NM_181673.1 | AW002377.1 | 0.6 | CTCTGGTTGAAGCTTTGCTTATTG |
| | acetylgluco- | | | | TAACAGGCTTTTATTTCCAGGTAA |
| | samine | | | | TATGTCTTGGAAGACTTAATTCTG |
| | (GlcNAc) | | | | ATTAGAGATATAGATATTACTGGA |
| | transferase | | | | AACTAATTGTTTTTTTTCTATTGT |
| | | | | | ACTCTGCTTTATCAAAGAAGTAAA |
| | | | | | ACATTTAAATCGTACTACAGAAAT |
| | | | | | TAAGATGTTGTCTTGCGATCCTTA |
| | | | | | ATAAAT |
| 379 | Homo sapiens | AK026373.1 | BG699574.1 | 0.7 | TTTTCCTTTGGCAGGaaggtgtcttgctgc |
| | cDNA: | | | | |
| | FLJ22720 | | | | |
| | fis, clone | | | | |
| | HSI14320 | | | | |
| 380 | cellular | NM_001997.2 | AW795076.1 | 0.8 | CTGGAGGGCATTGCCCCGGAAGAT |
| | homolog of | | | | CAAGTCGTGCTCCTGGCAGGCGCG |
| | the fox | | | | CCCCTGGAGGATGAGGCCACTCTG |
| | sequence in | | | | GGCCAGTGCGGGGTGGAGGCCCTG |
| | the Finkel- | | | | ACTACCCTGGAAGTAGCAGGCCGC |
| | Biskis- | | | | ATGCTTGGAGGTGAGTGAGAGAGG |
| | Reilly | | | | AATGTTCTTTGAAGTACCGGTAAG |
| | murine | | | | CGTCTAGTGAGTGTGGGGTGCATA |
| | sarcoma | | | | GTCCTGACAGCTGAGTGTCACACC |
| | virus | | | | TATGGTAATAGAGTACTTCTCACT |
| | | | | | GTCTTCAGTTCAGAGTGATTCTTC |
| | | | | | CTGTTTACATCCCTCATGTTGAAC |
| | | | | | ACAGACGTCCATGGGAGACTGAGC |
| | | | | | CAGAGTGTAGTTGTATTTCAGTCA |
| | | | | | CATCACGAGATCCTAGTCTGGTTA |
| | | | | | TCAGCTTCCACACTAAAATTAGGT |
| | | | | | CAGACCAGGGCCCCCAAAGTGCTC |
| | | | | | TATAAAATTAGAAGCTGGAAGATC |
| | | | | | CTGAAATGAAACTTAAGATTTCAA |
| | | | | | GGTCAAATATCTGCAACTTTGTTC |
| | | | | | TCATTACCTATTGGGCGCAG |
| 381 | cellular | NM_001997.2 | W05251.1 | 0.7 | TTCCCTGGCCCGTGCTGGAAAAGT |
| | homolog of | | | | GAGAGGTCAGACTCCTAAGGTGAG |
| | the fox | | | | TGAGAGTATTAGTGGTCATGGTGT |
| | sequence in | | | | TAGGACCTTTTTTCCTTTCACAGC |
| | the Finkel- | | | | TAAACCAAGTCCCTGGGCTCTTAC |
| | Biskis- | | | | TCGGTTTGCCTTCTCCCTCCCTGG |
| | Reilly | | | | AGATGAGCCTGAGGGAAGGGATGC |
| | murine | | | | TAGGTGTGGAAGACAGGAACCAGG |
| | sarcoma | | | | GCCTGATTAACCTTCCCTTCTCCA |
| | virus | | | | GGTGGCCAAACAGGAGAAGAAGAA |
| | | | | | GAAGAAGACAGGTCGGGCTAAGCG |
| | | | | | GCGGATGCAGTACAACCGGCGCTT |
| | | | | | TGTCAACGTTGTGCCCACCTTTGG |
| | | | | | CAAGAAGAAGGGCCCCAATGCCAA |
| | | | | | CTCTTAAGTCTTTTGTAATTCTGG |
| | | | | | CTTTCTCTAATAAAAAAGCCACTT |
| | | | | | AGTTCA |
| 382 | Pyruvate | NM_002654.3 | AI186154.1 | 1.2 | TTGCCATGAATGTTGgcaaggcccgaggct |
| | kinase, | | | | |
| | muscle | | | | |
| | (PKM2), | | | | |
| | transcript | | | | |
| | variant 1 | | | | |
| 383 | transla- | NM_003295.1 | AA223997.1 | 0.6 | AGATTACATGAAATCaatcaaaggggaact |
| | tionally- | | | | |
| | controlled | | | | |
| | tumor | | | | |
| | protein 1 | | | | |
| 384 | transla- | NM_003295.1 | BC040008.1 | 0.6 | TGAAGAACAGAGACCAGAAAGAGT |
| | tionally- | | | | AAAACCTTTTATGACAGGGGCTGC |
| | controlled | | | | AGAACAAATCAAGCACATCCTTGC |
| | tumor | | | | TAAT |
| | protein 1 | | | | |
| 385 | lysozyme | NM_000239.1 | BE720647.1 | 0.9 | AGGCATTAGAGCATGggtggcatggagaaa |
| 386 | cold shock | NM_003651.3 | BC009744.1 | 1.6 | GGCCCTCCCCGGAATgctggtgagattgga |
| | domain | | | | |
| | protein A | | | | |
| 387 | beta-2- | NM_004048.2 | AV734235.1 | 0.8 | AGCAGCATCATGGAGgtttgaagatgccgc |
| | micro- | | | | |
| | globulin | | | | |
| 388 | Homo sapiens | NM_004368.1 | BX371275.1 | 1.1 | CCTCCTGAGTAGCTAGGACTGCAG |
| | calponin 2 | | | | GTGCTCCACCACGCCCGGCTAATT |
| | (CNN2), | | | | TTTGTATTTTTAGTAGAGATGGGG |
| | transcript | | | | TTTCCCCATGTTGGCCAGGCTGGT |
| | variant 1 | | | | CTCGAACTCCTGGCCTCAGGTGTG |
| | | | | | ATCCGCCCGCCTCCGCCTCCCCAA |
| | | | | | GCGCTGAGATTACAGGTGTGAGCC |
| | | | | | ACCGTGCCCAGGCCCTCAGTAGGT |
| | | | | | TTT |
| 389 | PBX/knotted 1 | NM_004571.2 | NM_197976.1 | 1.1 | GCTGGGATTACAGGTgtgagccaccgcgcc |
| | homeobox 1 | | | | |
| | (PKNOX1), | | | | |
| | transcript | | | | |
| | variant 1 | | | | |
| 390 | cytochrome c | NM_004718.2 | BG496431.1 | 0.7 | CCCTTTGAAGTTCCTTTTTCATTG |
| | oxidase | | | | TTAAATTAAAATTTTTTTTTTTAC |
| | subunit | | | | TTGGATGGCTTAACATTTTTGCAA |
| | VIIa poly- | | | | GAAAAATAGGAAGATATGAAGATG |
| | peptide 2 | | | | ATGTTTTGGTTTGTTTATGAAATG |
| | like | | | | CATATGGCTTGTCAGAGCTCATTC |
| | (COX7A2L) | | | | GACAGTTAAAGCCATTGTTTAAAG |
| | | | | | AAATGGTGCTTTGCTCTGTGTTTG |
| | | | | | TGCTCCTGATTTCCCTGGAGGTTC |
| | | | | | TGGATGAAGGCTGAACACAGGCTT |
| | | | | | GTTAATGTCAGTCTGTGCTGAGGA |
| | | | | | CCTCAGGGACTTGAGGTTGCATTT |
| | | | | | TTGAGCATGGGGTGCAGGAGCCTT |
| | | | | | TCTGGATTTGGATGTGGCTATGGA |
| | | | | | AAGAACACAGAAGCCAAGGTCATG |
| | | | | | TGCATGAAATGAGGAGTTTGAGTT |
| | | | | | AGTCACCTCGGGGATTTTTTCCAT |
| | | | | | TTTGCAGTAAAATGTTAAATTAAT |
| | | | | | GTAGCCTGCCTCTATTTGTTGGGC |
| | | | | | AGGTAATTTCAAAGGGTTATTTGC |
| | | | | | CTCATCTCCTATCTTTAGTG |
| 391 | receptor | NM_005669.3 | BF589237.1 | 0.7 | TATATTAAATTCTGAATGCAATTT |
| | accessory | | | | TTTTTTGTTCCCTTGAGACCAAAA |
| | protein 5 | | | | TTTAAGTTAACTGTTGCTGGCAGT |
| | (REEP5), | | | | CTAAGTGTAAATGTTAACAGCAGG |
| | | | | | AGAAGTTAAGAATTGAGCAGTTCT |
| | | | | | GTTGCATGATTTCCCAAATGAAAT |
| | | | | | ACTGCCTTGGCTAGAGTTTGAAAA |
| | | | | | ACTAATTGAGCC |
| 392 | receptor | NM_005669.3 | BF589237.1 | 0.6 | TGTGCCTGGCTAGAAaacaagcgtttattt |
| | accessory | | | | |
| | protein 5 | | | | |
| | (REEP5), | | | | |
| 393 | nuclear | NM_006163.1 | BC005044.1 | 1.5 | TGGGCTTTCCGGGAACCTGGACCA |
| | factor | | | | GACTCTGGCCCAGTAGGATGTCCC |
| | (erythroid- | | | | CGTGTCCTCCCCAGCAGAGCAGGA |
| | derived 2), | | | | ACAGGGTGATACAGCTGTCCACTT |
| | 45 kDa | | | | CAGAGCTAGGAGAGATGGAACTGA |
| | (NFE2), | | | | CTTGGCAGGAGATCATGTCCATCA |
| | | | | | CCGAGCTGCAG |
| 394 | nuclear | NM_006163.1 | BC005044.1 | 1.3 | GTGACTCCACCACAGgtttctagagccatc |
| | factor | | | | |
| | (erythroid- | | | | |
| | derived 2), | | | | |
| | 45 kDa | | | | |
| | (NFE2), | | | | |
| 395 | F-box | NM_012179.2 | BF727126.1 | 2.1 | AATGACGACAGTATGttagggcctagtcaa |
| | protein 7 | | | | |
| 396 | cornichon | NM_014184.1 | BI196635.1 | 0.8 | ATACATTATGGTGCCGAGTGGTAAC |
| | homolog 4 | | | | ATGGGAGTGTTTGATCCAACAGAAA |
| | (Drosophila) | | | | TACACAATCGAGGGCAGCTGAAGTC |
| | (CNIH4) | | | | ACACATGAAAGAAGCCATGATCAAG |
| | | | | | CTTGGTTTCCACTTGCTCTGCTTCT |
| | | | | | T |
| 397 | cornichon | NM_014184.1 | BI752106.1 | 0.7 | GGCAGCTGAAGTCACACATGAAAGA |
| | homolog 4 | | | | AGCCATGATCAAGCTTGGTTTCCAC |
| | (Drosophila) | | | | TTGCTCTGCTTCTTCATGTATCTTT |
| | (CNIH4) | | | | ATAG |
| 398 | lysosomal- | NM_014713.2 | BE314894.1 | 0.8 | GTAGTAAACCTATTGATGGCAATTT |
| | associated | | | | TGCTGACTGTGGAAGTGACTCATCC |
| | protein | | | | AAACTCCATGCCAGCTGTCAACATT |
| | transmembrane | | | | C |
| | 4 alpha | | | | |
| 399 | BAT2 domain | NM_015172.1 | AB029019.2 | 1.2 | CCCAGCTCCAACCCCCATCCTTGCC |
| | containing 1 | | | | TCAGTTTCAACCCCAGCTTCTGTCA |
| | | | | | CCATTCTTGCCTCAGCCTCAATTCC |
| | | | | | CATTCTTGCTTCAGCCCTAGCATCA |
| | | | | | ACTTCAGCTCCAACGCCAGCCCCAG |
| | | | | | CAGCCTCTTCCCCAGCTGCCCCAGT |
| | | | | | CATCACAGCACCAACTATCCCAGCC |
| | | | | | TCAGCCCCAACTGCCTCAGTCCCAC |
| | | | | | TTGCCCCTGCCTCAGCTTCAGCCCC |
| | | | | | AGCCCCAGCCCCTACCCCAGTCTCA |
| | | | | | GCCCCAAATCCTGCCCCACCTGCCC |
| | | | | | CAGCCCAGACTCAGGCACAGACCCA |
| | | | | | CAAACCAGTCCAGAATCCACTACAG |
| | | | | | ACTACATCTCAGTCTTCAAAACAAC |
| | | | | | CACCACCATCAATTAGGCTGCCTTC |
| | | | | | AGCTCAAACACCTAATGGCACAGAT |
| | | | | | TATGTAGCCTCAGGAAAATCCATCC |
| | | | | | AGACCCCACAGTCACATGGCACTCT |
| | | | | | GACAGCTGAATTATGGGATAACAAG |
| | | | | | GTGGCCCCACCAGCTGTGCTGAATG |
| 400 | adenosine | NM_015841.1 | BQ448703.1 | 1.7 | TTCTGAGATTCTTTCcttgtgatctgaatg |
| | deaminase, | | | | |
| | RNA- | | | | |
| | specific | | | | |
| 401 | Microtubule | BC048206.1 | AW015234.1 | 1.1 | GTATTTGTGCAGATCCTGGCCAGTA |
| | associated | | | | CAAAGTCGTTGCTCTTGTCTTATCT |
| | monoxygenase, | | | | TCTCTTACAGAGTCTCCCTCCCTTT |
| | calponin and | | | | ATAGAATGTCAACCAAAGAGTGCCC |
| | LIM domain | | | | TCCTCCCCTCTCAGCCTCCTCTTTA |
| | containing 2 | | | | GCTAGCCTCCCCATCTCATCACAAC |
| | | | | | GCATGTCTGTGACCTTTGGTAATCA |
| | | | | | TTTACAGTGCCACACGGAACCCTGT |
| | | | | | ATTTTGCACACAGCAAAACAAACAA |
| | | | | | TGTTTAGCTTTATTTATGGTATTTG |
| | | | | | ATGCTGTAAATGGAAATAAAT |
| 402 | Microtubule | BC048206.1 | AW015234.1 | 1.4 | TGCTGCACGCTCACTgtatttgtgcagatc |
| | associated | | | | |
| | monoxygenase, | | | | |
| | calponin and | | | | |
| | LIM domain | | | | |
| | containing 2 | | | | |
| 403 | major histo- | NM_019111.2 | AV704276.1 | 0.8 | AGTTTGATGCTCCAAGCCCTCTCCC |
| | compatibility | | | | AGAGACTACAGAGAACGTGGTGTGT |
| | complex, | | | | GCCCTGGGCCTGACTGTGGGTCTGG |
| | class II, | | | | TGGGCATCATTATTGGGACCATCTT |
| | DR alpha | | | | CATC |
| 404 | Homo sapiens | NM_021038.1 | BM083621.1 | 0.6 | TCAGTGGTTTATTGTTCACAAAAAA |
| | muscleblind- | | | | ATCTTCAAAACAAGTATTGACTTTC |
| | like | | | | ACAAAATTTAAATCATAAACAGGCA |
| | (Drosophila) | | | | AACCAAACAGCACACTGTAGCTATA |
| | (MBNL1), | | | | GTTGTTATGTGATTGTTTTTTAATT |
| | transcript | | | | GCTGTAGGATCCTGTTCTTTCAGCA |
| | variant 1 | | | | GGTGAAAAATAAAACGCAGTTCAAA |
| | | | | | TTTCATGGTTTTAATTTTCAACTCA |
| | | | | | GAAGCACTCAAAAATGCAAAATGTG |
| | | | | | ATAATGGGCACTTGTTTAAAAGAAT |
| | | | | | TAGTGTATCCAGCCTTCACTCCAGC |
| | | | | | TGGTTAAAAATGTTGCACTTATCAG |
| | | | | | CAACCCTACCACTTTCATCTGCTGA |
| | | | | | AAGGACAAATGTGCTTGGTTTTACT |
| | | | | | ATTATGTAATCACAACTTACTTTCT |
| | | | | | GCTTGTAGTTGCTTAAAATTATGTA |
| | | | | | TTTTGTCTTGGGCTGCAATTTGTTT |
| | | | | | TATGCTTATTTTATTATTACTGCAG |
| | | | | | TAGTTGACTTTGCTGTATGGAAAAA |
| | | | | | TAAAGTGAAATTGCCCTAATAAAAC |
| 405 | membrane- | NM_022349.2 | NM_152851.1 | 0.5 | TCTGACTTCCCTGGGagtgtacttttcctg |
| | spanning 4- | | | | |
| | domains, | | | | |
| | subfamily A, | | | | |
| | member 6A | | | | |
| 406 | bromodomain | NM_033656.1 | NM_018963.2 | 0.8 | TTGCACTTGCTGGTCTTTGTAGCAG |
| | and WD | | | | CATTCAGCACAGGTGCCAAAATATG |
| | repeat | | | | CTTCATTTTGGGGGCAGATCTATTT |
| | domain | | | | TGACAGTATTTGACTACATATAGCA |
| | containing 1 | | | | AGAGTTTGAAATATGTTAAACACTA |
| | (BRWD1), | | | | GACATCCTGGTTATCAAAACCAATG |
| | transcript | | | | AGCATTACTTTCATGGCAGCAAGTG |
| | variant 2 | | | | |
| | | | | | TCATGCAGTTATTTTCTGAATTTGT |
| | | | | | CAAAGAGGCAGTAGTTTCTAACCCC |
| | | | | | TGTTCTATAGTAGTTACAACAATTT |
| | | | | | CACAACCTATGTTTACAGATTCTTC |
| | | | | | ATAAATACATGCATACTGACACTAT |
| | | | | | AATCATGGGAGGTGTAACCATGATT |
| | | | | | AGTAGGCGAGGTACCTACCACTTTT |
| | | | | | TTTTTTTTCTTCCCCTGGCTACTTG |
| | | | | | AGTAGAATGCATTATACCAGATCTG |
| | | | | | GTCACTTTCATTGAAATGGTTTCTA |
| | | | | | ATTTTCTTCCCAAGTGCTGTTGGGT |
| | | | | | TTTTTTCTTCTTAAGGAAAACGTTG |
| | | | | | TCACTTTTATGTTATAAACTTGAAT |
| 407 | chromosome | NM_138774.2 | AI375989.1 | 1.2 | GCCTCTGCCCTTGCACTACCTTGTC |
| | 19 open | | | | TGTCACCCCATCCCGTGTCCCCTCG |
| | reading | | | | TCCCCCAGCCTGACTCCTGCCTGAT |
| | frame 22 | | | | |
| | | | | | AGCTCCTGTGTCCCCATGCTGGTCC |
| | | | | | TCCTGGCCCAGGCTGCAGGAGCCAG |
| | | | | | GCTGGGGGGCCTCCGCACCCCCTTG |
| | | | | | CTGCGTGTGGGTAATTGTGTTTTGG |
| | | | | | GGGAAAGTGGGGAATTTAATAAATT |
| | | | | | TCTGGTGCT |
| 408 | CD97 antigen | NM_001784.2 | BI030515.1 | 1.3 | GGAGTCCACAGCCAGacgctttcccgattc |
| 409 | chromosome 11 | NM_170746.1 | BF311566.1 | 0.4 | gtagaagccctcatgctgagCTTTGTGTCC |
| | open reading | | | | |
| | frame 31 | | | | |
| | (C11orf31), | | | | |
| 410 | chromosome 11 | NM_170746.1 | BF311566.1 | 0.7 | CGTAGGGAGATTTGGgtagaagccctcatg |
| | open reading | | | | |
| | frame 31 | | | | |
| | (C11orf31), | | | | |
| 411 | chromosome 11 | NM_170746.1 | BF311566.1 | 0.7 | AGCCCTCATGCTGAGctttgtgtccctggt |
| | open reading | | | | |
| | frame 31 | | | | |
| | (C11orf31), | | | | |
| 412 | eukaryotic | nm_001402.4 | bm475798.1 | 0.8 | AGCAGCTGGCTTCACtgctcaggtgattat |
| | translation | | | | |
| | elongation | | | | |
| | factor 1 | | | | |
| | alpha 1 | | | | |
| 413 | eukaryotic | NM_001402.4 | BM999355.1 | 0.9 | GTGATTATCCTGAACCATCCAGGCC |
| | translation | | | | AAATAAGCGCCGGCTATGCCCCTGT |
| | elongation | | | | ATTGGATTGCCACACGGCTCACATT |
| | factor 1 | | | | GCATGCAAGTTTGCTGAGCTGAAGG |
| | alpha 1 | | | | AAAAGATTGATCGCCGTTCTGGTAA |
| | | | | | AAAGCTGGAAGATGGCCCTAAATTC |
| | | | | | T |
| 414 | eukaryotic | NM_001402.4 | AU120105.1 | 0.8 | GCTGACTGTGCTGTCCTGATTGTTG |
| | translation | | | | CTGCTGGTGTTGGTGAATTTGAAGC |
| | elongation | | | | TGGTATCTCCAAGAATGGGCAGACC |
| | factor 1 | | | | CGAGAGCATGCCCTTCTGGCTTACA |
| | alpha 1 | | | | CACTGGGTGTGAAACAACTAATTGT |
| | | | | | CGGTGTTAACAAAATGGATTCCACT |
| | | | | | GAGCCACCCTACAGCCAGAAGAGAT |
| | | | | | ATGAGGAAATTGTTAAG |
| 415 | adducin 1 | NM_176801.1 | AI962551.1 | 1.1 | TCATGTGGCATTCTCTCTGCTCAGT |
| | (alpha) | | | | GATCTCACTTAAATCTATATACAAA |
| | (ADD1), | | | | GCCTTGGTCCCGTGAAAACACTCGT |
| | transcript | | | | GTGCCCACCAGCGGCCTTGAAGAGG |
| | variant 4 | | | | CAGGTCTGGGCCAGATGCTGGGCAG |
| | | | | | GAAACCCCAGCGGCAGATGGGCCTG |
| | | | | | TGTGCACCCAACGTGATGCTATGCA |
| | | | | | TGTCTGACCGACGATCCCTCGACCA |
| | | | | | GAATCAGATTCAGGAGCTCAGTTTC |
| | | | | | TTTTTCACTTGGGTCTCTGGATTCC |
| | | | | | TGTCATAGGGAAGGTATATCAGGAG |
| | | | | | GGGAAGAGGCCTTTCTAGAATTTTC |
| | | | | | TTTGAGCAGGTTTACAATTTAGCTT |
| | | | | | ACATTTTTCGACTGTGAACGTGAAT |
| | | | | | AGGCTGCTTTTTGCTTTCTTCTTTC |
| | | | | | CAGACCCCACAGTAGAGCACTTTTC |
| | | | | | ACTTATTTGGGGGAGGCTTCAGGGG |
| | | | | | ACTGTTCTCACCTTAACTCAGCCAG |
| | | | | | AAAGATGCCCTAGTTGTGATCAAAG |
| | | | | | GTAACTCGAGGTGGAGGGTAGCCCT |
| 416 | CD97 antigen | NM_001784.2 | BF763029.1 | 1.3 | GCTCCGGGCAGCATCAGTGTGACAG |
| | | | | | CTCCACCGTCTGCTTCAACACCGTG |
| | | | | | GGTTCATACAGCTGCCGCTGCCGCC |
| | | | | | CAGGCTGGAAGCCCAGACACGGAAT |
| | | | | | CCCGAATAACCAAAAGGACACTGTC |
| | | | | | TGTGAAG |
| 417 | Homo sapiens | BC009917.1 | BI914208.1 | 1.1 | CCCCGGAAAGCCAGCGCCACATGC |
| | hypothetical | | | | AGTTCGGCCACAGCAGCAGCCTCC |
| | protein | | | | AGTGGCCTGGAGGAGTGGACTAGC |
| | DKFZp761A052 | | | | CGGTCCCCGCGGCAGCGGAGTTCA |
| | | | | | GCCTCGTCACCTGAGCACCCTGAG |
| | | | | | CTGCATGCTGAATTGGGCATGAAG |
| | | | | | CCCCCTTCCCCAGGCACTGTTTTA |
| | | | | | GCTCTTGCCAAACCTCCTT |
| 418 | transla- | NM_003295.1 | BI909906.1 | 0, 6 | AGATTACATGAAATCaatcaaagggaaact |
| | tionally | | | | |
| | controlled | | | | |
| | tumor | | | | |
| | protein 1 | | | | |
| 419 | cold shock | NM_003651.3 | BU665383.1 | 1, 8 | CCCACGACCTGCCCCAGCAGTTGG |
| | domain | | | | AGAGGCTGAAGATAAAGAAAATCA |
| | protein A | | | | GCAAGCCACCAGTGGTCCAAACCA |
| | | | | | GCCGTCTGTTCGCCGTGGATATCG |
| | | | | | GCGTCCCTACAATTACCGGCGTCG |
| | | | | | CCCGCGTCCTCCTAACGCTCCTTC |
| | | | | | ACAAGATGGCAAAGAG |
| 420 | cold shock | NM_003651.3 | BF740038.1 | 1, 5 | GGTGCAGAAGCTGCCAATGTGACT |
| | domain | | | | GGCCCGGATGGAGTTCCTGTGGAA |
| | protein A | | | | GGGAGTCGTTACGCTGCAGATCGG |
| | | | | | CGCCGTTACAGACGTGGCTACTAT |
| | | | | | GGAAGGCGCCGT |
| 421 | kynureninase | NM_003937.2 | BG220595.1 | 0, 8 | TCTGTGAGATGAATTTAAAAGTGC |
| | (L-kynurenine | | | | CTCAGAATGCAGTTGCCCTTATTG |
| | hydrolase) | | | | TG |
| | (KYNU), | | | | |
| | transcript | | | | |
| | variant 1 | | | | |
| 422 | tubulin, | NM_006000.1 | AL533321.2 | 1, 2 | CGTGAATGCATCTCAGTCCACGTG |
| | alpha 1 | | | | GGGCAGGCAGGTGTCCAGATGGGC |
| | (TUBA1), | | | | |
| | | | | | AATGCCTGCTGGGAGCTCTATTGC |
| | | | | | TTGGAACATGGGATTCAGCCTGAT |
| | | | | | GGGCAGATGCCCAGTGACAAGACC |
| | | | | | ATTGGTGGAGGGGACGACTCCTTC |
| | | | | | ACCACCTTCTTCTGTGAAACTGGT |
| | | | | | GCTGGAAAACACGTACCCCGGGCA |
| | | | | | GTTTTTGTGGATCTGG |
| 423 | syntaxin | NM_006949.1 | CD722977.1 | 1, 2 | CCTGCCCCGAGCCCCTGTTCAGTG |
| | binding | | | | AGCTAGGCCGCTCTCGTCTGGCAA |
| | protein 2 | | | | AGGTGGTGAAGACGTTGAAGGAGA |
| | | | | | TTCACCTTGCCTTCCTC |
| 424 | F-box | NM_012179.2 | BX648151.1 | 1, 3 | CTTTTACCCGACAAGcactgaacctaccag |
| | protein 7 | | | | |
| 425 | ribosomal | NM_001007.2 | CB150953.1 | 1, 1 | CCTGAGGAGGCCAAGtacaagttgtgcaaa |
| | protein | | | | |
| | S4, X- | | | | |
| | linked | | | | |
| | (RPS4X), | | | | |
| 426 | ribosomal | NM_001021.2 | AV752729.1 | 0, 5 | CAACAAGATAGCAGGttatgtcacgcatct |
| | protein S17 | | | | |
| 427 | actin, beta | NM_001101.2 | BE930510.1 | 1, 4 | AGCCTTCCTTCCTGGgcatggagtcctgtg |
| | (ACTB), | | | | |
| 428 | actin, beta | NM_001101.2 | BM927346.1 | 1, 3 | TTCCAGCCTTCCTTCcctgggcatggagtc |
| | (ACTB), | | | | |
| 429 | zinc finger, | NM_032226.1 | BQ422572.1 | 0, 8 | CGGGGTCAATGTCAGATTTAGTCAT |
| | CCHC domain | | | | GCTGTTATTTTAGCCCAGTGGTCCA |
| | containing 7 | | | | G |
| 430 | chromosome 11 | NM_170746.1 | AW873139.1 | 0, 8 | GTGCGGAGCTCTGGACTGGGATTAA |
| | open reading | | | | GAAGGGGCCCCCACGCAAACTCAAA |
| | frame 31 | | | | TTCCCTGAGCCTCAAGAGGTGGTGG |
| | (C11orf31), | | | | AAGAGTTGAAGAAGTACCTGTCGTA |
| | | | | | GGGAGATTTGGGTAGA |
| 431 | chromosome 11 | NM_170746.1 | AW873139.1 | 0, 7 | GCCCGGACGGCAGCAgtgcggagctctgga |
| | open reading | | | | |
| | frame 31 | | | | |
| | (C11orf31), | | | | |
| 432 | eukaryotic | NM_001402.4 | BX454657.1 | 0, 9 | CATCCAGGCCAAATAAGCGCCGGCT |
| | translation | | | | ATGCCCCTGTATTGGATTGCCACAC |
| | elongation | | | | GGCTCACATTGCATGCAAGTTTGCT |
| | factor 1 | | | | GAGCTGAAGGAAAAGATTGATCGCC |
| | alpha 1 | | | | GTTCTGGTAAAAAGCTGGAAGATGG |
| | | | | | CCCTAAATTCTTGAAGTCTGGTGAT |
| | | | | | GCTGCCATTGTTGATATGGTTCCTG |
| | | | | | GCAAGCCCATGTGTGTTGAGAGCTT |
| | | | | | CTCAGACTATCCACCTTTGG |
| 433 | cDNA | AK075218.1 | AV748031.1 | 0.8 | AAAATTATATAGATATTTGCTTTTC |
| | FLJ90737 | | | | TGCTGGTTTTTTTTTTTTAATTGCA |
| | fis, clone | | | | ACTGCTTTTCTGCCGTGCCTCTCTT |
| | PLACE1010827 | | | | CCCTACCCGTGATG |
| 434 | interleukin 2 | NM_000206.1 | EXH- | 1.2 | GACGATGCCCCGAATTCCCACCCTG |
| | receptor, | | PBMO0419-01 | | AAGAACCTAGAGGATCTTGTTACTG |
| | gamma (IL2RG) | | | | AATAC |
| 435 | interleukin 2 | NM_000206.1 | EXH- | 1.3 | CACGGGAACTTTTCGgcctggagtggtgtg |
| | receptor, | | PBMO0419-01 | | |
| | gamma (IL2RG) | | | | |
| 436 | transla- | NM_003295.1 | PBMNOP_C1221_1 | 0.5 | AGGCATTGTTTTTAAGAAAAACATGT |
| | tionally- | | | | CATGTAGGTTGTCTAAAAATAAAATG |
| | controlled | | | | CATTT |
| | tumor | | | | |
| | protein 1 | | | | |
| 437 | transla- | NM_003295.1 | PBMNOP_C1221_1 | 0.4 | AAACTCATTTGAGAGaatgccttttagttt |
| | tionally- | | | | |
| | controlled | | | | |
| | tumor | | | | |
| | protein 1 | | | | |

The variants SEQ ID Nos: 308-318 correspond to novel ESTs

TABLE 2
Panel of 100 markers expressed differentially in breast cancers (PANEL 7)

| | | | | | |
|---|---|---|---|---|---|
| | | | | C I/II | |
| SEQ | Description of | Genbank N^{o} | Genbank N^{o} | vs | |
| ID N^{o} | sequence | (reference) | (variant) | Healthy | Target Sequence |

| | | | | | |
|---|---|---|---|---|---|
| 3 | cDNA | AL832453.2 | BU634341.1 | 0.8 | GAATGAATTTTCTTGctgctatgcct |
| | DKFZp451G151 | | | | ttCt |
| | (Leucine-rich repeat | | | | |
| | kinase 2) | | | | |
| 5 | cDNA DKFZp6670093 | AL832878.1 | A1223 156.1 | 0.7 | TGCAGATTTGATGGCctactgtga |
| | (Guanine nucleotide | | | | agcaca |
| | binding protein, | | | | |
| | gamma 2) | | | | |
| 7 | Homo sapiens | BC009917.1 | BC028225.1 | 1.4 | ATGAAGAAAAACAAAgtgcacag |
| | hypothetical protein | | | | agacccg |
| | DKEZp761A052 | | | | |
| 12 | Microtubule associated | BC048206.1 | AF052170.1 | 1.4 | TGGGAGGGTCGACCTTGATC |
| | monoxygenase, | | | | ATGAAACAATACCATGAGGGG |
| | calponin and LIM | | | | GCCTCTGTCACCTTTGAAAAG |
| | domain containing 2 | | | | AACACTTTTTGAGCAGCCTCA |
| | | | | | AAAAGCTCATACATACCAGCG |
| | | | | | CCTTCTTAAATTGGCTCTAATG |
| | | | | | TAAAGATTGTTAATGTCATTTA |
| | | | | | TCAAAACCATAGGTGATTATTT |
| | | | | | GGAGGGATTTAAAAAACTTAA |
| | | | | | TTACTCTCAGGCCTCATCCCA |
| | | | | | AGCTTGACACATGCTCTGTAG |
| | | | | | GTTGAACACATAATCACAAAT |
| | | | | | ATTCTAGCAAATGCTGCCTTG |
| | | | | | GTTGCAGCCTGCACTGTAGAC |
| | | | | | CCAAGGGTTTTGCTGTGGCTC |
| | | | | | TTCTTATCTCCCTTGGCTCATA |
| | | | | | AAGCCCCAGATGATGCCAGA |
| | | | | | GCTTCAATTAGAGCCATCATC |
| | | | | | ATCCCAGGCAGGGATATCTTT |
| | | | | | GAGAAATGACTCAGTTCAGCC |
| | | | | | CCAGGCCCCTGTGACTCTGCT |
| | | | | | TAAAGCACACATTTCTGCTGA |
| | | | | | CTCTTGTACCTGGGGCAGCA |
| | | | | | GGATAATCACCAACAC |
| 14 | cellular homolog of | NM_001997.2 | W17004.1 | 0.6 | GGCCGCATGCTTGGAaggtaaa |
| | the fox sequence in the | | | | gtccatgg |
| | Finkel-Biskis-Reilly | | | | |
| | murine sarcoma virus | | | | |
| 16 | cellular homolog of | NM_001997.2 | AA063591.1 | 0.7 | TGACCGGCCAGGAAAcggtcgcc |
| | the fox sequence in the | | | | cagatca |
| | Finkel-Biskis-Reilly | | | | |
| | murine sarcoma virus | | | | |
| 17 | cellular homolog of | NM_001997.2 | AA094898.1 | 0.6 | CAGGCCGCATGCTTGaggtaaa |
| | the fox sequence in the | | | | gtccatgg |
| | Finkel-Biskis-Reilly | | | | |
| | murine sarcoma virus | | | | |
| 18 | cellular homolog of | NM_001997.2 | AA187006.1 | 0.7 | AGGCCGCATGCTTGGaggtaaa |
| | the fox sequence in the | | | | gtccatgg |
| | Finkel-Biskis-Reilly | | | | |
| | murine sarcoma virus | | | | |
| 19 | cellular homolog of | NM_001997.2 | AA225636.1 | 0.6 | GGCCAGGAAACGGTCgcccaga |
| | the fox sequence in the | | | | tcaaggta |
| | Finkel-Biskis--Reilly | | | | |
| | murine sarcoma virus | | | | |
| 23 | cellular homolog of | NM_001997.2 | BU603086.1 | 0.6 | GAAGTAGCAGGCCGCatgcttgg |
| | the fox sequence in the | | | | aggtaaa |
| | Finkel-Biskis-Reilly | | | | |
| | murine sarcoma virus | | | | |
| 24 | cellular homolog of | NM_001997.2 | BF218408.1 | 0.6 | AGGCCGATGCTTGGAggtaaagt |
| | the fox sequence in the | | | | ccatggt |
| | Finkel-Biskis-Reilly | | | | |
| | murine sarcoma virus | | | | |
| 25 | cellular homolog of | NM_001997.2 | AI499403.1 | 0.7 | CCCGCATGCTTGGAGgtaaagtc |
| | the fox sequence in the | | | | catggtt |
| | Finkel-Biskis-Reilly | | | | |
| | murine sarcoma virus | | | | |
| 26 | cellular homolog of | NM_001997.2 | AW795076.1 | 0.6 | CGGTCGCCCAGATCAaggctcat |
| | the fox sequence in the | | | | gtagcct |
| | Finkel-Biskis-Reilly | | | | |
| | murine sarcoma virus | | | | |
| 35 | SEC14 like 1 | NM_003003.1 | AK130317.1 | 1.3 | TAGGGCTAGTAGGTAGGGCT |
| | (S. cerevisiae) | | | | AGTAGGTAGGGCTAGTAGGTA |
| | | | | | GGGCTAGTAGGTAGGGCTAG |
| | | | | | TAGGTAGGGCTAGTAGGTAG |
| | | | | | GGCTAGTAGGTAGGGTTCGTA |
| | | | | | GGTAGGGTTCGTAGGTAGGG |
| | | | | | TTCGTAGGTAGGGTTAGTAGC |
| | | | | | GCGTCTGTGCTGCTTCCACCT |
| | | | | | GGTGCTTCCTGTTCCCAAATC |
| | | | | | ACAAGGGCCTGAAGGTGGTC |
| | | | | | CCTGCTTTCTCTTTCTCTTTCT |
| | | | | | CTGTGTCTCAGATGGCGATTT |
| | | | | | TGCTGACAGCTGCCAAGAAAA |
| | | | | | TGCTTCACTCAACAGTCCTCA |
| | | | | | TGTGCCCAGAGATGTTTATAG |
| | | | | | AACTGTTTGAATTGCAGCCAT |
| | | | | | CCCCTGCCCCCTCCCAGGCT |
| | | | | | GAAGATCTGTTCTTTTTAAGTT |
| | | | | | GATTCGGGAGTGGCATTCTTT |
| | | | | | TATACCCAAAGACTGTAGTGC |
| | | | | | ATCTTGAAGAGCTCAAAGCAC |
| | | | | | ATGACCGCACAAATGCTTACA |
| | | | | | GGGTTTCCTCCCGAGTAATCC |
| | | | | | AATCTCACTCCCCTTGTAAGG |
| 43 | nuclear factor | NM_003204.1 | BM973053.1 | 1.5 | CGGGTCAGTGTACAGgaagagg |
| | (erythroid-derived 2)- | | | | caggcact |
| | like 1 | | | | |
| 44 | nuclear factor | NM_003204.1 | BM973053.1 | 1.5 | TGCTGTGAGGCAGAGgaatgatg |
| | (erythroid-derived 2)- | | | | gagaatc |
| | like 1 | | | | |
| 46 | synuclein, alpha (non | NM_000345.2 | NM_007308.1 | 1.4 | CCACAGGAAGGAATTCTGGAA |
| | A4 component of | | | | GATATGCCTGTGGATCCTGAC |
| | amyloid precursor) | | | | AATGAGGCTTAT |
| 47 | synuclein, alpha (non | NM_000345.2 | NM_007308.1 | 1.5 | GACCAGTTGGGCAAGaatgaag |
| | A4 component of | | | | aaggagcc |
| | amyloid precursor) | | | | |
| 51 | translationally | NM_003295.1 | CA848049.1 | 0.7 | AGAtcgcggacgggttgtgcctggaggT |
| | controlled tumor | | | | GG |
| | protein 1 | | | | |
| 52 | translationally | NM_003295.1 | CA848049.1 | 0.5 | TCCGACATCTACAAGatccggga |
| | controlled tumor | | | | gatcgcg |
| | protein 1 | | | | |
| 53 | translationally | NM_003295.1 | BC022436.1 | 0.6 | CCGACATCTACAAGATCCGGG |
| | controlled tumor | | | | AGATCGCGGACGGGTTGTGC |
| | protein 1 | | | | CTG |
| 54 | translationally | NM_003295.1 | BC040008.1 | 0.5 | GCGCCGCTCCGGCTGCACCG |
| | controlled tumor | | | | CGCTCGCTCCGAGTTTCAGG |
| | protein 1 | | | | CTCGTGCTAAGCTAGCGCCGT |
| | | | | | CGTCGTCTCCCTTCAGTCGCC |
| | | | | | ATCATGATTATCTACC |
| 55 | TYRO protein tyrosine | NM_003332.1 | BF092099.1 | 0.8 | GAGGGGCTGCGGAGGcagcga |
| | kinase binding protein | | | | cccggaaac |
| 60 | cold shock domain | NM_003651.3 | BC009744.1 | 1.5 | CCCAACAGAATACAGgctggtga |
| | protein A | | | | gattgga |
| 64 | beta2-microglobulin | NM_004048.2 | AV734235.1 | 0.7 | ATTTGGATTGGATGAATTCCA |
| | | | | | AATTCTGCTTGCTTGCTTTTTA |
| | | | | | ATATTGATATGCTTATACACTT |
| | | | | | ACACTTTATGCACAAAATGTA |
| | | | | | GGGTTATAATAATGTTAACAT |
| | | | | | GGACATGATCTTCTTTATAATT |
| | | | | | CTACTTTGAGTGCTGTCTCCA |
| | | | | | TGTTTGATGTATCTGAGCAGG |
| | | | | | TTGCTCCACAGGTAGCTCTAG |
| | | | | | GAGGGCTGGCAAC |
| 69 | SEC24 related gene | NM_004922.1 | AW449995.1 | 1.8 | GCAGCTGTCTGGAATgcagagg |
| | family, member C | | | | cagctgga |
| | (S. cerevisiae) | | | | |
| 71 | hemoglobin, alpha 1/2 | NM_000517.3 | H78334.1 | 1.4 | CGGTCAACTTTCAAGctccttaag |
| | | | | | ccactg |
| 72 | hemoglobin, alpha 1/2 | NM_000517.3 | H55830.1 | 1.4 | TCAAGGCCGCCTGGGgatgttcct |
| | | | | | gtcctt |
| 80 | hemoglobin, alpha 1/2 | NM_000558.3 | R91899.1 | 1.7 | ACCAAGACCTACTTCccggtcaac |
| | | | | | ttcaag |
| 81 | hemoglobin, alpha 1/2 | NM_000558.3 | H58664.1 | 1.7 | GGAGGCCCTGGAGAGctcctaag |
| | | | | | ccactgc |
| 85 | CD164 antigen, | NM_006016.3 | AF299342.1 | 0.8 | CTGTGACTCCAACCTCACAAC |
| | sialomucin | | | | CTGTGCGAAAGTCTACCTTTG |
| | | | | | ATGCAGCCAGTTTCATTGGAG |
| | | | | | GAATTGTCCTGGTCTTGGGTG |
| | | | | | TGCAGGCTGTAATTTTCTTTCT |
| | | | | | TTATAAATTCTGCAAATCTAAA |
| | | | | | GAACGAAATTACCACACTCTG |
| | | | | | TAAACAGACCCATTGAATTAAT |
| | | | | | AAGGACTGGTGATTCATTTGT |
| | | | | | GTAACTCACTGAAGCCAAAAT |
| | | | | | ACTATCTTTTAAGATGTCCCAC |
| | | | | | ATGGAAGACGCTATTCCAGGA |
| | | | | | TCTTTAAATTTCCATGGATGCA |
| | | | | | TATAGGATGTTTGGGAGCATC |
| | | | | | ATCCGTGAAGAAAAAATCAAT |
| | | | | | TAAATCATTGTGTTCAACAGG |
| | | | | | AATATTTAAAATATTCTGCATG |
| | | | | | AATCCTGTGGCTGTCTTATTTT |
| | | | | | AAATAGCTGCTGCTGTGGGAT |
| | | | | | TATATTTTTTTTCCTTAACATG |
| | | | | | CCAAATATAACTTTCTGAAAGT |
| | | | | | GATGGAAAATGTTGTCTTGTG |
| | | | | | CAGACAACATCATGGCTCTTG |
| | | | | | GCAGTTTA |
| 86 | CD164 antigen, | NM_006016.3 | AF299343.1 | 0.8 | CAGCCAATTCTACAGctaaaccc |
| | sialomucin | | | | acagttc |
| 88 | talin 1 | NM_006289.2 | A1393487.1 | 1.4 | CCCAGAGTATTAACGCTCCAA |
| | | | | | GAGTATTATTAACGCTGCTGT |
| | | | | | ACCTCGATCTGAATCTGCCGG |
| | | | | | GGCCCCAGCCCACTCCACCC |
| | | | | | TGCCAGCAGCTTCCAGCCAGT |
| | | | | | CCCCACAGCCTCATCAGCTCT |
| | | | | | CTTCACCGTTTTTTGATACTAT |
| | | | | | CTTCCCCCACCCCCAGCTACC |
| | | | | | CATAGGGGCTGCAGAGTTATA |
| | | | | | AGCCCCAAACAGGTCATGCTC |
| | | | | | CAATAAAAATGATTCTACCTAC |
| | | | | | AA |
| 89 | talin 1 | NM_006289.2 | A1393487.1 | 1.4 | TGCTTCGGAAGGAACgagagctg |
| | | | | | gaagagg |
| 90 | talin 1 | NM_006289.2 | A1417760.1 | 1.4 | AGGAAGAAATGCTTCggaagga |
| | | | | | acgagagc |
| 95 | acidic (leucinerich) | NM_006401.1 | Y07570.1 | 1.5 | GAAGTCAGTGAGGAGgaagaag |
| | nuclear | | | | aatttgga |
| | phosphoprotein 32 | | | | |
| | family, member B | | | | |
| 102 | soluble galactoside | NM_009587.1 | BG698264.1 | 0.8 | CTCCAGTGGAACCAGgtttgctgtg |
| | binding lectin 9 | | | | aactt |
| | (galectin 9) | | | | |
| 116 | Homocysteine | NM_014685.1 | BG828243.1 | 0.7 | GGAAAACATCTCAAGgcctgaag |
| | inducible, endoplasmic | | | | ctgccca |
| | reticulum stress | | | | |
| | inducible, ubiquitin | | | | |
| | like domain member 1 | | | | |
| 120 | ring finger protein 10 | NM_014868.3 | BU626650.1 | 1.5 | CGAGAGCGCAGGATTGAGAT |
| | | | | | AGAGGAGAACA |
| 121 | ring finger protein 10 | NM_014868.3 | BU626650.1 | 1.5 | AGAAACAGGGCAAGTacccaga |
| | | | | | agtccaca |
| 125 | ribosomal protein L4 | NM_000968.2 | CB141160.1 | 0.7 | GTCATCAGACTAGTGCTGAGT |
| | | | | | CTTGGGGTACTGGCAGAGCT |
| | | | | | GTGGCTCGAATTCCCAGAGTT |
| | | | | | CGAGGTGGTGGGACTCACCG |
| | | | | | CTCTGGCCAGGGTGCTTTTGG |
| | | | | | AAACatgtgtcgtg |
| 126 | ribosomal protein L4 | NM_000968.2 | CD686462.1 | 0.6 | GCGTGTGCTCGCCCACTGATA |
| | | | | | TCGGTGTACTCCGAAAAGGG |
| | | | | | GGAGTCATCTGGCAAAAATGT |
| | | | | | CACTTTGCCTGCTGTATTCAA |
| | | | | | GGCTCCTATTCGACCAGATAT |
| | | | | | TGTGAACTTTGTTCACACCAA |
| | | | | | CTTGCGCAAAAACAACAGACA |
| | | | | | GCCCTATGCTG |
| 137 | ubiquitin B | NM_018955.2 | AA206538.1 | 1.4 | CAGGTCAAAATGCAGatcttcgtg |
| | | | | | aaaacc |
| 138 | ubiquitin B | NM_018955.2 | AA206538.1 | 1.3 | CAGGTCAAAATGCAGatcttcgtg |
| | | | | | aagacc |
| 139 | ubiquitin B | NM_018955.2 | AA340917.1 | 1.4 | AGATCTTCGTGAAGACCCTGA |
| | | | | | CCGGCAAGACCATCACTCTG |
| | | | | | GAGGTGGAGCCCAGTGACAC |
| | | | | | CATCGAAAATGTGAAGGCCAA |
| | | | | | GATCCAAGATAAAGAAGGCAT |
| | | | | | CCCCCCCGACCAGCAGAGGC |
| | | | | | TCATCTTTGCAGGCAAGCAGC |
| | | | | | TGGAAGATGGCCGCACTCTTT |
| | | | | | CTGACTACAACATCCAGAAAG |
| | | | | | AGTCGACCCTGCACCTGGTC |
| | | | | | CTGCGCCTGAGGGGTGGCTG |
| | | | | | TTAATTCTTCAGTCATGGCATT |
| | | | | | CGCAGTGCCCAGTGATGGCA |
| | | | | | TTACTCTGCACTATAGCCATTT |
| | | | | | GCCCCAACTTAAGTTTAGAAA |
| | | | | | TTACAAGTTTCAGTAATAGCT |
| | | | | | GAACCTGTTCAAAATGTTAATA |
| | | | | | AAGG |
| 140 | ubiquitin B | NM_018955.2 | BU661443.1 | 1.4 | TCCCTGTGGGTGGACGTGGT |
| | | | | | TGGTGATTGGCAGGATCCT |
| 141 | ubiguitin B | NM_018955.2 | BU661443.1 | 1.5 | GGTTGGCTTTGTTGGgtgagcttg |
| | | | | | tttgtg |
| 145 | major | NM_019111.2 | CD686254.1 | 0.7 | TCAGAGACAGTCTTCCTGCCC |
| | histocompatibility | | | | AGGGAAGACCACCTTTTCCGC |
| | complex, class II, DR | | | | AAGTTCCACTATCTCCCCTTC |
| | alpha | | | | CTGCCCTCAACTGAGGACGTT |
| | | | | | TACGACTGCAGGGTGGAGCA |
| | | | | | CTGGGGCTTGGATGAGCCTC |
| | | | | | TTCTCAAGCACTGGGagtttgatg |
| | | | | | ctccaagccctctc |
| 147 | KIAA1191 protein | NM_020444.2 | B1254429.1 | 1.5 | GCAGCAGGATCACAGaacagac |
| | | | | | ccaggaaa |
| 148 | mesoderm induction | NM_020948.1 | AY124186.1 | 0.7 | TTTCTCACACAGGCAtactccaaa |
| | early response 1 | | | | tgcttc |
| 151 | myelin protein zero- | NM_024569.2 | A1693779.1 | 0.9 | TGCCCTGGCATTCTGGCAGA |
| | like 1 | | | | GAATCCTCACCAGTTCTCACC |
| | | | | | AACCTTCCCCCCAGGCAAGG |
| | | | | | GCAGCTGCCAGCATGGTGCT |
| | | | | | CTGCCAGGACAGGTTTCCCTG |
| | | | | | AAGGAAGCTGCTCACACTGAG |
| | | | | | ATGAGCCTCTCAGGGCAGGA |
| | | | | | CCTCTTCCCAAGCCCTGCACA |
| | | | | | CCCACCCCTGCAGCCCTTTTG |
| | | | | | GCTC |
| 152 | likely ortholog of | NM_030915.1 | W45195.1 | 1.1 | GCTTCAGTCCGCCGAgagcagta |
| | mouse limbbud and | | | | ccgtgtg |
| | heart gene | | | | |
| 155 | caspase 4, apoptosis- | NM_033306.1 | NM_001225.2 | 1.2 | TGTTCCCTATGGCAGaaggcaac |
| | related cysteine | | | | cacagaa |
| | protease | | | | |
| 158 | major | NM_033554.2 | BU621846.1 | 0.7 | AAGGAGCCTGTGGAGctgggcca |
| | histocompatibility | | | | gcccaac |
| | complex, class II, DP | | | | |
| | alpha 1 | | | | |
| 159 | FK506 binding protein | NM_054014.1 | NM_000801.2 | 0.8 | GCTCCCTGTTCTTGGatctgccat |
| | 1A, 12kDa | | | | ggaggg |
| 160 | chloride intracellular | NM_001288.3 | BG491600.1 | 0.8 | CCAGGGACGGCCACTTCCTG |
| | channel 1 | | | | GTCCCCGACGCAACCATGGC |
| | | | | | TGAAGAACAACCGCAGGTCG |
| | | | | | AATTGTTCGTGAAG |
| 161 | chloride intracellular | NM_001288.3 | AV683308.1 | 0.7 | GGGCAGCTCCCATTCctgctgtat |
| | channel 1 | | | | ggcact |
| 164 | chromosome 19 open | NM_138774.2 | A1375989.1 | 1.4 | ACCTCATCTCGGCCAgtgctgacc |
| | reading frame 22 | | | | tggagg |
| 177 | tropomyosin 3 | NM_153649.1 | BM006741.1 | 1.3 | TGATGAGAGTGAGAGgcagaga |
| | | | | | cccgtgct |
| 184 | eukaryotic translation | NM_001404.3 | AA206367.1 | 0.7 | CTGAGTCCAGATTGGCAGGT |
| | elongation factor 1 | | | | GGACTACGAGTCATACACATG |
| | gamma | | | | GCGGAAACTGGATCCTGGCA |
| | | | | | GCGAGGAGACCCAGACGCTG |
| | | | | | GTT |
| 185 | eukaryotic translation | NM_001404.3 | AA206367.1 | 0.6 | CAGCATGTGGGCAAAGCCTTC |
| | elongation factor 1 | | | | AATCAGGGCAAGATCTTCAAG |
| | gamma | | | | TGAACATCTCTTGCCATCACC |
| | | | | | TAG |
| 187 | eukaryotic translation | NM_001404.3 | BU783548.1 | 0.6 | ATTTAAGCGCAAGTACTCCAA |
| | elongation factor 1 | | | | TGAGGACACACTCTCTGTGGC |
| | gamma | | | | ACTGCCATATTTCTGGGAGCA |
| | | | | | CTTTGATAAGGACGGCTGGTC |
| | | | | | CCTGTGGTACTCAGAGTATCG |
| | | | | | CTTCCCTGAAGAACTCACTCA |
| | | | | | GACCTTCATGAGCTGCAATCT |
| | | | | | CATCACTG |
| 188 | eukaryotic translation | NM_001404.3 | BE502067.1 | 0.6 | TGGACAAGCTGAGGAAGAAT |
| | elongation factor 1 | | | | GCCTTCGCCAGTGTCATCCTT |
| | gamma | | | | TTTGGAACCAACAATAGCAGC |
| | | | | | TCCATTTCTGGAGTCTGGGTC |
| | | | | | TTCCGAGGCCAG |
| 189 | eukaryotic translation | NM_001404.3 | BE502067.1 | 0.6 | CAGGTGGACTACGAGTCATAC |
| | elongation factor 1 | | | | ACATGGCGGAAACTGGATCCT |
| | gamma | | | | GGCAGCGAGGAGACCCAGAC |
| | | | | | GCTGGTTCGAGAGTACTTTTC |
| | | | | | CTGGGAGGGGGCCTTCCAGC |
| | | | | | ATGTGGGCAAAGCCTTCAA |
| 190 | eukaryotic translation | NM_001404.3 | BE502067.1 | 0.6 | GAGCTTGCCTTTCCGctgagtcca |
| | elongation factor 1 | | | | gattgg |
| | gamma | | | | |
| 191 | eukaryotic translation | NM_001404.3 | BG533219.1 | 0.8 | AAGGAGGAGAAAAAGGCGGC |
| | elongation factor 1 | | | | TGCCCCTGCTCCTGAGGAGG |
| | gamma | | | | AGATGGATGAATGTGAGCAG |
| | | | | | GCGCTGGCTGCTGAGCCCAA |
| | | | | | GGCCAAGGACCCCTTCGCTC |
| | | | | | ACCTGCCCAAGAG |
| 203 | calpain, small subunit | NM_001749.1 | BE907701.1 | 1.4 | CCTTTGAGGCAGCAGatgaaagt |
| | 1/calpain, small | | | | gggaaca |
| | subunit 1 | | | | |
| 204 | Cysteinyl-tRNA | NM_001751.3 | AK125503.1 | 1.5 | AAACAGGAACAAGAAgcagcaaa |
| | synthetase | | | | gctggcc |
| 222 | prohibitin | NM_002634.2 | BE536369.1 | 1.4 | GCGAGGAGAGTGCGTgtgtgaga |
| | | | | | gggtcca |
| 225 | translationally | NM_003295.1 | BG284235.1 | 0.6 | TTCTTTATTGGTGAAAACATGA |
| | controlled tumor | | | | ATCCAGATGGCATGGTTGCTC |
| | protein 1 | | | | TATTGGACTACCGTGAGGATG |
| | | | | | GTGTGACCCCATATATGATTT |
| | | | | | TCTTTAAGGATGGTTTA |
| 227 | translationally | NM_003295.1 | CD641954.1 | 0.7 | TTATTTTGGATCTATCACCTGT |
| | controlled tumor | | | | CATCATAACTGGCTTCTGCTT |
| | protein 1 | | | | GTCATCCACACAACACCAGGA |
| | | | | | CTTAAGACAAATGGGACTGAT |
| | | | | | GTCATCTTGAGCTCTTCATTTA |
| | | | | | TTTTGACTGTGATTTATTTGGA |
| | | | | | GTGGAGGCATTGTTTTTAAGA |
| | | | | | AAAACATGTCATGTAGGTTGT |
| | | | | | CTAAAAATAAAATGCATTTAAA |
| | | | | | C |
| 228 | translationally | NM_003295.1 | AV749932.1 | 0.6 | CGTCGTCTCCCTTCAGTCGCC |
| | controlled tumor | | | | ATCATGATTATCTACC |
| | protein 1 | | | | |
| 229 | translationally | NM_003295.1 | AV749932.1 | 0.6 | GGGACCTCATCAGCCacgatgag |
| | controlled tumor | | | | atgttct |
| | protein 1 | | | | |
| 232 | cold shock domain | NM_003651.3 | BE935120.1 | 1.5 | AATAACCCACGGAAATATCTG |
| | protein A | | | | CGCAGTGTAGGAGATGGAGA |
| | | | | | AACTGTAGAGTTTGATGTGGT |
| | | | | | TGAAGGAGAGAA |
| 236 | Homo sapiens vesicle | NM_003761.2 | BG623073.1 | 0.7 | GATCTGGAAGCCACAtctgagca |
| | associated membrane | | | | cttcaag |
| | protein 8 (endobrevin) | | | | |
| 237 | CASP8 and FADD | NM_003879.3 | BI871546.1 | 1.3 | AGTACAAGCAGTCTGgtggatgg |
| | like apoptosis | | | | aatggaa |
| | regulator | | | | |
| 239 | beta2-microglobulin | NM_004048.2 | BM831738.1 | 0.7 | TGGAGGCTATCCAGCgtactcca |
| | | | | | aagattc |
| 240 | Guanine nucleotide | NM_004125.2 | BC015206.1 | 0.6 | GTGGAGAGGATCAAGgtctctcag |
| | binding protein, | | | | gcagct |
| | gamma 10 | | | | |
| 242 | c-src tyrosine kinase | NM_004383.1 | BG953215.1 | 1.1 | CCTCAAGTTCTCGCTagatgtctg |
| | | | | | cgaggc |
| 248 | hemoglobin, alpha 1/2 | NM_000517.3 | AA343446.1 | 1.7 | TGCTTCTCCCCGCAGgatgttcct |
| | | | | | gtcctt |
| 253 | membrane component, | NM_005898.2 | AA437165.1 | 0.8 | AACAGCTTCAAACAGtggttggca |
| | chromosome 11, | | | | cttacc |
| | surface marker 1 | | | | |
| 254 | capping protein (actin | NM_006136.1 | BG702980.1 | 0.7 | AGCAAAAAATTTTTGgaatggtcgt |
| | filament) muscle Z- | | | | tggag |
| | line, alpha 2 | | | | |
| 258 | acidic (leucinerich) | NM_006401.1 | A1446778.1 | 1.3 | AGGAGGAGGACGAAGaaggag |
| | nuclear | | | | aagatgagg |
| | phosphoprotein 32 | | | | |
| | family, member B | | | | |
| 261 | lysosomal associated | NM_006762.1 | BQ006415.1 | 1.5 | AAGATGCTCCAGAAGgtgagtgtg |
| | multispanning | | | | gctgca |
| | membrane protein 5 | | | | |
| 266 | chromosome 11 open | NM_014206.1 | BE041814.1 | 0.8 | TCGCGGGGCAAAATGgagctcga |
| | reading frame 10 | | | | ggccatg |
| 269 | ribosomal protein L4 | NM_000968.2 | CB164625.1 | 0.7 | GGTGCTTTTGGAAACatgtgtcgtg |
| | | | | | gaggc |
| 276 | ankyrin repeat domain | NM_017664.1 | BC039715.1 | 0.7 | GGAGCATTCCATATAGAAACT |
| | 10 | | | | GCTGAAACTGCCACAGGTGCT |
| | | | | | TCTCCGAAAACCTTACAGTTG |
| | | | | | TGGCATTGAATGTTCAGTATC |
| | | | | | GCTTCCTTTCTGCACACG |
| 280 | ribosomal protein S17 | NM_001021.2 | BE731466.1 | 0.5 | AATTATGTTCCTGAGgtctcagcct |
| | | | | | tggat |
| 281 | ribosomal protein S17 | NM_001021.2 | AV752729.1 | 0.6 | GCCGCGTTCCACCAAAACCGT |
| | | | | | GAAGAAAGCGGCCCGGGTCA |
| | | | | | TCATAGAAAAGTACTACACGC |
| | | | | | GCCTGGGCAACGACTTCCAC |
| | | | | | ACGAACAAGCGCGTGTGCGA |
| | | | | | GGAGATCGCCATTATCCCCAG |
| | | | | | CAAAAAGCTCCG |
| 282 | sulfatase 2 | NM_018837.1 | AB033073.2 | 1.5 | GCGAGAGTGTGTCGAgtgagtgt |
| | | | | | gcgtctg |
| 283 | ubiguitin B | NM_018955.2 | BG286180.1 | 1.6 | GAAGGCGGAAAAGAGgtcaaaat |
| | | | | | gcagatc |
| 284 | ubiquitin B | NM_018955.2 | BG286180.1 | 1.4 | GGTATCCGCTAACAGgtcaaaat |
| | | | | | gcagatc |
| 290 | hypothetical protein | NM_024841.1 | BQ919225.1 | 1.4 | ATTGTTGAACTGGATgcggctgttg |
| | FLJ14213 | | | | aagag |
| 298 | chloride intracellular | NM_001288.3 | AA126087.1 | 0.8 | CCTGAGTCCAACACAGCTGG |
| | channel 1 | | | | GCTGGACATATTTGCCAAATT |
| | | | | | TTCTGCCTACATCAAGAATTC |
| | | | | | AAACCCAGCACTCAATGACA |
| 299 | chloride intracellular | NM_001288.3 | AA126087.1 | 0.7 | CCCAGGTACCCCAAGctggcagc |
| | channel 1 | | | | tctgaac |
| 300 | chloride intracellular | NM_001288.3 | AA126087.1 | 0.8 | GCTGTGCCCTCCCAGgtacccca |
| | channel 1 | | | | agctggc |
| 307 | arachidonate 5- | NM_001629.2 | BF892107.1 | 0.9 | AAGTGGAGCACGAAAGCAGG |
| | lipoxygenase- | | | | ACCCAGAATGGGAGGAGCTT |
| | activating protein | | | | CCAGAGGACCGGAACACTTG |
| | | | | | CCTTTGAGCGGGTCTACACTG |
| | | | | | CCAA |
| 309 | ribosomal protein L41 | NM_021104.1 | EXH-002 | 0.6 | GAAGCGAATGCGCAGgctgaag |
| | | | | | cgcaaaag |
| 312 | cyclin T2 | NM_001241.2 | EXH-005 | 0.5 | ttgtgtgagctattcaaactcttcaacccctga |
| 314 | zinc finger protein 384 | NM_133476.2 | EXH007 | 1.5 | AGTTCAGGAGCCCTGGAAAG |
| | | | | | GAGAAGGAATAAGACGGCAG |
| | | | | | GAGGAAGAGA |

TABLE 3
Panel of 66 markers expressed differentially in breast cancers (PANEL 6)

| | | | | | |
|---|---|---|---|---|---|
| | | | | C I/II | |
| SEQ | Description of | Genbank N^{o} | Genbank N^{o} | vs | |
| ID N^{o} | sequence | (reference) | (variant) | Healthy | Target Sequence |

| | | | | | |
|---|---|---|---|---|---|
| 5 | cDNA DKFZp667I093 | AL832878.1 | A1223156.1 | 0.7 | TGCAGATTTGATGGCctactgtga |
| | (Guanine nucleotide | | | | agcaca |
| | binding protein, | | | | |
| | gamma 2) | | | | |
| 7 | Homo sapiens | BC009917.1 | BC028225.1 | 1.4 | ATGAAGAAAAACAAAgtgcacag |
| | hypothetical protein | | | | agacccg |
| | DKFZp761A052 | | | | |
| 13 | cellular homolog of | NM_001997.2 | NM_001997.2 | 0.5 | GTCGCCCAGATCAAGgctcatgta |
| | the fox sequence in the | | | | gcctca |
| | Finkel-Biskis-Reilly | | | | |
| | murine sarcoma virus | | | | |
| 14 | cellular homolog of | NM_001997.2 | W17004.1 | 0.6 | GGCCGCATGCTTGGAaggtaaa |
| | the fox sequence in the | | | | gtccatgg |
| | Finkel-Biskis-Reilly | | | | |
| | murine sarcoma virus | | | | |
| 16 | cellular homolog of | NM_001997.2 | AA063591.1 | 0.7 | TGACCGGCCAGGAAAcggtcgcc |
| | the fox sequence in the | | | | cagatca |
| | Finkel-Biskis-Reilly | | | | |
| | murine sarcoma virus | | | | |
| 17 | cellular homolog of | NM_001997.2 | AA094898.1 | 0.6 | CAGGCCGCATGCTTGaggtaaa |
| | the fox sequence in the | | | | gtccatgg |
| | Finkel-Biskis-Reilly | | | | |
| | murine sarcoma virus | | | | |
| 18 | cellular homolog of | NM_001997.2 | AA187006.1 | 0.7 | AGGCCGCATGCTTGGaggtaaa |
| | the fox sequence in the | | | | gtccatgg |
| | Finkel-Biskis-Reilly | | | | |
| | murine sarcoma virus | | | | |
| 19 | cellular homolog of | NM_001997.2 | AA225636.1 | 0.6 | GGCCAGGAAACGGTCgcccaga |
| | the fox sequence in the | | | | tcaaggta |
| | Finkel-Biskis-Reilly | | | | |
| | murine sarcoma virus | | | | |
| 20 | cellular homolog of | NM_001997.2 | BM820687.1 | 0.5 | GGTCGCCCAGATCAAgctcatgta |
| | the fox sequence in the | | | | gcctca |
| | Finkel-Biskis-Reilly | | | | |
| | murine sarcoma virus | | | | |
| 23 | cellular homolog of | NM_001997.2 | BU603086.1 | 0.6 | GAAGTAGCAGGCCGCatgcttgg |
| | the fox sequence in the | | | | aggtaaa |
| | Finkel-Biskis-Reilly | | | | |
| | murine sarcoma virus | | | | |
| 24 | cellular homolog of | NM_001997.2 | BF218408.1 | 0.6 | AGGCCGATGCTTGGAggtaaagt |
| | the fox sequence in the | | | | ccatggt |
| | Finkel-Biskis-Reilly | | | | |
| | murine sarcoma virus | | | | |
| 25 | cellular homolog of | NM_001997.2 | A1499403.1 | 0.7 | CCCGCATGCTTGGAGgtaaagtc |
| | the fox sequence in the | | | | catggtt |
| | Finkel-Biskis-Reilly | | | | |
| | murine sarcoma virus | | | | |
| 26 | cellular homolog of | NM_001997.2 | AW795076.1 | 0.6 | CGGTCGCCCAGATCAaggctcat |
| | the fox sequence in the | | | | gtagcct |
| | Finkel-Biskis-Reilly | | | | |
| | murine sarcoma virus | | | | |
| 27 | cellular homolog of | NM_001997.2 | D52122.1 | 0.6 | GGCCGCATGCTTGGggtaaagtc |
| | the fox sequence in the | | | | catggtt |
| | Finkel-Biskis-Reilly | | | | |
| | murine sarcoma virus | | | | |
| 28 | cellular homolog of | NM_001997.2 | BE535673.1 | 0.6 | GCCGCATGCTTGGAGgtaacagt |
| | the fox sequence in the | | | | ccatggt |
| | Finkel-Biskis-Reilly | | | | |
| | murine sarcoma virus | | | | |
| 47 | synuclein, alpha (non | NM_000345.2 | NM 007308.1 | 1.5 | GACCAGTTGGGCAAGaatgaag |
| | A4 component of | | | | aaggagcc |
| | amyloid precursor) | | | | |
| 51 | translationally | NM_003295.1 | CA848049.1 | 0.7 | AGAtcgcggacgggttgtgcctggaggT |
| | controlled tumor | | | | GG |
| | protein 1 | | | | |
| 52 | translationally | NM_003295.1 | CA848049.1 | 0.5 | TCCGACATCTACAAGatccggga |
| | controlled tumor | | | | gatcgcg |
| | protein 1 | | | | |
| 53 | translationally | NM_003295.1 | BC022436.1 | 0.6 | CCGACATCTACAAGATCCGGG |
| | controlled tumor | | | | AGATCGCGGACGGGTTGTGC |
| | protein 1 | CTG | | | |
| 54 | translationally | NM_003295.1 | BC040008.1 | 0.5 | GCGCCGCTCCGGCTGCACCG |
| | controlled tumor | | | | CGCTCGCTCCGAGTTTCAGG |
| | protein 1 | | | | CTCGTGCTAAGCTAGCGCCGT |
| | | | | | CGTCGTCTCCCTTCAGTCGCC |
| | | | | | ATCATGATTATCTACC |
| 55 | TYRO protein tyrosine | NM_003332.1 | BF092099.1 | 0.8 | GAGGGGCTGCGGAGGcagcga |
| | kinase binding protein | | | | cccggaaac |
| 58 | uroporphyrinogen | NM_000374.2 | BQ008745.1 | 1.5 | GTGTGCCGCTGATTGtggaccct |
| | decarboxylase | | | | gatgaca |
| 64 | beta 2-microglobulin | NM_004048.2 | AV734235.1 | 0.7 | ATTTGGATTGGATGAATTCCA |
| | | | | | AATTCTGCTTGCTTGCTTTTTA |
| | | | | | ATATTGATATGCTTATACACTT |
| | | | | | ACACTTTATGCACAAAATGTA |
| | | | | | GGGTTATAATAATGTTAACAT |
| | | | | | GGACATGATCTTCTTTATAATT |
| | | | | | CTACTTTGAGTGCTGTCTCCA |
| | | | | | TGTTTGATGTATCTGAGCAGG |
| | | | | | TTGCTCCACAGGTAGCTCTAG |
| | | | | | GAGGGCTGGCAAC |
| 69 | SEC24 related gene | NM_004922.1 | AW449995.1 | 1.8 | GCAGCTGTCTGGAATgcagagg |
| | family, member C | | | | cagctgga |
| | (S. cerevisiae) | | | | |
| 80 | hemoglobin, alpha 1/2 | NM_000558.3 | R91899.1 | 1.7 | ACCAAGACCTACTTCccggtcaac |
| | | | | | ttcaag |
| 81 | hemoglobin, alpha 1/2 | NM_000558.3 | H58664.1 | 1.7 | GGAGGCCCTGGAGAGctcctaag |
| | | | | | ccactgc |
| 88 | talin 1 | NM_006289.2 | A1393487.1 | 1.4 | CCCAGAGTATTAACGCTCCAA |
| | | | | | GAGTATTATTAACGCTGCTGT |
| | | | | | ACCTCGATCTGAATCTGCCGG |
| | | | | | GGCCCCAGCCCACTCCACCC |
| | | | | | TGCCAGCAGCTTCCAGCCAGT |
| | | | | | CCCCACAGCCTCATCAGCTCT |
| | | | | | CTTCACCGTTTTTTGATACTAT |
| | | | | | CTTCCCCCACCCCCAGCTACC |
| | | | | | CATAGGGGCTGCAGAGTTATA |
| | | | | | AGCCCCAAACAGGTCATGCTC |
| | | | | | CAATAAAAATGATTCTACCTAC |
| | | | | | AA |
| 89 | talin 1 | NM_006289.2 | A1393487.1 | 1.4 | TGCTTCGGAAGGAACgagagctg |
| | | | | | gaagagg |
| 90 | talin 1 | NM_006289.2 | A1417760.1 | 1.4 | AGGAAGAAATGCTTCggaagga |
| | | | | | acgagagc |
| 116 | Homocysteine- | NM_014685.1 | BG828243.1 | 0.7 | GGAAAACATCTCAAGgcctgaag |
| | inducible, endoplasmic | | | | ctgccca |
| | reticulum stress | | | | |
| | inducible, ubiquitin | | | | |
| | like domain member 1 | | | | |
| 121 | ring finger protein 10 | NM_014868.3 | BU626650.1 | 1.5 | AGAAACAGGGCAAGTacccaga |
| | | | | | agtccaca |
| 125 | ribosomal protein L4 | NM_000968.2 | CB141160.1 | 0.7 | GTCATCAGACTAGTGCTGAGT |
| | | | | | CTTGGGGTACTGGCAGAGCT |
| | | | | | GTGGCTCGAATTCCCAGAGTT |
| | | | | | CGAGGTGGTGGGACTCACCG |
| | | | | | CTCTGGCCAGGGTGCTTTTGG |
| | | | | | AAACatgtgtcgtg |
| 137 | ubiquitin B | NM_018955.2 | AA206538.1 | 1.4 | CAGGTCAAAATGCAGatcttcgtg |
| | | | | | aaaacc |
| 139 | ubiquitin B | NM_018955.2 | AA340917.1 | 1.4 | AGATCTTCGTGAAGACCCTGA |
| | | | | | CCGGCAAGACCATCACTCTG |
| | | | | | GAGGTGGAGCCCAGTGACAC |
| | | | | | CATCGAAAATGTGAAGGCCAA |
| | | | | | GATCCAAGATAAAGAAGGCAT |
| | | | | | CCCCCCCGACCAGCAGAGGC |
| | | | | | TCATCTTTGCAGGCAAGCAGC |
| | | | | | TGGAAGATGGCCGCACTCTTT |
| | | | | | CTGACTACAACATCCAGAAAG |
| | | | | | AGTCGACCCTGCACCTGGTC |
| | | | | | CTGCGCCTGAGGGGTGGCTG |
| | | | | | TTAATTCTTCAGTCATGGCATT |
| | | | | | CGCAGTGCCCAGTGATGGCA |
| | | | | | TTACTCTGCACTATAGCCATTT |
| | | | | | GCCCCAACTTAAGTTTAGAAA |
| | | | | | TTACAAGTTTCAGTAATAGCT |
| | | | | | GAACCTGTTCAAAATGTTAATA |
| | | | | | AAGG |
| 145 | major | NM_019111.2 | CD686254.1 | 0.7 | TCAGAGACAGTCTTCCTGCCC |
| | histocompatibility | | | | AGGGAAGACCACCTTTTCCGC |
| | complex, class II, DR | | | | AAGTTCCACTATCTCCCCTTC |
| | alpha | | | | CTGCCCTCAACTGAGGACGTT |
| | | | | | TACGACTGCAGGGTGGAGCA |
| | | | | | CTGGGGCTTGGATGAGCCTC |
| | | | | | TTCTCAAGCACTGGGagtttgatg |
| | | | | | ctccaagccctctc |
| 148 | mesoderm induction | NM_020948.1 | AY124186.1 | 0.7 | TTTCTCACACAGGCAtactccaaa |
| | early response 1 | | | | tgcttc |
| 158 | major | NM_033554.2 | BU621846.1 | 0.7 | AAGGAGCCTGTGGAGctgggcca |
| | histocompatibility | | | | gcccaac |
| | complex, class II, DP | | | | |
| | alpha 1 | | | | |
| 160 | chloride intracellular | NM_001288.3 | BG491600.1 | 0.8 | CCAGGGACGGCCACTTCCTG |
| | channel 1 | | | | GTCCCCGACGCAACCATGGC |
| | | | | | TGAAGAACAACCGCAGGTCG |
| | | | | | AATTGTTCGTGAAG |
| 161 | chloride intracellular | NM_001288.3 | AV683308.1 | 0.7 | GGGCAGCTCCCATTCCtgctgtat |
| | channel 1 | | | | ggcact |
| 164 | chromosome 19 open | NM_138774.2 | A1375989.1 | 1.4 | ACCTCATCTCGGCCAgtgctgacc |
| | reading frame 22 | | | | tggagg |
| 188 | eukaryotic translation | NM_001404.3 | BE502067.1 | 0.6 | TGGACAAGCTGAGGAAGAAT |
| | elongation factor 1 | | | | GCCTTCGCCAGTGTCATCCTT |
| | gamma | | | | TTTGGAACCAACAATAGCAGC |
| | | | | | TCCATTTCTGGAGTCTGGGTC |
| | | | | | TTCCGAGGCCAG |
| 189 | eukaryotic translation | NM_001404.3 | BE502067.1 | 0.6 | CAGGTGGACTACGAGTCATAC |
| | elongation factor 1 | | | | ACATGGCGGAAACTGGATCCT |
| | gamma | | | | GGCAGCGAGGAGACCCAGAC |
| | | | | | GCTGGTTCGAGAGTACTTTTC |
| | | | | | CTGGGAGGGGGCCTTCCAGC |
| | | | | | ATGTGGGCAAAGCCTTCAA |
| 190 | eukaryotic translation | NM_001404.3 | BE502067.1 | 0.6 | GAGCTTGCCTTTCCGctgagtcca |
| | elongation factor 1 | | | | gattgg |
| | gamma | | | | |
| 191 | eukaryotic translation | NM_001404.3 | BG533219.1 | 0.8 | AAGGAGGAGAAAAAGGCGGC |
| | elongation factor 1 | | | | TGCCCCTGCTCCTGAGGAGG |
| | gamma | | | | AGATGGATGAATGTGAGCAG |
| | | | | | GCGCTGGCTGCTGAGCCCAA |
| | | | | | GGCCAAGGACCCCTTCGCTC |
| | | | | | ACCTGCCCAAGAG |
| 208 | ferritin, heavy | NM_002032.1 | BG248923.1 | 0.6 | TGTCTCTGGGGATCCCTAGTA |
| | polypeptide 1 | | | | TAACACATGCA |
| 222 | prohibitin | NM_002634.2 | BE536369.1 | 1.4 | GCGAGGAGAGTGCGTgtgtgaga |
| | | | | | gggtcca |
| 225 | translationally | NM_003295.1 | BG284235.1 | 0.6 | TTCTTTATTGGTGAAAACATGA |
| | controlled tumor | | | | ATCCAGATGGCATGGTTGCTC |
| | protein 1 | | | | TATTGGACTACCGTGAGGATG |
| | | | | | GTGTGACCCCATATATGATTT |
| | | | | | TCTTTAAGGATGGTTTA |
| 228 | translationally | NM_003295.1 | AV749932.1 | 0.6 | CGTCGTCTCCCTTCAGTCGCC |
| | controlled tumor | | | | ATCATGATTATCTACC |
| | protein 1 | | | | |
| 229 | translationally | NM_003295.1 | AV749932.1 | 0.6 | GGGACCTCATCAGCCacgatgag |
| | controlled tumor | | | | atgttct |
| | protein 1 | | | | |
| 236 | Homo sapiens vesicle | NM_003761.2 | BG623073.1 | 0.7 | GATCTGGAAGCCACAtctgagca |
| | associated membrane | | | | cttcaag |
| | protein 8 (endobrevin) | | | | |
| 240 | Guanine nucleotide | NM_004125.2 | BC015206.1 | 0.6 | GTGGAGAGGATCAAGgtctctcag |
| | binding protein, | | | | gcagct |
| | gamma 10 | | | | |
| 242 | c-src tyrosine kinase | NM_004383.1 | BG953215.1 | 1.1 | CCTCAAGTTCTCGCTagatgtctg |
| | | | | | cgaggc |
| 245 | family with sequence | NM_004699.1 | AA584911.1 | 1.6 | TCAAGAGTGAGTGTTtgcggagtc |
| | similarity 50, member | | | | agacgc |
| | A | | | | |
| 248 | hemoglobin, alpha 1/2 | NM_000517.3 | AA343446.1 | 1.7 | TGCTTCTCCCCGCAGgatgttcct |
| | | | | | gtcctt |
| 252 | putative translation | NM_005801.2 | BG655736.1 | 0.7 | CCTTTGTGCTTGCAGaagtttgcct |
| | initiation factor | | | | gcaat |
| 280 | ribosomal protein S17 | NM_001021.2 | BE731466.1 | 0.5 | AATTATGTTCCTGAGgtctcagcct |
| | | | | | tggat |
| 281 | ribosomal protein S17 | NM_001021.2 | AV752729.1 | 0.6 | GCCGCGTTCCACCAAAACCGT |
| | | | | | GAAGAAAGCGGCCCGGGTCA |
| | | | | | TCATAGAAAAGTACTACACGC |
| | | | | | GCCTGGGCAACGACTTCCAC |
| | | | | | ACGAACAAGCGCGTGTGCGA |
| | | | | | GGAGATCGCCATTATCCCCAG |
| | | | | | CAAAAAGCTCCG |
| 284 | ubiguitin B | NM_018955.2 | BG286180.1 | 1.4 | GGTATCCGCTAACAGgtcaaaat |
| | | | | | gcagatc |
| 290 | hypothetical protein | NM_024841.1 | BQ919225.1 | 1.4 | ATTGTTGAACTGGATgcggctgttg |
| | FLJ14213 | | | | aagag |
| 298 | chloride intracellular | NM_001288.3 | AA126087.1 | 0.8 | CCTGAGTCCAACACAGCTGG |
| | channel 1 | | | | GCTGGACATATTTGCCAAATT |
| | | | | | TTCTGCCTACATCAAGAATTC |
| | | | | | AAACCCAGCACTCAATGACA |
| 299 | chloride intracellular | NM_001288.3 | AA126087.1 | 0.7 | CCCAGGTACCCCAAGctggcagc |
| | channel 1 | | | | tctgaac |
| 300 | chloride intracellular | NM_001288.3 | AA126087.1 | 0.8 | GCTGTGCCCTCCCAGgtacccca |
| | channel 1 | | | | agctggc |
| 309 | ribosomal protein L41 | NM_021104.1 | EXH-002 | 0.6 | GAAGCGAATGCGCAGgctgaag |
| | | | | | cgcaaaag |
| 310 | translationally | NM_003295.1 | EXH-003 | 0.5 | AATGCATATTTAAACTAAATTG |
| | controlled tumor | | | | ATCCTGTAGTGTTCCTGGAGA |
| | protein 1 | | | | AGCTAGAGCCTGATTGTAGGC |
| | | | | | TACTACTCATCAATTAACTTCT |
| | | | | | ACAGTGGAGACTACTTCTGGG |
| | | | | | ACTGGAATATAAAAA |
| 311 | G1 to S phase | NM_002094.1 | EXH-004 | 1.5 | ATTACCGTTTATTCCATATCTG |
| | transition 1/G1 to S | | | | GATAATTTGCCGAACTTCAAT |
| | phase transition 1 | | | | AGATCAGTTGATGGACCAATC |
| | | | | | AGGCTGCCAATTGTGG |
| 312 | cyclin T2 | NM_001241.2 | EXH-005 | 0.5 | ttgtgtgagctattcaaactcttcaacccctga |

TABLE 4
Summary table of Panels 1-11 of markers expressed
differentially in breast cancers

| 1 | 2 | 3 | 4 | 5 | 6 | 7 | 8 | 9 | 10 | 11 | SEQ ID N° |
|---|---|---|---|---|---|---|---|---|---|---|---|
| | | | | | | | | * | | | 1 |
| | | | | | | | * | | | | 2 |
| | | | | | | * | * | | | | 3 |
| | | | | | * | * | * | | | | 5 |
| | | | | * | * | * | * | | | | 7 |
| | | | | | | | | | * | | 11 |
| | | | | | | * | * | | * | | 12 |
| | | | * | * | * | | | | | | 13 |
| | | | | * | * | * | * | | | | 14 |
| * | | | * | * | * | * | * | * | | | 16 |
| * | | | * | * | * | * | * | * | | | 17 |
| | * | * | * | * | * | * | | | * | | 18 |
| * | * | * | * | * | * | * | * | * | | | 19 |
| | | | | | * | | | | | | 20 |
| * | * | * | * | * | * | * | * | * | * | * | 23 |
| * | | | | * | * | * | * | * | | | 24 |
| | | | | * | * | * | * | | | | 25 |
| * | * | * | * | * | * | * | * | * | | | 26 |
| | | * | * | * | * | | | | | | 27 |
| | | | * | * | * | | | | | | 28 |
| | | | | | | | | * | | | 33 |
| | | | | | | * | * | | | | 35 |
| | | | | | | | * | | | | 37 |
| | | | | | | | | * | | | 40 |
| | | | | | | | | * | | | 41 |
| | | | | | | * | | | | | 43 |
| * | | | | | | * | * | * | | | 44 |
| | | | | | | * | * | | | | 46 |
| | | | * | * | * | * | * | | | | 47 |
| | | | | | | | | * | | | 50 |
| | * | * | * | * | * | * | * | | * | | 51 |
| * | * | * | * | * | * | * | * | * | * | * | 52 |
| * | * | * | * | * | * | * | * | * | * | * | 53 |
| * | * | * | * | * | * | * | * | * | | | 54 |
| | * | * | * | * | * | * | | | | | 55 |
| | | | * | * | * | | | | | | 58 |
| | | | | | | | | | * | | 59 |
| | | | | | | * | * | | * | | 60 |
| | | | | | * | * | * | | | | 64 |
| | * | * | * | * | * | * | * | | | | 69 |
| | | | | | | * | | | | | 71 |
| | | | | | | * | * | | | | 72 |
| | | | | | | | | * | | | 75 |
| | | | | | | | | * | | | 79 |
| | * | * | * | * | * | * | * | | | | 80 |
| | | | * | * | * | * | * | | | | 81 |
| | | | | | | * | * | | | | 85 |
| | | | | | | * | * | | | | 86 |
| | | | | | * | * | * | | | | 88 |
| | | | * | * | * | * | | * | | | 89 |
| | | | | | * | * | * | | | | 90 |
| | | | | | | * | * | | | | 95 |
| | | | | | | * | | | | | 102 |
| | | | | | | | | * | | | 104 |
| | | | | | | | | | * | | 105 |
| | | | | | | | * | | | | 107 |
| | | | * | * | * | * | * | | | | 116 |
| | | | | | | | * | * | | | 119 |
| * | | | | | | * | * | * | | | 120 |
| * | | | * | * | * | * | * | * | | | 121 |
| | * | * | * | * | * | * | * | | | | 125 |
| * | | | | | | * | * | * | | | 126 |
| | | | | | | | | * | | | 135 |
| | | | | | | | * | | | | 136 |
| * | | | | * | * | * | * | * | | | 137 |
| | | | | | | * | * | | | | 138 |
| * | | | | * | * | * | * | * | | | 139 |
| | | | | | | * | * | | | | 140 |
| | | | | | | * | * | | | | 141 |
| | | | | | | | | * | | | 144 |
| * | * | * | * | * | * | * | * | * | | | 145 |
| | | | | | | * | * | | | | 147 |
| * | * | * | * | * | * | * | * | * | * | * | 148 |
| | | | | | | | | | * | | 150 |
| | | | | | | * | * | | | | 151 |
| | | | | | | * | * | | | | 152 |
| | | | | | | * | * | | | | 155 |
| | | | * | * | * | * | * | | | | 158 |
| | | | | | | * | * | | | | 159 |
| | | | * | * | * | * | * | | | | 160 |
| | | * | * | * | * | * | * | | | | 161 |
| | | | | | | | * | | | | 163 |
| | | | * | * | * | * | * | | | | 164 |
| | | | | | | | * | * | | | 171 |
| | | | | | | | | * | | | 172 |
| | | | | | | * | * | | | | 177 |
| | | | | | | | | * | | | 178 |
| | | | | | | | | * | | | 182 |
| | | | | | | * | | | | | 184 |
| | | | | | | * | * | | | | 185 |
| * | | | | | | * | * | * | | | 187 |
| | | * | | | * | * | * | | | | 188 |
| * | | | * | * | * | * | * | * | | | 189 |
| * | | | * | * | * | * | * | * | | | 190 |
| | | | | | * | * | * | | * | | 191 |
| | | | | | | | | * | | | 192 |
| | | | | | | | | * | | | 194 |
| | | | | | | | | * | * | | 195 |
| | | | | | | | | * | | | 201 |
| | | | | | | * | | | | | 203 |
| | | | | | | * | | | | | 204 |
| | | | | | | | * | | * | | 206 |
| | | | | | * | | | | | | 208 |
| | | | | | | | * | | | | 216 |
| | | | | | | | | * | | | 217 |
| | | | | | * | * | * | | | | 222 |
| * | * | * | * | * | * | * | * | * | * | * | 225 |
| | | | | | | | | | * | | 226 |
| | | | | | | * | | * | * | | 227 |
| * | * | * | | * | * | * | * | * | | | 228 |
| * | | | * | * | * | * | * | * | * | | 229 |
| | | | | | | * | * | | | | 232 |
| | | | | | | | | * | | | 235 |
| | | | | | * | * | | | | | 236 |
| | | | | | | * | * | | | | 237 |
| | | | | | | * | * | | | | 239 |
| * | * | * | * | * | * | * | * | * | | | 240 |
| | | | | | * | * | * | | | | 242 |
| | | | | * | * | | | | | | 245 |
| | | | | | | | | * | | | 246 |
| | | | * | * | * | * | * | | | | 248 |
| | | | | | | | | * | | | 250 |
| | | | | * | * | | | | | | 252 |
| | | | | | | * | * | | | | 253 |
| * | | | | | | * | * | * | | | 254 |
| | | | | | | * | * | | | | 258 |
| | | | | | | * | | | | | 261 |
| | | | | | | * | | | | | 266 |
| | | | | | | | | * | | | 267 |
| | | | | | | * | | * | | | 269 |
| * | | | | | | * | * | * | | | 276 |
| | | | | | | | | * | | | 278 |
| | | * | * | * | * | * | * | | * | | 280 |
| * | | | * | * | * | * | * | * | * | | 281 |
| * | | | | | | * | * | * | | | 282 |
| * | | | | | | * | * | * | | | 283 |
| | | | * | * | * | * | * | | | | 284 |
| | | | | * | * | * | * | | | | 290 |
| | | | | * | * | * | * | | | | 298 |
| | | | * | * | * | * | * | | | | 299 |
| | | | * | * | * | * | * | | | | 300 |
| | | | | | | | | * | | | 306 |
| | | | | | | * | * | | | | 307 |
| | | | | | | | | | * | | 308 |
| | | | | | * | * | * | | * | | 309 |
| | | | * | * | * | | | | * | | 310 |
| | | | | | * | | * | | | | 311 |
| | * | * | * | * | * | * | * | | * | | 312 |
| | | | | | | * | * | | | | 314 |
| | | | | | | | * | | | | 315 |
| | | | | | | | * | * | | | 319 |
| | | | | | | | | * | | | 320 |
| | | | | | | | | * | | | 321 |
| | | | | | | | | * | | | 322 |
| | | | | | | | | * | | | 323 |
| | | | | | | | | * | | | 324 |
| | | | | | | | | * | | | 325 |
| | | | | | | | | * | | | 326 |
| | | | | | | | * | | * | | 327 |
| | | | | | | | | * | | | 328 |
| | | | | | | | | * | | | 329 |
| | | | | | | | | * | | | 330 |
| | | | | | | | * | | | | 331 |
| | | | | | | | | * | | | 332 |
| | | | | | | | | * | | | 333 |
| | | | | | | | | * | | | 334 |
| | | | | | | | | * | | | 335 |
| | | | | | | | * | | | | 336 |
| | | | | | | | | * | | | 337 |
| | | | | | | | | * | | | 338 |
| | | | | | | | * | | | | 339 |
| | | | | | | | | * | | | 340 |
| | | | | | | | | * | | | 341 |
| | | | | | | | | * | | | 342 |
| | | | | | | | | * | * | | 343 |
| | | | | | | | | * | | | 344 |
| | | | | | | | | * | | | 345 |
| | | | | | | | | * | | | 346 |
| | | | | | | | | * | | | 347 |
| | | | | | | | | * | | | 348 |
| | | | | | | | | * | | | 349 |
| | | | | | | | | * | | | 350 |
| | | | | | | | | * | | | 351 |
| | | | | | | | | * | | | 352 |
| | | | | | | | | * | | | 353 |
| | | | | | | | | * | | | 354 |
| | | | | | | | | * | | | 355 |
| | | | | | | | | * | | | 356 |
| | | | | | | | | * | | | 357 |
| | | | | | | | | * | | | 358 |
| | | | | | | | | * | | | 359 |
| | | | | | | | | * | * | | 360 |
| | | | | | | | | * | | | 361 |
| | | | | | | | | * | | | 362 |
| | | | | | | | * | | | | 363 |
| | | | | | | | | * | | | 364 |
| | | | | | | | | * | | | 365 |
| | | | | | | | | * | | | 366 |
| | | | | | | | | * | * | | 367 |
| | | | | | | | * | | | | 368 |
| | | | | | | | | * | | | 369 |
| | | | | | | | | * | | | 370 |
| | | | | | | | | * | | | 371 |
| | | | | | | | | * | | | 372 |
| | | | | | | | | * | | | 373 |
| | | | | | | | | * | | | 374 |
| | | | | | | | | * | | | 375 |
| | | | | | | | | * | | | 376 |
| | | | | | | | | | * | | 377 |
| | | | | | | | | | * | | 378 |
| | | | | | | | | | * | | 379 |
| | | | | | | | | | * | | 380 |
| | | | | | | | | | * | | 381 |
| | | | | | | | | | * | | 382 |
| | | | | | | | | | * | | 383 |
| | | | | | | | | | * | | 384 |
| | | | | | | | | | * | | 385 |
| | | | | | | | | | * | | 386 |
| | | | | | | | | | * | | 387 |
| | | | | | | | | | * | | 388 |
| | | | | | | | | | * | | 389 |
| | | | | | | | | | * | | 390 |
| | | | | | | | | | * | | 391 |
| | | | | | | | | | * | | 392 |
| | | | | | | | | | * | | 393 |
| | | | | | | | | | * | | 394 |
| | | | | | | | | | * | | 395 |
| | | | | | | | | | * | | 396 |
| | | | | | | | | | * | | 397 |
| | | | | | | | | | * | | 398 |
| | | | | | | | | | * | | 399 |
| | | | | | | | | | * | | 400 |
| | | | | | | | | | * | | 401 |
| | | | | | | | | | * | | 402 |
| | | | | | | | | | * | | 403 |
| | | | | | | | | | * | | 404 |
| | | | | | | | | | * | | 405 |
| | | | | | | | | | * | | 406 |
| | | | | | | | | | * | | 407 |
| | | | | | | | | | * | | 408 |
| | | | | | | | | | * | | 409 |
| | | | | | | | | | * | | 410 |
| | | | | | | | | | * | | 411 |
| | | | | | | | | | * | | 412 |
| | | | | | | | | | * | | 413 |
| | | | | | | | | | * | | 414 |
| | | | | | | | | | * | | 415 |
| | | | | | | | | | * | | 416 |
| | | | | | | | | | * | | 417 |
| | | | | | | | | | * | | 418 |
| | | | | | | | | | * | | 419 |
| | | | | | | | | | * | | 420 |
| | | | | | | | | | * | | 421 |
| | | | | | | | | | * | | 422 |
| | | | | | | | | | * | | 423 |
| | | | | | | | | | * | | 424 |
| | | | | | | | | | * | | 425 |
| | | | | | | | | | * | | 426 |
| | | | | | | | | | * | | 427 |
| | | | | | | | | | * | | 428 |
| | | | | | | | | | * | | 429 |
| | | | | | | | | | * | | 430 |
| | | | | | | | | | * | | 431 |
| | | | | | | | | | * | | 432 |
| | | | | | | | | | * | | 433 |
| | | | | | | | | | * | | 434 |
| | | | | | | | | | * | | 435 |
| | | | | | | | | | * | | 436 |
| | | | | | | | | | * | | 437 |

TABLE 5
List of genes/transcripts identified among DATAS banks compiled by
profiling the blood samples of breast cancer patients, as being
associated with immunity signalling pathways.

| Representative | |
|---|---|
| transcript | Name of gene |
| NM_000061 | Homo sapiens Bruton agammaglobulinemia tyrosine |
| | kinase (BTK), mRNA. November 2005 |
| NM_002661 | Homo sapiens phospholipase C, gamma 2 |
| | (phosphatidylinositol-specific) (PLCG2), mRNA. |
| | November 2005 |
| NM_003177 | Homo sapiens spleen tyrosine kinase (SYK), mRNA. |
| | November 2005 |
| NM_021601 | Homo sapiens CD79A antigen (immunoglobulin- |
| | associated alpha) (CD79A), transcript variant 2, |
| | mRNA. October 2005 |
| NM_080548 | Homo sapiens protein tyrosine phosphatase, non- |
| | receptor type 6 (PTPN6), transcript variant 2, mRNA. |
| | November 2005 |
| AB209585 | Homo sapiens mRNA for Fc fragment of IgG, low |
| | affinity IIb, receptor for (CD32) isoform 1 variant |
| | protein. March 2005 |
| AF025529 | Homo sapiens leucocyte immunoglobulin-like |
| | receptor-6b (LIR-6) mRNA, complete cds. September 2002 |
| AJ001685 | Homo sapiens NKG2E gene. April 2005 |
| AL353611 | Human DNA sequence from clone RP11-447M12 on |
| | chromosome 9 Contains the FCN1 gene for ficolin |
| | (collagen/fibrinogen domain containing) 1 and a novel |
| | gene, complete sequence. May 2005 |
| AL591704 | Human DNA sequence from clone RP1-128L15 on |
| | chromosome 1q21.1-21.3 Contains the 5′ end of the |
| | gene for peptidoglycan recognition protein-I-alpha |
| | (PGLYRPIalpha), the PGLYRP4 gene for |
| | peptidoglycan recognition protein 4, the S100A9 gen. . . . |
| | May 2005 |
| NM_000239 | Homo sapiens lysozyme (renal amyloidosis) (LYZ), |
| | mRNA. November 2005 |
| NM_000442 | Homo sapiens platelet/endothelial cell |
| | adhesion molecule (CD31 antigen) (PECAM1), mRNA. November 2005 |
| NM_000566 | Homo sapiens Fc fragment of IgG, high affinity la, |
| | receptor (CD64) (FCGR1A), mRNA. October 2005 |
| NM_000570 | Homo sapiens Fc fragment of IgG, low affinity IIIb, |
| | receptor (CD16b) (FCGR3B), mRNA. November 2005 |
| NM_000616 | Homo sapiens CD4 antigen (p55) (CD4), mRNA. |
| | October 2005 |
| NM_000629 | Homo sapiens interferon (alpha, beta and omega) |
| | receptor 1 (IFNAR1), mRNA. November 2005 |
| NM_001066 | Homo sapiens tumor necrosis factor receptor |
| | superfamily, member 1B (TNFRSF1B), mRNA. |
| | November 2005 |
| NM_002121 | Homo sapiens major histocompatibility complex, class |
| | II, DP beta 1 (HLA-DPB1), mRNA. November 2005 |
| NM_002535 | Homo sapiens 2′-5′-oligoadenylate synthetase 2, |
| | 69/71 kDa (OAS2), transcript variant 2, mRNA. September 2005 |
| NM_003190 | Homo sapiens TAP binding protein (tapasin) |
| | (TAPBP), transcript variant 1, mRNA. October 2005 |
| NM_003332 | Homo sapiens TYRO protein tyrosine kinase binding |
| | protein (TYROBP), transcript variant 1, mRNA. |
| | October 2005 |
| NM_005514 | Homo sapiens major histocompatibility complex, class |
| | I, B (HLA-B), mRNA. November 2005 |
| NM_005810 | Homo sapiens killer cell lectin-like receptor subfamily |
| | G, member 1 (KLRG1), mRNA. November 2005 |
| NM_005874 | Homo sapiens leukocyte immunoglobulin-like |
| | receptor, subfamily B (with TM and ITIM domains), |
| | member 2 (LILRB2), mRNA. October 2005 |
| NM_006433 | Homo sapiens granulysin (GNLY), transcript variant |
| | NKG5, mRNA. October 2005 |
| NM_021642 | Homo sapiens Fc fragment of IgG, low affinity IIa, |
| | receptor (CD32) (FCGR2A), mRNA. October 2005 |
| NM_022555 | Homo sapiens major histocompatibility complex, class |
| | II, DR beta 3 (HLA-DRB3), mRNA. October 2005 |
| NM_033130 | Homo sapiens sialic acid binding Ig-like lectin 10 |
| | (SIGLEC10), mRNA. September 2005 |
| NM_152855 | Homo sapiens immunoglobulin lambda-like |
| | polypeptide 1 (IGLL1), transcript variant 2, mRNA. |
| | September 2005 |
| NM_173065 | Homo sapiens interleukin 28 receptor, alpha |
| | (interferon, lambda receptor) (IL28RA), transcript |
| | variant 3, mRNA. October 2005 |
| NM_174892 | Homo sapiens CD300 antigen like family member B |
| | (CD300LB), mRNA. September 2005 |
| NM_181985 | Homo sapiens leukocyte immunoglobulin-like |
| | receptor, subfamily A (with TM domain), member 5 |
| | (LILRA5), transcript variant 2, mRNA. September 2005 |
| NM_203458 | Homo sapiens Notch homolog 2 (Drosophila) N- |
| | terminal like (NOTCH2NL), mRNA. September 2005 |
| X96735 | H. sapiens FALL-39 gene. September 2004 |
| BC070085 | Homo sapiens colony stimulating factor 2 receptor, |
| | beta, low-affinity (granulocyte-macrophage), mRNA |
| | (cDNA clone MGC: 87425 IMAGE: 30344148), |
| | complete cds. July 2005 |
| NM_000211 | Homo sapiens integrin, beta 2 (antigen CD18 (p95), |
| | lymphocyte function-associated antigen 1; |
| | macrophage antigen 1 (mac-1) beta subunit) |
| | (ITGB2), mRNA. November 2005 |
| NM_000560 | Homo sapiens CD53 antigen (CD53), mRNA. |
| | October 2005 |
| NM_001838 | Homo sapiens chemokine (C-C motif) receptor 7 |
| | (CCR7), mRNA. November 2005 |
| NM_002983 | Homo sapiens chemokine (C-C motif) ligand 3 |
| | (CCL3), mRNA. October 2005 |
| NM_014358 | Homo sapiens C-type lectin domain family 4, |
| | member E (CLEC4E), mRNA. September 2005 |
| NM_173216 | Homo sapiens ST6 beta-galactosamide alpha-2,6- |
| | sialyltranferase 1 (ST6GAL1), transcript variant 1, |
| | mRNA. November 2005 |
| AB209647 | Homo sapiens mRNA for Neutrophil cytosol factor 2 |
| | variant protein. March 2005 |
| AB209656 | Homo sapiens mRNA for colony stimulating factor 3 |
| | receptor isoform c precursor variant protein. March 2005 |
| AC079855 | Homo sapiens BAC clone RP11-332L16 from 7, |
| | complete sequence. January 2004 |
| AK127905 | Homo sapiens cDNA FLJ46012 fis, clone |
| | SPLEN2007689, highly similar to Neutrophil cytosol |
| | factor 1. February 2004 |
| AL390725 | Human DNA sequence from clone RP11-373C9 on |
| | chromosome 1 Contains the 3′ end of the ZNF364 |
| | gene for zinc finger protein 364, the CD160 gene for |
| | CD160 antigen, the PDZK1 gene for PDZ domain |
| | containing 1, the 3′ end of the gene for put. .. May 2005 |
| AY131997 | Homo sapiens tumor necrosis factor receptor |
| | superfamily, member 1A (TNFRSF1A) gene, |
| | complete cds. July 2002 |
| AY692262 | Homo sapiens interleukin 2 receptor, gamma (severe |
| | combined immunodeficiency) (IL2RG) gene, complete |
| | cds. August 2004 |
| D14041 | Homo sapiens mRNA for H-2K binding factor-2, |
| | complete cds. February 1999 |
| NM_000246 | Homo sapiens class II, major histocompatibility |
| | complex, transactivator (CIITA), mRNA. November 2005 |
| NM_000295 | Homo sapiens serpin peptidase inhibitor, clade A |
| | (alpha-1 antiproteinase, antitrypsin), member 1 |
| | (SERPINA1), transcript variant 1, mRNA. November 2005 |
| NM_000544 | Homo sapiens transporter 2, ATP-binding cassette, |
| | sub-family B (MDR/TAP) (TAP2), transcript variant 1, |
| | mRNA. November 2005 |
| NM_000591 | Homo sapiens CD14 antigen (CD14), mRNA. |
| | November 2005 |
| NM_000593 | Homo sapiens transporter 1, ATP-binding cassette, |
| | sub-family B (MDR/TAP) (TAP1), mRNA. November 2005 |
| NM_000611 | Homo sapiens CD59 antigen p18-20 (antigen |
| | identified by monoclonal antibodies 16.3A5, EJ16, |
| | EJ30, EL32 and G344) (CD59), transcript variant 2, |
| | mRNA. October 2005 |
| NM_000634 | Homo sapiens interleukin 8 receptor, alpha (IL8RA), |
| | mRNA. November 2005 |
| NM_000660 | Homo sapiens transforming growth factor, beta 1 |
| | (Camurati-Engelmann disease) (TGFB1), mRNA. |
| | November 2005 |
| NM_001001887 | Homo sapiens interferon-induced protein with |
| | tetratricopeptide repeats 1 (IFIT1), transcript variant |
| | 1, mRNA. October 2005 |
| NM_001008540 | Homo sapiens chemokine (C—X—C motif) receptor 4 |
| | (CXCR4), transcript variant 1, mRNA. November 2005 |
| NM_001013255 | Homo sapiens lymphocyte-specific protein 1 (LSP1), |
| | transcript variant 4, mRNA. October 2005 |
| NM_001018076 | Homo sapiens nuclear receptor subfamily 3, group C, |
| | member 1 (glucocorticoid receptor) (NR3C1), |
| | transcript variant 4, mRNA. November 2005 |
| NM_001157 | Homo sapiens annexin A11 (ANXA11), transcript |
| | variant a, mRNA. October 2005 |
| NM_001175 | Homo sapiens Rho GDP dissociation inhibitor (GDI) |
| | beta (ARHGDIB), mRNA. October 2005 |
| NM_001421 | Homo sapiens E74-like factor 4 (ets domain |
| | transcription factor) (ELF4), mRNA. October 2005 |
| NM_001465 | Homo sapiens FYN binding protein (FYB-120/130) |
| | (FYB), transcript variant 1, mRNA. November 2005 |
| NM_001557 | Homo sapiens interleukin 8 receptor, beta (IL8RB), |
| | mRNA. November 2005 |
| NM_001629 | Homo sapiens arachidonate 5-lipoxygenase- |
| | activating protein (ALOX5AP), mRNA. November 2005 |
| NM_001637 | Homo sapiens acyloxyacyl hydrolase (neutrophil) |
| | (AOAH), mRNA. September 2005 |
| NM_001760 | Homo sapiens cyclin D3 (CCND3), mRNA. October 2005 |
| NM_001784 | Homo sapiens CD97 antigen (CD97), transcript |
| | variant 2, mRNA. October 2005 |
| NM_002032 | Homo sapiens ferritin, heavy polypeptide 1 (FTH1), |
| | mRNA. November 2005 |
| NM_002221 | Homo sapiens inositol 1,4,5-trisphosphate 3-kinase |
| | B (ITPKB), mRNA. October 2005 |
| NM_002468 | Homo sapiens myeloid differentiation primary |
| | response gene (88) (MYD88), mRNA. November 2005 |
| NM_002756 | Homo sapiens mitogen-activated protein kinase |
| | kinase 3 (MAP2K3), transcript variant A, mRNA. |
| | October 2005 |
| NM_002985 | Homo sapiens chemokine (C-C motif) ligand 5 |
| | (CCL5), mRNA. November 2005 |
| NM_003110 | Homo sapiens Sp2 transcription factor (SP2), mRNA. |
| | October 2005 |
| NM_003204 | Homo sapiens nuclear factor (erythroid-derived 2)- |
| | like 1 (NFE2L1), mRNA. October 2005 |
| NM_003407 | Homo sapiens zinc finger protein 36, C3H type, |
| | homolog (mouse) (ZFP36), mRNA. November 2005 |
| NM_003820 | Homo sapiens tumor necrosis factor receptor |
| | superfamily, member 14 (herpesvirus entry mediator) |
| | (TNFRSF14), mRNA. November 2005 |
| NM_003853 | Homo sapiens interleukin 18 receptor accessory |
| | protein (IL18RAP), mRNA. October 2005 |
| NM_004120 | Homo sapiens guanylate binding protein 2, |
| | interferon-inducible (GBP2), mRNA. September 2005 |
| NM_004356 | Homo sapiens CD81 antigen (target of |
| | antiproliferative antibody 1) (CD81), mRNA. October 2005 |
| NM_004688 | Homo sapiens N-myc (and STAT) interactor (NMI), |
| | mRNA. November 2005 |
| NM_005621 | Homo sapiens S100 calcium binding protein A12 |
| | (calgranulin C) (S100A12), mRNA. October 2005 |
| NM_005745 | Homo sapiens B-cell receptor-associated protein 31 |
| | (BCAP31), mRNA. October 2005 |
| NM_005849 | Homo sapiens immunoglobulin superfamily, member |
| | 6 (IGSF6), mRNA. October 2005 |
| NM_005902 | Homo sapiens SMAD, mothers against DPP homolog |
| | 3 (Drosophila) (SMAD3), mRNA. November 2005 |
| NM_006263 | Homo sapiens proteasome (prosome, macropain) |
| | activator subunit 1 (PA28 alpha) (PSME1), transcript |
| | variant 1, mRNA. October 2005 |
| NM_006665 | Homo sapiens heparanase (HPSE), mRNA. November 2005 |
| NM_012072 | Homo sapiens complement component 1, q |
| | subcomponent, receptor 1 (C1QR1), mRNA. October 2005 |
| NM_013237 | Homo sapiens px19-like protein (PX19), mRNA. |
| | April 2005 |
| NM_014146 | Homo sapiens linker for activation of T cells family, |
| | member 2 (LAT2), transcript variant 3, mRNA. |
| | October 2005 |
| NM_020980 | Homo sapiens aquaporin 9 (AQP9), mRNA. October 2005 |
| NM_021649 | Homo sapiens toll-like receptor adaptor molecule 2 |
| | (TICAM2), mRNA. October 2005 |
| NM_032924 | Homo sapiens zinc finger protein 3 (A8-51) (ZNF3), |
| | transcript variant 2, mRNA. September 2005 |
| NM_052942 | Homo sapiens guanylate binding protein 5 (GBP5), |
| | mRNA. October 2005 |
| NM_133437 | Homo sapiens titin (TTN), transcript variant novex-2, |
| | mRNA. November 2005 |
| NM_145640 | Homo sapiens apolipoprotein L, 3 (APOL3), transcript |
| | variant alpha/d, mRNA. October 2005 |
| NM_148919 | Homo sapiens proteasome (prosome, macropain) |
| | subunit, beta type, 8 (large multifunctional peptidase |
| | 7) (PSMB8), transcript variant 2, mRNA. October 2005 |
| NM_172373 | Homo sapiens E74-like factor 1 (ets domain |
| | transcription factor) (ELF1), mRNA. October 2005 |
| NM_002463 | Homo sapiens myxovirus (influenza virus) resistance |
| | 2 (mouse) (MX2), mRNA. September 2005 |
| NM_003264 | Homo sapiens toll-like receptor 2 (TLR2), mRNA. |
| | November 2005 |
| NM_004946 | Homo sapiens dedicator of cytokinesis 2 (DOCK2), |
| | mRNA. October 2005 |
| BC013629 | Homo sapiens WNK lysine deficient protein kinase 1, |
| | mRNA (cDNA clone IMAGE: 3445410), partial cds. |
| | January 2005 |
| NM_000733 | Homo sapiens CD3E antigen, epsilon polypeptide |
| | (TiT3 complex) (CD3E), mRNA. October 2005 |
| NM_001556 | Homo sapiens inhibitor of kappa light polypeptide |
| | gene enhancer in B-cells, kinase beta (IKBKB), |
| | mRNA. November 2005 |
| NM_001743 | Homo sapiens calmodulin 2 (phosphorylase kinase, |
| | delta) (CALM2), mRNA. October 2005 |
| NM_002122 | Homo sapiens major histocompatibility complex, class |
| | II, DQ alpha 1 (HLA-DQA1), mRNA. November 2005 |
| NM_002576 | Homo sapiens p21/Cdc42/Rac1-activated kinase 1 |
| | (STE20 homolog, yeast) (PAK1), mRNA. November 2005 |
| NM_002647 | Homo sapiens phosphoinositide-3-kinase, class 3 |
| | (PIK3C3), mRNA. October 2005 |
| NM_002745 | Homo sapiens mitogen-activated protein kinase 1 |
| | (MAPK1), transcript variant 1, mRNA. November 2005 |
| NM_002872 | Homo sapiens ras-related C3 botulinum toxin |
| | substrate 2 (rho family, small GTP binding protein |
| | Rac2) (RAC2), mRNA. October 2005 |
| NM_003998 | Homo sapiens nuclear factor of kappa light |
| | polypeptide gene enhancer in B-cells 1 (p105) |
| | (NFKB1), mRNA. November 2005 |
| NM_004048 | Homo sapiens beta-2-microglobulin (B2M), mRNA. |
| | November 2005 |
| NM_004383 | Homo sapiens c-src tyrosine kinase (CSK), mRNA. |
| | November 2005 |
| NM_005026 | Homo sapiens phosphoinositide-3-kinase, catalytic, |
| | delta polypeptide (PIK3CD), mRNA. November 2005 |
| NM_005252 | Homo sapiens v-fos FBJ murine osteosarcoma viral |
| | oncogene homolog (FOS), mRNA. November 2005 |
| NM_005428 | Homo sapiens vav 1 oncogene (VAV1), mRNA. |
| | November 2005 |
| NM_005565 | Homo sapiens lymphocyte cytosolic protein 2 (SH2 |
| | domain containing leukocyte protein of 76 kDa) |
| | (LCP2), mRNA. October 2005 |
| NM_006139 | Homo sapiens CD28 antigen (Tp44) (CD28), mRNA. |
| | November 2005 |
| NM_006257 | Homo sapiens protein kinase C, theta (PRKCQ), |
| | mRNA. November 2005 |
| NM_019111 | Homo sapiens major histocompatibility complex, class |
| | II, DR alpha (HLA-DRA), mRNA. October 2005 |
| NM_020529 | Homo sapiens nuclear factor of kappa light |
| | polypeptide gene enhancer in B-cells inhibitor, alpha |
| | (NFKBIA), mRNA. October 2005 |
| NM_033554 | Homo sapiens major histocompatibility complex, class |
| | II, DP alpha 1 (HLA-DPA1), mRNA. October 2005 |
| NM_182811 | Homo sapiens phospholipase C, gamma 1 (PLCG1), |
| | transcript variant 2, mRNA. October 2005 |
| AB209870 | Homo sapiens mRNA for HLA class I |
| | histocompatibility antigen, E alpha chain precursor |
| | variant protein. March 2005 |
| AF110908 | Homo sapiens TNF-receptor associated factor-3 |
| | (TRAF-3) mRNA, partial cds; and 3′UTR. May 1999 |
| BC025727 | Homo sapiens T cell receptor alpha variable 20, |
| | mRNA (cDNA clone MGC: 34712 IMAGE: 5201547), |
| | complete cds. June 2005 |
| BC028068 | Homo sapiens Janus kinase 3 (a protein tyrosine |
| | kinase, leukocyte), mRNA (cDNA clone MGC: 39993 |
| | IMAGE: 5212575), complete cds. October 2003 |
| K02885 | Homo sapiens T-cell receptor active beta-chain V- |
| | D-J-beta-1.2-C-beta-1 (TCRB) mRNA, partial cds. |
| | March 1999 |
| L34703 | Homo sapiens T-cell receptor alpha chain (TCRA) |
| | mRNA (HLA-A1, 24; B7, 8; DR 1, 3), complete cds. |
| | December 2001 |
| M12886 | Human T-cell receptor active beta-chain mRNA, |
| | complete cds. January 1995 |
| NM_000416 | Homo sapiens interferon gamma receptor 1 |
| | (IFNGR1), mRNA. October 2005 |
| NM_000433 | Homo sapiens neutrophil cytosolic factor 2 (65 kDa, |
| | chronic granulomatous disease, autosomal 2) |
| | (NCF2), mRNA. October 2005 |
| NM_001010972 | Homo sapiens zyxin (ZYX), transcript variant 2, |
| | mRNA. October 2005 |
| NM_001012631 | Homo sapiens interleukin 32 (IL32), transcript variant |
| | 1, mRNA. October 2005 |
| NM_001558 | Homo sapiens interleukin 10 receptor, alpha |
| | (IL10RA), mRNA. October 2005 |
| NM_002227 | Homo sapiens Janus kinase 1 (a protein tyrosine |
| | kinase) (JAK1), mRNA. November 2005 |
| NM_002298 | Homo sapiens lymphocyte cytosolic protein 1 (L- |
| | plastin) (LCP1), mRNA. October 2005 |
| NM_002923 | Homo sapiens regulator of G-protein signalling 2, |
| | 24 kDa (RGS2), mRNA. November 2005 |
| NM_003877 | Homo sapiens suppressor of cytokine signaling 2 |
| | (SOCS2), mRNA. October 2005 |
| NM_004117 | Homo sapiens FK506 binding protein 5 (FKBP5), |
| | mRNA. October 2005 |
| NM_004489 | Homo sapiens G protein pathway suppressor 2 |
| | (GPS2), mRNA. October 2005 |
| NM_006058 | Homo sapiens TNFAIP3 interacting protein 1 (TNIP1), |
| | mRNA. October 2005 |
| NM_006449 | Homo sapiens CDC42 effector protein (Rho GTPase |
| | binding) 3 (CDC42EP3), mRNA. September 2005 |
| NM_006726 | Homo sapiens LPS-responsive vesicle trafficking, |
| | beach and anchor containing (LRBA), mRNA. |
| | October 2005 |
| NM_006779 | Homo sapiens CDC42 effector protein (Rho GTPase |
| | binding) 2 (CDC42EP2), mRNA. September 2005 |
| NM_014663 | Homo sapiens jumonji domain containing 2A |
| | (JMJD2A), mRNA. November 2005 |
| NM_015015 | Homo sapiens jumonji domain containing 2B |
| | (JMJD2B), mRNA. October 2005 |
| NM_054014 | Homo sapiens FK506 binding protein 1A, 12 kDa |
| | (FKBP1A), transcript variant 12A, mRNA. November 2005 |
| NM_172313 | Homo sapiens colony stimulating factor 3 receptor |
| | (granulocyte) (CSF3R), transcript variant 4, mRNA. |
| | October 2005 |
| NM_203346 | Homo sapiens high density lipoprotein binding protein |
| | (vigilin) (HDLBP), mRNA. October 2005 |

TABLE 6
List of genes/transcripts associated with immunity signalling pathways

| Reference | | |
|---|---|---|
| Enter | Representative | |
| Gene | transcript | Name of gene |
| 1 | NM_130786 | alpha-1-B glycoprotein; A1BG |
| 12 | NM_001085 | Alpha-1-antichymotrypsin precursor (ACT) [Contains: |
| | | Alpha-1-antichymotrypsin His-Pro-less]. |
| | | [Source: Uniprot/SWISSPROT; Acc: P01011] |
| 23 | NM_001025091 | ATP-binding cassette sub-family F member 1 (ATP-binding |
| | | cassette 50) (TNF-alpha-stimulated ABC protein). |
| | | [Source: Uniprot/SWISSPROT; Acc: Q8NE71] |
| 100 | NM_000022 | Adenosine deaminase (EC 3.5.4.4) (Adenosine |
| | | aminohydrolase). [Source: Uniprot/SWISSPROT; Acc: P00813] |
| 103 | NM_001111 | Double-stranded RNA-specific adenosine deaminase (EC |
| | | 3.5.4.—) (DRADA) (136 kDa double-stranded RNA binding |
| | | protein) (P136) (K88DSRBP) (Interferon-inducible protein 4) |
| | | (IFI-4 protein). [Source: Uniprot/SWISSPROT; Acc: P55265] |
| 134 | NM_000674 | Adenosine A1 receptor. |
| | | [Source: Uniprot/SWISSPROT; Acc: P30542] |
| 135 | NM_000675 | Adenosine A2a receptor. |
| | | [Source: Uniprot/SWISSPROT; Acc: P29274] |
| 136 | NM_000676 | Adenosine A2b receptor. |
| | | [Source: Uniprot/SWISSPROT; Acc: P29275] |
| 140 | NM_020683; NM_000677 | adenosine A3 receptor; ADORA3 |
| 143 | NM_006437 | Poly [ADP-ribose] polymerase 4 (EC 2.4.2.30) (PARP-4) |
| | | (Vault poly(ADP-ribose) polymerase) (VPARP) (193-kDa |
| | | vault protein) (PARP-related/IalphaI-related H5/proline- |
| | | rich) (PH5P). [Source: Uniprot/SWISSPROT; Acc: Q9UKK3] |
| 174 | NM_001134 | Alpha-fetoprotein precursor (Alpha-fetoglobulin) (Alpha-1- |
| | | fetoprotein). [Source: Uniprot/SWISSPROT; Acc: P02771] |
| 177 | NM_172197; NM_001136 | advanced glycosylation end product-specific receptor; AGER |
| 182 | NM_000214 | jagged 1 (Alagille syndrome); JAG1 |
| 197 | NM_001622 | Alpha-2-HS-glycoprotein precursor (Fetuin-A) (Alpha-2-Z- |
| | | globulin) (Ba-alpha-2-glycoprotein) [Contains: Alpha-2- |
| | | HS-glycoprotein chain A; Alpha-2-HS-glycoprotein chain B]. |
| | | [Source: Uniprot/SWISSPROT; Acc: P02765] |
| 199 | NM_001623 | Allograft inflammatory factor 1 (AIF-1) (Ionized calcium- |
| | | binding adapter molecule 1) (G1 protein). |
| | | [Source: Uniprot/SWISSPROT; Acc: P55008] |
| 207 | NM_001014431; NM_001014432; | v-akt murine thymoma viral oncogene homolog 1; AKT1 |
| | NM_005163 | |
| 208 | NM_001626 | v-akt murine thymoma viral oncogene homolog 2; AKT2 |
| 214 | NM_001627 | CD166 antigen precursor (Activated leukocyte-cell adhesion |
| | | molecule) (ALCAM). |
| | | [Source: Uniprot/SWISSPROT; Acc: Q13740] |
| 240 | NM_000698 | Arachidonate 5-lipoxygenase (EC 1.13.11.34) (5- |
| | | lipoxygenase) (5-LO). |
| | | [Source: Uniprot/SWISSPROT; Acc: P09917] |
| 241 | NM_001629 | Arachidonate 5-lipoxygenase-activating protein (FLAP) |
| | | (MK-886-binding protein). |
| | | [Source: Uniprot/SWISSPROT; Acc: P20292] |
| 246 | NM_001140 | Arachidonate 15-lipoxygenase (EC 1.13.11.33) |
| | | (Arachidonate omega-6 lipoxygenase) (15-LOX). |
| | | [Source: Uniprot/SWISSPROT; Acc: P16050] |
| 259 | NM_001633 | AMBP protein precursor [Contains: Alpha-1-microglobulin |
| | | (Protein HC) (Complex-forming glycoprotein heterogeneous |
| | | in charge) (Alpha-1 microglycoprotein); Inter-alpha-trypsin |
| | | inhibitor light chain (ITI-LC) (Bikunin) (HI-30)]. |
| | | [Source: Uniprot/SWISSPROT; Acc: P |
| 283 | NM_001145 | Ribonuclease 4 precursor (EC 3.1.27.—) (RNase 4). |
| | | [Source: Uniprot/SWISSPROT; Acc: P34096] |
| 301 | NM_000700 | Annexin A1 (Annexin I) (Lipocortin I) (Calpactin II) |
| | | (Chromobindin-9) (p35) (Phospholipase A2 inhibitory |
| | | protein). [Source: Uniprot/SWISSPROT; Acc: P04083] |
| 311 | NM_145869 | Annexin A11 (Annexin XI) (Calcyclin-associated annexin 50) |
| | | (CAP-50) (56 kDa autoantigen). |
| | | [Source: Uniprot/SWISSPROT; Acc: P50995] |
| 313 | NM_001637 | Acyloxyacyl hydrolase precursor (EC 3.1.1.77) [Contains: |
| | | Acyloxyacyl hydrolase small subunit; Acyloxyacyl hydrolase |
| | | large subunit]. [Source: Uniprot/SWISSPROT; Acc: P28039] |
| 316 | NM_001159 | Aldehyde oxidase (EC 1.2.3.1). |
| | | [Source: Uniprot/SWISSPROT; Acc: Q06278] |
| 325 | NM_001639 | amyloid P component, serum; APCS |
| 326 | NM_000383 | Autoimmune regulator (Autoimmune polyendocrinopathy |
| | | candidiasis ectodermal dystrophy protein) (APECED protein). |
| | | [Source: Uniprot/SWISSPROT; Acc: O43918] |
| 336 | NM_001643 | Apolipoprotein A-II precursor (Apo-AII) (ApoA-II) |
| | | [Contains: Apolipoprotein A-II(1-76)]. |
| | | [Source: Uniprot/SWISSPROT; Acc: P02652] |
| 355 | NM_000043 | Tumor necrosis factor receptor superfamily member 6 |
| | | precursor (FASL receptor) (Apoptosis-mediating surface |
| | | antigen FAS) (Apo-1 antigen) (CD95 antigen). |
| | | [Source: Uniprot/SWISSPROT; Acc: P25445] |
| 356 | NM_000639 | Tumor necrosis factor ligand superfamily member 6 (FAS |
| | | antigen ligand) (Apoptosis antigen ligand) (APTL) (CD178 |
| | | antigen) [Contains: Tumor necrosis factor ligand superfamily |
| | | member 6, membrane form; Tumor necrosis factor ligand |
| | | superfamily member 6, solubl |
| 366 | NM_020980 | Aquaporin-9 (AQP-9) (Small solute channel 1). |
| | | [Source: Uniprot/SWISSPROT; Acc: O43315] |
| 369 | NM_001654 | v-raf murine sarcoma 3611 viral oncogene homolog; ARAF |
| 397 | NM_001175 | Rho GDP-dissociation inhibitor 2 (Rho GDI 2) (Rho-GDI |
| | | beta) (Ly-GDI). [Source: Uniprot/SWISSPROT; Acc: P52566] |
| 399 | NM_004310 | Rho-related GTP-binding protein RhoH (GTP-binding |
| | | protein TTF). [Source: Uniprot/SWISSPROT; Acc: Q15669] |
| 563 | NM_001185 | alpha-2-glycoprotein 1, zinc; AZGP1 |
| 566 | NM_001700 | azurocidin 1 (cationic antimicrobial protein 37); AZU1 |
| 567 | NM_004048 | beta-2-microglobulin; B2M |
| 572 | NM_032989 | Bcl2-antagonist of cell death (BAD) (Bcl-2 binding |
| | | component 6) (Bcl-XL/Bcl-2-associated death promoter) |
| | | (Bcl-2-like 8 protein). |
| | | [Source: Uniprot/SWISSPROT; Acc: Q92934] |
| 596 | NM_000657 | Apoptosis regulator Bcl-2. |
| | | [Source: Uniprot/SWISSPROT; Acc: P10415] |
| 604 | NM_001706 | B-cell lymphoma 6 protein (BCL-6) (Zinc finger protein 51) |
| | | (LAZ-3 protein) (BCL-5) (Zinc finger and BTB domain- |
| | | containing protein 27). |
| | | [Source: Uniprot/SWISSPROT; Acc: P41182] |
| 608 | NM_001192 | Tumor necrosis factor receptor superfamily member 17 (B- |
| | | cell maturation protein) (CD269 antigen). |
| | | [Source: Uniprot/SWISSPROT; Acc: Q02223] |
| 623 | NM_000710 | B1 bradykinin receptor (BK-1 receptor) (B1R). |
| | | [Source: Uniprot/SWISSPROT; Acc: P46663] |
| 624 | NM_000623 | B2 bradykinin receptor (BK-2 receptor) (B2R). |
| | | [Source: Uniprot/SWISSPROT; Acc: P30411] |
| 629 | NM_001710 | B-factor, properdin; BF |
| 640 | NM_001715 | B lymphoid tyrosine kinase; BLK |
| 641 | NM_000057 | Bloom's syndrome protein (EC 3.6.1.—) (RecQ protein-like 3) |
| | | (DNA helicase, RecQ-like type 2). |
| | | [Source: Uniprot/SWISSPROT; Acc: P54132] |
| 643 | NM_032966 | C—X—C chemokine receptor type 5 (CXC-R5) (CXCR-5) |
| | | (Burkitt'S lymphoma receptor 1) (Monocyte-derived receptor |
| | | 15) (MDR15) (CD185 antigen). |
| | | [Source: Uniprot/SWISSPROT; Acc: P32302] |
| 648 | NM_005180 | Polycomb group RING finger protein 4 (Polycomb complex |
| | | protein BMI-1) (RING finger protein 51). |
| | | [Source: Uniprot/SWISSPROT; Acc: P35226] |
| 660 | NM_203281; NM_001721 | BMX non-receptor tyrosine kinase; BMX |
| 671 | NM_001725 | bactericidal/permeability-increasing protein; BPI |
| 673 | NM_004333 | v-raf murine sarcoma viral oncogene homolog B1; BRAF |
| 683 | NM_004334 | ADP-ribosyl cyclase 2 precursor (EC 3.2.2.5) (Cyclic ADP- |
| | | ribose hydrolase 2) (cADPr hydrolase 2) (Bone marrow |
| | | stromal antigen 1) (BST-1) (CD157 antigen). |
| | | [Source: Uniprot/SWISSPROT; Acc: Q10588] |
| 684 | NM_004335 | Bone marrow stromal antigen 2 (BST-2) (CD317 antigen). |
| | | [Source: Uniprot/SWISSPROT; Acc: Q10589] |
| 695 | NM_000061 | Bruton agammaglobulinemia tyrosine kinase; BTK |
| 708 | NM_001212 | complement component 1, q subcomponent binding |
| | | protein; C1QBP |
| 710 | NM_000062 | Plasma protease C1 inhibitor precursor (C1 Inh) (C1Inh). |
| | | [Source: Uniprot/SWISSPROT; Acc: P05155] |
| 712 | NM_015991 | complement component 1, q subcomponent, alpha |
| | | polypeptide; C1QA |
| 713 | NM_000491 | complement component 1, q subcomponent, beta |
| | | polypeptide; C1QB |
| 714 | NM_172369 | complement component 1, q subcomponent, gamma |
| | | polypeptide; C1QG |
| 715 | NM_001733 | complement component 1, r subcomponent; C1R |
| 716 | NM_0017342; NM_201442 | complement component 1, s subcomponent; C1S |
| 717 | NM_000063 | complement component 2; C2 |
| 718 | NM_000064 | complement component 3; C3 |
| 719 | NM_004054 | C3a anaphylatoxin chemotactic receptor (C3a-R) (C3AR). |
| | | [Source: Uniprot/SWISSPROT; Acc: Q16581] |
| 720 | NM_007293 | complement component 4A; C4A |
| 721 | NM_000592 | complement component 4B; C4B |
| 722 | NM_000715 | complement component 4 binding protein, alpha; C4BPA |
| 725 | NM_001017364; NM_001017366; | complement component 4 binding protein, beta; C4BPB |
| | NM_001017365; | |
| | NM_000716; NM_001017367 | |
| 727 | NM_001735 | complement component 5; C5 |
| 728 | NM_001736 | C5a anaphylatoxin chemotactic receptor (C5a-R) (C5aR) |
| | | (CD88 antigen). [Source: Uniprot/SWISSPROT; Acc: P21730] |
| 729 | NM_000065 | complement component 6; C6 |
| 730 | NM_000587 | complement component 7; C7 |
| 731 | NM_000562 | complement component 8, alpha polypeptide; C8A |
| 732 | NM_000066 | complement component 8, beta polypeptide; C8B |
| 733 | NM_000606 | Complement component C8 gamma chain precursor. |
| | | [Source: Uniprot/SWISSPROT; Acc: P07360] |
| 735 | NM_001737 | complement component 9; C9 |
| 796 | NM_001741 | Calcitonin precursor [Contains: Calcitonin; Katacalcin |
| | | (Calcitonin carboxyl-terminal peptide) (CCP) (PDN-21)]. |
| | | [Source: Uniprot/SWISSPROT; Acc: P01258] |
| 801 | NM_006888 | calmodulin 1 (phosphorylase kinase, delta); CALM1 |
| 805 | NM_001743 | calmodulin 2 (phosphorylase kinase, delta); CALM2 |
| 808 | NM_005184 | calmodulin 3 (phosphorylase kinase, delta); CALM3 |
| 810 | NM_005185 | calmodulin-like 3; CALML3 |
| 820 | NM_004345 | cathelicidin antimicrobial peptide; CAMP |
| 836 | NM_004346 | Caspase-3 precursor (EC 3.4.22.—) (CASP-3) (Apopain) |
| | | (Cysteine protease CPP32) (Yama protein) (CPP-32) (SREBP |
| | | cleavage activity 1) (SCA-1) [Contains: Caspase-3 p17 |
| | | subunit; Caspase-3 p12 subunit]. |
| | | [Source: Uniprot/SWISSPROT; Acc: P42574] |
| 865 | NM_001755 | Core-binding factor, beta subunit (CBF-beta) (Polyomavirus |
| | | enhancer binding protein 2 beta subunit) (PEBP2-beta) |
| | | (PEA2-beta) (SL3-3 enhancer factor 1 beta subunit) |
| | | (SL3/AKV core-binding factor beta subunit). |
| | | [Source: Uniprot/SWISSPROT; Acc: Q13951] |
| 896 | NM_001760 | G1/S-specific cyclin-D3. |
| | | [Source: Uniprot/SWISSPROT; Acc: P30281] |
| 909 | NM_001763 | CD1a antigen; CD1A |
| 910 | NM_001764 | CD1b antigen; CD1B |
| 911 | NM_001765 | CD1C antigen, c polypeptide; CD1C |
| 912 | NM_001766 | CD1D antigen, d polypeptide; CD1D |
| 913 | NM_030893 | CD1E antigen, e polypeptide; CD1E |
| 914 | NM_001767 | CD2 antigen (p50), sheep red blood cell receptor; CD2 |
| 915 | NM_000732 | CD3D antigen, delta polypeptide (TiT3 complex); CD3D |
| 916 | NM_000733 | CD3E antigen, epsilon polypeptide (TiT3 complex); CD3E |
| 917 | NM_000073 | CD3G antigen, gamma polypeptide (TiT3 complex); CD3G |
| 919 | NM_1980534; NM_000734 | CD3Z antigen, zeta polypeptide (TiT3 complex); CD3Z |
| 920 | NM_000616 | CD4 antigen (p55); CD4 |
| 922 | NM_005894 | CD5 antigen-like precursor (SP-alpha) (CT-2) (IgM- |
| | | associated peptide). |
| | | [Source: Uniprot/SWISSPROT; Acc: O43866] |
| 923 | NM_006725 | T-cell differentiation antigen CD6 precursor (T12) (TP120). |
| | | [Source: Uniprot/SWISSPROT; Acc: P30203] |
| 924 | NM_006137 | CD7 antigen (p41); CD7 |
| 925 | NM_001768; NM_171827 | CD8 antigen, alpha polypeptide (p32); CD8A |
| 926 | NM_172101; NM_172213; | CD8 antigen, beta polypeptide 1 (p37); CD8B1 |
| | NM_172099; NM_004931; | |
| | NM_172102 | |
| 929 | NM_000591 | Monocyte differentiation antigen CD14 precursor (Myeloid |
| | | cell-specific leucine-rich glycoprotein) [Contains: Monocyte |
| | | differentiation antigen CD14, urinary form; Monocyte |
| | | differentiation antigen CD14, membrane-bound form]. |
| | | [Source: Uniprot/SWISSPROT; Acc: P |
| 930 | NM_001770 | CD19 antigen; CD19 |
| 931 | NM_152866 | B-lymphocyte antigen CD20 (B-lymphocyte surface antigen |
| | | B1) (Leu-16) (Bp35). |
| | | [Source: Uniprot/SWISSPROT; Acc: P11836] |
| 933 | NM_001771 | CD22 antigen; CD22 |
| 934 | NM_013230 | Signal transducer CD24 precursor. |
| | | [Source: Uniprot/SWISSPROT; Acc: P25063] |
| 939 | NM_001242 | Tumor necrosis factor receptor superfamily member 7 |
| | | precursor (CD27L receptor) (T-cell activation antigen CD27) |
| | | (T14). [Source: Uniprot/SWISSPROT; Acc: P26842] |
| 940 | NM_006139 | CD28 antigen (Tp44); CD28 |
| 941 | NM_005191 | CD80 antigen (CD28 antigen ligand 1, B7-1 antigen); CD80 |
| 942 | NM_006889; NM_175862 | CD86 antigen (CD28 antigen ligand 2, B7-2 antigen); CD86 |
| 944 | NM_001244 | Tumor necrosis factor ligand superfamily member 8 (CD30 |
| | | ligand) (CD30-L) (CD153 antigen). |
| | | [Source: Uniprot/SWISSPROT; Acc: P32971] |
| 945 | NM_001772 | CD33 antigen (gp67); CD33 |
| 946 | NM_198845; NM_001245; | sialic acid binding Ig-like lectin 6; SIGLEC6 |
| | NM_198846 | |
| 953 | NM_001776 | Ectonucleoside triphosphate diphosphohydrolase 1 (EC |
| | | 3.6.1.5) (NTPDase1) (Ecto-ATP diphosphohydrolase) |
| | | (ATPDase) (Lymphoid cell activation antigen) (Ecto-apyrase) |
| | | (CD39 antigen). [Source: Uniprot/SWISSPROT; Acc: P49961] |
| 958 | NM_001250 | Tumor necrosis factor receptor superfamily member 5 |
| | | precursor (CD40L receptor) (B-cell surface antigen CD40) |
| | | (CDw40) (Bp50). [Source: Uniprot/SWISSPROT; Acc: P25942] |
| 959 | NM_000074 | Tumor necrosis factor ligand superfamily member 5 (CD40 |
| | | ligand) (CD40-L) (TNF-related activation protein) (TRAP) |
| | | (T cell antigen Gp39) (CD154 antigen) [Contains: Tumor |
| | | necrosis factor ligand superfamily member 5, membrane |
| | | form; Tumor necrosis factor liga |
| 961 | NM_001777; NM_001025080; | CD47 antigen (Rh-related antigen, integrin-associated |
| | NM_001025079; NM_198793 | signal transducer); CD47 |
| 962 | NM_001778 | CD48 antigen (B-cell membrane protein); CD48 |
| 963 | NM_000560 | Leukocyte surface antigen CD53 (Cell surface glycoprotein |
| | | CD53) (Tetraspanin-25) (Tspan-25). |
| | | [Source: Uniprot/SWISSPROT; Acc: P19397] |
| 965 | NM_001779 | Lymphocyte function-associated antigen 3 precursor (Ag3) |
| | | (Antigen CD58) (Surface glycoprotein LFA-3). |
| | | [Source: Uniprot/SWISSPROT; Acc: P19256] |
| 966 | NM_203330 | CD59 glycoprotein precursor (Membrane attack complex |
| | | inhibition factor) (MACIF) (MAC-inhibitory protein) (MAC-IP) |
| | | (Protectin) (MEM43 antigen) (Membrane inhibitor of reactive |
| | | lysis) (MIRL) (20 kDa homologous restriction factor) (HRF- |
| | | 20) (HRF20) (1F5 antige |
| 969 | NM_001781 | CD69 antigen (p60, early T-cell activation antigen); CD69 |
| 970 | NM_001252 | Tumor necrosis factor ligand superfamily member 7 (CD27 |
| | | ligand) (CD27-L) (CD70 antigen). |
| | | [Source: Uniprot/SWISSPROT; Acc: P32970] |
| 971 | NM_001782 | B-cell differentiation antigen CD72 (Lyb-2). |
| | | [Source: Uniprot/SWISSPROT; Acc: P21854] |
| 972 | NM_001025159; NM_004355; | CD74 antigen (invariant polypeptide of major |
| | NM_001025158 | histocompatibility complex, class II antigen- |
| | | associated); CD74 |
| 973 | NM_001783; NM_021601 | CD79A antigen (immunoglobulin-associated alpha); CD79A |
| 974 | NM_000626; NM_021602 | CD79B antigen (immunoglobulin-associated beta); CD79B |
| 975 | NM_004356 | CD81 antigen (26 kDa cell surface protein TAPA-1) (Target |
| | | of the antiproliferative antibody 1) (Tetraspanin-28) |
| | | (Tspan-28). [Source: Uniprot/SWISSPROT; Acc: P60033] |
| 976 | NM_078481 | CD97 antigen precursor (Leukocyte antigen CD97). |
| | | [Source: Uniprot/SWISSPROT; Acc: P48960] |
| 987 | NM_006726 | LPS-responsive vesicle trafficking, beach and anchor |
| | | containing (LRBA) |
| 998 | NM_001791; NM_044472 | cell division cycle 42 (GTP binding protein, 25 kDa); CDC42 |
| 1026 | NM_078467 | Cyclin-dependent kinase inhibitor 1 (p21) (CDK-interacting |
| | | protein 1) (Melanoma differentiation-associated protein 6) |
| | | (MDA-6). [Source: Uniprot/SWISSPROT; Acc: P38936] |
| 1051 | NM_005194 | CCAAT/enhancer binding protein beta (C/EBP beta) (Nuclear |
| | | factor NF-IL6) (Transcription factor 5). |
| | | [Source: Uniprot/SWISSPROT; Acc: P17676] |
| 1053 | NM_001805 | CCAAT/enhancer binding protein epsilon (C/EBP epsilon). |
| | | [Source: Uniprot/SWISSPROT; Acc: Q15744] |
| 1075 | NM_001814 | Dipeptidyl-peptidase I precursor (EC 3.4.14.1) (DPP-I) |
| | | (DPPI) (Cathepsin C) (Cathepsin J) (Dipeptidyl transferase) |
| | | [Contains: Dipeptidyl-peptidase I exclusion domain chain; |
| | | Dipeptidyl-peptidase I heavy chain; Dipeptidyl-peptidase I |
| | | light chain]. [Source: U |
| 1088 | NM_001816 | Carcinoembryonic antigen-related cell adhesion molecule 8 |
| | | precursor (Carcinoembryonic antigen CGM6) (Nonspecific |
| | | cross-reacting antigen NCA-95) (Antigen CD67) (CD66b |
| | | antigen). [Source: Uniprot/SWISSPROT; Acc: P31997] |
| 1118 | NM_003465 | Chitotriosidase-1 precursor (EC 3.2.1.14) (Chitinase-1). |
| | | [Source: Uniprot/SWISSPROT; Acc: Q13231] |
| 1130 | NM_001005736 | Lysosomal trafficking regulator (Beige homolog). |
| | | [Source: Uniprot/SWISSPROT; Acc: Q99698] |
| 1147 | NM_001278 | conserved helix-loop-helix ubiquitous kinase; CHUK |
| 1178 | NM_001828 | Eosinophil lysophospholipase (EC 3.1.1.5) (Charcot-Leyden |
| | | crystal protein) (Lysolecithin acylhydrolase) (CLC) (Galactin- |
| | | 10). [Source: Uniprot/SWISSPROT; Acc: Q05315] |
| 1191 | NM_203339 | Clusterin precursor (Complement-associated protein SP- |
| | | 40,40) (Complement cytolysis inhibitor) (CLI) (NA1/NA2) |
| | | (Apolipoprotein J) (Apo-J) (Testosterone-repressed prostate |
| | | message 2) (TRPM-2) [Contains: Clusterin beta chain |
| | | (ApoJalpha) (Complement cytolysis |
| 1230 | NM_001295 | C-C chemokine receptor type 1 (C-C CKR-1) (CC-CKR-1) |
| | | (CCR-1) (CCR1) (Macrophage inflammatory protein 1-alpha |
| | | receptor) (MIP-1alpha-R) (RANTES-R) (HM145) (LD78 |
| | | receptor) (CD191 antigen). |
| | | [Source: Uniprot/SWISSPROT; Acc: P32246] |
| 1231 | NM_000647 | C-C chemokine receptor type 2 (C-C CKR-2) (CC-CKR-2) |
| | | (CCR-2) (CCR2) (Monocyte chemoattractant protein 1 |
| | | receptor) (MCP-1-R) (CD192 antigen). |
| | | [Source: Uniprot/SWISSPROT; Acc: P41597] |
| 1232 | NM_001837; NM_178329 | C-C chemokine receptor type 3 (C-C CKR-3) (CC-CKR-3) |
| | | (CCR-3) (CCR3) (CKR3) (Eosinophil eotaxin receptor) |
| | | (CD193 antigen). [Source: Uniprot/SWISSPROT; Acc: P51677] |
| 1233 | NM_005508 | C-C chemokine receptor type 4 (C-C CKR-4) (CC-CKR-4) |
| | | (CCR-4) (CCR4) (K5-5). |
| | | [Source: Uniprot/SWISSPROT; Acc: P51679] |
| 1234 | NM_000579 | C-C chemokine receptor type 5 (C-C CKR-5) (CC-CKR-5) |
| | | (CCR-5) (CCR5) (HIV-1 fusion coreceptor) (CHEMR13) |
| | | (CD195 antigen). [Source: Uniprot/SWISSPROT; Acc: P51681] |
| 1235 | NM_031409; NM_004367 | C-C chemokine receptor type 6 (C-C CKR-6) (CC-CKR-6) |
| | | (CCR-6) (LARC receptor) (GPR-CY4) (GPRCY4) (Chemokine |
| | | receptor-like 3) (CKR-L3) (DRY6) (G-protein coupled |
| | | receptor 29) (Antigen CD196). |
| | | [Source: Uniprot/SWISSPROT; Acc: P51684] |
| 1236 | NM_001838 | C-C chemokine receptor type 7 precursor (C-C CKR-7) (CC- |
| | | CKR-7) (CCR-7) (MIP-3 beta receptor) (EBV-induced G- |
| | | protein coupled receptor 1) (EBI1) (BLR2) (CD197 antigen) |
| | | (CDw197). [Source: Uniprot/SWISSPROT; Acc: P32248] |
| 1237 | NM_005201 | C-C chemokine receptor type 8 (C-C CKR-8) (CC-CKR-8) |
| | | (CCR-8) (GPR-CY6) (GPRCY6) (Chemokine receptor-like 1) |
| | | (CKR-L1) (TER1) (CMKBRL2) (CC-chemokine receptor |
| | | CHEMR1) (CDw198 antigen). |
| | | [Source: Uniprot/SWISSPROT; Acc: P51685] |
| 1238 | NM_001296 | Chemokine-binding protein 2 (Chemokine-binding protein |
| | | D6) (C-C chemokine receptor D6) (Chemokine receptor |
| | | CCR-9) (Chemokine receptor CCR-10). |
| | | [Source: Uniprot/SWISSPROT; Acc: O00590] |
| 1240 | NM_004072 | Chemokine receptor-like 1 (G-protein coupled receptor |
| | | DEZ) (G-protein coupled receptor ChemR23). |
| | | [Source: Uniprot/SWISSPROT; Acc: Q99788] |
| 1241 | NM_181657 | Leukotriene B4 receptor 1 (LTB4-R 1) (P2Y purinoceptor 7) |
| | | (P2Y7) (Chemoattractant receptor-like 1) (G-protein |
| | | coupled receptor 16). |
| | | [Source: Uniprot/SWISSPROT; Acc: Q15722] |
| 1269 | NM_001841 | Cannabinoid receptor 2 (CB2) (CB-2) (CX5). |
| | | [Source: Uniprot/SWISSPROT; Acc: P34972] |
| 1316 | NM_001008490 | Core promoter element-binding protein (Kruppel-like factor |
| | | 6) (B-cell derived protein 1) (Proto-oncogene BCD1) |
| | | (Transcription factor Zf9) (GC-rich sites binding factor GBF). |
| | | [Source: Uniprot/SWISSPROT; Acc: Q99612] |
| 1378 | NM_000651; NM_000573 | complement component (3b/4b) receptor 1, including Knops |
| | | blood group system; CR1 |
| 1379 | XM_114735 | complement component (3b/4b) receptor 1-like; CR1L |
| 1380 | NM_001006658; NM_001877 | complement component (3d/Epstein Barr virus) receptor |
| | | 2; CR2 |
| 1392 | NM_000756 | Corticoliberin precursor (Corticotropin-releasing factor) |
| | | (CRF) (Corticotropin-releasing hormone). |
| | | [Source: Uniprot/SWISSPROT; Acc: P06850] |
| 1396 | NM_001311 | Cysteine-rich protein 1 (Cysteine-rich intestinal protein) |
| | | (CRIP) (Cysteine-rich heart protein) (hCRHP). |
| | | [Source: Uniprot/SWISSPROT; Acc: P50238] |
| 1401 | NM_000567 | C-reactive protein, pentraxin-related; CRP |
| 1432 | NM_139013; NM_001315; | mitogen-activated protein kinase 14; MAPK14 |
| | NM_139012; NM_139014 | |
| 1436 | NM_005211 | Macrophage colony-stimulating factor 1 receptor precursor |
| | | (CSF-1-R) (EC 2.7.1.112) (Fms proto-oncogene) (c-fms) |
| | | (CD115 antigen). [Source: Uniprot/SWISSPROT; Acc: P07333] |
| 1437 | NM_000758 | Granulocyte-macrophage colony-stimulating factor |
| | | precursor (GM-CSF) (Colony-stimulating factor) (CSF) |
| | | (Sargramostim) (Molgramostin). |
| | | [Source: Uniprot/SWISSPROT; Acc: P04141] |
| 1439 | NM_000395 | Cytokine receptor common beta chain precursor (GM- |
| | | CSF/IL-3/IL-5 receptor common beta-chain) (CD131 |
| | | antigen) (CDw131). |
| | | [Source: Uniprot/SWISSPROT; Acc: P32927] |
| 1440 | NM_000759 | Granulocyte colony-stimulating factor precursor (G-CSF) |
| | | (Pluripoietin) (Filgrastim) (Lenograstim). |
| | | [Source: Uniprot/SWISSPROT; Acc: P09919] |
| 1441 | NM_156039 | Granulocyte colony-stimulating factor receptor precursor |
| | | (G-CSF-R) (CD114 antigen). |
| | | [Source: Uniprot/SWISSPROT; Acc: Q99062] |
| 1445 | NM_004383 | c-src tyrosine kinase; CSK |
| 1493 | NM_005214 | Cytotoxic T-lymphocyte protein 4 precursor (Cytotoxic T- |
| | | lymphocyte-associated antigen 4) (CTLA-4) (CD152 |
| | | antigen). [Source: Uniprot/SWISSPROT; Acc: P16410] |
| 1511 | NM_001911 | Cathepsin G precursor (EC 3.4.21.20) (CG). |
| | | [Source: Uniprot/SWISSPROT; Acc: P08311] |
| 1520 | NM_004079 | Cathepsin S precursor (EC 3.4.22.27). |
| | | [Source: Uniprot/SWISSPROT; Acc: P25774] |
| 1521 | NM_001335 | Cathepsin W precursor (EC 3.4.22.—) (Lymphopain). |
| | | [Source: Uniprot/SWISSPROT; Acc: P56202] |
| 1524 | NM_001337 | CX3C chemokine receptor 1 (C-X3-C CKR-1) (CX3CR1) |
| | | (Fractalkine receptor) (G-protein coupled receptor 13) (V28) |
| | | (Beta chemokine receptor-like 1) (CMK-BRL-1) (CMKBLR1). |
| | | [Source: Uniprot/SWISSPROT; Acc: P49238] |
| 1536 | NM_000397 | Cytochrome B-245 heavy chain (P22 phagocyte B- |
| | | cytochrome) (Neutrophil cytochrome B, 91 kDa polypeptide) |
| | | (CGD91-PHOX) (GP91-PHOX) (GP91-1) (Heme binding |
| | | membrane glycoprotein GP91PHOX) (Cytochrome B(558) |
| | | beta chain) (Superoxide-generating NADPH oxidase hea |
| 1604 | NM_000574 | decay accelerating factor for complement (CD55, Cromer |
| | | blood group system); DAF |
| 1670 | NM_021010 | Defensin 5 precursor (Defensin, alpha 5). |
| | | [Source: Uniprot/SWISSPROT; Acc: Q01523] |
| 1671 | NM_001926 | Defensin 6 precursor (Defensin, alpha 6). |
| | | [Source: Uniprot/SWISSPROT; Acc: Q01524] |
| 1672 | NM_005218 | defensin, beta 1; DEFB1 |
| 1673 | NM_004942 | defensin, beta 4; DEFB4 |
| 1675 | NM_001928 | D component of complement (adipsin); DF |
| 1755 | NM_017579 | Deleted in malignant brain tumors 1 protein precursor |
| | | (Glycoprotein 340) (Gp-340) (Surfactant pulmonary- |
| | | associated D-binding protein). |
| | | [Source: Uniprot/SWISSPROT; Acc: Q9UGM3] |
| 1791 | NM_001017520 | DNA nucleotidylexotransferase (EC 2.7.7.31) (Terminal |
| | | addition enzyme) (Terminal deoxynucleotidyltransferase) |
| | | (Terminal transferase). |
| | | [Source: Uniprot/SWISSPROT; Acc: P04053] |
| 1794 | NM_004946 | Dedicator of cytokinesis protein 2. |
| | | [Source: Uniprot/SWISSPROT; Acc: Q92608] |
| 1803 | NM_001935 | Dipeptidyl peptidase 4 (EC 3.4.14.5) (Dipeptidyl peptidase |
| | | IV) (DPP IV) (T-cell activation antigen CD26) (TP103) |
| | | (Adenosine deaminase complexing protein 2) (ADABP) |
| | | [Contains: Dipeptidyl peptidase 4 membrane form |
| | | (Dipeptidyl peptidase IV membrane form); Di |
| 1880 | NM_004951 | EBV-induced G-protein coupled receptor 2 (EBI2). |
| | | [Source: Uniprot/SWISSPROT; Acc: P32249] |
| 1896 | NM_001005610 | Ectodysplasin A (Ectodermal dysplasia protein) (EDA |
| | | protein) [Contains: Ectodysplasin A, membrane form; |
| | | Ectodysplasin A, secreted form]. |
| | | [Source: Uniprot/SWISSPROT; Acc: Q92838] |
| 1903 | NM_005226 | Sphingosine 1-phosphate receptor Edg-3 (S1P receptor |
| | | Edg-3) (Endothelial differentiation G-protein coupled |
| | | receptor 3) (Sphingosine 1-phosphate receptor 3) (S1P3). |
| | | [Source: Uniprot/SWISSPROT; Acc: Q99500] |
| 1947 | NM_004429 | Ephrin-B1 precursor (EPH-related receptor tyrosine kinase |
| | | ligand 2) (LERK-2) (ELK ligand) (ELK-L). |
| | | [Source: Uniprot/SWISSPROT; Acc: P98172] |
| 1958 | NM_001964 | Early growth response protein 1 (EGR-1) (Krox-24 protein) |
| | | (Transcription factor Zif268) (Nerve growth factor-induced |
| | | protein A) (NGFI-A) (Transcription factor ETR103) (Zinc |
| | | finger protein 225) (AT225). |
| | | [Source: Uniprot/SWISSPROT; Acc: P18146] |
| 1997 | NM_172373 | ETS-related transcription factor Elf-1 (E74-like factor 1). |
| | | [Source: Uniprot/SWISSPROT; Acc: P32519] |
| 2000 | | ETS-related transcription factor Elf-4 (E74-like factor 4) |
| | | (Myeloid Elf-1-like factor). |
| | | [Source: Uniprot/SWISSPROT; Acc: Q99607] |
| 2053 | NM_001979 | Epoxide hydrolase 2 (EC 3.3.2.3) (Soluble epoxide |
| | | hydrolase) (SEH) (Epoxide hydratase) (Cytosolic epoxide |
| | | hydrolase) (CEH). [Source: Uniprot/SWISSPROT; Acc: P34913] |
| 2113 | NM_005238 | C-ets-1 protein (p54). |
| | | [Source: Uniprot/SWISSPROT; Acc: P14921] |
| 2147 | NM_000506 | Prothrombin precursor (EC 3.4.21.5) (Coagulation factor II) |
| | | [Contains: Activation peptide fragment 1; Activation peptide |
| | | fragment 2; Thrombin light chain; Thrombin heavy chain]. |
| | | [Source: Uniprot/SWISSPROT; Acc: P00734] |
| 2152 | NM_001993 | coagulation factor III (thromboplastin, tissue factor); F3 |
| 2157 | NM_000132 | Coagulation factor VIII precursor (Procoagulant component) |
| | | (Antihemophilic factor) (AHF) [Contains: Factor VIIIa heavy |
| | | chain, 200 kDa isoform; Factor VIIIa heavy chain, 92 kDa |
| | | isoform; Factor VIII B chain; Factor VIIIa light chain]. |
| | | [Source: Uniprot/SWISSP |
| 2158 | NM_000133 | coagulation factor IX (plasma thromboplastic component, |
| | | Christmas disease, hemophilia B); F9 |
| 2159 | NM_000504 | coagulation factor X; F10 |
| 2165 | NM_001994 | coagulation factor XIII, B polypeptide; F13B |
| 2204 | NM_133271; NM_133272; | Fc fragment of IgA, receptor for; FCAR |
| | NM_133269; NM_133278; | |
| | NM_002000; NM_133273; | |
| | NM_133277; NM_133279; | |
| | NM_133280; NM_133274 | |
| 2205 | NM_002001 | Fc fragment of IgE, high affinity I, receptor for; alpha |
| | | polypeptide; FCER1A |
| 2206 | NM_000139 | membrane-spanning 4-domains, subfamily A, member 2 (Fc |
| | | fragment of IgE, high affinity I, receptor for; beta |
| | | polypeptide); MS4A2 |
| 2207 | NM_004106 | Fc fragment of IgE, high affinity I, receptor for; gamma |
| | | polypeptide; FCER1G |
| 2208 | NM_002002 | Fc fragment of IgE, low affinity II, receptor for |
| | | (CD23A); FCER2 |
| 2209 | NM_000566 | Fc fragment of IgG, high affinity Ia, receptor |
| | | (CD64); FCGR1A |
| 2212 | NM_021642 | Fc fragment of IgG, low affinity IIa, receptor |
| | | (CD32); FCGR2A |
| 2213 | NM_004001; NM_001002275; | Fc fragment of IgG, low affinity IIb, receptor |
| | NM_001002274; NM_001002273 | (CD32); FCGR2B |
| 2214 | NM_000569 | Fc fragment of IgG, low affinity IIIa, receptor |
| | | (CD16a); FCGR3A |
| 2215 | NM_000570 | Fc fragment of IgG, low affinity IIIb, receptor |
| | | (CD16b); FCGR3B |
| 2217 | NM_004107 | Fc fragment of IgG, receptor, transporter, alpha; FCGRT |
| 2219 | NM_002003 | ficolin (collagen/fibrinogen domain containing) 1; FCN1 |
| 2220 | NM_015837; NM_004108 | ficolin (collagen/fibrinogen domain containing lectin) 2 |
| | | (hucolin); FCN2 |
| 2255 | NM_004465 | Fibroblast growth factor 10 precursor (FGF-10) |
| | | (Keratinocyte growth factor 2). |
| | | [Source: Uniprot/SWISSPROT; Acc: O15520] |
| 2268 | NM_005248 | Gardner-Rasheed feline sarcoma viral (v-fgr) oncogene |
| | | homolog; FGR |
| 2280 | NM_054014 | FK506 binding protein 1A, 12 kDa (FKBP1A) |
| 2289 | NM_004117 | FK506 binding protein 5 (FKBP5) |
| 2322 | NM_004119 | FL cytokine receptor precursor (EC 2.7.1.112) (Tyrosine- |
| | | protein kinase receptor FLT3) (Stem cell tyrosine kinase 1) |
| | | (STK-1) (CD135 antigen). |
| | | [Source: Uniprot/SWISSPROT; Acc: P36888] |
| 2323 | NM_001459 | SL cytokine precursor (Fms-related tyrosine kinase 3 ligand) |
| | | (Flt3 ligand) (Flt3L). |
| | | [Source: Uniprot/SWISSPROT; Acc: P49771] |
| 2335 | NM_054034 | Fibronectin precursor (FN) (Cold-insoluble globulin) (CIG). |
| | | [Source: Uniprot/SWISSPROT; Acc: P02751] |
| 2353 | NM_005252 | v-fos FBJ murine osteosarcoma viral oncogene |
| | | homolog; FOS |
| 2357 | NM_002029 | fMet-Leu-Phe receptor (fMLP receptor) (N-formyl peptide |
| | | receptor) (FPR) (N-formylpeptide chemoattractant |
| | | receptor). [Source: Uniprot/SWISSPROT; Acc: P21462] |
| 2358 | NM_001005738 | FMLP-related receptor I (FMLP-R-I) (Lipoxin A4 receptor) |
| | | (LXA4 receptor) (Formyl peptide receptor-like 1) (RFP) |
| | | (HM63). [Source: Uniprot/SWISSPROT; Acc: P25090] |
| 2444 | NM_002031 | fyn-related kinase; FRK |
| 2495 | NM_002032 | Ferritin heavy chain (EC 1.16.3.1) (Ferritin H subunit) |
| | | (Proliferation-inducing gene 15 protein). |
| | | [Source: Uniprot/SWISSPROT; Acc: P02794] |
| 2532 | NM_002036 | Duffy antigen/chemokine receptor (Fy glycoprotein) (GpFy) |
| | | (Glycoprotein D) (Plasmodium vivax receptor) (CD234 |
| | | antigen). [Source: Uniprot/SWISSPROT; Acc: Q16570] |
| 2533 | NM_001465 | FYN-binding protein (FYN-T-binding protein) (FYB- |
| | | 120/130) (p120/p130) (SLP-76 associated phosphoprotein) |
| | | (SLAP-130). [Source: Uniprot/SWISSPROT; Acc: O15117] |
| 2534 | NM_153048; NM_002037; | FYN oncogene related to SRC, FGR, YES; FYN |
| | NM_153047 | |
| 2537 | NM_002038 | Interferon-induced protein 6-16 precursor (Ifi-6-16). |
| | | [Source: Uniprot/SWISSPROT; Acc: P09912] |
| 2543 | NM_001468 | GAGE-1 protein (G antigen 1) (MZ2-F antigen). |
| | | [Source: Uniprot/SWISSPROT; Acc: Q13065] |
| 2574 | NM_012196 | GAGE-1 protein (G antigen 1) (MZ2-F antigen). |
| | | [Source: Uniprot/SWISSPROT; Acc: Q13065] |
| 2633 | NM_002053 | Interferon-induced guanylate-binding protein 1 (GTP- |
| | | binding protein 1) (Guanine nucleotide-binding protein 1) |
| | | (HuGBP-1). [Source: Uniprot/SWISSPROT; Acc: P32455] |
| 2634 | NM_004120 | Interferon-induced guanylate-binding protein 2 (GTP- |
| | | binding protein 2) (Guanine nucleotide-binding protein 2) |
| | | (HuGBP-2). [Source: Uniprot/SWISSPROT; Acc: P32456] |
| 2635 | NM_018284 | guanylate binding protein 3 |
| | | [Source: RefSeq_peptide; Acc: NP_060754] |
| 2637 | NM_001485 | Homeobox protein GBX-2 (Gastrulation and brain-specific |
| | | homeobox protein 2). |
| | | [Source: Uniprot/SWISSPROT; Acc: P52951] |
| 2669 | NM_181702 | GTP-binding protein GEM (GTP-binding mitogen-induced T- |
| | | cell protein) (RAS-like protein KIR). |
| | | [Source: Uniprot/SWISSPROT; Acc: P55040] |
| 2687 | NM_004121 | Gamma-glutamyltransferase 5 precursor (EC 2.3.2.2) |
| | | (Gamma-glutamyltranspeptidase 5) (Gamma- |
| | | glutamyltransferase-like activity 1) (GGT-rel) [Contains: |
| | | Gamma-glutamyltransferase 5 heavy chain; Gamma- |
| | | glutamyltransferase 5 light chain]. [Source: Uniprot/SWISS |
| 2821 | | Glucose-6-phosphate isomerase (EC 5.3.1.9) (GPI) |
| | | (Phosphoglucose isomerase) (PGI) (Phosphohexose |
| | | isomerase) (PHI) (Neuroleukin) (NLK) (Sperm antigen 36) |
| | | (SA-36). [Source: Uniprot/SWISSPROT; Acc: P06744] |
| 2829 | NM_001024644 | Chemokine XC receptor 1 (XC chemokine receptor 1) |
| | | (Lymphotactin receptor) (G-protein coupled receptor 5). |
| | | [Source: Uniprot/SWISSPROT; Acc: P46094] |
| 2833 | NM_001504 | C—X—C chemokine receptor type 3 (CXC-R3) (CXCR-3) |
| | | (Interferon-inducible protein 10 receptor) (IP-10 receptor) |
| | | (CKR-L2) (CD183 antigen) (G protein-coupled receptor 9). |
| | | [Source: Uniprot/SWISSPROT; Acc: P49682] |
| 2874 | NM_004489 | G protein pathway suppressor 2 (GPS2) |
| 2885 | NM_002086; NM_203506 | growth factor receptor-bound protein 2; GRB2 |
| 2908 | NM_001018076 | Glucocorticoid receptor (GR). |
| | | [Source: Uniprot/SWISSPROT; Acc: P04150] |
| 2919 | NM_001511 | Growth regulated protein alpha precursor (CXCL1) |
| | | (Melanoma growth stimulatory activity) (MGSA) (Neutrophil- |
| | | activating protein 3) (NAP-3) (GRO-alpha(1-73)) [Contains: |
| | | GRO-alpha(4-73); GRO-alpha(5-73); GRO-alpha(6-73)]. |
| | | [Source: Uniprot/SWISSPROT; Acc: P09341 |
| 2920 | NM_002089 | Macrophage inflammatory protein 2-alpha precursor (MIP2- |
| | | alpha) (CXCL2) (Growth regulated protein beta) (Gro-beta) |
| | | [Contains: GRO-beta(5-73) (GRO-beta-T) (SB-251353) |
| | | (Hematopoietic synergistic factor) (HSF)]. |
| | | [Source: Uniprot/SWISSPROT; Acc: P19875] |
| 2921 | NM_002090 | Macrophage inflammatory protein 2-beta precursor (MIP2- |
| | | beta) (CXCL3) (Growth regulated protein gamma) (GRO- |
| | | gamma) (GRO-gamma(1-73)) [Contains: GRO-gamma(5- |
| | | 73)]. [Source: Uniprot/SWISSPROT; Acc: P19876] |
| 3001 | NM_006144 | Granzyme A precursor (EC 3.4.21.78) (Cytotoxic T- |
| | | lymphocyte proteinase 1) (Hanukkah factor) (H factor) (HF) |
| | | (Granzyme-1) (CTL tryptase) (Fragmentin-1). |
| | | [Source: Uniprot/SWISSPROT; Acc: P12544] |
| 3055 | NM_002110 | hemopoietic cell kinase; HCK |
| 3069 | NM_203346 | High density lipoprotein binding protein (vigilin) (HDLBP) |
| 3070 | NM_018063 | helicase, lymphoid-specific |
| | | [Source: RefSeq_peptide; Acc: NP_060533] |
| 3075 | NM_000186; NM_001014975 | complement factor H; CFH |
| 3077 | NM_139006 | Hereditary hemochromatosis protein precursor (HLA-H). |
| | | [Source: Uniprot/SWISSPROT; Acc: Q30201] |
| 3078 | NM_002113 | complement factor H-related 1; CFHL1 |
| 3080 | NM_005666 | complement factor H-related 2; CFHL2 |
| 3082 | NM_000601; NM_001010932; | hepatocyte growth factor (hepapoietin A; scatter |
| | NM_001010931; NM_001010933; | factor); HGF |
| | NM_001010934 | |
| 3087 | NM_002729 | Homeobox protein PRH (Hematopoietically expressed |
| | | homeobox) (Homeobox protein HEX). |
| | | [Source: Uniprot/SWISSPROT; Acc: Q03014] |
| 3105 | NM_002116 | major histocompatibility complex, class I, A; HLA-A |
| 3106 | NM_005514 | major histocompatibility complex, class I, B; HLA-B |
| 3107 | NM_002117 | major histocompatibility complex, class I, C; HLA-C |
| 3108 | NM_006120 | major histocompatibility complex, class II, DM alpha; HLA- |
| | | DMA |
| 3109 | NM_002118 | major histocompatibility complex, class II, DM beta; HLA- |
| | | DMB |
| 3110 | NM_005515 | Homeobox protein HB9. |
| | | [Source: Uniprot/SWISSPROT; Acc: P50219] |
| 3111 | NM_002119 | major histocompatibility complex, class II, DO alpha; HLA- |
| | | DOA |
| 3112 | NM_002120 | major histocompatibility complex, class II, DO beta; HLA- |
| | | DOB |
| 3113 | NM_033554 | major histocompatibility complex, class II, DP alpha 1; HLA- |
| | | DPA1 |
| 3115 | NM_002121 | major histocompatibility complex, class II, DP beta 1; HLA- |
| | | DPB1 |
| 3117 | NM_002122 | major histocompatibility complex, class II, DQ alpha 1; HLA- |
| | | DQA1 |
| 3118 | NM_020056 | major histocompatibility complex, class II, DQ alpha 2; HLA- |
| | | DQA2 |
| 3119 | NM_002123 | major histocompatibility complex, class II, DQ beta 1; HLA- |
| | | DQB1 |
| 3120 | NM_182549 | major histocompatibility complex, class II, DQ beta 2; HLA- |
| | | DQB2 |
| 3122 | NM_019111 | major histocompatibility complex, class II, DR alpha; HLA- |
| | | DRA |
| 3123 | NM_002124 | major histocompatibility complex, class II, DR beta 1; HLA- |
| | | DRB1 |
| 3124 | NM_001037638 | major histocompatibility complex, class II, DR beta 2; HLA- |
| | | DRB2 |
| 3125 | NM_022555 | major histocompatibility complex, class II, DR beta 3; HLA- |
| | | DRB3 |
| 3126 | NM_021983 | major histocompatibility complex, class II, DR beta 4; HLA- |
| | | DRB4 |
| 3127 | NM_002125 | major histocompatibility complex, class II, DR beta 5; HLA- |
| | | DRB5 |
| 3133 | NM_005516 | major histocompatibility complex, class I, E; HLA-E |
| 3134 | NM_018950 | major histocompatibility complex, class I, F; HLA-F |
| 3135 | NM_002127 | HLA-G histocompatibility antigen, class I, G; HLA-G |
| 3140 | NM_001531 | major histocompatibility complex, class I-related; MR1 |
| 3149 | NM_005342 | High mobility group protein 4 (HMG-4) (High mobility group |
| | | protein 2a) (HMG-2a). |
| | | [Source: Uniprot/SWISSPROT; Acc: O15347] |
| 3265 | NM_176795; NM_005343 | v-Ha-ras Harvey rat sarcoma viral oncogene homolog; HRAS |
| 3269 | NM_000861 | Histamine H1 receptor. |
| | | [Source: Uniprot/SWISSPROT; Acc: P35367] |
| 3274 | NM_022304 | Histamine H2 receptor (H2R) (Gastric receptor I). |
| | | [Source: Uniprot/SWISSPROT; Acc: P25021] |
| 3320 | NM_005348 | Heat shock protein HSP 90-alpha (HSP 86). |
| | | [Source: Uniprot/SWISSPROT; Acc: P07900] |
| 3324 | NM_005348 | Heat shock protein HSP 90-alpha (HSP 86). |
| | | [Source: Uniprot/SWISSPROT; Acc: P07900] |
| 3394 | NM_002163 | Interferon regulatory factor 8 (IRF-8) (Interferon consensus |
| | | sequence binding protein) (ICSBP). |
| | | [Source: Uniprot/SWISSPROT; Acc: Q02556] |
| 3426 | NM_000204 | I factor (complement); IF |
| 3430 | NM_005533 | Interferon-induced 35 kDa protein (IFP 35). |
| | | [Source: Uniprot/SWISSPROT; Acc: P80217] |
| 3433 | NM_001547 | Interferon-induced protein with tetratricopeptide repeats 2 |
| | | (IFIT-2) (Interferon-induced 54 kDa protein) (IFI-54K) |
| | | (ISG-54 K). [Source: Uniprot/SWISSPROT; Acc: P09913] |
| 3434 | NM_001548 | Interferon-induced protein with tetratricopeptide repeats 1 |
| | | (IFIT-1) (Interferon-induced 56 kDa protein) (IFI-56K). |
| | | [Source: Uniprot/SWISSPROT; Acc: P09914] |
| 3437 | NM_001549 | Interferon-induced protein with tetratricopeptide repeats 3 |
| | | (IFIT-3) (IFIT-4) (Interferon-induced 60 kDa protein) (IFI- |
| | | 60K) (ISG-60) (CIG49) (Retinoic acid-induced gene G |
| | | protein) (RIG-G). [Source: Uniprot/SWISSPROT; Acc: O14879] |
| 3440 | NM_000605 | Interferon alpha-2 precursor (Interferon alpha-A) (LeIF A). |
| | | [Source: Uniprot/SWISSPROT; Acc: P01563] |
| 3454 | NM_000629 | interferon (alpha, beta and omega) receptor 1; IFNAR1 |
| 3456 | NM_002176 | Interferon beta precursor (IFN-beta) (Fibroblast interferon). |
| | | [Source: Uniprot/SWISSPROT; Acc: P01574] |
| 3458 | NM_000619 | Interferon gamma precursor (IFN-gamma) (Immune |
| | | interferon). [Source: Uniprot/SWISSPROT; Acc: P01579] |
| 3459 | NM_000416 | Interferon gamma receptor 1 (IFNGR1) |
| 3460 | NM_005534 | interferon gamma receptor 2 (interferon gamma transducer |
| | | 1); IFNGR2 |
| 3476 | NM_001551 | immunoglobulin (CD79A) binding protein 1; IGBP1 |
| 3512 | NM_144646 | Immunoglobulin J chain. |
| | | [Source: Uniprot/SWISSPROT; Acc: P01591] |
| 3516 | | Recombining binding protein suppressor of hairless (J |
| | | kappa-recombination signal binding protein) (RBP-J kappa) |
| | | (RBP-J) (RBP-JK) (CBF-1). |
| | | [Source: Uniprot/SWISSPROT; Acc: Q06330] |
| 3543 | NM_020070; NM_152855 | immunoglobulin lambda-like polypeptide 1; IGLL1 |
| 3547 | NM_001555; NM_205833 | immunoglobulin superfamily, member 1; IGSF1 |
| 3550 | NM_006083 | Red protein (RER protein) (IK factor) (Cytokine IK). |
| | | [Source: Uniprot/SWISSPROT; Acc: Q13123] |
| 3551 | NM_001556 | inhibitor of kappa light polypeptide gene enhancer in B- |
| | | cells, kinase beta; IKBKB |
| 3552 | NM_000575 | Interleukin-1 alpha precursor (IL-1 alpha) (Hematopoietin- |
| | | 1). [Source: Uniprot/SWISSPROT; Acc: P01583] |
| 3553 | NM_000576 | Interleukin-1 beta precursor (IL-1 beta) (Catabolin). |
| | | [Source: Uniprot/SWISSPROT; Acc: P01584] |
| 3554 | NM_000877 | Interleukin-1 receptor type I precursor (IL-1R-1) (IL-1RT1) |
| | | (IL-1R-alpha) (p80) (Antigen CD121a). |
| | | [Source: Uniprot/SWISSPROT; Acc: P14778] |
| 3556 | NM_134470 | Interleukin-1 receptor accessory protein precursor (IL-1 |
| | | receptor accessory protein) (IL-1RAcP). |
| | | [Source: Uniprot/SWISSPROT; Acc: Q9NPH3] |
| 3557 | NM_000577 | Interleukin-1 receptor antagonist protein precursor (IL-1ra) |
| | | (IRAP) (IL1 inhibitor) (IL-1RN) (ICIL-1RA). |
| | | [Source: Uniprot/SWISSPROT; Acc: P18510] |
| 3558 | NM_000586 | Interleukin-2 precursor (IL-2) (T-cell growth factor) (TCGF) |
| | | (Aldesleukin). [Source: Uniprot/SWISSPROT; Acc: P60568] |
| 3559 | NM_000417 | Interleukin-2 receptor alpha chain precursor (IL-2 receptor |
| | | alpha subunit) (IL-2-RA) (IL2-RA) (p55) (TAC antigen) |
| | | (CD25 antigen). [Source: Uniprot/SWISSPROT; Acc: P01589] |
| 3560 | NM_000878 | Interleukin-2 receptor beta chain precursor (IL-2 receptor) |
| | | (P70-75) (p75) (High affinity IL-2 receptor beta subunit) |
| | | (CD122 antigen). [Source: Uniprot/SWISSPROT; Acc: P14784] |
| 3561 | NM_000206 | Cytokine receptor common gamma chain precursor |
| | | (Gamma-C) (Interleukin-2 receptor gamma chain) (IL-2R |
| | | gamma chain) (P64) (CD132 antigen). |
| | | [Source: Uniprot/SWISSPROT; Acc: P31785] |
| 3562 | NM_000588 | Interleukin-3 precursor (IL-3) (Multipotential colony- |
| | | stimulating factor) (Hematopoietic growth factor) (P-cell- |
| | | stimulating factor) (Mast-cell growth factor) (MCGF). |
| | | [Source: Uniprot/SWISSPROT; Acc: P08700] |
| 3565 | NM_000589 | Interleukin-4 precursor (IL-4) (B-cell stimulatory factor 1) |
| | | (BSF-1) (Lymphocyte stimulatory factor 1). |
| | | [Source: Uniprot/SWISSPROT; Acc: P05112] |
| 3566 | NM_001008699 | Interleukin-4 receptor alpha chain precursor (IL-4R-alpha) |
| | | (CD124 antigen) [Contains: Soluble interleukin-4 receptor |
| | | alpha chain (sIL4Ralpha/prot) (IL-4-binding protein) (IL4- |
| | | BP)]. [Source: Uniprot/SWISSPROT; Acc: P24394] |
| 3567 | NM_000879 | Interleukin-5 precursor (IL-5) (T-cell replacing factor) |
| | | (TRF) (Eosinophil differentiation factor) (B cell differentiation |
| | | factor I). [Source: Uniprot/SWISSPROT; Acc: P05113] |
| 3569 | NM_000600 | Interleukin-6 precursor (IL-6) (B-cell stimulatory factor 2) |
| | | (BSF-2) (Interferon beta-2) (Hybridoma growth factor) (CTL |
| | | differentiation factor) (CDF). |
| | | [Source: Uniprot/SWISSPROT; Acc: P05231] |
| 3570 | NM_000565 | Interleukin-6 receptor alpha chain precursor (IL-6R-alpha) |
| | | (IL-6R 1) (Membrane glycoprotein 80) (gp80) (CD126 |
| | | antigen). [Source: Uniprot/SWISSPROT; Acc: P08887] |
| 3572 | NM_002184 | Interleukin-6 receptor beta chain precursor (IL-6R-beta) |
| | | (Interleukin 6 signal transducer) (Membrane glycoprotein |
| | | 130) (gp130) (Oncostatin M receptor) (CD130 antigen) |
| | | (CDw130). [Source: Uniprot/SWISSPROT; Acc: P40189] |
| 3574 | NM_000880 | Interleukin-7 precursor (IL-7). |
| | | [Source: Uniprot/SWISSPROT; Acc: P13232] |
| 3575 | NM_002185 | Interleukin-7 receptor alpha chain precursor (IL-7R-alpha) |
| | | (CD127 antigen) (CDw127). |
| | | [Source: Uniprot/SWISSPROT; Acc: P16871] |
| 3576 | NM_000584 | Interleukin-8 precursor (IL-8) (CXCL8) (Monocyte-derived |
| | | neutrophil chemotactic factor) (MDNCF) (T-cell chemotactic |
| | | factor) (Neutrophil-activating protein 1) (NAP-1) (Protein |
| | | 3-10C) (Granulocyte chemotactic protein 1) (GCP-1) |
| | | (Monocyte derived neutrophil |
| 3577 | NM_000634 | High affinity interleukin-8 receptor A (IL-8R A) (IL-8 |
| | | receptor type 1) (CXCR-1) (CD181 antigen) (CDw128a |
| | | antigen). [Source: Uniprot/SWISSPROT; Acc: P25024] |
| 3578 | NM_000590 | Interleukin-9 precursor (IL-9) (T-cell growth factor P40) |
| | | (P40 cytokine). [Source: Uniprot/SWISSPROT; Acc: P15248] |
| 3579 | NM_001557 | High affinity interleukin-8 receptor B (IL-8R B) (CXCR-2) |
| | | (GRO/MGSA receptor) (IL-8 receptor type 2) (CD182 |
| | | antigen) (CDw128b antigen). |
| | | [Source: Uniprot/SWISSPROT; Acc: P25025] |
| 3586 | NM_000572 | Interleukin-10 precursor (IL-10) (Cytokine synthesis |
| | | inhibitory factor) (CSIF). |
| | | [Source: Uniprot/SWISSPROT; Acc: P22301] |
| 3587 | NM_001558 | Interleukin 10 receptor, alpha (IL10RA) |
| 3588 | NM_000628 | interleukin 10 receptor, beta; IL10RB |
| 3589 | NM_000641 | Interleukin-11 precursor (IL-11) (Adipogenesis inhibitory |
| | | factor) (AGIF) (Oprelvekin). |
| | | [Source: Uniprot/SWISSPROT; Acc: P20809] |
| 3590 | | Interleukin-11 receptor alpha chain precursor (IL-11R- |
| | | alpha) (IL-11RA). |
| | | [Source: Uniprot/SWISSPROT; Acc: Q14626] |
| 3592 | NM_000882 | Interleukin-12 alpha chain precursor (IL-12A) (Cytotoxic |
| | | lymphocyte maturation factor 35 kDa subunit) (CLMF p35) |
| | | (NK cell stimulatory factor chain 1) (NKSF1). |
| | | [Source: Uniprot/SWISSPROT; Acc: P29459] |
| 3593 | NM_002187 | Interleukin-12 beta chain precursor (IL-12B) (IL-12 p40) |
| | | (Cytotoxic lymphocyte maturation factor 40 kDa subunit) |
| | | (CLMF p40) (NK cell stimulatory factor chain 2) (NKSF2). |
| | | [Source: Uniprot/SWISSPROT; Acc: P29460] |
| 3594 | NM_153701; NM_005535 | interleukin 12 receptor, beta 1; IL12RB1 |
| 3596 | NM_002188 | Interleukin-13 precursor (IL-13). |
| | | [Source: Uniprot/SWISSPROT; Acc: P35225] |
| 3600 | NM_000585 | Interleukin-15 precursor (IL-15). |
| | | [Source: Uniprot/SWISSPROT; Acc: P40933] |
| 3603 | | Interleukin-16 precursor (IL-16) (Lymphocyte |
| | | chemoattractant factor) (LCF). |
| | | [Source: Uniprot/SWISSPROT; Acc: Q14005] |
| 3604 | NM_001561 | Tumor necrosis factor receptor superfamily member 9 |
| | | precursor (4-1BB ligand receptor) (T-cell antigen 4-1BB |
| | | homolog) (T-cell antigen ILA) (CD137 antigen). |
| | | [Source: Uniprot/SWISSPROT; Acc: Q07011] |
| 3605 | NM_002190 | Interleukin-17 precursor (IL-17) (IL-17A) (Cytotoxic T |
| | | lymphocyte-associated antigen 8) (CTLA-8). |
| | | [Source: Uniprot/SWISSPROT; Acc: Q16552] |
| 3606 | NM_001562 | Interleukin-18 precursor (IL-18) (Interferon-gamma- |
| | | inducing factor) (IFN-gamma-inducing factor) (Interleukin- |
| | | 1 gamma) (IL-1 gamma). |
| | | [Source: Uniprot/SWISSPROT; Acc: Q14116] |
| 3608 | NM_004515 | Interleukin enhancer-binding factor 2 (Nuclear factor of |
| | | activated T-cells 45 kDa). |
| | | [Source: Uniprot/SWISSPROT; Acc: Q12905] |
| 3614 | NM_000883 | Inosine-5′-monophosphate dehydrogenase 1 (EC 1.1.1.205) |
| | | (IMP dehydrogenase 1) (IMPDH-I) (IMPD 1). |
| | | [Source: Uniprot/SWISSPROT; Acc: P20839] |
| 3615 | NM_000884 | Inosine-5′-monophosphate dehydrogenase 2 (EC 1.1.1.205) |
| | | (IMP dehydrogenase 2) (IMPDH-II) (IMPD 2). |
| | | [Source: Uniprot/SWISSPROT; Acc: P12268] |
| 3620 | NM_002164 | Indoleamine 2,3-dioxygenase (EC 1.13.11.42) (IDO) |
| | | (Indoleamine-pyrrole 2,3-dioxygenase). |
| | | [Source: Uniprot/SWISSPROT; Acc: P14902] |
| 3623 | NM_002191 | Inhibin alpha chain precursor. |
| | | [Source: Uniprot/SWISSPROT; Acc: P05111] |
| 3624 | NM_002192 | Inhibin beta A chain precursor (Activin beta-A chain) |
| | | (Erythroid differentiation protein) (EDF). |
| | | [Source: Uniprot/SWISSPROT; Acc: P08476] |
| 3625 | NM_002193 | Inhibin beta B chain precursor (Activin beta-B chain). |
| | | [Source: Uniprot/SWISSPROT; Acc: P09529] |
| 3627 | NM_001565 | Small inducible cytokine B10 precursor (CXCL10) (10 kDa |
| | | interferon-gamma-induced protein) (Gamma-IP10) (IP- |
| | | 10) [Contains: CXCL10(1-73)]. |
| | | [Source: Uniprot/SWISSPROT; Acc: P02778] |
| 3630 | NM_000207 | Insulin precursor [Contains: Insulin B chain; Insulin A |
| | | chain]. [Source: Uniprot/SWISSPROT; Acc: P01308] |
| 3635 | NM_005541 | SH2 containing inositol phosphatase isoform a |
| | | [Source: RefSeq_peptide; Acc: NP_001017915] |
| 3656 | NM_001570 | Interleukin-1 receptor-associated kinase-like 2 (IRAK-2). |
| | | [Source: Uniprot/SWISSPROT; Acc: O43187] |
| 3659 | NM_002198 | Interferon regulatory factor 1 (IRF-1). |
| | | [Source: Uniprot/SWISSPROT; Acc: P10914] |
| 3660 | NM_002199 | Interferon regulatory factor 2 (IRF-2). |
| | | [Source: Uniprot/SWISSPROT; Acc: P14316] |
| 3662 | NM_002460 | Interferon regulatory factor 4 (IRF-4) (Lymphocyte-specific |
| | | interferon regulatory factor) (LSIRF) (NF-EM5) (Multiple |
| | | myeloma oncogene 1). |
| | | [Source: Uniprot/SWISSPROT; Acc: Q15306] |
| 3665 | NM_001572 | Interferon regulatory factor 7 (IRF-7). |
| | | [Source: Uniprot/SWISSPROT; Acc: Q92985] |
| 3672 | NM_181501 | Pelota homolog. [Source: Uniprot/SWISSPROT; Acc: Q9BRX2] |
| 3681 | NM_005353 | Integrin alpha-D precursor (Leukointegrin alpha D) (CD11d) |
| | | (ADB2). [Source: Uniprot/SWISSPROT; Acc: Q13349] |
| 3683 | NM_002209 | Integrin alpha-L precursor (Leukocyte adhesion glycoprotein |
| | | LFA-1 alpha chain) (LFA-1A) (Leukocyte function associated |
| | | molecule 1, alpha chain) (CD11a). |
| | | [Source: Uniprot/SWISSPROT; Acc: P20701] |
| 3684 | NM_000632 | Integrin alpha-M precursor (Cell surface glycoprotein MAC-1 |
| | | alpha subunit) (CR-3 alpha chain) (CD11b) (Leukocyte |
| | | adhesion receptor MO1) (Neutrophil adherence receptor). |
| | | [Source: Uniprot/SWISSPROT; Acc: P11215] |
| 3688 | NM_002211 | Integrin beta-1 precursor (Fibronectin receptor beta |
| | | subunit) (Integrin VLA-4 beta subunit) (CD29 antigen). |
| | | [Source: Uniprot/SWISSPROT; Acc: P05556] |
| 3689 | NM_000211 | Integrin beta-2 precursor (Cell surface adhesion |
| | | glycoproteins LFA-1/CR3/p150,95 beta-subunit) (CD18) |
| | | (Complement receptor C3 beta-subunit). |
| | | [Source: Uniprot/SWISSPROT; Acc: P05107] |
| 3697 | NM_002215 | Inter-alpha-trypsin inhibitor heavy chain H1 precursor (ITI |
| | | heavy chain H1) (Inter-alpha-inhibitor heavy chain 1) |
| | | (Inter-alpha-trypsin inhibitor complex component III) |
| | | (Serum-derived hyaluronan-associated protein) (SHAP). |
| | | [Source: Uniprot/SWISSPROT; Acc: P19 |
| 3700 | NM_002218 | Inter-alpha-trypsin inhibitor heavy chain H4 precursor (ITI |
| | | heavy chain H4) (Inter-alpha-inhibitor heavy chain 4) |
| | | (Inter-alpha-trypsin inhibitor family heavy chain-related |
| | | protein) (IHRP) (Plasma kallikrein sensitive glycoprotein |
| | | 120) (PK-120) (GP120) [Co |
| 3702 | NM_005546 | Tyrosine-protein kinase ITK/TSK (EC 2.7.1.112) (T-cell- |
| | | specific kinase) (Tyrosine-protein kinase Lyk) (Kinase EMT). |
| | | [Source: Uniprot/SWISSPROT; Acc: Q08881] |
| 3707 | NM_002221 | Inositol-trisphosphate 3-kinase B (EC 2.7.1.127) (Inositol |
| | | 1,4,5-trisphosphate 3-kinase B) (IP3K B) (IP3 3-kinase B) |
| | | (IP3K-B). [Source: Uniprot/SWISSPROT; Acc: P27987] |
| 3708 | NM_002222 | inositol 1,4,5-triphosphate receptor, type 1; ITPR1 |
| 3709 | NM_002223 | inositol 1,4,5-triphosphate receptor, type 2; ITPR2 |
| 3710 | NM_002224 | inositol 1,4,5-triphosphate receptor, type 3; ITPR3 |
| 3714 | NM_002226; NM_145159 | jagged 2; JAG2 |
| 3716 | NM_002227 | Janus kinase 1 (a protein tyrosine kinase) (JAK1) |
| 3718 | BC028068 | Janus kinase 3 (a protein tyrosine kinase, leukocyte) |
| 3725 | NM_002228 | v-jun sarcoma virus 17 oncogene homolog (avian); JUN |
| 3802 | NM_014218 | Killer cell immunoglobulin-like receptor 2DL1 precursor |
| | | (MHC class I NK cell receptor) (Natural killer associated |
| | | transcript 1) (NKAT-1) (p58 natural killer cell receptor |
| | | clones CL-42/47.11) (p58 NK receptor) (p58.1 MHC class-I- |
| | | specific NK receptor). [Sou |
| 3803 | NM_015868 | Killer cell immunoglobulin-like receptor 2DL2 precursor |
| | | (MHC class I NK cell receptor) (Natural killer associated |
| | | transcript 6) (NKAT-6) (p58 natural killer cell receptor clone |
| | | CL-43) (p58 NK receptor). |
| | | [Source: Uniprot/SWISSPROT; Acc: P43627] |
| 3804 | NM_015868 | Killer cell immunoglobulin-like receptor 2DL2 precursor |
| | | (MHC class I NK cell receptor) (Natural killer associated |
| | | transcript 6) (NKAT-6) (p58 natural killer cell receptor clone |
| | | CL-43) (p58 NK receptor). |
| | | [Source: Uniprot/SWISSPROT; Acc: P43627] |
| 3805 | NM_002255 | killer cell immunoglobulin-like receptor, two domains, long |
| | | cytoplasmic tail, 4; KIR2DL4 |
| 3806 | NM_012313 | Killer cell immunoglobulin-like receptor 2DS1 precursor |
| | | (MHC class I NK cell receptor Eb6 ActI). |
| | | [Source: Uniprot/SWISSPROT; Acc: Q14954] |
| 3807 | | Killer cell immunoglobulin-like receptor 3DL3 precursor |
| | | (Killer cell inhibitory receptor 1). |
| | | [Source: Uniprot/SWISSPROT; Acc: Q8N743] |
| 3808 | NM_012313 | Killer cell immunoglobulin-like receptor 2DS1 precursor |
| | | (MHC class I NK cell receptor Eb6 ActI). |
| | | [Source: Uniprot/SWISSPROT; Acc: Q14954] |
| 3809 | NM_178228; NM_012314 | killer cell immunoglobulin-like receptor, two domains, short |
| | | cytoplasmic tail, 4; KIR2DS4 |
| 3810 | | Killer cell immunoglobulin-like receptor 3DL3 precursor |
| | | (Killer cell inhibitory receptor 1). |
| | | [Source: Uniprot/SWISSPROT; Acc: Q8N743] |
| 3811 | NM_013289 | killer cell immunoglobulin-like receptor, three domains, long |
| | | cytoplasmic tail, 1; KIR3DL1 |
| 3812 | NM_006737 | killer cell immunoglobulin-like receptor, three domains, long |
| | | cytoplasmic tail, 2; KIR3DL2 |
| 3818 | NM_000892 | Plasma kallikrein precursor (EC 3.4.21.34) (Plasma |
| | | prekallikrein) (Kininogenin) (Fletcher factor) [Contains: |
| | | Plasma kallikrein heavy chain; Plasma kallikrein light chain]. |
| | | [Source: Uniprot/SWISSPROT; Acc: P03952] |
| 3820 | NM_002258 | killer cell lectin-like receptor subfamily B, member 1; KLRB1 |
| 3821 | NM_002259; NM_213657; | killer cell lectin-like receptor subfamily C, member 1; KLRC1 |
| | NM_007328; NM_213658 | |
| 3822 | NM_002260 | killer cell lectin-like receptor subfamily C, member 2; KLRC2 |
| 3823 | NM_007333; NM_002261 | killer cell lectin-like receptor subfamily C, member 3; KLRC3 |
| 3824 | NM_002262; NM_007334 | killer cell lectin-like receptor subfamily D, member 1; KLRD1 |
| 3845 | NM_033360; NM_004985 | v-Ki-ras2 Kirsten rat sarcoma viral oncogene homolog; KRAS |
| 3848 | NM_006121 | Keratin, type II cytoskeletal 1 (Cytokeratin-1) (CK-1) |
| | | (Keratin-1) (K1) (67 kDa cytokeratin) (Hair alpha protein). |
| | | [Source: Uniprot/SWISSPROT; Acc: P04264] |
| 3902 | NM_002286 | Lymphocyte activation gene 3 protein precursor (LAG-3) |
| | | (FDC protein) (CD223 antigen). |
| | | [Source: Uniprot/SWISSPROT; Acc: P18627] |
| 3903 | NM_021708; NM_002287; | leukocyte-associated Ig-like receptor 1; LAIR1 |
| | NM_021706 | |
| 3904 | NM_002288; NM_021270 | leukocyte-associated Ig-like receptor 2; LAIR2 |
| 3929 | NM_004139 | lipopolysaccharide binding protein; LBP |
| 3932 | NM_005356 | lymphocyte-specific protein tyrosine kinase; LCK |
| 3933 | NM_002297 | Lipocalin-1 precursor (Von Ebner gland protein) (VEG |
| | | protein) (Tear prealbumin) (TP) (Tear lipocalin) (Tlc). |
| | | [Source: Uniprot/SWISSPROT; Acc: P31025] |
| 3936 | NM_002298 | Lymphocyte cytosolic protein 1 (L-plastin) (LCP1) |
| 3937 | NM_005565 | lymphocyte cytosolic protein 2 (SH2 domain containing |
| | | leukocyte protein of 76 kDa); LCP2 |
| 3959 | NM_005567 | Galectin-3 binding protein precursor (Lectin galactoside- |
| | | binding soluble 3 binding protein) (Mac-2 binding protein) |
| | | (Mac-2 BP) (MAC2BP) (Tumor-associated antigen 90K). |
| | | [Source: Uniprot/SWISSPROT; Acc: Q08380] |
| 3976 | NM_002309 | Leukemia inhibitory factor precursor (LIF) (Differentiation- |
| | | stimulating factor) (D factor) (Melanoma-derived LPL |
| | | inhibitor) (MLPLI) (Emfilermin). |
| | | [Source: Uniprot/SWISSPROT; Acc: P15018] |
| 4046 | NM_001013253 | Lymphocyte-specific protein 1 (Protein pp52) (52 kDa |
| | | phosphoprotein) (Lymphocyte-specific antigen WP34) (47 kDa |
| | | actin binding protein). |
| | | [Source: Uniprot/SWISSPROT; Acc: P33241] |
| 4048 | NM_000895 | Leukotriene A-4 hydrolase (EC 3.3.2.6) (LTA-4 hydrolase) |
| | | (Leukotriene A(4) hydrolase). |
| | | [Source: Uniprot/SWISSPROT; Acc: P09960] |
| 4049 | NM_000595 | Lymphotoxin-alpha precursor (LT-alpha) (TNF-beta) |
| | | (Tumor necrosis factor ligand superfamily member 1). |
| | | [Source: Uniprot/SWISSPROT; Acc: P01374] |
| 4050 | NM_002341 | Lymphotoxin-beta (LT-beta) (Tumor necrosis factor C) |
| | | (TNF-C) (Tumor necrosis factor ligand superfamily member |
| | | 3). [Source: Uniprot/SWISSPROT; Acc: Q06643] |
| 4055 | NM_002342 | Tumor necrosis factor receptor superfamily member 3 |
| | | precursor (Lymphotoxin-beta receptor) (Tumor necrosis |
| | | factor receptor 2 related protein) (Tumor necrosis factor C |
| | | receptor). [Source: Uniprot/SWISSPROT; Acc: P36941] |
| 4057 | NM_002343 | Lactotransferrin precursor (EC 3.4.21.—) (Lactoferrin) |
| | | [Contains: Kaliocin-1; Lactoferroxin A; Lactoferroxin B; |
| | | Lactoferroxin C]. [Source: Uniprot/SWISSPROT; Acc: P02788] |
| 4061 | NM_002346 | Lymphocyte antigen 6 complex, locus E (LY6E) |
| 4062 | NM_002347 | Lymphocyte antigen Ly-6H precursor. |
| | | [Source: Uniprot/SWISSPROT; Acc: O94772] |
| 4063 | NM_001033667; NM_002348 | lymphocyte antigen 9; LY9 |
| 4064 | NM_005582 | CD180 antigen precursor (Lymphocyte antigen 64) |
| | | (Radioprotective 105 kDa protein). |
| | | [Source: Uniprot/SWISSPROT; Acc: Q99467] |
| 4065 | NM_002349 | Lymphocyte antigen 75 precursor (DEC-205) (CD205 |
| | | antigen) (gp200-MR6). |
| | | [Source: Uniprot/SWISSPROT; Acc: O60449] |
| 4067 | NM_002350 | v-yes-1 Yamaguchi sarcoma viral related oncogene |
| | | homolog; LYN |
| 4068 | NM_002351 | SH2 domain protein 1A (Signaling lymphocyte activation |
| | | molecule-associated protein) (SLAM-associated protein) (T |
| | | cell signal transduction molecule SAP) (Duncan disease |
| | | SH2-protein). [Source: Uniprot/SWISSPROT; Acc: O60880] |
| 4069 | NM_000239 | lysozyme (renal amyloidosis); LYZ |
| 4088 | NM_005902 | Mothers against decapentaplegic homolog 3 (SMAD 3) |
| | | (Mothers against DPP homolog 3) (Mad3) (hMAD-3) (JV15- |
| | | 2) (hSMAD3). [Source: Uniprot/SWISSPROT; Acc: P84022] |
| 4153 | NM_000242 | mannose-binding lectin (protein C) 2, soluble (opsonic |
| | | defect); MBL2 |
| 4155 | NM_001025090 | Myelin basic protein (MBP) (Myelin A1 protein) (Myelin |
| | | membrane encephalitogenic protein). |
| | | [Source: Uniprot/SWISSPROT; Acc: P02686] |
| 4179 | NM_172359; NM_172356; | membrane cofactor protein (CD46, trophoblast-lymphocyte |
| | NM_172351; NM_172353; | cross-reactive antigen); MCP |
| | NM_002389; NM_172350; | |
| | NM_153826; NM_172361; | |
| | NM_172354; NM_172360; | |
| | NM_172355; NM_172358; | |
| | NM_172357; NM_172352 | |
| 4210 | NM_000243 | Pyrin (Marenostrin). |
| | | [Source: Uniprot/SWISSPROT; Acc: O15553] |
| 4215 | NM_203351; NM_002401 | mitogen-activated protein kinase kinase kinase 3; MAP3K3 |
| 4258 | NM_002413 | Microsomal glutathione S-transferase 2 (EC 2.5.1.18) |
| | | (Microsomal GST-2) (Microsomal GST-II). |
| | | [Source: Uniprot/SWISSPROT; Acc: Q99735] |
| 4259 | NM_004528 | Microsomal glutathione S-transferase 3 (EC 2.5.1.18) |
| | | (Microsomal GST-3) (Microsomal GST-III). |
| | | [Source: Uniprot/SWISSPROT; Acc: O14880] |
| 4261 | NM_000246 | MHC class II transactivator (CIITA). |
| | | [Source: Uniprot/SWISSPROT; Acc: P33076] |
| 4276 | NM_000247 | MHC class I polypeptide-related sequence A; MICA |
| 4277 | NM_005931 | MHC class I polypeptide-related sequence B; MICB |
| 4282 | NM_002415 | Macrophage migration inhibitory factor (MIF) |
| | | (Phenylpyruvate tautomerase) (EC 5.3.2.1) (Glycosylation- |
| | | inhibiting factor) (GIF). |
| | | [Source: Uniprot/SWISSPROT; Acc: P14174] |
| 4283 | NM_002416 | Small inducible cytokine B9 precursor (CXCL9) (Gamma |
| | | interferon-induced monokine) (MIG). |
| | | [Source: Uniprot/SWISSPROT; Acc: Q07325] |
| 4332 | NM_002432 | Myeloid cell nuclear differentiation antigen. |
| | | [Source: Uniprot/SWISSPROT; Acc: P41218] |
| 4345 | NM_001004196; NM_005944; | CD200 antigen; CD200 |
| | NM_001004197 | |
| 4481 | NM_002445; NM_138715; | macrophage scavenger receptor 1; MSR1 |
| | NM_138716 | |
| 4485 | NM_020998 | macrophage stimulating 1 (hepatocyte growth factor- |
| | | like); MST1 |
| 4599 | NM_002462 | Interferon-induced GTP-binding protein Mx1 (Interferon- |
| | | regulated resistance GTP-binding protein MxA) (Interferon- |
| | | induced protein p78) (IFI-78K). |
| | | [Source: Uniprot/SWISSPROT; Acc: P20591] |
| 4600 | NM_002463 | Interferon-induced GTP-binding protein Mx2 (Interferon- |
| | | regulated resistance GTP-binding protein MxB) (p78-related |
| | | protein). [Source: Uniprot/SWISSPROT; Acc: P20592] |
| 4615 | NM_002468 | Myeloid differentiation primary response protein MyD88. |
| | | [Source: Uniprot/SWISSPROT; Acc: Q99836] |
| 4687 | NM_000265 | Neutrophil cytosol factor 1 (NCF-1) (Neutrophil NADPH |
| | | oxidase factor 1) (47 kDa neutrophil oxidase factor) (p47- |
| | | phox) (NCF-47K) (47 kDa autosomal chronic granulomatous |
| | | disease protein) (NOXO2). |
| | | [Source: Uniprot/SWISSPROT; Acc: P14598] |
| 4688 | NM_000433 | Neutrophil cytosol factor 2 (NCF-2) (Neutrophil NADPH |
| | | oxidase factor 2) (67 kDa neutrophil oxidase factor) (p67- |
| | | phox) (NOXA2). [Source: Uniprot/SWISSPROT; Acc: P19878] |
| 4690 | NM_006153 | NCK adaptor protein 1; NCK1 |
| 4772 | NM_172387; NM_006162; | nuclear factor of activated T-cells, cytoplasmic, calcineurin- |
| | NM_172388; NM_172390; | dependent 1; NFATC1 |
| | NM_172389 | |
| 4773 | NM_173091; NM_012340 | nuclear factor of activated T-cells, cytoplasmic, calcineurin- |
| | | dependent 2; NFATC2 |
| 4775 | NM_173164; NM_004555; | nuclear factor of activated T-cells, cytoplasmic, calcineurin- |
| | NM_173165; NM_173163 | dependent 3; NFATC3 |
| 4776 | NM_004554 | nuclear factor of activated T-cells, cytoplasmic, calcineurin- |
| | | dependent 4; NFATC4 |
| 4779 | NM_003204 | Nuclear factor erythroid 2 related factor 1 (NF-E2 related |
| | | factor 1) (NFE2-related factor 1) (Nuclear factor, erythroid |
| | | derived 2, like 1) (Transcription factor 11) (Transcription |
| | | factor HBZ17) (Transcription factor LCR-F1) (Locus control |
| | | region-factor 1) |
| 4783 | NM_005384 | nuclear factor, interleukin 3 regulated |
| | | [Source: RefSeq_peptide; Acc: NP_005375] |
| 4790 | NM_003998 | nuclear factor of kappa light polypeptide gene enhancer in |
| | | B-cells 1 (p105); NFKB1 |
| 4791 | NM_002502 | nuclear factor of kappa light polypeptide gene enhancer in |
| | | B-cells 2 (p49/p100); NFKB2 |
| 4792 | NM_020529 | nuclear factor of kappa light polypeptide gene enhancer in |
| | | B-cells inhibitor, alpha; NFKBIA |
| 4793 | NM_002503; NM_001001716 | nuclear factor of kappa light polypeptide gene enhancer in |
| | | B-cells inhibitor, beta; NFKBIB |
| 4795 | NM_005007 | nuclear factor of kappa light polypeptide gene enhancer in |
| | | B-cells inhibitor-like 1; NFKBIL1 |
| 4843 | NM_000625 | Nitric oxide synthase, inducible (EC 1.14.13.39) (NOS type |
| | | II) (Inducible NO synthase) (Inducible NOS) (iNOS) |
| | | (Hepatocyte NOS) (HEP-NOS). |
| | | [Source: Uniprot/SWISSPROT; Acc: P35228] |
| 4851 | NM_017617 | Notch homolog 1, translocation-associated |
| | | (Drosophila); NOTCH1 |
| 4853 | NM_024408 | Notch homolog 2 (Drosophila); NOTCH2 |
| 4854 | NM_000435 | Notch homolog 3 (Drosophila); NOTCH3 |
| 4855 | NM_004557 | Notch homolog 4 (Drosophila); NOTCH4 |
| 4884 | NM_002522 | neuronal pentraxin I; NPTX1 |
| 4885 | NM_002523 | neuronal pentraxin II; NPTX2 |
| 4893 | NM_002524 | neuroblastoma RAS viral (v-ras) oncogene homolog; NRAS |
| 4929 | NM_173172 | Orphan nuclear receptor NR4A2 (Orphan nuclear receptor |
| | | NURR1) (Immediate-early response protein NOT) |
| | | (Transcriptionally inducible nuclear receptor). |
| | | [Source: Uniprot/SWISSPROT; Acc: P43354] |
| 4938 | NM_001032409; NM_016816; | 2′,5′-oligoadenylate synthetase 1, 40/46 kDa; OAS1 |
| | NM_002534 | |
| 4939 | NM_016817; NM_001032731; | 2′-5′-oligoadenylate synthetase 2, 69/71 kDa; OAS2 |
| | NM_002535 | |
| 4940 | NM_006187 | 2′-5′-oligoadenylate synthetase 3, 100 kDa; OAS3 |
| 4973 | NM_002543 | Oxidized low-density lipoprotein receptor 1 (Ox-LDL |
| | | receptor 1) (Lectin-type oxidized LDL receptor 1) (Lectin- |
| | | like oxidized LDL receptor 1) (Lectin-like oxLDL receptor 1) |
| | | (LOX-1) (hLOX-1) [Contains: Oxidized low-density |
| | | lipoprotein receptor 1, soluble for |
| 4985 | NM_000911 | Delta-type opioid receptor (DOR-1). |
| | | [Source: Uniprot/SWISSPROT; Acc: P41143] |
| 4986 | NM_000912 | Kappa-type opioid receptor (KOR-1). |
| | | [Source: Uniprot/SWISSPROT; Acc: P41145] |
| 5004 | NM_000607 | Alpha-1-acid glycoprotein 2 precursor (AGP 2) |
| | | (Orosomucoid-2) (OMD 2). |
| | | [Source: Uniprot/SWISSPROT; Acc: P19652] |
| 5005 | NM_000608 | Alpha-1-acid glycoprotein 2 precursor (AGP 2) |
| | | (Orosomucoid-2) (OMD 2). |
| | | [Source: Uniprot/SWISSPROT; Acc: P19652] |
| 5008 | NM_020530 | Oncostatin M precursor (OSM). |
| | | [Source: Uniprot/SWISSPROT; Acc: P13725] |
| 5058 | NM_002576 | p21/Cdc42/Rac1-activated kinase 1 (STE20 homolog, |
| | | yeast); PAK1 |
| 5062 | NM_002577 | p21 (CDKN1A)-activated kinase 2; PAK2 |
| 5063 | NM_002578 | p21 (CDKN1A)-activated kinase 3; PAK3 |
| 5068 | NM_002580 | Regenerating islet-derived protein 3 alpha precursor (Reg |
| | | III-alpha) (Pancreatitis-associated protein 1). |
| | | [Source: Uniprot/SWISSPROT; Acc: Q06141] |
| 5074 | NM_002583 | PRKC apoptosis WT1 regulator protein (Prostate apoptosis |
| | | response-4 protein) (Par-4). |
| | | [Source: Uniprot/SWISSPROT; Acc: Q96IZ0] |
| 5079 | NM_016734 | Paired box protein Pax-5 (B-cell-specific transcription |
| | | factor) (BSAP). [Source: Uniprot/SWISSPROT; Acc: Q02548] |
| 5133 | NM_005018 | Programmed cell death protein 1 precursor (Protein PD-1) |
| | | (hPD-1) (CD279 antigen). |
| | | [Source: Uniprot/SWISSPROT; Acc: Q15116] |
| 5153 | AB209200 | Phosphodiesterase 1B, |
| | | calmodulin-dependent variant protein. |
| 5175 | NM_000442 | platelet/endothelial cell adhesion molecule (CD31 |
| | | antigen); PECAM1 |
| 5196 | NM_002619 | Platelet factor 4 precursor (PF-4) (CXCL4) (Oncostatin A) |
| | | (Iroplact). [Source: Uniprot/SWISSPROT; Acc: P02776] |
| 5197 | NM_002620 | Platelet factor 4 variant precursor (PF4var1) (PF4alt) |
| | | (CXCL4L1) [Contains: Platelet factor 4 variant(4-74); |
| | | Platelet factor 4 variant(5-74); Platelet factor 4 variant(6- |
| | | 74)]. [Source: Uniprot/SWISSPROT; Acc: P10720] |
| 5199 | NM_002621 | Properdin precursor (Factor P). |
| | | [Source: Uniprot/SWISSPROT; Acc: P27918] |
| 5265 | NM_001002236 | Alpha-1-antitrypsin precursor (Alpha-1 protease inhibitor) |
| | | (Alpha-1-antiproteinase). |
| | | [Source: Uniprot/SWISSPROT; Acc: P01009] |
| 5284 | NM_002644 | polymeric immunoglobulin receptor; PIGR |
| 5286 | NM_002645 | phosphoinositide-3-kinase, class 2, alpha |
| | | polypeptide; PIK3C2A |
| 5287 | NM_002646 | phosphoinositide-3-kinase, class 2, beta |
| | | polypeptide; PIK3C2B |
| 5288 | NM_004570 | phosphoinositide-3-kinase, class 2, gamma |
| | | polypeptide; PIK3C2G |
| 5289 | NM_002647 | phosphoinositide-3-kinase, class 3; PIK3C3 |
| 5290 | NM_006218 | phosphoinositide-3-kinase, catalytic, alpha |
| | | polypeptide; PIK3CA |
| 5291 | NM_006219 | phosphoinositide-3-kinase, catalytic, beta |
| | | polypeptide; PIK3CB |
| 5293 | NM_005026 | phosphoinositide-3-kinase, catalytic, delta |
| | | polypeptide; PIK3CD |
| 5294 | NM_002649 | phosphoinositide-3-kinase, catalytic, gamma |
| | | polypeptide; PIK3CG |
| 5295 | NM_181523; NM_181504; | phosphoinositide-3-kinase, regulatory subunit 1 (p85 |
| | NM_181524 | alpha); PIK3R1 |
| 5296 | NM_005027 | phosphoinositide-3-kinase, regulatory subunit 2 (p85 |
| | | beta); PIK3R2 |
| 5330 | NM_004573 | Phospholipase C, beta 2 (PLCB2) |
| 5335 | NM_002660; NM_182811 | phospholipase C, gamma 1; PLCG1 |
| 5336 | NM_002661 | phospholipase C, gamma 2 (phosphatidylinositol- |
| | | specific); PLCG2 |
| 5345 | NM_000934 | Alpha-2-antiplasmin precursor (Alpha-2-plasmin inhibitor) |
| | | (Alpha-2-PI) (Alpha-2-AP). |
| | | [Source: Uniprot/SWISSPROT; Acc: P08697] |
| 5360 | NM_182676; NM_006227 | phospholipid transfer protein; PLTP |
| 5393 | NM_001034194 | Exosome complex exonuclease RRP45 (EC 3.1.13.—) |
| | | (Exosome component 9) (Polymyositis/scleroderma |
| | | autoantigen 1) (Autoantigen PM/Scl 1) |
| | | (Polymyositis/scleroderma autoantigen 75 kDa) (PM/Scl-75) |
| | | (P75 polymyositis-scleroderma overlap syndrome associated |
| | | autoa |
| 5408 | NM_005396 | Pancreatic lipase-related protein 2 precursor (EC 3.1.1.3). |
| | | [Source: Uniprot/SWISSPROT; Acc: P54317] |
| 5450 | NM_006235 | POU domain class 2, associating factor 1 (B-cell-specific |
| | | coactivator OBF-1) (OCT binding factor 1) (BOB-1) (OCA- |
| | | B). [Source: Uniprot/SWISSPROT; Acc: Q16633] |
| 5452 | NM_002698 | POU domain, class 2, transcription factor 2 (Octamer- |
| | | binding transcription factor 2) (Oct-2) (OTF-2) (Lymphoid- |
| | | restricted immunoglobulin octamer binding protein NF-A2). |
| | | [Source: Uniprot/SWISSPROT; Acc: P09086] |
| 5468 | NM_005037 | Peroxisome proliferator-activated receptor gamma (PPAR- |
| | | gamma). [Source: Uniprot/SWISSPROT; Acc: P37231] |
| 5473 | NM_002704 | Platelet basic protein precursor (PBP) (Small inducible |
| | | cytokine B7) (CXCL7) (Leukocyte-derived growth factor) |
| | | (LDGF) (Macrophage-derived growth factor) (MDGF) |
| | | [Contains: Connective tissue-activating peptide III (CTAP- |
| | | III) (Low-affinity platelet factor IV |
| 5479 | NM_000942 | Peptidylprolyl isomerase B (cyclophilin B) (PPIB) |
| 5530 | NM_000944 | protein phosphatase 3 (formerly 2B), catalytic subunit, alpha |
| | | isoform (calcineurin A alpha); PPP3CA |
| 5532 | NM_021132 | protein phosphatase 3 (formerly 2B), catalytic subunit, beta |
| | | isoform (calcineurin A beta); PPP3CB |
| 5533 | NM_005605 | protein phosphatase 3 (formerly 2B), catalytic subunit, |
| | | gamma isoform (calcineurin A gamma); PPP3CC |
| 5551 | NM_005041 | Perforin 1 precursor (P1) (Lymphocyte pore forming protein) |
| | | (PFP) (Cytolysin). [Source: Uniprot/SWISSPROT; Acc: P14222] |
| 5553 | NM_002728 | proteoglycan 2, bone marrow (natural killer cell activator, |
| | | eosinophil granule major basic protein); PRG2 |
| 5578 | NM_002737 | protein kinase C, alpha; PRKCA |
| 5579 | NM_212535; NM_002738 | protein kinase C, beta 1; PRKCB1 |
| 5580 | NM_006254; NM_212539 | protein kinase C, delta; PRKCD |
| 5581 | NM_005400 | protein kinase C, epsilon; PRKCE |
| 5582 | NM_002739 | protein kinase C, gamma; PRKCG |
| 5583 | NM_006255 | protein kinase C, eta; PRKCH |
| 5588 | NM_006257 | protein kinase C, theta; PRKCQ |
| 5594 | NM_002745; NM_138957 | mitogen-activated protein kinase 1; MAPK1 |
| 5595 | NM_002746 | mitogen-activated protein kinase 3; MAPK3 |
| 5599 | NM_139046; NM_002750; | mitogen-activated protein kinase 8; MAPK8 |
| | NM_139049; NM_139047 | |
| 5600 | NM_138993; NM_002751 | mitogen-activated protein kinase 11; MAPK11 |
| 5601 | NM_139069; NM_139070; | mitogen-activated protein kinase 9; MAPK9 |
| | NM_139068; NM_002752 | |
| 5602 | NM_138980; NM_002753; | mitogen-activated protein kinase 10; MAPK10 |
| | NM_138982; NM_138981 | |
| 5603 | NM_002754 | mitogen-activated protein kinase 13; MAPK13 |
| 5604 | NM_002755 | mitogen-activated protein kinase kinase 1; MAP2K1 |
| 5605 | NM_030662 | mitogen-activated protein kinase kinase 2; MAP2K2 |
| 5606 | NM_145110 | Dual specificity mitogen-activated protein kinase kinase 3 |
| | | (EC 2.7.1.—) (MAP kinase kinase 3) (MAPKK 3) (MAPK/ERK |
| | | kinase 3). [Source: Uniprot/SWISSPROT; Acc: P46734] |
| 5610 | NM_002759 | Interferon-induced, double-stranded RNA-activated protein |
| | | kinase (EC 2.7.1.—) (Interferon-inducible RNA-dependent |
| | | protein kinase) (p68 kinase) (P1/eIF-2A protein kinase). |
| | | [Source: Uniprot/SWISSPROT; Acc: P19525] |
| 5618 | NM_000949 | Prolactin receptor precursor (PRL-R). |
| | | [Source: Uniprot/SWISSPROT; Acc: P16471] |
| 5624 | NM_000312 | protein C (inactivator of coagulation factors Va and |
| | | VIIIa); PROC |
| 5648 | NM_001879 | Complement-activating component of Ra-reactive factor |
| | | precursor (EC 3.4.21.—) (Ra-reactive factor serine protease |
| | | p100) (RaRF) (Mannan-binding lectin serine protease 1) |
| | | (Mannose-binding protein associated serine protease) |
| | | (MASP-1) [Contains: Complement-ac |
| 5696 | NM_148919 | Proteasome subunit beta type 8 precursor (EC 3.4.25.1) |
| | | (Proteasome component C13) (Macropain subunit C13) |
| | | (Multicatalytic endopeptidase complex subunit C13). |
| | | [Source: Uniprot/SWISSPROT; Acc: P28062] |
| 5698 | NM_002800 | Proteasome subunit beta type 9 precursor (EC 3.4.25.1) |
| | | (Proteasome chain 7) (Macropain chain 7) (Multicatalytic |
| | | endopeptidase complex chain 7) (RING12 protein) (Low |
| | | molecular mass protein 2). |
| | | [Source: Uniprot/SWISSPROT; Acc: P28065] |
| 5699 | NM_002801 | Proteasome subunit beta type 10 precursor (EC 3.4.25.1) |
| | | (Proteasome MECl-1) (Macropain subunit MECl-1) |
| | | (Multicatalytic endopeptidase complex subunit MECl-1). |
| | | [Source: Uniprot/SWISSPROT; Acc: P40306] |
| 5720 | NM_176783 | Proteasome activator complex subunit 1 (Proteasome |
| | | activator 28-alpha subunit) (PA28alpha) (PA28a) (Activator |
| | | of multicatalytic protease subunit 1) (11S regulator complex |
| | | alpha subunit) (REG-alpha) (Interferon gamma up- |
| | | regulated I-5111 protein) (IGUP I-51 |
| 5721 | NM_002818 | Proteasome activator complex subunit 2 (Proteasome |
| | | activator 28-beta subunit) (PA28beta) (PA28b) (Activator of |
| | | multicatalytic protease subunit 2) (11S regulator complex |
| | | beta subunit) (REG-beta). |
| | | [Source: Uniprot/SWISSPROT; Acc: Q9UL46] |
| 5724 | NM_000952 | Platelet-activating factor receptor (PAF-R). |
| | | [Source: Uniprot/SWISSPROT; Acc: P25105] |
| 5733 | NM_000957 | Prostaglandin E2 receptor, EP3 subtype (Prostanoid EP3 |
| | | receptor) (PGE receptor, EP3 subtype) (PGE2-R). |
| | | [Source: Uniprot/SWISSPROT; Acc: P43115] |
| 5734 | NM_000958 | Prostaglandin E2 receptor, EP4 subtype (Prostanoid EP4 |
| | | receptor) (PGE receptor, EP4 subtype). |
| | | [Source: Uniprot/SWISSPROT; Acc: P35408] |
| 5743 | NM_000963 | Prostaglandin G/H synthase 2 precursor (EC 1.14.99.1) |
| | | (Cyclooxygenase-2) (COX-2) (Prostaglandin-endoperoxide |
| | | synthase 2) (Prostaglandin H2 synthase 2) (PGH synthase 2) |
| | | (PGHS-2) (PHS II). |
| | | [Source: Uniprot/SWISSPROT; Acc: P35354] |
| 5763 | NM_002824 | Parathymosin. [Source: Uniprot/SWISSPROT; Acc: P20962] |
| 5777 | NM_002831; NM_080548; | protein tyrosine phosphatase, non-receptor type 6; PTPN6 |
| | NM_080549 | |
| 5788 | NM_080921; NM_080922; | protein tyrosine phosphatase, receptor type, C; PTPRC |
| | NM_002838; NM_080923 | |
| 5806 | NM_002852 | pentraxin-related gene, rapidly induced by IL-1 beta; PTX3 |
| 5817 | NM_006505 | poliovirus receptor; PVR |
| 5818 | NM_002855; NM_203285; | poliovirus receptor-related 1 (herpesvirus entry mediator C; |
| | NM_203286 | nectin); PVRL1 |
| 5819 | NM_002856 | poliovirus receptor-related 2 (herpesvirus entry mediator |
| | | B); PVRL2 |
| 5871 | NM_004579 | Mitogen-activated protein kinase kinase kinase kinase 2 (EC |
| | | 2.7.1.37) (MAPK/ERK kinase kinase kinase 2) (MEK kinase |
| | | kinase 2) (MEKKK 2) (Germinal center kinase) (GC kinase) |
| | | (Rab8 interacting protein) (B lymphocyte serine/threonine- |
| | | protein kinase). [Source |
| 5879 | NM_006908; NM_018890; | ras-related C3 botulinum toxin substrate 1 (rho family, small |
| | NM_198829 | GTP binding protein Rac1); RAC1 |
| 5880 | NM_002872 | ras-related C3 botulinum toxin substrate 2 (rho family, small |
| | | GTP binding protein Rac2); RAC2 |
| 5881 | NM_005052 | ras-related C3 botulinum toxin substrate 3 (rho family, small |
| | | GTP binding protein Rac3); RAC3 |
| 5894 | NM_002880 | v-raf-1 murine leukemia viral oncogene homolog 1; RAF1 |
| 5896 | NM_000448 | V(D)J recombination-activating protein 1 (RAG-1) (RING |
| | | finger protein 74). |
| | | [Source: Uniprot/SWISSPROT; Acc: P15918] |
| 5897 | NM_000536 | V(D)J recombination-activating protein 2 (RAG-2). |
| | | [Source: Uniprot/SWISSPROT; Acc: P55895] |
| 5970 | NM_021975 | Transcription factor p65 (Nuclear factor NF-kappa-B p65 |
| | | subunit). [Source: Uniprot/SWISSPROT; Acc: Q04206] |
| 5971 | NM_006509 | Transcription factor RelB (I-Rel). |
| | | [Source: Uniprot/SWISSPROT; Acc: Q01201] |
| 5989 | NM_002918 | MHC class II regulatory factor RFX1 (RFX) (Enhancer factor |
| | | C) (EF-C). [Source: Uniprot/SWISSPROT; Acc: P22670] |
| 5996 | NM_002922 | Regulator of G-protein signaling 1 (RGS1) (Early response |
| | | protein 1R20) (B-cell activation protein BL34). |
| | | [Source: Uniprot/SWISSPROT; Acc: Q08116] |
| 5997 | NM_002923 | Regulator of G-protein signalling 2, 24 kDa (RGS2) |
| 6091 | NM_002941; NM_133631 | roundabout, axon guidance receptor, homolog 1 |
| | | (Drosophila); ROBO1 |
| 6092 | NM_002942 | roundabout, axon guidance receptor, homolog 2 |
| | | (Drosophila); ROBO2 |
| 6237 | NM_006270 | related RAS viral (r-ras) oncogene homolog; RRAS |
| 6279 | NM_002964 | Calgranulin A (Migration inhibitory factor-related protein 8) |
| | | (MRP-8) (Cystic fibrosis antigen) (CFAG) (P8) (Leukocyte L1 |
| | | complex light chain) (S100 calcium-binding protein A8) |
| | | (Calprotectin L1L subunit) (Urinary stone protein band A). |
| | | [Source: Uniprot/SWI |
| 6280 | NM_002965 | Calgranulin B (Migration inhibitory factor-related protein 14) |
| | | (MRP-14) (P14) (Leukocyte L1 complex heavy chain) (S100 |
| | | calcium-binding protein A9) (Calprotectin L1H subunit). |
| | | [Source: Uniprot/SWISSPROT; Acc: P06702] |
| 6283 | NM_005621 | Calgranulin C (CAGC) (CGRP) (Neutrophil S100 protein) |
| | | (Calcium-binding protein in amniotic fluid 1) (CAAF1) (p6) |
| | | [Contains: Calcitermin]. |
| | | [Source: Uniprot/SWISSPROT; Acc: P80511] |
| 6285 | NM_006272 | S-100 calcium-binding protein beta subunit (S-100 protein, |
| | | beta chain). [Source: Uniprot/SWISSPROT; Acc: P04271] |
| 6288 | NM_199161; NM_000331 | serum amyloid A1; SAA1 |
| 6289 | NM_030754 | serum amyloid A2; SAA2 |
| 6291 | NM_006512 | serum amyloid A4, constitutive; SAA4 |
| 6300 | NM_002969 | mitogen-activated protein kinase 12; MAPK12 |
| 6318 | NM_002974 | Squamous cell carcinoma antigen 1 (SCCA-1) (Protein T4- |
| | | A). [Source: Uniprot/SWISSPROT; Acc: P29508] |
| 6346 | NM_002981 | Small inducible cytokine A1 precursor (CCL1) (T |
| | | lymphocyte-secreted protein I-309). |
| | | [Source: Uniprot/SWISSPROT; Acc: P22362] |
| 6347 | NM_002982 | Small inducible cytokine A2 precursor (CCL2) (Monocyte |
| | | chemotactic protein 1) (MCP-1) (Monocyte chemoattractant |
| | | protein 1) (Monocyte chemotactic and activating factor) |
| | | (MCAF) (Monocyte secretory protein JE) (HC11). |
| | | [Source: Uniprot/SWISSPROT; Acc: P13500] |
| 6348 | NM_002983 | Small inducible cytokine A3 precursor (CCL3) (Macrophage |
| | | inflammatory protein 1-alpha) (MIP-1-alpha) (Tonsillar |
| | | lymphocyte LD78 alpha protein) (G0/G1 switch regulatory |
| | | protein 19-1) (G0S19-1 protein) (SIS-beta) (PAT 464.1) |
| | | [Contains: MIP-1-alpha(4-69) (LD |
| 6349 | NM_021006 | Small inducible cytokine A3-like 1 precursor (Tonsillar |
| | | lymphocyte LD78 beta protein) (LD78-beta(1-70)) (G0/G1 |
| | | switch regulatory protein 19-2) (G0S19-2 protein) (PAT |
| | | 464.2) [Contains: LD78-beta(3-70); LD78-beta(5-70)]. |
| | | [Source: Uniprot/SWISSPROT; Acc: P1661 |
| 6351 | NM_002984 | Small inducible cytokine A4 precursor (CCL4) (Macrophage |
| | | inflammatory protein 1-beta) (MIP-1-beta) (MIP-1-beta(1- |
| | | 69)) (T-cell activation protein 2) (ACT-2) (PAT 744) (H400) |
| | | (SIS-gamma) (Lymphocyte activation gene 1 protein) (LAG- |
| | | 1) (HC21) (G-26 T lymphocy |
| 6352 | NM_002985 | Small inducible cytokine A5 precursor (CCL5) (T-cell-specific |
| | | RANTES protein) (SIS-delta) (T cell-specific protein P228) |
| | | (TCP228) [Contains: RANTES(3-68); RANTES(4-68)]. |
| | | [Source: Uniprot/SWISSPROT; Acc: P13501] |
| 6354 | NM_006273 | Small inducible cytokine A7 precursor (CCL7) (Monocyte |
| | | chemotactic protein 3) (MCP-3) (Monocyte chemoattractant |
| | | protein 3) (NC28). |
| | | [Source: Uniprot/SWISSPROT; Acc: P80098] |
| 6355 | NM_005623 | Small inducible cytokine A8 precursor (CCL8) (Monocyte |
| | | chemotactic protein 2) (MCP-2) (Monocyte chemoattractant |
| | | protein 2) (HC14) [Contains: MCP-2(6-76)]. |
| | | [Source: Uniprot/SWISSPROT; Acc: P80075] |
| 6356 | NM_002986 | Eotaxin precursor (Small inducible cytokine A11) (CCL11) |
| | | (Eosinophil chemotactic protein). |
| | | [Source: Uniprot/SWISSPROT; Acc: P51671] |
| 6357 | NM_005408 | Small inducible cytokine A13 precursor (CCL13) (Monocyte |
| | | chemotactic protein 4) (MCP-4) (Monocyte chemoattractant |
| | | protein 4) (CK-beta-10) (NCC-1) [Contains: Small inducible |
| | | cytokine A13, long isoform; Small inducible cytokine A13, |
| | | medium isoform; Small in |
| 6359 | NM_004167 | Small inducible cytokine A15 precursor (CCL15) |
| | | (Macrophage inflammatory protein 5) (MIP-5) (Chemokine |
| | | CC-2) (HCC-2) (NCC-3) (MIP-1 delta) (Leukotactin-1) |
| | | (LKN-1) (Mrp-2b) [Contains: CCL15(22-92); CCL15(25-92); |
| | | CCL15(29-92)]. [Source: Uniprot/SWISSPROT; Acc |
| 6360 | NM_004590 | Small inducible cytokine A16 precursor (CCL16) (IL-10- |
| | | inducible chemokine) (Chemokine LEC) (Liver-expressed |
| | | chemokine) (Monotactin-1) (MTN-1) (Chemokine CC-4) |
| | | (HCC-4) (NCC-4) (Lymphocyte and monocyte |
| | | chemoattractant) (LMC) (LCC-1). [Source: Uniprot/SWISSPR |
| 6361 | NM_002987 | Small inducible cytokine A17 precursor (CCL17) (Thymus |
| | | and activation-regulated chemokine) (CC chemokine |
| | | TARC). [Source: Uniprot/SWISSPROT; Acc: Q92583] |
| 6362 | NM_002988 | Small inducible cytokine A18 precursor (CCL18) |
| | | (Macrophage inflammatory protein 4) (MIP-4) (Pulmonary |
| | | and activation-regulated chemokine) (CC chemokine PARC) |
| | | (Alternative macrophage activation-associated CC |
| | | chemokine 1) (AMAC-1) (Dendritic cell chemokine |
| 6363 | NM_006274 | Small inducible cytokine A19 precursor (CCL19) |
| | | (Macrophage inflammatory protein 3 beta) (MIP-3-beta) |
| | | (EBI1-ligand chemokine) (ELC) (Beta chemokine exodus-3) |
| | | (CK beta-11). [Source: Uniprot/SWISSPROT; Acc: Q99731] |
| 6364 | NM_004591 | Small inducible cytokine A20 precursor (CCL20) |
| | | (Macrophage inflammatory protein 3 alpha) (MIP-3-alpha) |
| | | (Liver and activation-regulated chemokine) (CC chemokine |
| | | LARC) (Beta chemokine exodus-1) [Contains: CCL20(1-67); |
| | | CCL20(1-64); CCL20(2-70)]. [Source: Uni |
| 6366 | NM_002989 | Small inducible cytokine A21 precursor (CCL21) (Beta |
| | | chemokine exodus-2) (6Ckine) (Secondary lymphoid-tissue |
| | | chemokine) (SLC). |
| | | [Source: Uniprot/SWISSPROT; Acc: O00585] |
| 6367 | NM_002990 | Small inducible cytokine A22 precursor (CCL22) |
| | | (Macrophage-derived chemokine) (MDC(1-69)) (Stimulated |
| | | T cell chemotactic protein 1) (CC chemokine STCP-1) |
| | | [Contains: MDC(3-69); MDC(5-69); MDC(7-69)]. |
| | | [Source: Uniprot/SWISSPROT; Acc: O00626] |
| 6368 | NM_005064 | Small inducible cytokine A23 precursor (CCL23) |
| | | (Macrophage inflammatory protein 3) (MIP-3) (Myeloid |
| | | progenitor inhibitory factor 1) (MPIF-1) (CK-beta-8) (CKB- |
| | | 8) [Contains: CCL23(19-99); CCL23(22-99); CCL23(27-99); |
| | | CCL23(30-99)]. [Source: Uniprot/SWISSPROT; |
| 6369 | NM_002991 | Small inducible cytokine A24 precursor (CCL24) (Myeloid |
| | | progenitor inhibitory factor 2) (MPIF-2) (CK-beta-6) |
| | | (Eosinophil chemotactic protein 2) (Eotaxin-2). |
| | | [Source: Uniprot/SWISSPROT; Acc: O00175] |
| 6370 | NM_005624 | Small inducible cytokine A25 precursor (CCL25) (Chemokine |
| | | TECK) (Thymus expressed chemokine). |
| | | Source: Uniprot/SWISSPROT; Acc: O15444] |
| 6372 | NM_002993 | Small inducible cytokine B6 precursor (CXCL6) (Granulocyte |
| | | chemotactic protein 2) (GCP-2) (Chemokine alpha 3) (CKA- |
| | | 3) [Contains: Small inducible cytokine B6, N-processed |
| | | variant 1; Small inducible cytokine B6, N-processed variant |
| | | 2; Small inducible cytoki |
| 6373 | NM_005409 | Small inducible cytokine B11 precursor (CXCL11) |
| | | (Interferon-inducible T-cell alpha chemoattractant) (I-TAC) |
| | | (Interferon-gamma-inducible protein 9) (IP-9) (H174) |
| | | (Beta-R1). [Source: Uniprot/SWISSPROT; Acc: O14625] |
| 6374 | NM_002994 | Small inducible cytokine B5 precursor (CXCL5) (Epithelial- |
| | | derived neutrophil-activating protein 78) (Neutrophil- |
| | | activating peptide ENA-78) (ENA-78(1-78)) [Contains: |
| | | ENA-78(8-78); ENA-78(9-78)]. |
| | | [Source: Uniprot/SWISSPROT; Acc: P42830] |
| 6375 | NM_002995 | Lymphotactin precursor (XCL1) (Cytokine SCM-1) (ATAC) |
| | | (Lymphotaxin) (SCM-1-alpha) (Small inducible cytokine C1) |
| | | (XC chemokine ligand 1). |
| | | [Source: Uniprot/SWISSPROT; Acc: P47992] |
| 6376 | NM_002996 | Fractalkine precursor (CX3CL1) (Neurotactin) (CX3C |
| | | membrane-anchored chemokine) (Small inducible cytokine |
| | | D1). [Source: Uniprot/SWISSPROT; Acc: P78423] |
| 6383 | NM_002998 | syndecan 2 (heparan sulfate proteoglycan 1, cell surface- |
| | | associated, fibroglycan); SDC2 |
| 6385 | NM_002999 | syndecan 4 (amphiglycan, ryudocan); SDC4 |
| 6387 | NM_000609 | Stromal cell-derived factor 1 precursor (SDF-1) (CXCL12) |
| | | (Pre-B cell growth-stimulating factor) (PBSF) (hIRH) |
| | | [Contains: SDF-1-beta(3-72); SDF-1-alpha(3-67)]. |
| | | [Source: Uniprot/SWISSPROT; Acc: P48061] |
| 6398 | NM_003004 | Secreted and transmembrane protein 1 precursor (Protein |
| | | K12). [Source: Uniprot/SWISSPROT; Acc: Q8WVN6] |
| 6401 | NM_000450 | E-selectin precursor (Endothelial leukocyte adhesion |
| | | molecule 1) (ELAM-1) (Leukocyte-endothelial cell adhesion |
| | | molecule 2) (LECAM2) (CD62E antigen). |
| | | [Source: Uniprot/SWISSPROT; Acc: P16581] |
| 6403 | NM_003005 | P-selectin precursor (Granule membrane protein 140) |
| | | (GMP-140) (PADGEM) (CD62P antigen) (Leukocyte- |
| | | endothelial cell adhesion molecule 3) (LECAM3). |
| | | [Source: Uniprot/SWISSPROT; Acc: P16109] |
| 6435 | NM_005411 | surfactant, pulmonary-associated protein A1; SFTPA1 |
| 6436 | NM_006926 | surfactant, pulmonary-associated protein A2; SFTPA2 |
| 6441 | NM_003019 | surfactant, pulmonary-associated protein D; SFTPD |
| 6461 | NM_003028 | SHB (Src homology 2 domain containing) adaptor protein B |
| | | [Source: RefSeq_peptide; Acc: NP_003019] |
| 6480 | NM_173217 | CMP-N-acetylneuraminate-beta-galactosamide-alpha-2,6- |
| | | sialyltransferase (EC 2.4.99.1) (Beta-galactoside alpha-2,6- |
| | | sialyltransferase) (Alpha 2,6-ST) (Sialyltransferase 1) |
| | | (ST6Gal I) (B-cell antigen CD75). |
| | | [Source: Uniprot/SWISSPROT; Acc: P15907] |
| 6504 | NM_003037 | signaling lymphocytic activation molecule family member |
| | | 1; SLAMF1 |
| 6556 | AF229163 | Natural resistance-associated macrophage protein 1 |
| | | (SLC11A1) |
| 6614 | NM_023068 | Sialoadhesin precursor (Sialic acid-binding Ig-like lectin-1) |
| | | (Siglec-1) (CD169 antigen). |
| | | [Source: Uniprot/SWISSPROT; Acc: Q9BZZ2] |
| 6648 | NM_001024465 | Superoxide dismutase [Mn], mitochondrial precursor (EC |
| | | 1.15.1.1). [Source: Uniprot/SWISSPROT; Acc: P04179] |
| 6654 | NM_005633 | son of sevenless homolog 1 (Drosophila); SOS1 |
| 6655 | NM_006939 | son of sevenless homolog 2 (Drosophila); SOS2 |
| 6668 | NM_003110 | Transcription factor Sp2. |
| | | [Source: Uniprot/SWISSPROT; Acc: Q02086] |
| 6670 | NM_001017371 | Transcription factor Sp3 (SPR-2). |
| | | [Source: Uniprot/SWISSPROT; Acc: Q02447] |
| 6688 | NM_003120 | Transcription factor PU.1 (31 kDa transforming protein). |
| | | [Source: Uniprot/SWISSPROT; Acc: P17947] |
| 6693 | NM_001030288 | Leukosialin precursor (Leucocyte sialoglycoprotein) |
| | | (Sialophorin) (CD43 antigen) (Galactoglycoprotein) (GALGP). |
| | | [Source: Uniprot/SWISSPROT; Acc: P16150] |
| 6696 | NM_000582 | secreted phosphoprotein 1 (osteopontin, bone sialoprotein I, |
| | | early T-lymphocyte activation 1); SPP1 |
| 6714 | NM_005417; NM_198291 | v-src sarcoma (Schmidt-Ruppin A-2) viral oncogene |
| | | homolog (avian); SRC |
| 6774 | NM_139276 | Signal transducer and activator of transcription 3 (Acute- |
| | | phase response factor). |
| | | [Source: Uniprot/SWISSPROT; Acc: P40763] |
| 6776 | NM_003152 | Signal transducer and activator of transcription 5A. |
| | | [Source: Uniprot/SWISSPROT; Acc: P42229] |
| 6777 | NM_012448 | Signal transducer and activator of transcription 5B. |
| | | [Source: Uniprot/SWISSPROT; Acc: P51692] |
| 6846 | NM_003175 | Cytokine SCM-1 beta precursor (XCL2) (XC chemokine |
| | | ligand 2). [Source: Uniprot/SWISSPROT; Acc: Q9UBD3] |
| 6850 | NM_003177 | spleen tyrosine kinase; SYK |
| 6863 | NM_003182 | Protachykinin 1 precursor (PPT) [Contains: Substance P; |
| | | Neurokinin A (NKA) (Substance K) (Neuromedin L); |
| | | Neuropeptide K (NPK); Neuropeptide gamma; C-terminal |
| | | flanking peptide]. [Source: Uniprot/SWISSPROT; Acc: P20366] |
| 6869 | NM_015727 | Substance-P receptor (SPR) (NK-1 receptor) (NK-1R) |
| | | (Tachykinin receptor 1). |
| | | [Source: Uniprot/SWISSPROT; Acc: P25103] |
| 6890 | NM_000593 | Antigen peptide transporter 1 (APT1) (Peptide transporter |
| | | TAP1) (ATP-binding cassette sub-family B member 2) |
| | | (Peptide transporter PSF1) (Peptide supply factor 1) (PSF-1) |
| | | (Peptide transporter involved in antigen processing 1). |
| | | [Source: Uniprot/SWISSPROT; Ac |
| 6891 | NM_018833 | Antigen peptide transporter 2 (APT2) (Peptide transporter |
| | | TAP2) (Peptide transporter PSF2) (Peptide supply factor 2) |
| | | (PSF-2) (Peptide transporter involved in antigen processing |
| | | 2). [Source: Uniprot/SWISSPROT; Acc: Q03519] |
| 6892 | NM_003190; NM_172209; | TAP binding protein (tapasin); TAPBP |
| | NM_172208 | |
| 6932 | NM_201633 | Transcription factor 7 (T-cell-specific transcription factor 1) |
| | | (TCF-1) (T-cell factor 1). |
| | | [Source: Uniprot/SWISSPROT; Acc: P36402] |
| 6935 | NM_030751 | Transcription factor 8 (NIL-2-A zinc finger protein) |
| | | (Negative regulator of IL2). |
| | | [Source: Uniprot/SWISSPROT; Acc: P37275] |
| 6938 | NM_003205 | Transcription factor 12 (Transcription factor HTF-4) (E-box- |
| | | binding protein) (DNA-binding protein HTF4). |
| | | [Source: Uniprot/SWISSPROT; Acc: Q99081] |
| 6955 | BC100294 | T cell receptor alpha locus |
| 6957 | K02885 | T-cell receptor beta locus |
| 7006 | NM_003215 | tec protein tyrosine kinase; TEC |
| 7031 | NM_003225 | trefoil factor 1 (breast cancer, estrogen-inducible sequence |
| | | expressed in); TFF1 |
| 7040 | NM_000660 | Transforming growth factor beta-1 precursor (TGF-beta-1). |
| | | [Source: Uniprot/SWISSPROT; Acc: P01137] |
| 7096 | NM_003263 | Toll-like receptor 1 precursor (Toll/interleukin-1 receptor- |
| | | like protein) (TIL) (CD281 antigen). |
| | | [Source: Uniprot/SWISSPROT; Acc: Q15399] |
| 7097 | NM_003264 | Toll-like receptor 2 precursor (Toll/interleukin 1 receptor- |
| | | like protein 4) (CD282 antigen). |
| | | [Source: Uniprot/SWISSPROT; Acc: O60603] |
| 7098 | NM_003265 | Toll-like receptor 3 precursor (CD283 antigen). |
| | | [Source: Uniprot/SWISSPROT; Acc: O15455] |
| 7099 | NM_138554 | Toll-like receptor 4 precursor (hToll) (CD284 antigen). |
| | | [Source: Uniprot/SWISSPROT; Acc: O00206] |
| 7100 | NM_003268 | Toll-like receptor 5 precursor (Toll/interleukin-1 receptor- |
| | | like protein 3). [Source: Uniprot/SWISSPROT; Acc: O60602] |
| 7124 | NM_000594 | Tumor necrosis factor precursor (TNF-alpha) (Tumor |
| | | necrosis factor ligand superfamily member 2) (TNF-a) |
| | | (Cachectin) [Contains: Tumor necrosis factor, membrane |
| | | form; Tumor necrosis factor, soluble form]. |
| | | [Source: Uniprot/SWISSPROT; Acc: P01375] |
| 7130 | NM_007115 | Tumor necrosis factor-inducible protein TSG-6 precursor |
| | | (TNF-stimulated gene 6 protein) (Hyaluronate-binding |
| | | protein). [Source: Uniprot/SWISSPROT; Acc: P98066] |
| 7132 | NM_001065 | Tumor necrosis factor receptor superfamily member 1A |
| | | precursor (p60) (TNF-R1) (TNF-RI) (TNFR-I) (p55) |
| | | (CD120a antigen) [Contains: Tumor necrosis factor receptor |
| | | superfamily member 1A, membrane form; Tumor necrosis |
| | | factor-binding protein 1 (TBPI)]. [Source |
| 7133 | NM_001066 | tumor necrosis factor receptor superfamily, member |
| | | 1B; TNFRSF1B |
| 7177 | NM_003294 | Tryptase beta-1 precursor (EC 3.4.21.59) (Tryptase-1) |
| | | (Tryptase I). [Source: Uniprot/SWISSPROT; Acc: Q15661] |
| 7187 | AF110908 | TNF-receptor associated factor-3 (TRAF-3) |
| 7189 | NM_145803 | TNF receptor-associated factor 6 (Interleukin 1 signal |
| | | transducer) (RING finger protein 85). |
| | | [Source: Uniprot/SWISSPROT; Acc: Q9Y4K3] |
| 7273 | NM_133379 | titin isoform novex-3 |
| | | [Source: RefSeq_peptide; Acc: NP_596870] |
| 7292 | NM_003326 | Tumor necrosis factor ligand superfamily member 4 (OX40 |
| | | ligand) (OX40L) (Glycoprotein GP34) (TAX transcriptionally- |
| | | activated glycoprotein 1) (CD252 antigen). |
| | | [Source: Uniprot/SWISSPROT; Acc: P23510] |
| 7293 | NM_003327 | Tumor necrosis factor receptor superfamily member 4 |
| | | precursor (OX40L receptor) (ACT35 antigen) (TAX |
| | | transcriptionally-activated glycoprotein 1 receptor) (CD134 |
| | | antigen). [Source: Uniprot/SWISSPROT; Acc: P43489] |
| 7294 | NM_003328 | TXK tyrosine kinase; TXK |
| 7305 | NM_198125; NM_003332 | TYRO protein tyrosine kinase binding protein; TYROBP |
| 7369 | NM_003361 | Uromodulin precursor (Tamm-Horsfall urinary glycoprotein) |
| | | (THP). [Source: Uniprot/SWISSPROT; Acc: P07911] |
| 7409 | NM_005428 | vav 1 oncogene; VAV1 |
| 7410 | NM_003371 | vav 2 oncogene; VAV2 |
| 7422 | NM_001025366 | Vascular endothelial growth factor A precursor (VEGF-A) |
| | | (Vascular permeability factor) (VPF). |
| | | [Source: Uniprot/SWISSPROT; Acc: P15692] |
| 7433 | NM_004624 | Vasoactive intestinal polypeptide receptor 1 precursor (VIP- |
| | | R-1) (Pituitary adenylate cyclase-activating polypeptide type |
| | | II receptor) (PACAP type II receptor) (PACAP-R-2). |
| | | [Source: Uniprot/SWISSPROT; Acc: P32241] |
| 7441 | NM_007128 | pre-B lymphocyte gene 1; VPREB1 |
| 7448 | NM_000638 | Vitronectin precursor (Serum spreading factor) (S-protein) |
| | | (V75) [Contains: Vitronectin V65 subunit; Vitronectin V10 |
| | | subunit; Somatomedin B]. |
| | | [Source: Uniprot/SWISSPROT; Acc: P04004] |
| 7454 | NM_000377 | Wiskott-Aldrich syndrome (eczema-thrombocytopenia); WAS |
| 7462 | NM_022040 | Linker for activation of T-cells family member 2 (Non-T-cell |
| | | activation linker) (Linker for activation of B-cells) |
| | | (Membrane-associated adapter molecule) (Williams-Beuren |
| | | syndrome critical region 15 protein). |
| | | [Source: Uniprot/SWISSPROT; Acc: Q9GZY6] |
| 7494 | NM_005080 | X box binding protein 1 (XBP-1) (Tax-responsive element- |
| | | binding protein 5). |
| | | [Source: Uniprot/SWISSPROT; Acc: P17861] |
| 7525 | NM_005433 | v-yes-1 Yamaguchi sarcoma viral oncogene homolog |
| | | 1; YES1 |
| 7528 | NM_003403 | Transcriptional repressor protein YY1 (Yin and yang 1) (YY- |
| | | 1) (Delta transcription factor) (NF-E1). |
| | | [Source: Uniprot/SWISSPROT; Acc: P25490] |
| 7535 | NM_001079; NM_207519 | zeta-chain (TCR) associated protein kinase 70 kDa; ZAP70 |
| 7538 | NM_003407 | Tristetraproline (TTP) (Zinc finger protein 36 homolog) (Zfp- |
| | | 36) (TIS11A protein) (TIS11) (Growth factor-inducible |
| | | nuclear protein NUP475) (G0/G1 switch regulatory protein |
| | | 24). [Source: Uniprot/SWISSPROT; Acc: P26651] |
| 7551 | NM_032924 | Zinc finger protein 38 (Zinc finger protein KOX25) (Zinc |
| | | finger protein HF.12) (Zinc finger protein 3) (HZF3.1 |
| | | protein). [Source: Uniprot/SWISSPROT; Acc: P17036] |
| 7707 | NM_021964 | Zinc finger protein 148 (Zinc finger DNA binding protein 89) |
| | | (Transcription factor ZBP-89). |
| | | [Source: Uniprot/SWISSPROT; Acc: Q9UQR1] |
| 7716 | NM_007146 | Zinc finger protein 161 (Putative transcription factor DB1). |
| | | [Source: Uniprot/SWISSPROT; Acc: Q14119] |
| 7732 | NM_007148 | Zinc finger protein 179 (Brain finger protein) (RING finger |
| | | protein 112). [Source: Uniprot/SWISSPROT; Acc: Q9ULX5] |
| 7791 | NM_001010972 | Zyxin (ZYX) |
| 7837 | | PXDN protein (Fragment). |
| | | [Source: Uniprot/SPTREMBL; Acc: Q4KMG2] |
| 7850 | NM_173343 | Interleukin-1 receptor type II precursor (IL-1R-2) (IL-1R- |
| | | beta) (Antigen CD121b) (Antigen CDw121b). |
| | | [Source: Uniprot/SWISSPROT; Acc: P27930] |
| 7852 | NM_001008540 | C—X—C chemokine receptor type 4 (CXC-R4) (CXCR-4) |
| | | (Stromal cell-derived factor 1 receptor) (SDF-1 receptor) |
| | | (Fusin) (Leukocyte-derived seven transmembrane domain |
| | | receptor) (LESTR) (LCR1) (FB22) (NPYRL) (HM89) (CD184 |
| | | antigen). [Source: Uniprot/SWISSPROT; Ac |
| 7940 | NM_007161 | Leukocyte-specific transcript 1 protein (B144 protein). |
| | | [Source: Uniprot/SWISSPROT; Acc: O00453] |
| 7941 | NM_005084 | Platelet-activating factor acetylhydrolase precursor (EC |
| | | 3.1.1.47) (PAF acetylhydrolase) (PAF 2-acylhydrolase) |
| | | (LDL-associated phospholipase A2) (LDL-PLA(2)) (2-acetyl- |
| | | 1-alkylglycerophosphocholine esterase) (1-alkyl-2- |
| | | acetylglycerophosphocholine esterase) |
| 8061 | NM_005438 | Fos-related antigen 1 (FRA-1). |
| | | [Source: Uniprot/SWISSPROT; Acc: P15407] |
| 8111 | NM_003485 | Sphingosylphosphorylcholine receptor (Ovarian cancer G- |
| | | protein coupled receptor 1) (OGR-1) (G-protein coupled |
| | | receptor 68) (GPR12A). |
| | | [Source: Uniprot/SWISSPROT; Acc: Q15743] |
| 8174 | NM_130761 | Mucosal addressin cell adhesion molecule 1 precursor |
| | | (MAdCAM-1) (hMAdCAM-1). |
| | | [Source: Uniprot/SWISSPROT; Acc: Q13477] |
| 8227 | NM_005088 | B-lymphocyte antigen precursor (B-lymphocyte surface |
| | | antigen) (721P) (Protein XE7). |
| | | [Source: Uniprot/SWISSPROT; Acc: Q02040] |
| 8302 | NM_013431 | killer cell lectin-like receptor subfamily C, member 4; KLRC4 |
| 8440 | NM_001004720; NM_003581; | NCK adaptor protein 2; NCK2 |
| | NM_001004722 | |
| 8455 | NM_139321 | Attractin precursor (Mahogany homolog) (DPPT-L). |
| | | [Source: Uniprot/SWISSPROT; Acc: O75882] |
| 8460 | NM_003596 | Protein-tyrosine sulfotransferase 1 (EC 2.8.2.20) |
| | | (Tyrosylprotein sulfotransferase-1) (TPST-1). |
| | | [Source: Uniprot/SWISSPROT; Acc: O60507] |
| 8477 | NM_003608 | Psychosine receptor (G-protein coupled receptor 65) (T cell- |
| | | death associated protein 8). |
| | | [Source: Uniprot/SWISSPROT; Acc: Q8IYL9] |
| 8482 | NM_003612 | Semaphorin-7A precursor (Semaphorin L) (Sema L) |
| | | (Semaphorin K1) (Sema K1) (John-Milton-Hargen human |
| | | blood group Ag) (JMH blood group antigen) (CD108 antigen) |
| | | (CDw108). [Source: Uniprot/SWISSPROT; Acc: O75326] |
| 8517 | NM_003639 | NF-kappa-B essential modulator (NEMO) (NF-kappa-B |
| | | essential modifier) (Inhibitor of nuclear factor kappa-B |
| | | kinase gamma subunit) (IkB kinase gamma subunit) (I- |
| | | kappa-B kinase gamma) (IKK-gamma) (IKKG) (IkB kinase- |
| | | associated protein 1) (IKKAP1) (FIP-3). [So |
| 8518 | NM_003640 | IkappaB kinase complex-associated protein (IKK complex- |
| | | associated protein) (p150). |
| | | [Source: Uniprot/SWISSPROT; Acc: O95163] |
| 8519 | NM_003641 | Interferon-induced transmembrane protein 1 (Interferon- |
| | | induced protein 17) (Interferon-inducible protein 9-27) |
| | | (Leu-13 antigen) (CD225 antigen). |
| | | [Source: Uniprot/SWISSPROT; Acc: P13164] |
| 8527 | NM_152879 | Diacylglycerol kinase, delta (EC 2.7.1.107) (Diglyceride |
| | | kinase) (DGK-delta) (DAG kinase delta) (130 kDa |
| | | diacylglycerol kinase). |
| | | [Source: Uniprot/SWISSPROT; Acc: Q16760] |
| 8530 | NM_003650 | Cystatin F precursor (Leukocystatin) (Cystatin-7) (Cystatin- |
| | | like metastasis-associated protein) (CMAP). |
| | | [Source: Uniprot/SWISSPROT; Acc: O76096] |
| 8534 | NM_003654 | Carbohydrate sulfotransferase 1 (EC 2.8.2.21) (Keratan |
| | | sulfate Gal-6 sulfotransferase) (KSST) (KSGal6ST) (KS6ST) |
| | | (Galactose/N-acetylglucosamine/N-acetylglucosamine 6-O- |
| | | sulfotransferase 1) (GST-1). |
| | | [Source: Uniprot/SWISSPROT; Acc: O43916] |
| 8546 | NM_003664 | Adapter-related protein complex 3 beta 1 subunit (Beta3A- |
| | | adaptin) (Adaptor protein complex AP-3 beta-1 subunit) |
| | | (AP-3 complex beta-1 subunit) (Clathrin assembly protein |
| | | complex 3 beta-1 large chain). |
| | | [Source: Uniprot/SWISSPROT; Acc: O00203] |
| 8547 | NM_173452; NM_003665 | ficolin (collagen/fibrinogen domain containing) 3 (Hakata |
| | | antigen); FCN3 |
| 8575 | NM_003690 | protein kinase, interferon-inducible double stranded RNA |
| | | dependent activator; PRKRA |
| 8600 | NM_003701 | Tumor necrosis factor ligand superfamily member 11 |
| | | (Receptor activator of nuclear factor kappa B ligand) |
| | | (RANKL) (TNF-related activation-induced cytokine) |
| | | (TRANCE) (Osteoprotegerin ligand) (OPGL) (Osteoclast |
| | | differentiation factor) (ODF) (CD254 antigen) |
| 8605 | NM_003706 | Cytosolic phospholipase A2 gamma (EC 3.1.1.4) (cPLA2- |
| | | gamma) (Phospholipase A2 group IVC). |
| | | [Source: Uniprot/SWISSPROT; Acc: Q9UP65] |
| 8625 | NM_003721 | DNA-binding protein RFXANK (Regulatory factor X subunit |
| | | B) (RFX-B) (Ankyrin repeat family A protein 1). |
| | | [Source: Uniprot/SWISSPROT; Acc: O14593] |
| 8631 | NM_003726 | src family associated phosphoprotein 1 |
| | | [Source: RefSeq_peptide; Acc: NP_003717] |
| 8638 | NM_003733; NM_198213 | 2′-5′-oligoadenylate synthetase-like; OASL |
| 8639 | NM_003734 | Membrane copper amine oxidase (EC 1.4.3.6) |
| | | (Semicarbazide-sensitive amine oxidase) (SSAO) (Vascular |
| | | adhesion protein 1) (VAP-1) (HPAO). |
| | | [Source: Uniprot/SWISSPROT; Acc: Q16853] |
| 8678 | NM_003766 | Beclin-1 (Coiled-coil myosin-like BCL2-interacting protein) |
| | | (Protein GT197). [Source: Uniprot/SWISSPROT; Acc: Q14457] |
| 8681 | NM_005090 | phospholipase A2, group IVB |
| | | [Source: RefSeq_peptide; Acc: NP_005081] |
| 8698 | NM_003775 | Sphingosine 1-phosphate receptor Edg-6 (S1P receptor |
| | | Edg-6) (Endothelial differentiation G-protein coupled |
| | | receptor 6) (Sphingosine 1-phosphate receptor 4) (S1P4). |
| | | [Source: Uniprot/SWISSPROT; Acc: O95977] |
| 8712 | NM_003785 | G antigen family B 1 protein (Prostate-associated gene |
| | | protein 1) (PAGE-1) (GAGE-9) (AL5). |
| | | [Source: Uniprot/SWISSPROT; Acc: O75459] |
| 8718 | NM_148972 | Tumor necrosis factor receptor superfamily member 25 |
| | | precursor (WSL-1 protein) (Apoptosis-mediating receptor |
| | | DR3) (Apoptosis-mediating receptor TRAMP) (Death domain |
| | | receptor 3) (WSL protein) (Apoptosis-inducing receptor |
| | | AIR) (Apo-3) (Lymphocyte associate |
| 8740 | NM_003807 | Tumor necrosis factor ligand superfamily member 14 |
| | | (Herpesvirus entry mediator-ligand) (HVEM-L) (CD258 |
| | | antigen) [Contains: Tumor necrosis factor ligand superfamily |
| | | member 14, membrane form; Tumor necrosis factor ligand |
| | | superfamily member 14, soluble form] |
| 8741 | NM_172088 | Tumor necrosis factor ligand superfamily member 13 |
| | | precursor (A proliferation-inducing ligand) (APRIL) (TNF- |
| | | and APOL-related leukocyte expressed ligand 2) (TALL-2) |
| | | (TNF-related death ligand-1) (TRDL-1) (CD256 antigen). |
| | | [Source: Uniprot/SWISSPROT; Acc: O75888 |
| 8742 | NM_003809 | Tumor necrosis factor ligand superfamily member 13 |
| | | precursor (A proliferation-inducing ligand) (APRIL) (TNF- |
| | | and APOL-related leukocyte expressed ligand 2) (TALL-2) |
| | | (TNF-related death ligand-1) (TRDL-1) (CD256 antigen). |
| | | [Source: Uniprot/SWISSPROT; Acc: O75888 |
| 8743 | NM_003810 | Tumor necrosis factor ligand superfamily member 10 (TNF- |
| | | related apoptosis-inducing ligand) (TRAIL protein) (Apo-2 |
| | | ligand) (Apo-2L) (CD253 antigen). |
| | | [Source: Uniprot/SWISSPROT; Acc: P50591] |
| 8744 | NM_003811 | Tumor necrosis factor ligand superfamily member 9 (4-1BB |
| | | ligand) (4-1BBL). |
| | | [Source: Uniprot/SWISSPROT; Acc: P41273] |
| 8764 | NM_003820 | Tumor necrosis factor receptor superfamily member 14 |
| | | precursor (Herpesvirus entry mediator A) (Tumor necrosis |
| | | factor receptor-like 2) (TR2). |
| | | [Source: Uniprot/SWISSPROT; Acc: Q92956] |
| 8767 | NM_003821 | Receptor-interacting serine/threonine-protein kinase 2 (EC |
| | | 2.7.1.37) (RIP-like interacting CLARP kinase) (Receptor- |
| | | interacting protein 2) (RIP-2) (CARD-containing interleukin- |
| | | 1 beta-converting enzyme-associated kinase) (CARD- |
| | | containing IL-1 beta ICE-kina |
| 8772 | NM_003824 | FADD protein (FAS-associating death domain-containing |
| | | protein) (Mediator of receptor induced toxicity). |
| | | [Source: Uniprot/SWISSPROT; Acc: Q13158] |
| 8778 | NM_003830 | sialic acid binding Ig-like lectin 5; SIGLEC5 |
| 8792 | NM_003839 | Tumor necrosis factor receptor superfamily member 11A |
| | | precursor (Receptor activator of NF-KB) (Osteoclast |
| | | differentiation factor receptor) (ODFR) (CD265 antigen). |
| | | [Source: Uniprot/SWISSPROT; Acc: Q9Y6Q6] |
| 8807 | NM_003853 | Interleukin-18 receptor accessory protein precursor (IL-18 |
| | | receptor accessory protein) (IL-18RAcP) (Interleukin-18 |
| | | receptor accessory protein-like) (IL-18Rbeta) (IL-1R |
| | | accessory protein like) (IL-1RAcPL) (Accessory protein-like) |
| | | (AcPL) (IL-1R7) (CDw218b a |
| 8808 | NM_003854 | Interleukin-1 receptor-like 2 precursor (IL-1Rrp2) |
| | | (Interleukin-1 receptor-related protein 2) (IL1R-rp2). |
| | | [Source: Uniprot/SWISSPROT; Acc: Q9HB29] |
| 8809 | NM_003855 | Interleukin-18 receptor 1 precursor (IL1 receptor-related |
| | | protein) (IL-1Rrp) (CDw218a antigen). |
| | | [Source: Uniprot/SWISSPROT; Acc: Q13478] |
| 8832 | NM_003874 | CD84 antigen (leukocyte antigen); CD84 |
| 8835 | NM_003877 | Suppressor of cytokine signaling 2 (SOCS2) |
| 8841 | NM_003883 | Histone deacetylase 3 (HDAC3) |
| 8862 | NM_017413 | Apelin precursor (APJ endogenous ligand) [Contains: Apelin- |
| | | 36; Apelin-31; Apelin-28; Apelin-13]. |
| | | [Source: Uniprot/SWISSPROT; Acc: Q9ULZ1] |
| 8878 | NM_003900 | Sequestosome-1 (Phosphotyrosine-independent ligand for |
| | | the Lck SH2 domain of 62 kDa) (Ubiquitin-binding protein |
| | | p62) (EBI3-associated protein of 60 kDa) (p60) (EBIAP). |
| | | [Source: Uniprot/SWISSPROT; Acc: Q13501] |
| 8915 | | B cell lymphoma/leukemia 10 (B-cell CLL/lymphoma 10) |
| | | (Bcl-10) (CED-3/ICH-1 prodomain homologous E10-like |
| | | regulator) (CIPER) (CARD-containing molecule enhancing |
| | | NFkappaB) (Cellular homolog of vCARMEN) (cCARMEN) |
| | | (Mammalian CARD-containing adapter molecule |
| 8976 | NM_003941 | Wiskott-Aldrich syndrome-like; WASL |
| 8991 | NM_003944 | selenium binding protein 1; SELENBP1 |
| 8993 | NM_005091 | peptidoglycan recognition protein 1; PGLYRP1 |
| 8995 | NM_005092 | Tumor necrosis factor ligand superfamily member 18 |
| | | (Glucocorticoid-induced TNF-related ligand) (hGITRL) |
| | | (Activation-inducible TNF-related ligand) (AITRL). |
| | | [Source: Uniprot/SWISSPROT; Acc: Q9UNG2] |
| 9103 | NM_001005411; NM_201563; | Fc fragment of IgG, low affinity IIc, receptor for |
| | NM_001005410; NM_001005412 | (CD32); FCGR2C |
| 9111 | NM_004688 | N-myc-interactor (Nmi) (N-myc and STAT interactor). |
| | | [Source: Uniprot/SWISSPROT; Acc: Q13287] |
| 9173 | NM_016232 | Interleukin-1 receptor-like 1 precursor (ST2 protein). |
| | | [Source: Uniprot/SWISSPROT; Acc: Q01638] |
| 9214 | NM_005449 | Fas apoptotic inhibitory molecule 3 |
| | | [Source: RefSeq_peptide; Acc: NP_005440] |
| 9235 | NM_001012631 | Interleukin 32 (IL32) |
| 9240 | NM_006029 | paraneoplastic antigen MA1; PNMA1 |
| 9244 | NM_004750 | Cytokine receptor-like factor 1 precursor (Cytokine-like |
| | | factor 1) (CLF-1) (ZcytoR5). |
| | | [Source: Uniprot/SWISSPROT; Acc: O75462] |
| 9255 | | Multisynthetase complex auxiliary component p43 [Contains: |
| | | Endothelia monocyte-activating polypeptide II (EMAP-II) |
| | | (Small inducible cytokine subfamily E member 1)]. |
| | | [Source: Uniprot/SWISSPROT; Acc: Q12904] |
| 9308 | NM_004233 | CD83 antigen precursor (Cell surface protein HB15) (B-cell |
| | | activation protein). |
| | | [Source: Uniprot/SWISSPROT; Acc: Q01151] |
| 9332 | NM_004244 | CD163 antigen isoform a |
| | | [Source: RefSeq_peptide; Acc: NP_004235] |
| 9373 | NM_001031689 | Phospholipase A-2-activating protein (PLAP) (PLA2P). |
| | | [Source: Uniprot/SWISSPROT; Acc: Q9Y263] |
| 9398 | NM_004258 | immunoglobulin superfamily, member 2 |
| | | [Source: RefSeq_peptide; Acc: NP_004249] |
| 9402 | NM_004810 | GRB2-related adaptor protein 2; GRAP2 |
| 9435 | NM_004267 | Carbohydrate sulfotransferase 2 (EC 2.8.2.—) (N- |
| | | acetylglucosamine 6-O-sulfotransferase 1) (GlcNAc6ST-1) |
| | | (Gn6ST) (Galactose/N-acetylglucosamine/N- |
| | | acetylglucosamine 6-O-sulfotransferase 2) (GST-2). |
| | | [Source: Uniprot/SWISSPROT; Acc: Q9Y4C5] |
| 9436 | NM_004828 | Natural cytotoxicity triggering receptor 2 precursor (Natural |
| | | killer cell p44-related protein) (NKp44) (NK-p44) (NK cell- |
| | | activating receptor) (Lymphocyte antigen 95 homolog) |
| | | (CD336 antigen). [Source: Uniprot/SWISSPROT; Acc: O95944] |
| 9437 | NM_004829 | natural cytotoxicity triggering receptor 1; NCR1 |
| 9447 | NM_004833 | Interferon-inducible protein AIM2 (Absent in melanoma 2). |
| | | [Source: Uniprot/SWISSPROT; Acc: O14862] |
| 9450 | NM_004271 | Lymphocyte antigen 86 precursor (MD-1 protein). |
| | | [Source: Uniprot/SWISSPROT; Acc: O95711] |
| 9466 | NM_004843 | interleukin 27 receptor, alpha; IL27RA |
| 9536 | NM_004878 | Prostaglandin E synthase (EC 5.3.99.3) (Microsomal |
| | | glutathione S-transferase 1-like 1) (MGST1-L1) (p53- |
| | | induced apoptosis protein 12). |
| | | [Source: Uniprot/SWISSPROT; Acc: O14684] |
| 9547 | NM_004887 | Small inducible cytokine B14 precursor (CXCL14) |
| | | (Chemokine BRAK). |
| | | [Source: Uniprot/SWISSPROT; Acc: O95715] |
| 9560 | NM_001001435 | chemokine (C-C motif) ligand 4-like 2 precursor |
| | | [Source: RefSeq_peptide; Acc: NP_996890] |
| 9567 | NM_004286 | GTP-binding protein 1 (G-protein 1) (GP-1) (GP1). |
| | | [Source: Uniprot/SWISSPROT; Acc: O00178] |
| 9636 | NM_005101 | Interferon-induced 17 kDa protein precursor [Contains: |
| | | Ubiquitin cross-reactive protein (Interferon-induced 15 kDa |
| | | protein)]. [Source: Uniprot/SWISSPROT; Acc: P05161] |
| 9641 | NM_014002 | Inhibitor of nuclear factor kappa-B kinase epsilon subunit |
| | | (EC 2.7.1.—) (I kappa-B kinase epsilon) (IkBKE) (IKK- |
| | | epsilon) (IKK-E) (Inducible I kappa-B kinase) (IKK-i). |
| | | [Source: Uniprot/SWISSPROT; Acc: Q14164] |
| 9655 | NM_014011 | Cytokine inducible SH2-containing protein 5 (Suppressor of |
| | | cytokine signaling 5) (SOCS-5) (Cytokine-inducible SH2 |
| | | protein 6) (CIS-6). |
| | | [Source: Uniprot/SWISSPROT; Acc: O75159] |
| 9682 | NM_014663 | Jumonji domain containing 2A (JMJD2A) |
| 9734 | NM_178423 | Histone deacetylase 9 (HD9) (HD7B) (HD7). |
| | | [Source: Uniprot/SWISSPROT; Acc: Q9UKV0] |
| 9759 | NM_006037 | Histone deacetylase 4 (HD4). |
| | | [Source: Uniprot/SWISSPROT; Acc: P56524] |
| 9966 | NM_005118 | tumor necrosis factor (ligand) superfamily, member 15 |
| | | [Source: RefSeq_peptide; Acc: NP_005109] |
| 9976 | NM_005127 | C-type lectin domain family 2, member B; CLEC2B |
| 10000 | NM_181690; NM_005465 | v-akt murine thymoma viral oncogene homolog 3 (protein |
| | | kinase B, gamma); AKT3 |
| 10005 | NM_005469 | Peroxisomal acyl-coenzyme A thioester hydrolase 1 (EC |
| | | 3.1.2.2) (Peroxisomal long-chain acyl-coA thioesterase 1) |
| | | (Acyl-CoA thioesterase 8) (HIV-Nef associated acyl coA |
| | | thioesterase) (Thioesterase II) (hTE) (hACTEIII) (hACTE- |
| | | III) (PTE-2). [Source: Uniprot/SW |
| 10014 | NM_001015053 | Histone deacetylase 5 (HD5) (Antigen NY-CO-9). |
| | | [Source: Uniprot/SWISSPROT; Acc: Q9UQL6] |
| 10068 | | Interleukin-18-binding protein precursor (IL-18BP) |
| | | (Tadekinig-alfa). [Source: Uniprot/SWISSPROT; Acc: O95998] |
| 10087 | NM_005713 | Goodpasture antigen-binding protein (EC 2.7.1.37) (GPBP) |
| | | (Collagen type IV alpha 3 binding protein) (StAR-related |
| | | lipid transfer protein 11) (StARD11) (START domain- |
| | | containing protein 11). |
| | | [Source: Uniprot/SWISSPROT; Acc: Q9Y5P4] |
| 10134 | NM_005745 | B-cell receptor-associated protein 31 (BCR-associated |
| | | protein Bap31) (p28 Bap31) (CDM protein) (6C6-AG tumor- |
| | | associated antigen) (DXS1357E). |
| | | [Source: Uniprot/SWISSPROT; Acc: P51572] |
| 10148 | NM_005755 | Interleukin-27 beta chain precursor (IL-27B) (Epstein-Barr |
| | | virus-induced gene 3 protein) (EBV-induced gene 3 |
| | | protein). [Source: Uniprot/SWISSPROT; Acc: Q14213] |
| 10164 | NM_005769 | Carbohydrate sulfotransferase 4 (EC 2.8.2.—) (N- |
| | | acetylglucosamine 6-O-sulfotransferase 2) (GlcNAc6ST-2) |
| | | (High endothelial cells N-acetylglucosamine 6-O- |
| | | sulfotransferase) (HEC-GlcNAc6ST) (L-selectin ligand |
| | | sulfotransferase) (LSST) (Galactose/N-acetylgluc |
| 10175 | NM_001009551 | Cornichon homolog (TGAM77). |
| | | [Source: Uniprot/SWISSPROT; Acc: O95406] |
| 10178 | NM_014253 | odz, odd Oz/ten-m homolog 1 |
| | | [Source: RefSeq_peptide; Acc: NP_055068] |
| 10219 | NM_005810 | killer cell lectin-like receptor subfamily G, member 1; KLRG1 |
| 10225 | NM_005816; NM_198196 | CD96 antigen; CD96 |
| 10261 | NM_005849 | immunoglobulin superfamily, member 6 |
| | | [Source: RefSeq_peptide; Acc: NP_005840] |
| 10288 | NM_005874 | leukocyte immunoglobulin-like receptor, subfamily B (with |
| | | TM and ITIM domains), member 2; LILRB2 |
| 10298 | NM_001014832; NM_001014835; | p21(CDKN1A)-activated kinase 4; PAK4 |
| | NM_001014833; | |
| | NM_001014831; NM_005884; | |
| | NM_001014834 | |
| 10312 | NM_006053 | Vacuolar proton translocating ATPase 116 kDa subunit a |
| | | isoform 3 (V-ATPase 116-kDa isoform a3) (Osteoclastic |
| | | proton pump 116 kDa subunit) (OC-116 kDa) (OC116) (T- |
| | | cell immune regulator 1) (T cell immune response cDNA7 |
| | | protein) (TIRC7). [Source: Uniprot/SWI |
| 10318 | NM_006058 | TNFAIP3 interacting protein 1 (TNIP1) |
| 10321 | NM_006061 | Cysteine-rich secretory protein 3 precursor (CRISP-3) |
| | | (SGP28 protein). [Source: Uniprot/SWISSPROT; Acc: P54108] |
| 10332 | NM_214679 | CD209 antigen-like protein 1 (Dendritic cell-specific ICAM- |
| | | 3-grabbing nonintegrin 2) (DC-SIGN2) (DC-SIGN-related |
| | | protein) (DC-SIGNR) (Liver/lymph node-specific ICAM-3- |
| | | grabbing nonintegrin) (L-SIGN). |
| | | [Source: Uniprot/SWISSPROT; Acc: Q9H2X3] |
| 10333 | NM_006068 | Toll-like receptor 6 precursor. |
| | | [Source: Uniprot/SWISSPROT; Acc: Q9Y2C9] |
| 10344 | NM_006072 | Small inducible cytokine A26 precursor (CCL26) (Eotaxin-3) |
| | | (Macrophage inflammatory protein 4-alpha) (MIP-4-alpha) |
| | | (Thymic stroma chemokine-1) (TSC-1) (CC chemokine |
| | | IMAC). [Source: Uniprot/SWISSPROT; Acc: Q9Y258] |
| 10379 | NM_006084 | Transcriptional regulator ISGF3 gamma subunit (Interferon |
| | | regulatory factor 9) (IRF-9) (IFN-alpha-responsive |
| | | transcription factor subunit) (Interferon-stimulated gene |
| | | factor 3 gamma) (ISGF3 p48 subunit) (ISGF-3 gamma). |
| | | [Source: Uniprot/SWISSPROT; Acc: Q0097 |
| 10394 | NM_006093 | proteoglycan 3; PRG3 |
| 10410 | NM_021034 | Interferon-induced transmembrane protein 3 (Interferon- |
| | | inducible protein 1-8U). |
| | | [Source: Uniprot/SWISSPROT; Acc: Q01628] |
| 10417 | NM_012445 | Spondin-2 precursor (Mindin) (Differentially expressed in |
| | | cancerous and noncancerous lung cells 1) (DIL-1). |
| | | [Source: Uniprot/SWISSPROT; Acc: Q9BUD6] |
| 10421 | NM_006110 | CD2 antigen cytoplasmic tail-binding protein 2 (CD2 |
| | | cytoplasmic domain binding protein) (CD2 tail binding |
| | | protein). [Source: Uniprot/SWISSPROT; Acc: O95400] |
| 10435 | NM_006779 | CDC42 effector protein (Rho GTPase binding) 2 |
| | | (CDC42EP2) |
| 10437 | NM_006332 | Gamma-interferon inducible lysosomal thiol reductase |
| | | precursor (Gamma-interferon-inducible protein IP-30). |
| | | [Source: Uniprot/SWISSPROT; Acc: P13284] |
| 10451 | NM_006113 | vav 3 oncogene; VAV3 |
| 10462 | NM_182906 | C-type lectin domain family 10 member A (C-type lectin, |
| | | superfamily member 14) (Macrophage lectin 2) (CD301 |
| | | antigen). [Source: Uniprot/SWISSPROT; Acc: Q8IUN9] |
| 10507 | NM_006378 | Semaphorin-4D precursor (Leukocyte activation antigen |
| | | CD100) (BB18) (A8) (GR3). |
| | | [Source: Uniprot/SWISSPROT; Acc: Q92854] |
| 10512 | NM_006379 | Semaphorin-3C precursor (Semaphorin E) (Sema E). |
| | | [Source: Uniprot/SWISSPROT; Acc: Q99985] |
| 10537 | NM_006398 | Gamma-aminobutyric acid type B receptor, subunit 1 |
| | | precursor (GABA-B receptor 1) (GABA-B-R1) (Gb1). |
| | | [Source: Uniprot/SWISSPROT; Acc: Q9UBS5] |
| 10538 | NM_006399 | ATF-like basic leucine zipper transcriptional factor B-ATF |
| | | (SF-HT-activated gene 2) (SFA-2). |
| | | [Source: Uniprot/SWISSPROT; Acc: Q16520] |
| 10544 | NM_006404 | Endothelial protein C receptor precursor (Endothelial cell |
| | | protein C receptor) (Activated protein C receptor) (APC |
| | | receptor) (CD201 antigen). |
| | | [Source: Uniprot/SWISSPROT; Acc: Q9UNN8] |
| 10563 | NM_006419 | Small inducible cytokine B13 precursor (CXCL13) (B |
| | | lymphocyte chemoattractant) (CXC chemokine BLC) (B cell- |
| | | attracting chemokine 1) (BCA-1) (ANGIE). |
| | | [Source: Uniprot/SWISSPROT; Acc: O43927] |
| 10578 | NM_006433; NM_012483 | granulysin; GNLY |
| 10581 | NM_006435 | Interferon-induced transmembrane protein 2 (Interferon- |
| | | inducible protein 1-8D). |
| | | [Source: Uniprot/SWISSPROT; Acc: Q01629] |
| 10584 | NM_006438 | collectin sub-family member 10 (C-type lectin); COLEC10 |
| 10602 | NM_006449 | CDC42 effector protein (Rho GTPase binding) 3 |
| | | (CDC42EP3) |
| 10666 | NM_006566 | CD226 antigen precursor (DNAX accessory molecule 1) |
| | | (DNAM-1). [Source: Uniprot/SWISSPROT; Acc: Q15762] |
| 10673 | NM_006573 | Tumor necrosis factor ligand superfamily member 13B |
| | | (TNF-and APOL-related leukocyte expressed ligand 1) |
| | | (TALL-1) (B lymphocyte stimulator) (BLyS) (B cell-activating |
| | | factor) (BAFF) (Dendritic cell-derived TNF-like molecule) |
| | | (CD257 antigen) [Contains: Tum |
| 10687 | NM_007257 | paraneoplastic antigen MA2; PNMA2 |
| 10746 | NM_006609 | mitogen-activated protein kinase kinase kinase 2; MAP3K2 |
| 10747 | NM_006610 | Mannan-binding lectin serine protease 2 precursor (EC |
| | | 3.4.21.104) (Mannose-binding protein associated serine |
| | | protease 2) (MASP-2) (MBL-associated serine protease 2) |
| | | [Contains: Mannan-binding lectin serine protease 2 A chain; |
| | | Mannan-binding lectin serine |
| 10748 | NM_006611 | killer cell lectin-like receptor subfamily A, member 1 |
| | | [Source: RefSeq_peptide; Acc: NP_006602] |
| 10750 | NM_006613 | GRB2-related adaptor protein; GRAP |
| 10758 | NM_147200 | Adapter protein CIKS (Connection to IKK and SAPK/JNK) |
| | | (TRAF3-interacting protein 2) (Nuclear factor NF-kappa-B |
| | | activator 1) (ACT1). |
| | | [Source: Uniprot/SWISSPROT; Acc: O43734] |
| 10803 | NM_031200 | C-C chemokine receptor type 9 (C-C CKR-9) (CC-CKR-9) |
| | | (CCR-9) (GPR-9-6) (G-protein coupled receptor 28) |
| | | (CDw199 antigen). |
| | | [Source: Uniprot/SWISSPROT; Acc: P51686] |
| 10849 | NM_012099 | CD3E antigen, epsilon polypeptide associated protein |
| | | [Source: RefSeq_peptide; Acc: NP_036231] |
| 10850 | NM_006664 | Small inducible cytokine A27 precursor (CCL27) (CC |
| | | chemokine ILC) (IL-11 R-alpha-locus chemokine) |
| | | (Skinkine) (ESkine) (Cuteaneous T-cell attracting |
| | | chemokine) (CTACK). |
| | | [Source: Uniprot/SWISSPROT; Acc: Q9Y4X3] |
| 10855 | NM_006665 | Heparanase precursor (EC 3.2.—.—) (Heparanase-1) (Hpa1) |
| | | (Endo-glucoronidase) [Contains: Heparanase 8 kDa |
| | | subunit; Heparanase 50 kDa subunit]. |
| | | [Source: Uniprot/SWISSPROT; Acc: Q9Y251] |
| 10859 | NM_006669 | leukocyte immunoglobulin-like receptor, subfamily B (with |
| | | TM and ITIM domains), member 1; LILRB1 |
| 10871 | NM_006678 | CD300C antigen; CD300C |
| 10877 | NM_006684 | complement factor H-related 4; CFHL4 |
| 10878 | NM_021023 | complement factor H-related 3; CFHL3 |
| 10882 | NM_006688 | complement component 1, q subcomponent-like 1; C1QL1 |
| 10912 | NM_006705 | Growth arrest and DNA-damage-inducible protein GADD45 |
| | | gamma (Cytokine-responsive protein CR6). |
| | | [Source: Uniprot/SWISSPROT; Acc: O95257] |
| 10964 | NM_006820 | histocompatibility 28 |
| | | [Source: RefSeq_peptide; Acc: NP_006811] |
| 10982 | NM_014268 | Microtubule-associated protein RP/EB family member 2 |
| | | (APC-binding protein EB2) (End-binding protein 2) (EB2). |
| | | [Source: Uniprot/SWISSPROT; Acc: Q15555] |
| 10990 | NM_006840 | leukocyte immunoglobulin-like receptor, subfamily B (with |
| | | TM and ITIM domains), member 5; LILRB5 |
| 11006 | NM_006847 | Leukocyte immunoglobulin-like receptor subfamily B |
| | | member 4 precursor (Leukocyte immunoglobulin-like |
| | | receptor 5) (LIR-5) (Immunoglobulin-like transcript 3) |
| | | (ILT-3) (Monocyte inhibitory receptor HM18) (CD85k |
| | | antigen). [Source: Uniprot/SWISSPROT; Acc: Q8NHJ6] |
| 11009 | NM_006850 | Interleukin-24 precursor (Suppression of tumorigenicity 16 |
| | | protein) (Melanoma differentiation-associated gene 7 |
| | | protein) (MDA-7). |
| | | [Source: Uniprot/SWISSPROT; Acc: Q13007] |
| 11024 | NM_006863 | leukocyte immunoglobulin-like receptor, subfamily A (with |
| | | TM domain), member 1; LILRA1 |
| 11025 | NM_006864 | leukocyte immunoglobulin-like receptor, subfamily B (with |
| | | TM and ITIM domains), member 3; LILRB3 |
| 11026 | NM_006865 | leukocyte immunoglobulin-like receptor, subfamily A |
| | | (without TM domain), member 3; LILRA3 |
| 11027 | NM_006866 | leukocyte immunoglobulin-like receptor, subfamily A (with |
| | | TM domain), member 2; LILRA2 |
| 11059 | | NEDD4-like E3 ubiquitin-protein ligase WWP1 (EC 6.3.2.—) |
| | | (WW domain-containing protein 1) (Atropin-1 interacting |
| | | protein 5) (AIP5). |
| | | [Source: Uniprot/SWISSPROT; Acc: Q9H0M0] |
| 11126 | NM_007053 | CD160 antigen precursor (Natural killer cell receptor BY55). |
| | | [Source: Uniprot/SWISSPROT; Acc: O95971] |
| 11146 | NM_053274 | Glomulin (FKBP-associated protein) (FK506-binding protein- |
| | | associated protein) (FAP). |
| | | [Source: Uniprot/SWISSPROT; Acc: Q92990] |
| 11251 | NM_004778 | Putative G-protein coupled receptor 44 (Chemoattractant |
| | | receptor-homologous molecule expressed on Th2 cells) |
| | | (CD294 antigen). [Source: Uniprot/SWISSPROT; Acc: Q9Y5Y4] |
| 11311 | NM_007259 | Vacuolar protein sorting-associated protein 45 (h-VPS45) |
| | | (hIVps45). [Source: Uniprot/SWISSPROT; Acc: Q9NRW7] |
| 11314 | NM_007261 | CD300A antigen; CD300A |
| 11343 | NM_007283 | Monoglyceride lipase (EC 3.1.1.23) (HU-K5) |
| | | (Lysophospholipase homolog) (Lysophospholipase-like). |
| | | [Source: Uniprot/SWISSPROT; Acc: Q99685] |
| 22800 | NM_012250 | related RAS viral (r-ras) oncogene homolog 2; RRAS2 |
| 22914 | NM_007360 | killer cell lectin-like receptor subfamily K, member 1; KLRK1 |
| 22918 | NM_012072 | Complement component C1q receptor precursor |
| | | (Complement component 1, q subcomponent, receptor 1) |
| | | (C1qRp) (C1qR(p)) (C1q/MBL/SPA receptor) (CD93 antigen) |
| | | (CDw93). [Source: Uniprot/SWISSPROT; Acc: Q9NPY3] |
| 23030 | NM_015015 | Jumonji domain containing 2B (JMJD2B) |
| 23075 | | SWAP-70 protein [Source: RefSeq_peptide; Acc: NP_055870] |
| 23139 | NM_015112 | Microtubule-associated serine/threonine-protein kinase 2 |
| | | (EC 2.7.1.37). [Source: Uniprot/SWISSPROT; Acc: Q6P0Q8] |
| 23166 | NM_015136 | Stabilin-1 precursor (FEEL-1 protein) (MS-1 antigen). |
| | | [Source: Uniprot/SWISSPROT; Acc: Q9NY15] |
| 23303 | | Kinesin-like protein KIF13B (Kinesin-like protein GAKIN). |
| | | [Source: Uniprot/SWISSPROT; Acc: Q9NQT8] |
| 23308 | NM_015259 | ICOS ligand precursor (B7 homolog 2) (B7-H2) (B7-like |
| | | protein GI50) (B7-related protein 1) (B7RP-1) (CD275 |
| | | antigen). [Source: Uniprot/SWISSPROT; Acc: O75144] |
| 23418 | NM_012076; NM_201253 | crumbs homolog 1 (Drosophila); CRB1 |
| 23430 | NM_012217 | Tryptase delta precursor (EC 3.4.21.59) (Delta tryptase) |
| | | (Mast cell mMCP-7-like) (Tryptase-3) (HmMCP-3-like |
| | | tryptase III). [Source: Uniprot/SWISSPROT; Acc: Q9BZJ3] |
| 23433 | NM_012249 | ras homolog gene family, member Q; RHOQ |
| 23467 | NM_014293; NM_058178 | neuronal pentraxin receptor; NPTXR |
| 23495 | NM_012452 | Tumor necrosis factor receptor superfamily member 13B |
| | | (Transmembrane activator and CAML interactor) (CD267 |
| | | antigen). [Source: Uniprot/SWISSPROT; Acc: O14836] |
| 23529 | NM_013246 | B cell-stimulating factor 3 precursor (BSF-3) (Novel |
| | | neurotrophin-1) (NNT-1) (Cardiotrophin-like cytokine). |
| | | [Source: Uniprot/SWISSPROT; Acc: Q9UBD9] |
| 23545 | NM_012463 | Vacuolar proton translocating ATPase 116 kDa subunit a |
| | | isoform 2 (V-ATPase 116-kDa isoform a2) (TJ6). |
| | | [Source: Uniprot/SWISSPROT; Acc: Q9Y487] |
| 23547 | NM_012276 | leukocyte immunoglobulin-like receptor, subfamily A (with |
| | | TM domain), member 4; LILRA4 |
| 23586 | NM_014314 | Probable ATP-dependent RNA helicase DDX58 (EC 3.6.1.—) |
| | | (DEAD-box protein 58) (Retinoic acid-inducible gene 1 |
| | | protein) (RIG-1) (RIG-I). |
| | | [Source: Uniprot/SWISSPROT; Acc: O95786] |
| 23601 | NM_013252 | C-type lectin domain family 5, member A; CLEC5A |
| 23643 | NM_015364 | Lymphocyte antigen 96 precursor (MD-2 protein) (ESOP-1). |
| | | [Source: Uniprot/SWISSPROT; Acc: Q9Y6Y9] |
| 23677 | NM_014521 | SH3-domain binding protein 4; SH3BP4 |
| 23780 | NM_145637 | Apolipoprotein-L2 (Apolipoprotein L-II) (ApoL-II). |
| | | [Source: Uniprot/SWISSPROT; Acc: Q9BQE5] |
| 24138 | NM_012420 | Interferon-induced protein with tetratricopeptide repeats 5 |
| | | (IFIT-5) (Retinoic acid- and interferon-inducible 58 kDa |
| | | protein). [Source: Uniprot/SWISSPROT; Acc: Q13325] |
| 25801 | NM_012198 | Grancalcin. [Source: Uniprot/SWISSPROT; Acc: P28676] |
| 25824 | NM_012094 | Peroxiredoxin 5, mitochondrial precursor (EC 1.11.1.15) |
| | | (Prx-V) (Peroxisomal antioxidant enzyme) (PLP) |
| | | (Thioredoxin reductase) (Thioredoxin peroxidase PMP20) |
| | | (Antioxidant enzyme B166) (AOEB166) (TPx type VI) (Liver |
| | | tissue 2D-page spot 71B) (Alu corepresso |
| 25923 | NM_015459 | atlastin-3 |
| 25939 | NM_015474 | SAM domain and HD domain 1; SAMHD1 |
| 25945 | NM_015480 | poliovirus receptor-related 3; PVRL3 |
| 25992 | XM_059482 | sushi, nidogen and EGF-like domains 1; SNED1 |
| 26133 | NM_015638 | Trpc4-associated protein (Short transient receptor potential |
| | | channel 4 associated protein) (Trp4-associated protein) |
| | | (TAP1 protein) (TNF-receptor ubiquitous |
| | | scaffolding/signaling protein) (TRUSS protein). |
| | | [Source: Uniprot/SWISSPROT; Acc: Q8TEL6] |
| 26228 | NM_012108 | Signal-transducing adaptor protein 1 (STAP-1) (Stem cell |
| | | adaptor protein 1) (BCR downstream signaling protein 1) |
| | | (Docking protein BRDG1). |
| | | [Source: Uniprot/SWISSPROT; Acc: Q9ULZ2] |
| 26253 | NM_014358 | C-type lectin domain family 4 member E (C-type lectin |
| | | superfamily member 9) (Macrophage-inducible C-type |
| | | lectin). [Source: Uniprot/SWISSPROT; Acc: Q9ULY5] |
| 26279 | NM_012400 | Group IID secretory phospholipase A2 precursor (EC |
| | | 3.1.1.4) (Phosphatidylcholine 2-acylhydrolase GIID) (GIID |
| | | sPLA2) (PLA2IID) (sPLA(2)-IID) (Secretory-type PLA, |
| | | stroma-associated homolog). |
| | | [Source: Uniprot/SWISSPROT; Acc: Q9UNK4] |
| 26525 | NM_173170 | Interleukin-1 family member 5 (IL-1F5) (Interleukin-1 |
| | | delta) (IL-1 delta) (FIL1 delta) (Interleukin-1-like protein 1) |
| | | (IL-1L1) (Interleukin-1 HY1) (IL-1HY1) (Interleukin-1 |
| | | receptor antagonist homolog 1) (IL-1ra homolog 1) (IL-1- |
| | | related protein 3) (IL-1RP3 |
| 26749 | NM_012196 | GAGE-1 protein (G antigen 1) (MZ2-F antigen). |
| | | [Source: Uniprot/SWISSPROT; Acc: Q13065] |
| 26762 | NM_012206 | hepatitis A virus cellular receptor 1; HAVCR1 |
| 27033 | NM_014383 | testis zinc finger protein |
| | | [Source: RefSeq_peptide; Acc: NP_055198] |
| 27036 | NM_016543; NM_014385 | sialic acid binding Ig-like lectin 7; SIGLEC7 |
| 27040 | | Linker for activation of T-cells family member 1 (36 kDa |
| | | phospho-tyrosine adapter protein) (pp36) (p36-38). |
| | | [Source: Uniprot/SWISSPROT; Acc: O43561] |
| 27159 | | Acidic mammalian chitinase precursor (EC 3.2.1.14) |
| | | (AMCase) (TSA1902). |
| | | [Source: Uniprot/SWISSPROT; Acc: Q9BZP6] |
| 27166 | NM_013237 | Px19-like protein (25 kDa protein of relevant evolutionary |
| | | and lymphoid interest) (PRELI). |
| | | [Source: Uniprot/SWISSPROT; Acc: Q9Y255] |
| 27177 | NM_014438 | Interleukin-1 family member 8 (IL-1F8) (Interleukin-1 eta) |
| | | (IL-1 eta) (FIL1 eta) (Interleukin-1 homolog 2) (IL-1H2). |
| | | [Source: Uniprot/SWISSPROT; Acc: Q9NZH7] |
| 27178 | NM_014439 | Interleukin-1 family member 7 precursor (IL-1F7) |
| | | (Interleukin-1 zeta) (IL-1 zeta) (FIL1 zeta) (Interleukin-1 |
| | | homolog 4) (IL-1H4) (Interleukin-1-related protein) (IL- |
| | | 1RP1) (IL-1X protein). |
| | | [Source: Uniprot/SWISSPROT; Acc: Q9NZH6] |
| 27179 | NM_014440 | Interleukin-1 family member 6 (IL-1F6) (Interleukin-1 |
| | | epsilon) (IL-1 epsilon) (FIL1 epsilon). |
| | | [Source: Uniprot/SWISSPROT; Acc: Q9UHA7] |
| 27180 | NM_014441 | sialic acid binding Ig-like lectin 9; SIGLEC9 |
| 27181 | NM_014442 | sialic acid binding Ig-like lectin 8; SIGLEC8 |
| 27189 | NM_013278 | Interleukin-17C precursor (IL-17C) (Cytokine CX2). |
| | | [Source: Uniprot/SWISSPROT; Acc: Q9P0M4] |
| 27190 | NM_014443 | Interleukin-17B precursor (IL-17B) (Cytokine-like protein |
| | | Zcyto7) (Neuronal interleukin-17-related factor) |
| | | (Interleukin-20) (IL-20). |
| | | [Source: Uniprot/SWISSPROT; Acc: Q9UHF5] |
| 27240 | NM_014450 | Signaling threshold-regulating transmembrane adapter 1 |
| | | precursor (Suppression-inducing transmembrane adapter 1) |
| | | (SHP2-interacting transmembrane adapter protein) |
| | | (gp30/40). [Source: Uniprot/SWISSPROT; Acc: Q9Y3P8] |
| 28303 | XM_496157 | immunoglobulin heavy variable 3/OR16-13; IGHV3OR16-13 |
| 28514 | NM_005618 | delta-like 1 (Drosophila); DLL1 |
| 28566 | BC036926 | T cell receptor beta variable 21-1 |
| 28619 | BC070387 | T cell receptor beta variable 3-1 |
| 28663 | BC025727 | T cell receptor alpha variable 20 |
| 28988 | NM_014063 | Drebrin-like protein (SH3 domain-containing protein 7) |
| | | (Drebrin F) (Cervical SH3P7) (HPK1-interacting protein of 55 kDa) |
| | | (HIP-55) (Cervical mucin-associated protein). |
| | | [Source: Uniprot/SWISSPROT; Acc: Q9UJU6] |
| 29108 | NM_013258 | Apoptosis-associated speck-like protein containing a CARD |
| | | (hASC) (PYD and CARD domain containing protein) (Target |
| | | of methylation-induced silencing 1) (Caspase recruitment |
| | | domain protein 5). |
| | | [Source: Uniprot/SWISSPROT; Acc: Q9ULZ3] |
| 29121 | NM_013269; NM_001004419; | C-type lectin domain family 2, member D; CLEC2D |
| | NM_001004420 | |
| 29126 | NM_014143 | Programmed cell death 1 ligand 1 precursor (Programmed |
| | | death ligand 1) (PD-L1) (PDCD1 ligand 1) (CD274 antigen) |
| | | (B7-homolog 1) (B7-H1). |
| | | [Source: Uniprot/SWISSPROT; Acc: Q9NZQ7] |
| 29760 | NM_013314 | B-cell linker; BLNK |
| 29802 | NM_013378 | pre-B lymphocyte gene 3; VPREB3 |
| 29851 | NM_012092 | Inducible T-cell co-stimulator precursor (Activation- |
| | | inducible lymphocyte immunomediatory molecule) (CD278 |
| | | antigen). [Source: Uniprot/SWISSPROT; Acc: Q9Y6W8] |
| 29949 | NM_153758 | Interleukin-19 precursor (IL-19) (Melanoma differentiation |
| | | associated protein-like protein) (NG.1). |
| | | [Source: Uniprot/SWISSPROT; Acc: Q9UHD0] |
| 29990 | NM_013440; NM_178238; | paired immunoglobin-like type 2 receptor beta; PILRB |
| | NM_175047 | |
| 29992 | NM_178273; NM_178272; | paired immunoglobin-like type 2 receptor alpha; PILRA |
| | NM_013439 | |
| 30009 | NM_013351 | T-box transcription factor TBX21 (T-box protein 21) |
| | | (Transcription factor TBLYM) (T-cell-specific T-box |
| | | transcription factor T-bet). |
| | | [Source: Uniprot/SWISSPROT; Acc: Q9UL17] |
| 30814 | NM_014589 | Group IIE secretory phospholipase A2 precursor (EC 3.1.1.4) |
| | | (Phosphatidylcholine 2-acylhydrolase GIIE) (GIIE sPLA2) |
| | | (sPLA(2)-IIE). [Source: Uniprot/SWISSPROT; Acc: Q9NZK7] |
| 30835 | NM_021155 | CD209 antigen (Dendritic cell-specific ICAM-3-grabbing |
| | | nonintegrin 1) (DC-SIGN1) (DC-SIGN). |
| | | [Source: Uniprot/SWISSPROT; Acc: Q9NNX6] |
| 50604 | NM_018724 | Interleukin-20 precursor (IL-20) (Four alpha helix cytokine |
| | | Zcyto10). [Source: Uniprot/SWISSPROT; Acc: Q9NYY1] |
| 50615 | NM_181078 | Interleukin-21 receptor precursor (IL-21R) (Novel |
| | | interleukin receptor). |
| | | [Source: Uniprot/SWISSPROT; Acc: Q9HBE5] |
| 50616 | NM_020525 | Interleukin-22 precursor (IL-22) (IL-10-related T-cell- |
| | | derived inducible factor) (IL-TIF). |
| | | [Source: Uniprot/SWISSPROT; Acc: Q9GZX6] |
| 50848 | NM_016946 | Junctional adhesion molecule A precursor (JAM-A) |
| | | (Junctional adhesion molecule 1) (JAM) (Platelet adhesion |
| | | molecule 1) (PAM-1) (Platelet F11 receptor) (CD321 |
| | | antigen). [Source: Uniprot/SWISSPROT; Acc: Q9Y624] |
| 50852 | NM_016388 | T-cell receptor-associated transmembrane adapter 1 (T-cell |
| | | receptor-interacting molecule) (TRIM) (pp29/30). |
| | | [Source: Uniprot/SWISSPROT; Acc: Q6PIZ9] |
| 50856 | NM_016184 | C-type lectin domain family 4 member A (C-type lectin |
| | | superfamily member 6) (Dendritic cell immunoreceptor) |
| | | (Lectin-like immunoreceptor) (C-type lectin DDB27) |
| | | (HDCGC13P). [Source: Uniprot/SWISSPROT; Acc: Q9UMR7] |
| 50943 | NM_014009 | Forkhead box protein P3 (Scurfin). |
| | | [Source: Uniprot/SWISSPROT; Acc: Q9BZS1] |
| 51062 | NM_015915 | Atlastin (GTP-binding protein 3) (Guanine nucleotide- |
| | | binding protein 3) (Brain-specific GTP-binding protein). |
| | | [Source: Uniprot/SWISSPROT; Acc: Q8WXF7] |
| 51110 | NM_016027 | lactamase, beta 2; LACTB2 |
| 51192 | NM_016951 | Chemokine-like factor (C32). |
| | | [Source: Uniprot/SWISSPROT; Acc: Q9UBR5] |
| 51206 | NM_016363 | glycoprotein VI (platelet); GP6 |
| 51266 | NM_016509 | C-type lectin domain family 1, member B; CLEC1B |
| 51284 | NM_016562 | Toll-like receptor 7 precursor. |
| | | [Source: Uniprot/SWISSPROT; Acc: Q9NYK1] |
| 51297 | NM_130852; NM_016583 | palate, lung and nasal epithelium carcinoma |
| | | associated; PLUNC |
| 51311 | NM_138636 | Toll-like receptor 8 precursor. |
| | | [Source: Uniprot/SWISSPROT; Acc: Q9NR97] |
| 51348 | NM_016523 | killer cell lectin-like receptor subfamily F, member 1; KLRF1 |
| 51435 | NM_016240; NM_182826 | scavenger receptor class A, member 3; SCARA3 |
| 51473 | NM_016356 | Doublecortin domain-containing protein 2 (RU2S protein). |
| | | [Source: Uniprot/SWISSPROT; Acc: Q9UHG0] |
| 51554 | NM_178445 | C-C chemokine receptor type 11 (C-C CKR-11) (CC-CKR- |
| | | 11) (CCR-11) (Chemokine receptor-like 1) (CCRL1) (CCX |
| | | CKR). [Source: Uniprot/SWISSPROT; Acc: Q9NPB9] |
| 51561 | NM_016584 | interleukin 23, alpha subunit p19 precursor |
| | | [Source: RefSeq_peptide; Acc: NP_057668] |
| 51564 | NM_015401 | Histone deacetylase 7a (HD7a). |
| | | [Source: Uniprot/SWISSPROT; Acc: Q8WUI4] |
| 51665 | NM_016114 | Ankyrin repeat and SOCS box protein 1 (ASB-1). |
| | | [Source: Uniprot/SWISSPROT; Acc: Q9Y576] |
| 51744 | NM_016382 | CD244 natural killer cell receptor 2B4; CD244 |
| 51752 | NM_016442 | Adipocyte-derived leucine aminopeptidase precursor (EC |
| | | 3.4.11.—) (A-LAP) (ARTS-1) (Aminopeptidase PILS) |
| | | (Puromycin-insensitive leucyl-specific aminopeptidase) |
| | | (PILS-AP) (Type 1 tumor necrosis factor receptor shedding |
| | | aminopeptidase regulator). [Source: U |
| 53335 | | B-cell lymphoma/leukemia 11A (B-cell CLL/lymphoma 11A) |
| | | (COUP-TF interacting protein 1) (Ecotropic viral integration |
| | | site 9 protein) (EVI-9). |
| | | [Source: Uniprot/SWISSPROT; Acc: Q9H165] |
| 53342 | NM_138284 | Interleukin-17D precursor (IL-17D) (Interleukin-27) (IL- |
| | | 27). [Source: Uniprot/SWISSPROT; Acc: Q8TAD2] |
| 53347 | NM_018961 | UBASH3A protein. |
| | | [Source: Uniprot/SWISSPROT; Acc: P57075] |
| 54106 | NM_017442 | Toll-like receptor 9 precursor (CD289 antigen). |
| | | [Source: Uniprot/SWISSPROT; Acc: Q9NR96] |
| 54209 | NM_018965 | Triggering receptor expressed on myeloid cells 2 precursor |
| | | (Triggering receptor expressed on monocytes 2) (TREM-2). |
| | | [Source: Uniprot/SWISSPROT; Acc: Q9NZC2] |
| 54210 | NM_018643 | Triggering receptor expressed on myeloid cells 1 precursor |
| | | (TREM-1) (Triggering receptor expressed on monocytes 1). |
| | | [Source: Uniprot/SWISSPROT; Acc: Q9NP99] |
| 54472 | NM_019009 | Toll-interacting protein. |
| | | [Source: Uniprot/SWISSPROT; Acc: Q9H0E2] |
| 54878 | | Dipeptidyl peptidase 8 (EC 3.4.14.5) (Dipeptidyl peptidase |
| | | VIII) (DP8) (Prolyl dipeptidase DPP8) (Dipeptidyl peptidase |
| | | IV-related protein 1) (DPRP-1). |
| | | [Source: Uniprot/SWISSPROT; Acc: Q6V1X1] |
| 54900 | NM_017773 | Lymphocyte transmembrane adapter 1 (Membrane- |
| | | associated adapter protein LAX) (Linker for activation of X |
| | | cells). [Source: Uniprot/SWISSPROT; Acc: Q8IWV1] |
| 54941 | NM_017831 | RING finger protein 125 (EC 6.3.2.—) (T-cell RING activation |
| | | protein 1) (TRAC-1). |
| | | [Source: Uniprot/SWISSPROT; Acc: Q96EQ8] |
| 55075 | NM_018003 | uveal autoantigen with coiled-coil domains and ankyrin |
| | | repeats isoform 1 [Source: RefSeq_peptide; Acc: NP_060473] |
| 55080 | NM_018009 | TAP binding protein-like; TAPBPL |
| 55611 | NM_017670 | Ubiquitin thiolesterase protein OTUB1 (EC 3.4.—.—) (Otubain |
| | | 1) (OTU domain-containing ubiquitin aldehyde-binding |
| | | protein 1) (Ubiquitin-specific processing protease OTUB1) |
| | | (Deubiquitinating enzyme OTUB1). |
| | | [Source: Uniprot/SWISSPROT; Acc: Q96FW1] |
| 55787 | NM_018360 | chromosome X open reading frame 15; CXorf15 |
| 55801 | NM_018402 | Interleukin-26 precursor (AK155 protein). |
| | | [Source: Uniprot/SWISSPROT; Acc: Q9NPH9] |
| 55824 | NM_018440 | Phosphoprotein associated with glycosphingolipid-enriched |
| | | microdomains 1 (Transmembrane adapter protein PAG) |
| | | (Csk-binding protein) (Transmembrane phosphoprotein |
| | | Cbp). [Source: Uniprot/SWISSPROT; Acc: Q9NWQ8] |
| 55894 | NM_018661 | defensin, beta 103A; DEFB103A |
| 56253 | NM_019604 | class-I MHC-restricted T cell associated molecule; CRTAM |
| 56269 | NM_019612 | immunity-related GTPase family, cinema 1 |
| | | [Source: RefSeq_peptide; Acc: NP_062558] |
| 56300 | NM_019618 | Interleukin-1 family member 9 (IL-1F9) (Interleukin-1 |
| | | homolog 1) (IL-1H1) (Interleukin-1 epsilon) (IL-1 epsilon) |
| | | (IL-1-related protein 2) (IL-1RP2). |
| | | [Source: Uniprot/SWISSPROT; Acc: Q9NZH8] |
| 56413 | NM_019839 | Leukotriene B4 receptor 2 (LTB4-R2) (Seven |
| | | transmembrane receptor BLTR2) (Leukotriene B4 receptor |
| | | BLT2) (LTB4 receptor JULF2). |
| | | [Source: Uniprot/SWISSPROT; Acc: Q9NPC1] |
| 56477 | NM_148672 | Small inducible cytokine A28 precursor (CCL28) (Mucosae- |
| | | associated epithelial chemokine) (MEC) (CCK1 protein). |
| | | [Source: Uniprot/SWISSPROT; Acc: Q9NRJ3] |
| 56832 | NM_020124 | Interferon kappa precursor (IFN-kappa). |
| | | [Source: Uniprot/SWISSPROT; Acc: Q9P0W0] |
| 56833 | NM_020125 | SLAM family member 8; SLAMF8 |
| 56924 | NM_020168 | p21(CDKN1A)-activated kinase 6; PAK6 |
| 57105 | NM_020377 | Cysteinyl leukotriene receptor 2 (CysLTR2) (HG57) |
| | | (HPN321) (hGPCR21). |
| | | [Source: Uniprot/SWISSPROT; Acc: Q9NS75] |
| 57115 | NM_020393 | peptidoglycan recognition protein 4; PGLYRP4 |
| 57144 | NM_020341; NM_177990 | p21(CDKN1A)-activated kinase 7; PAK7 |
| 57151 | NM_020426 | lysozyme-like 6; LYZL6 |
| 57292 | NM_020535 | killer cell immunoglobulin-like receptor, two domains, long |
| | | cytoplasmic tail, 5A; KIR2DL5A |
| 57379 | NM_020661 | Activation-induced cytidine deaminase (EC 3.5.4.5) (Cytidine |
| | | aminohydrolase). |
| | | [Source: Uniprot/SWISSPROT; Acc: Q9GZX7] |
| 57381 | NM_020663 | ras homolog gene family, member J; RHOJ |
| 57506 | NM_020746 | Mitochondrial antiviral signaling protein (Interferon-beta |
| | | promoter stimulator protein 1) (IPS-1) (Virus-induced |
| | | signaling adapter) (CARD adapter inducing interferon-beta) |
| | | (Cardif) (Putative NF-kappa-B-activating protein 031N). |
| | | [Source: Uniprot/SWISSPROT; |
| 57580 | NM_020820 | Phosphatidylinositol 3,4,5-trisphosphate-dependent Rac |
| | | exchanger 1 protein (P-Rex1 protein). |
| | | [Source: Uniprot/SWISSPROT; Acc: Q8TCU6] |
| 57817 | NM_021175 | Hepcidin precursor (Liver-expressed antimicrobial peptide) |
| | | (LEAP-1) (Putative liver tumor regressor) (PLTR) [Contains: |
| | | Hepcidin 25 (Hepc25); Hepcidin 20 (Hepc20)]. |
| | | [Source: Uniprot/SWISSPROT; Acc: P81172] |
| 57823 | NM_021181 | SLAM family member 7; SLAMF7 |
| 58191 | NM_022059 | Small inducible cytokine B16 precursor (Transmembrane |
| | | chemokine CXCL16) (SR-PSOX) (Scavenger receptor for |
| | | phosphatidylserine and oxidized low density lipoprotein). |
| | | [Source: Uniprot/SWISSPROT; Acc: Q9H2A7] |
| 59067 | NM_021803 | Interleukin-21 precursor (IL-21) (Za11). |
| | | [Source: Uniprot/SWISSPROT; Acc: Q9HBE4] |
| 59082 | NM_021571 | Caspase-1 inhibitor Iceberg. |
| | | [Source: Uniprot/SWISSPROT; Acc: P57730] |
| 59307 | NM_021805 | Single Ig IL-1-related receptor (Single Ig IL-1R-related |
| | | molecule) (Single immunoglobulin domain-containing IL1R- |
| | | related protein) (Toll/interleukin-1 receptor 8) (TIR8). |
| | | [Source: Uniprot/SWISSPROT; Acc: Q6IA17] |
| 59340 | NM_021624 | Histamine H4 receptor (HH4R) (GPRv53) (G-protein coupled |
| | | receptor 105) (GPCR105) (SP9144) (AXOR35). |
| | | [Source: Uniprot/SWISSPROT; Acc: Q9H3N8] |
| 60489 | | DNA dC->dU editing enzyme APOBEC-3G (EC 3.5.4.—) |
| | | (APOBEC-related cytidine deaminase) (ARCD) (APOBEC- |
| | | related protein) (ARP-9) (CEM15) (CEM-15). |
| | | [Source: Uniprot/SWISSPROT; Acc: Q9HC16] |
| 64112 | NM_022151 | modulator of apoptosis 1; MOAP1 |
| 64135 | NM_022168 | Interferon induced with helicase C domain protein 1 (EC |
| | | 3.6.1.—) (Helicase with 2 CARD domains) (Helicard) |
| | | (Melanoma differentiation-associated protein 5) (MDA-5) |
| | | (RNA helicase-DEAD box protein 116) (Murabutide-down- |
| | | regulated protein). [Source: Uniprot/SW |
| 64167 | NM_022350 | leukocyte-derived arginine aminopeptidase |
| | | [Source: RefSeq_peptide; Acc: NP_071745] |
| 64221 | NM_022370 | roundabout, axon guidance receptor, homolog 3 |
| | | (Drosophila); ROBO3 |
| 64225 | NM_022374 | ADP-ribosylation factor-like 6 interacting protein 2 |
| | | [Source: RefSeq_peptide; Acc: NP_071769] |
| 64332 | NM_001005474 | nuclear factor of kappa light polypeptide gene enhancer in |
| | | B-cells inhibitor, zeta isoform a |
| | | [Source: RefSeq_peptide; Acc: NP_113607] |
| 64342 | NM_022460 | HS1-binding protein 3 |
| | | [Source: RefSeq_peptide; Acc: NP_071905] |
| 64386 | NM_022468 | Matrix metalloproteinase-25 precursor (EC 3.4.24.—) (MMP- |
| | | 25) (Membrane-type matrix metalloproteinase 6) (MT-MMP |
| | | 6) (Membrane-type-6 matrix metalloproteinase) (MT6- |
| | | MMP) (Leukolysin). |
| | | [Source: Uniprot/SWISSPROT; Acc: Q9NPA2] |
| 64421 | NM_001033858 | Artemis protein (EC 3.1.—.—) (DNA cross-link repair 1C |
| | | protein) (SNM1-like protein) (A-SCID protein) (hSNM1C). |
| | | [Source: Uniprot/SWISSPROT; Acc: Q96SD1] |
| 64499 | NM_003294 | Tryptase beta-1 precursor (EC 3.4.21.59) (Tryptase-1) |
| | | (Tryptase I). [Source: Uniprot/SWISSPROT; Acc: Q15661] |
| 64581 | NM_197947; NM_022570; | C-type lectin domain family 7, member A; CLEC7A |
| | NM_197948; NM_197952; | |
| | NM_197950; NM_197949; | |
| | NM_197954; NM_197953; | |
| | NM_197951 | |
| 64806 | NM_172314 | Interleukin-17E precursor (IL-17E) (Interleukin-25) (IL- |
| | | 25). [Source: Uniprot/SWISSPROT; Acc: Q9H293] |
| 65125 | NM_018979 | WNK lysine deficient protein kinase 1; WNK1 |
| 65266 | NM_032387 | WNK lysine deficient protein kinase 4; WNK4 |
| 65267 | NM_001002838; NM_020922 | WNK lysine deficient protein kinase 3; WNK3 |
| 78989 | NM_024027; NM_199235 | collectin sub-family member 11; COLEC11 |
| 79168 | NM_024318 | leukocyte immunoglobulin-like receptor, subfamily A (with |
| | | TM domain), member 6; LILRA6 |
| 79368 | NM_030764; NM_138738 | Fc receptor-like 2; FCRL2 |
| 79400 | NM_024505 | NADPH oxidase, EF hand calcium-binding domain 5 |
| | | [Source: RefSeq_peptide; Acc: NP_078781] |
| 79465 | NM_024518 | UL16 binding protein 3; ULBP3 |
| 79589 | NM_194463 | ring finger protein 128 isoform 1 |
| | | [Source: RefSeq_peptide; Acc: NP_919445] |
| 79679 | NM_024626 | V-set domain containing T cell activation inhibitor 1 |
| | | [Source: RefSeq_peptide; Acc: NP_078902] |
| 79930 | BC004867 | Docking protein 3 |
| 80122 | NM_025052; NM_001018046 | Yeast Sps1/Ste20-related kinase 4 (S. cerevisiae); YSK4 |
| 80274 | NM_173050 | signal peptide-CUB domian-EGF-related 1 |
| | | [Source: RefSeq_peptide; Acc: NP_766638] |
| 80328 | NM_025217 | UL16 binding protein 2; ULBP2 |
| 80329 | NM_025218 | UL16 binding protein 1; ULBP1 |
| 80332 | NM_025220 | ADAM 33 precursor (EC 3.4.24.—) (A disintegrin and |
| | | metalloproteinase domain 33). |
| | | [Source: Uniprot/SWISSPROT; Acc: Q9BZ11] |
| 80341 | NM_025227 | bactericidal/permeability-increasing protein-like 1; BPIL1 |
| 80380 | NM_025239 | Programmed cell death 1 ligand 2 precursor (Programmed |
| | | death ligand 2) (PD-L2) (PD-1-ligand 2) (PDCD1 ligand 2) |
| | | (Butyrophilin B7-DC) (B7-DC) (CD273 antigen). |
| | | [Source: Uniprot/SWISSPROT; Acc: Q9BQ51] |
| 80381 | NM_025240 | CD276 antigen isoform a |
| | | [Source: RefSeq_peptide; Acc: NP_001019907] |
| 80833 | NM_145640 | Apolipoprotein-L3 (Apolipoprotein L-III) (ApoL-III) (TNF- |
| | | inducible protein CG12-1) (CG12_1). |
| | | [Source: Uniprot/SWISSPROT; Acc: O95236] |
| 80741 | NM_001002849 | Lymphocyte antigen 6 complex, locus G5C (LY6G5C) |
| 81035 | NM_030781; NM_130386 | collectin sub-family member 12; COLEC12 |
| 81494 | NM_030787 | complement factor H-related 5; CFHL5 |
| 81542 | NM_030755 | Thioredoxin domain-containing protein 1 precursor |
| | | (Transmembrane Trx-related protein) (Thioredoxin-related |
| | | transmembrane protein). |
| | | [Source: Uniprot/SWISSPROT; Acc: Q9H3N1] |
| 81607 | NM_030916 | poliovirus receptor-related 4; PVRL4 |
| 81793 | NM_030956 | Toll-like receptor 10 precursor. |
| | | [Source: Uniprot/SWISSPROT; Acc: Q9BXR5] |
| 83416 | NM_031281 | Fc receptor-like 5; FCRL5 |
| 83417 | NM_031282 | Fc receptor-like 4; FCRL4 |
| 83903 | NM_031965 | germ cell associated 2 (haspin); GSG2 |
| 83953 | NM_032029 | Fc receptor, IgA, IgM, high affinity; FCAMR |
| 84174 | | Src-like-adapter 2 (Src-like adapter protein 2) (SLAP-2) |
| | | (Modulator of antigen receptor signaling) (MARS). |
| | | [Source: Uniprot/SWISSPROT; Acc: Q9H6Q3] |
| 84569 | NM_032517 | lysozyme-like 1; LYZL1 |
| 84639 | NM_032556 | Interleukin-1 family member 10 (IL-1F10) (Interleukin-1 |
| | | receptor antagonist-like FIL1 theta) (Interleukin-1 theta) |
| | | (IL-1 theta) (FIL1 theta) (Interleukin-1 HY2) (IL-1HY2). |
| | | [Source: Uniprot/SWISSPROT; Acc: Q8WWZ1] |
| 84659 | NM_032572 | Ribonuclease 7 precursor (EC 3.1.27.—) (RNase 7) (Skin- |
| | | derived antimicrobial protein 2) (SAP-2). |
| | | [Source: Uniprot/SWISSPROT; Acc: Q9H1E1] |
| 84663 | NM_032576 | chromosome Y open reading frame 15B; CYorf15B |
| 84824 | NM_032738 | Fc receptor-like and mucin-like 1; FCRLM1 |
| 84868 | NM_032782 | hepatitis A virus cellular receptor 2; HAVCR2 |
| 84941 | NM_032855 | hematopoietic SH2 domain containing |
| | | [Source: RefSeq_peptide; Acc: NP_116244] |
| 84968 | NM_032882 | paraneoplastic antigen like 6A; PNMA6A |
| 89790 | NM_033130 | sialic acid binding Ig-like lectin 10; SIGLEC10 |
| 89858 | NM_033329; NM_053003 | sialic acid binding Ig-like lectin 12; SIGLEC12 |
| 89886 | NM_033438 | SLAM family member 9; SLAMF9 |
| 90925 | NM_175870 | hypothetical protein LOC90925; LOC90925 |
| 91353 | NM_001013618 | similar to omega protein; CTA-246H3.1 |
| 91662 | NM_144687 | NACHT-, LRR- and PYD-containing protein 12 (PYRIN- |
| | | containing APAF1-like protein 7) (Monarch-1) (Regulated |
| | | by nitric oxide). [Source: Uniprot/SWISSPROT; Acc: P59046] |
| 91937 | NM_138379 | T-cell immunoglobulin and mucin domain containing |
| | | 4; TIMD4 |
| 92747 | NM_033197 | chromosome 20 open reading frame 114; C20orf114 |
| 112744 | NM_052872 | Interleukin-17F precursor (IL-17F) (Interleukin-24) (IL-24) |
| | | (Cytokine ML-1). [Source: Uniprot/SWISSPROT; Acc: Q96PD4] |
| 114132 | NM_052884 | sialic acid binding Ig-like lectin 11; SIGLEC11 |
| 114548 | NM_004895 | Cold autoinflammatory syndrome 1 protein (Cryopyrin) |
| | | (NACHT-, LRR- and PYD-containing protein 3) (PYRIN- |
| | | containing APAF1-like protein 1) (Angiotensin/vasopressin |
| | | receptor AII/AVP-like). |
| | | [Source: Uniprot/SWISSPROT; Acc: Q96P20] |
| 114609 | NM_052887 | Toll-interleukin 1 receptor domain-containing adapter |
| | | protein (TIR domain-containing adapter protein) (MyD88 |
| | | adapter-like protein) (Adaptor protein Wyatt). |
| | | [Source: Uniprot/SWISSPROT; Acc: P58753] |
| 114771 | NM_052891 | peptidoglycan recognition protein 3; PGLYRP3 |
| 114824 | NM_052926 | paraneoplastic antigen like 5; PNMA5 |
| 114836 | NM_052931 | SLAM family member 6; SLAMF6 |
| 114897 | NM_198593; NM_198594; | C1q and tumor necrosis factor related protein 1; C1QTNF1 |
| | NM_030968 | |
| 114898 | NM_031908 | C1q and tumor necrosis factor related protein 2; C1QTNF2 |
| 114899 | NM_181435; NM_030945 | C1q and tumor necrosis factor related protein 3; C1QTNF3 |
| 114900 | NM_031909 | C1q and tumor necrosis factor related protein 4; C1QTNF4 |
| 114902 | NM_015645 | C1q and tumor necrosis factor related protein 5; C1QTNF5 |
| 114904 | NM_182486; NM_031910 | C1q and tumor necrosis factor related protein 6; C1QTNF6 |
| 114905 | NM_031911 | C1q and tumor necrosis factor related protein 7; C1QTNF7 |
| 115350 | NM_052938 | Fc receptor-like 1; FCRL1 |
| 115352 | NM_052939; NM_001024667 | Fc receptor-like 3; FCRL3 |
| 115361 | NM_052941 | Guanylate binding protein 4. |
| | | [Source: Uniprot/SWISSPROT; Acc: Q96PP9] |
| 115362 | NM_052942 | Interferon-induced guanylate-binding protein 5 (GTP- |
| | | binding protein 5) (Guanine nucleotide-binding protein 5) |
| | | (GBP-TA antigen). |
| | | [Source: Uniprot/SWISSPROT; Acc: Q96PP8] |
| 115650 | NM_052945 | Tumor necrosis factor receptor superfamily member 13C (B |
| | | cell-activating factor receptor) (BAFF receptor) (BAFF-R) |
| | | (BLyS receptor 3) (CD268 antigen). |
| | | [Source: Uniprot/SWISSPROT; Acc: Q96RJ3] |
| 115653 | NM_153443 | killer cell immunoglobulin-like receptor, three domains, long |
| | | cytoplasmic tail, 3; KIR3DL3 |
| 116379 | NM_052962; NM_181309; | interleukin 22 receptor, alpha 2; IL22RA2 |
| | NM_181310 | |
| 116449 | NM_052964 | mast cell immunoreceptor signal transducer |
| | | [Source: RefSeq_peptide; Acc: NP_443196] |
| 117285 | NM_054112 | Beta-defensin 118 precursor (Defensin, beta 118) (Beta- |
| | | defensin 18) (DEFB-18) (Epididymal secretory protein 13.6) |
| | | (ESP13.6). [Source: Uniprot/SWISSPROT; Acc: Q96PH6] |
| 119180 | NM_183058 | lysozyme-like 2; LYZL2 |
| 124599 | NM_174892 | CD300 antigen like family member B; CD300LB |
| 124912 | NM_173847 | sperm acrosome associated 3; SPACA3 |
| 126014 | NM_133168; NM_206818; | osteoclast-associated receptor; OSCAR |
| | NM_133169; NM_206817; | |
| | NM_130771 | |
| 127150 | XM_497717 | similar to mucosal pentraxin; LOC127150 |
| 127943 | NM_001002901 | Fc receptor-like and mucin-like 2; FCRLM2 |
| 128859 | NM_174897 | bactericidal/permeability-increasing protein-like 3; BPIL3 |
| 130120 | NM_198448 | Regenerating islet-derived protein 3 gamma precursor (Reg |
| | | III-gamma) (Pancreatitis-associated protein 1B) (PAP IB). |
| | | [Source: Uniprot/SWISSPROT; Acc: Q6UW15] |
| 131375 | NM_144634 | lysozyme-like 4; LYZL4 |
| 133396 | NM_139017 | gp130-like monocyte receptor |
| | | [Source: RefSeq_peptide; Acc: NP_620586] |
| 135250 | NM_139165 | retinoic acid early transcript 1E; RAET1E |
| 140850 | NM_139074 | Beta-defensin 127 precursor (Defensin, beta 127) (Beta- |
| | | defensin 27) (DEFB-27). |
| | | [Source: Uniprot/SWISSPROT; Acc: Q9H1M4] |
| 146722 | NM_139018 | CD300 antigen like family member F; CD300LF |
| 146894 | NM_145273 | CD300 antigen like family member G; CD300LG |
| 149954 | NM_182519 | chromosome 20 open reading frame 186; C20orf186 |
| 150372 | NM_145912 | NFAT activation molecule 1 precursor (Calcineurin/NFAT- |
| | | activating ITAM-containing protein) (NFAT-activating |
| | | protein with ITAM motif 1). |
| | | [Source: Uniprot/SWISSPROT; Acc: Q8NET5] |
| 151888 | NM_181780 | B and T lymphocyte attenuator precursor (B and T |
| | | lymphocyte-associated protein) (CD272 antigen). |
| | | [Source: Uniprot/SWISSPROT; Acc: Q7Z6A9] |
| 152028 | NM_144717 | fibronectin type III domain containing 6; FNDC6 |
| 154064 | NM_130900 | retinoic acid early transcript 1L; RAET1L |
| 163351 | NM_198460 | guanylate binding protein family, member 6 |
| | | [Source: RefSeq_peptide; Acc: NP_940862] |
| 163702 | NM_173065; NM_173064; | interleukin 28 receptor, alpha (interferon, lambda |
| | NM_170743 | receptor); IL28RA |
| 165257 | NM_182528 | complement component 1, q subcomponent-like 2; C1QL2 |
| 167838 | NM_153235 | taxilin beta; TXLNB |
| 170482 | NM_130441 | C-type lectin domain family 4 member C (C-type lectin |
| | | superfamily member 7) (Blood dendritic cell antigen 2 |
| | | protein) (BDCA-2) (Dendritic lectin) (CD303 antigen). |
| | | [Source: Uniprot/SWISSPROT; Acc: Q8WTT0] |
| 200081 | NM_175852 | taxilin alpha; TXLNA |
| 201633 | NM_173799 | hypothetical protein FLJ39873; FLJ39873 |
| 246126 | NM_001005852 | chromosome Y open reading frame 15A; CYorf15A |
| 246778 | NM_145659 | interleukin 27 [Source: RefSeq_peptide; Acc: NP_663634] |
| 254240 | NM_174932 | bactericidal/permeability-increasing protein-like 2; BPIL2 |
| 260434 | NM_152901 | pyrin domain containing 1 |
| | | [Source: RefSeq_peptide; Acc: NP_690865] |
| 284021 | XM_211305 | chromosome 17 open reading frame 60; C17orf60 |
| 284367 | XM_290822 | sialic acid binding Ig-like lectin, pseudogene 3; SIGLECP3 |
| 284415 | NM_198481 | LAIR hlog; UNQ3033 |
| 284988 | XM_209429 | similar to ARHQ protein; LOC284988 |
| 285830 | NM_001003807 | hypothetical protein FLJ35429; RP3-377H14.5 |
| 286204 | NM_173689 | crumbs homolog 2 (Drosophila); CRB2 |
| 286310 | NM_002297 | Lipocalin-1 precursor (Von Ebner gland protein) (VEG |
| | | protein) (Tear prealbumin) (TP) (Tear lipocalin) (Tlc). |
| | | [Source: Uniprot/SWISSPROT; Acc: P31025] |
| 338339 | NM_080387 | C-type lectin domain family 4 member D (C-type lectin |
| | | superfamily member 8) (C-type lectin-like receptor 6) |
| | | (CLEC-6). [Source: Uniprot/SWISSPROT; Acc: Q8WXI8] |
| 338761 | NM_001008223 | complement component 1, q subcomponent-like 4; C1QL4 |
| 338902 | XM_497405 | similar to Immunoglobulin-binding protein 1 (CD79a- |
| | | binding protein 1) (B cell signal transduction molecule alpha |
| | | 4) (Alpha 4 protein); LOC338902 |
| 339377 | XM_290866 | similar to 4930572L20Rik protein; LOC339377 |
| 339390 | NM_198492 | C-type lectin superfamily 4, member G; CLEC4G |
| 339562 | XM_291643 | similar to Ig kappa chain; LOC339562 |
| 342510 | NM_181449 | CD300 antigen like family member E; CD300LE |
| 343413 | NM_001004310 | Fc receptor-like 6; FCRL6 |
| 346689 | NM_198508 | FLJ44186 protein; FLJ44186 |
| 353091 | NM_001001788 | retinoic acid early transcript 1G; RAET1G |
| 353219 | NM_181337 | kidney associated antigen 1 |
| 353376 | NM_021649 | Transmembrane emp24 domain containing protein 7 |
| | | precursor. [Source: Uniprot/SWISSPROT; Acc: Q9Y3B3] |
| 353514 | NM_021250; NM_181879; | leukocyte immunoglobulin-like receptor, subfamily A (with |
| | NM_181985; NM_181986 | TM domain), member 5; LILRA5 |
| 359710 | NM_182658 | chromosome 20 open reading frame 185; C20orf185 |
| 378829 | AF304442 | B lymphocyte activation-related protein BC-1514 |
| 387836 | NM_207375 | C-type lectin domain family 2, member A; CLEC2A |
| 388077 | XM_370834 | immunoglobulin heavy variable 1/OR15-1; IGHV1OR15-1 |
| 388078 | XM_370835 | V-set and immunoglobulin domain containing 6; VSIG6 |
| 388372 | NM_001001435 | chemokine (C-C motif) ligand 4-like 2 precursor |
| | | [Source: RefSeq_peptide; Acc: NP_996890] |
| 388503 | NM_001013640 | similar to Complement C3 precursor; LOC388503 |
| 388646 | NM_207398 | Interferon-induced guanylate-binding protein 2 (GTP- |
| | | binding protein 2) (Guanine nucleotide-binding protein 2) |
| | | (HuGBP-2). [Source: Uniprot/SWISSPROT; Acc: P32456] |
| 388677 | NM_203458 | Notch homolog 2 (Drosophila) N-terminal like; NOTCH2NL |
| 389405 | XM_371829 | similar to Neurogenic locus Notch protein precursor; RP1- |
| | | 303F19.1 |
| 389852 | NM_205856 | PNPK6288; RP11-38O23.2 |
| 389941 | NM_001010908 | complement component 1, q subcomponent-like 3; C1QL3 |
| 389950 | XM_372307 | similar to immunoglobulin lambda-1 variable |
| | | region; LOC389950 |
| 390530 | XM_372543 | similar to immunoglobulin heavy-chain-2 light-chain-2 VH |
| | | segment; LOC390530 |
| 390531 | XM_496025 | V-set and immunoglobulin domain containing 7; VSIG7 |
| 390667 | NM_001013658 | similar to Neuronal pentraxin II precursor (NP-II) |
| | | (NP2); LOC390667 |
| 390712 | XM_372630 | similar to immunoglobulin M chain; LOC390712 |
| 390714 | XM_372632 | similar to Ig heavy chain V-III region VH26 |
| | | precursor; LOC390714 |
| 391105 | XM_497715 | similar to dJ801G22.2 (novel protein similar to |
| | | immunoglobulin gamma FC receptor 1 (FGCR1)); LOC391105 |
| 391142 | XM_496426 | hypothetical gene supported by AJ249778; |
| | | NM_001531; LOC391142 |
| 391405 | XM_372941 | similar to Ig kappa chain V region (Z4) - human; LOC391405 |
| 391427 | XM_372952 | similar to Ig kappa chain precursor V region (orphon V108) - |
| | | human (fragment); LOC391427 |
| 392217 | XM_373249 | similar to Ig lambda light chain leader and V- |
| | | region; LOC392217 |
| 392965 | XM_374637 | similar to alpha-2-glycoprotein 1, zinc; Alpha-2- |
| | | glycoprotein, zinc; LOC392965 |
| 400581 | XM_375418 | GRB2-related adaptor protein-like; LOC400581 |
| 400668 | NM_214710 | protease, serine-like 1; PRSSL1 |
| 400709 | XM_375634 | sialic acid binding Ig-like lectin, pseudogene 16; SIGLECP16 |
| 400759 | NM_002053 | Interferon-induced guanylate-binding protein 1 (GTP- |
| | | binding protein 1) (Guanine nucleotide-binding protein 1) |
| | | (HuGBP-1). [Source: Uniprot/SWISSPROT; Acc: P32455] |
| 400760 | NM_002053 | Interferon-induced guanylate-binding protein 1 (GTP- |
| | | binding protein 1) (Guanine nucleotide-binding protein 1) |
| | | (HuGBP-1). [Source: Uniprot/SWISSPROT; Acc: P32455] |
| 400792 | XM_375816 | similar to Slamf7 protein; LOC400792 |
| 400943 | NM_207480 | AILT5830 |
| 401262 | NM_206922 | cysteine-rich protein 3 |
| | | [Source: RefSeq_peptide; Acc: NP_996805] |
| 401393 | XM_376658 | similar to Zinc-alpha-2-glycoprotein precursor (Zn-alpha- |
| | | 2-glycoprotein) (Zn-alpha-2-GP); LOC401393 |
| 401845 | XM_377426 | similar to IGHV gene product; LOC401845 |
| 401887 | XM_497555 | similar to calmodulin - rabbit (tentative |
| | | sequence); LOC401887 |
| 402571 | XM_379897 | similar to Zinc-alpha-2-glycoprotein precursor (Zn-alpha- |
| | | 2-glycoprotein) (Zn-alpha-2-GP); LOC402571 |
| 404552 | NM_206998 | Secretoglobin family 1D member 4 precursor (IFN-gamma- |
| | | inducible secretoglobin) (IIS). |
| | | [Source: Uniprot/SWISSPROT; Acc: Q6XE38] |
| 407977 | NM_172089 | Tumor necrosis factor ligand superfamily member 13 |
| | | precursor (A proliferation-inducing ligand) (APRIL) (TNF- |
| | | and APOL-related leukocyte expressed ligand 2) (TALL-2) |
| | | (TNF-related death ligand-1) (TRDL-1) (CD256 antigen). |
| | | [Source: Uniprot/SWISSPROT; Acc: O75888 |
| 432395 | NM_001002029 | complement component 4B, telomeric; XXbac-BPG116M5.7 |
| 439957 | XM_495805 | similar to Ig kappa chain V region (Z4) - human; LOC439957 |
| 440361 | XM_496145 | similar to immunoglobulin M chain; LOC440361 |
| 440370 | XM_496158 | similar to immunoglobulin M chain; LOC440370 |
| 440508 | XM_496285 | similar to liver and lymph node sinusoidal endothelial cell C- |
| | | type lectin; DTTR431; LOC440508 |
| 440607 | NM_001017986; NM_001004340 | Fc-gamma receptor I B2; LOC440607 |
| 440786 | XM_496488 | similar to Ig kappa chain; LOC440786 |
| 440871 | XM_496558 | similar to Ig kappa variable region; LOC440871 |
| 440891 | XM_496578 | similar to Ig kappa light chain variable region; LOC440891 |
| 441140 | NM_001004349 | FLJ45422 protein; FLJ45422 |

1. Method to detect the presence or risk of developing a cancer in a mammal, comprising the detection, in a biological sample of the mammal:
a) of nucleic acids containing the sequences indicated in SEQ ID No: 23, 52, 53, 148 and 225 (PANEL 11), or a distinctive fragment thereof having at least 15, preferably at least 16, 17, 18, 19, 20, 25 or 30 consecutive bases, and/or
b) of nucleic acids having a sequence complementary to sequences according to a), and/or
c) of functional analogs of the nucleic acids according to a) or b) derived from another species, and/or
d) of polypeptides encoded by the nucleic acids according to a) to c),
the presence, absence or (relative) quantity of these target molecules in the sample being an indication of the presence or risk of developing a cancer in said mammal.

2. Method according to claim 1, characterized in that the nucleic acids according to a) are chosen from among the nucleic acids containing the sequences given in one of panels 1-10 in Table 4, or a distinctive fragment thereof having at least 15, preferably at least 16, 17, 18, 19, 20, 25 or 30 consecutive bases.

3. Method according to claim 1 or 2, characterized in that the nucleic acids according to a) comprise the nucleic acids containing the sequences given in panel 10 of Table 4, or a distinctive fragment thereof having at least 15, preferably at least 16, 17, 18, 19, 20, 25 or 30 consecutive bases.

4. Method for in vitro or ex vivo detection of the presence or risk of developing a cancer in a mammal, comprising the determination of the presence (or absence) in a biological sample of the mammal, preferably a blood (derived) sample), of an alteration in a gene or RNA involved in the stimulation of TLRs, in the secretion of cytokins, or in the activation of T lymphocytes, said gene or RNA advantageously being chosen from among receptors, adapters, enzymes, factors involved in the regulation of gene expression, chemokins, cytokins and interleukins, the presence of said alteration being indicative of the presence or risk of developing a cancer in this mammal.

5. Method according to claim 4, comprising the determination of the presence (or absence) in a biological sample of the mammal, preferably a blood (derived) sample, of an alteration in at least one, preferably at least 2, 3, 4, 5, 6, 7, 8, 9 or 10 genes or corresponding RNAs indicated in Table 5, in particular altered splicing of said gene or RNA, the presence of said alteration being an indication of the presence or risk of developing a cancer in this mammal.

6. Method according to claim 4, comprising the determination of the presence (or absence) in a biological sample of the mammal, preferably in a blood (derived) sample, of an alteration in at least one, preferably at least 2, 3, 4, 5, 6, 7, 8, 9 or 10 genes or corresponding RNAs indicated in Table 6, in particular altered splicing of said gene or RNA, the presence of said alteration being an indication of the presence or risk of developing a cancer in this mammal.

7. Method according to any of the preceding claims, comprising the detection of the presence or absence of a nucleic acid by selective hybridization or selective amplification.

8. Method according to any of the preceding claims to detect the presence or risk of developing a cancer in a mammal, comprising the contacting, under conditions allowing hybridization between complementary sequences, of the nucleic acids derived from a blood sample of the mammal with a set of probes specific to a group of target molecules chosen from among:
a) the nucleic acids of one of panels 1 to 11 defined in claims 1 and 2, or a distinctive fragment thereof having at least 15, preferably at least 16, 17, 18, 19, 20, 25 or 30 consecutive bases, and/or
b) the nucleic acids having a sequence complementary to sequences according to a), and/or
c) functional analogs of the nucleic acids according to a) or b) derived from another species,
to obtain a hybridization profile, the hybridization profile being chaacteristic of the presence or risk of developing a cancer in this mammal.

9. Method according to claim 8, comprising the contacting, under conditions allowing hybridization between complementary sequences, of the nucleic acids derived from a blood sample of the mammal with a set of probes specific to the following target molecules:
a) the nucleic acids of panel 11 or a distinctive fragment thereof having at least 15, preferably at least 16, 17, 18, 19, 20, 25 or 30 consecutive bases, and/or
b) the nucleic acids having a sequence complementary to sequences according to a), and/or
c) functional analogs of the nucleic acids according to a) or b) derived from another species,
to obtain a hybridization profile, the hybridization profile being characteristic of the presence or risk of developing a cancer in this mammal.

10. Method according to claim 5, comprising the contacting, under conditions allowing hybridization between complementary sequences, of the nucleic acids derived from a blood sample of the mammal with a set of probes specific to at least two genes or corresponding RNAs indicated in Tables 5 and 6.

11. Method according to any of claims 8 to 10, characterized in that the probes are immobilized on a carrier.

12. Method according to any of claims 1 to 8 to detect the presence or risk of developing a cancer in a mammal, comprising the contacting, under conditions allowing an amplification reaction, of the nucleic acids derived from a blood sample of the mammal with a set of primers specific to a group of target molecules chosen from among:
a) the nucleic acids of one of panels 1 to 11 defined in claims 1 and 2, or a distinctive fragment thereof having at least 15, preferably at least 16, 17, 18, 19, 20, 25 or 30 consecutive bases, and/or
b) the nucleic acids having a sequence complementary to sequences according to a), and/or
c) functional analogs of the nucleic acids according to a) or b) derived from another species,
to obtain an amplification profile, the amplification profile being characteristic of the presence or risk of developing a cancer in this mammal.

13. Method according to claim 6 or 7, comprising the contacting, under conditions allowing an amplification reaction, of the nucleic acids derived from a blood sample of the mammal with a set of primers specific to at least two genes or corresponding RNAs indicated in Tables 5 and 6.

14. Method according to any of claims 1 to 7, comprising the detection of the presence or absence of a polypeptide encoded by said genes or RNAs by means of a specific antibody or a fragment or derivative thereof.

15. Method according to any of the preceding claims, characterized in that the sample is a blood derived sample, preferably a sample of whole blood.

16. Method according to any of the preceding claims, to detect the presence of an early stage breast cancer of stage I or II.

17. Method according to any of claims 1 to 15, to detect the presence of an early stage breast cancer non-detectable by mammography.

18. Use of a nucleic probe specific to a target nucleic acid such as defined in any of claims 1 to 8, said probe comprising 15 to 400 bases, for the in vitro detection of a cancer in a human individual.

19. Use of a nucleic primer enabling the amplification of all or part of a target nucleic acid such as defined in any of claims 1 to 7, said primer being single-strand having a length of between 5 and 50 bases, for the in vitro detection of a cancer in a human individual.

20. Product comprising a carrier on which at least two separate nucleic acid probes are immobilized, comprising a sequence complementary to and/or specific to at least two separate target nucleic acids containing at least two sequences chosen from among SEQ ID NO: 1-437, or to at least one gene or RNA indicated in Table 5 or 6.

21. Product according to claim 20, comprising a carrier on which at least one set of separate nucleic acid probes is immobilized, containing a sequence complementary to and/or specific to the nucleic acids of one of panels 1 to 11 defined in claim 1.

22. Use of a product according to claim 20 for the in vitro or ex vivo detection of the presence or risk of developing a cancer in a mammal.

23. Use of a product according to claim 21 for the in vitro or ex vivo detection of the presence or risk of developing a breast cancer in a mammal.
